(19) United States
(12) Patent Application Publication
(10) Pub. No.: US 2024/0173034 A1
(43) Pub. Date: May 30, 2024

(54) CLIP AND METHOD FOR CLOSING LEFT ATRIAL APPENDAGE

(71) Applicant: EDWARDS LIFESCIENCES CORPORATION, Irvine, CA (US)

(72) Inventors: Harvey H. Chen, Irvine, CA (US); Manouchehr A. Miraki, Laguna Hills, CA (US); Rodolfo Rodriguez, San Luis Obispo, CA (US); Erin E. Castioni, Costa Mesa, CA (US); Maria L. Saravia, Irvine, CA (US); Stephen Epstein, Millersville, MD (US); Luke Anthony Zanetti, Parkton, MD (US); Ashley Nicolette Hinga (formerly Keffer), Eldersburg, MD (US); Stephen Cournane, Severn, MD (US); Felino V. Cortez, JR., Bowie, MD (US); Nancy Ling Chung, Edgewater, MD (US); Daniel Yasevac, Hull, MA (US); Andrew Ryan, Boston, MA (US); Slava Arabagi, San Diego, CA (US); Jaime L. Baluyot, Westminster, CA (US); Sooji Van Echten, Cerritos, CA (US); Da-Yu Chang, Irvine, CA (US); John Richard Carpenter, Santa Ana, CA (US); Sai Prasad Uppalapati, Plano, TX (US); Pui Tong Ho, Ladera Ranch, CA (US); Jason Thai Le, Garden Grove, CA (US); Adam J. Yestrepsky, Draper, UT (US)

(21) Appl. No.: 18/431,809

(22) Filed: Feb. 2, 2024

Related U.S. Application Data
(63) Continuation of application No. PCT/US2022/039491, filed on Aug. 4, 2022.
(60) Provisional application No. 63/366,598, filed on Jun. 17, 2022.
(60) Provisional application No. 63/366,519, filed on Jun. 16, 2022.
(60) Provisional application No. 63/365,693, filed on Jun. 1, 2022.
(60) Provisional application No. 63/365,468, filed on May 27, 2022.
(60) Provisional application No. 63/267,280, filed on Jan. 28, 2022.
(60) Provisional application No. 63/239,878, filed on Sep. 1, 2021.
(60) Provisional application No. 63/234,072, filed on Aug. 17, 2021.
(60) Provisional application No. 63/233,169, filed on Aug. 13, 2021.
(60) Provisional application No. 63/232,054, filed on Aug. 11, 2021.
(60) Provisional application No. 63/230,330, filed on Aug. 6, 2021.
(60) Provisional application No. 63/229,967, filed on Aug. 5, 2021.
(60) Provisional application No. 63/229,148, filed on Aug. 4, 2021.
(60) Provisional application No. 63/231,129, filed on Aug. 9, 2021.

(51) Int. Cl. A61B 17/122; A61B 17/00; A61B 17/128

(52) U.S. Cl. CPC A61B 17/1227; A61B 17/00234; A61B 17/1285; A61B 2017/00243

(57) ABSTRACT

Systems, apparatuses, and methods disclosed herein may be directed to clips for medical implementation, including clips for a portion of a heart. The clips may be configured to close the portion of the heart, to reduce blood flow therethrough as well as passage of clots or other undesired materials. In examples, the clips may be configured to close the left atrial appendage (LAA). The closure of the LAA may reduce the possibility of stroke or other maladies stemming from fluid flow with the LAA. In examples, the clips may be positioned exterior of the LAA, to extend over an outer surface of the LAA for closure.

### CROSS-REFERENCE TO RELATED APPLICATIONS

This application is a continuation of International Patent Application No. PCT/US2022/039491, filed Aug. 4, 2022, which claims the benefit of U.S. Patent Application No. 63/229,148, filed Aug. 4, 2021; U.S. Patent Application No. 63/229,967, filed Aug. 5, 2021; U.S. Patent Application No. 63/230,330, filed Aug. 6, 2021; U.S. Patent Application No. 63/231,129, filed Aug. 9, 2021; U.S. Patent Application No. 63/232,054, filed Aug. 11, 2021; U.S. Patent Application No. 63/233,169, filed Aug. 13, 2021; U.S. Patent Application No. 63/234,072, filed Aug. 17, 2021; U.S. Patent Application No. 63/239,878, filed Sep. 1, 2021; U.S. Patent Application No. 63/267,280, filed Jan. 28, 2022; U.S. Patent Application No. 63/365,468, filed May 27, 2022; U.S. Provisional Application No. 63/365,693, filed Jun. 1, 2022; U.S. Patent Application No. 63/366,519, filed Jun. 16, 2022; and U.S. Patent Application No. 63/366,598, filed Jun. 17, 2022, the entire disclosures all of which are incorporated by reference for all purposes.

### BACKGROUND

### Field

Various examples disclosed herein relate generally to clips or other methods for medical implementation. Some examples relate to clips for a left atrial appendage (LAA).

### Background

Cardiac arrhythmias are abnormal heart rhythms that can cause the heart to pump blood less effectively. Atrial fibrillation (AF) is one of the most common heart arrhythmia conditions. AF causes the left atrium to beat irregularly and reduces the efficiency of the “atrial kick” that helps to move blood into the left ventricle.

The left atrial appendage (LAA) is a muscular pouch located high on the free wall of the left atrium. The anatomy of the LAA is such that blood has a tendency to stagnate and form clots within the LAA. As blood flow is reduced with the progression of AF, the potential for clot formation increases tremendously.

Clots formed in the LAA can embolize into the bloodstream and move into the brain, where they can become lodged and eventually lead to stroke. It may be beneficial to close or occlude the LAA, to reduce the possibility of clots or other undesired materials from passing into the left atrium and into the bloodstream.

Left atrial appendage closure (also known as LAA closure or LAAC) is a minimally invasive procedure that is used to reduce the risk of stroke that comes as a result of atrial fibrillation.

### SUMMARY

Systems, apparatuses, and methods disclosed herein may be directed to clips for medical implementation, including clips for a portion of a heart. The clips may be configured to close the portion of the heart, to reduce blood flow therethrough as well as passage of clots or other undesired materials. In examples, the clips may be configured to close the left atrial appendage (LAA). The closure of the LAA may reduce the possibility of stroke or other maladies stemming from fluid flow with the LAA. In examples, the clips may be positioned exterior of the LAA, to extend over an outer surface of the LAA for closure.

Systems, apparatuses, and methods disclosed herein may be directed to medical implementation, including treating a portion of a heart. The portion of the heart to be treated may comprise the left atrial appendage (LAA).

Systems, apparatuses, and methods disclosed herein may be directed to forming a fold of at least a portion of a LAA. A first portion of a LAA may be folded with a second portion of a heart. The first portion may be bonded to the second portion to maintain the fold.

The portion of the LAA may be folded to reduce a volume of an inner chamber or cavity of the LAA. The reduced volume of the inner chamber of the LAA may reduce the possibility of stagnated blood flow within the inner chamber, which may reduce the possibility of clots or other undesired material from forming and releasing from the LAA.

The clips may be configured to exclude the full base of the LAA in patients that are undergoing a concomitant surgical cardiac procedure that may be at a high risk of stroke to eliminate the risk of LAA-originating embolic stroke. In examples, the clips may be utilized in other manners as desired.

In aspects, a clip for a portion of a heart, the clip comprising: a first jaw including a first arm and a second arm separated from the first arm with a gap.

The first arm and the second arm may each be configured to apply a compressive force to the portion of the heart.

A second jaw may be positioned offset from the first arm and the second arm and aligned with the gap and configured to apply a compressive force to the portion of the heart in a direction towards the gap.

In aspects, a method comprising: deploying a clip to close a portion of a heart.

The clip may include a first jaw including a first arm and a second arm separated from the first arm with a gap, the first arm and the second arm each configured to apply a compressive force to the portion of the heart.

A second jaw may be positioned offset from the first arm and the second arm and aligned with the gap and configured to apply a compressive force to the portion of the heart in a direction towards the gap.

In aspects, a clip for a portion of a heart, the clip comprising: a first jaw extending from a first end portion to a second end portion and including a central portion between the first end portion and the second end portion.

A second jaw may extend from a first end portion to a second end portion and including a central portion between the first end portion and the second end portion.

A spring may be coupled to the central portion of the first jaw and the central portion of the second jaw and configured to force the first jaw and the second jaw together to compress the portion of the heart between the first jaw and the second jaw.

In aspects, a method comprising: deploying a clip to close a portion of a heart.

The clip may include a first jaw extending from a first end portion to a second end portion and including a central portion between the first end portion and the second end portion.

A second jaw may extend from a first end portion to a second end portion and including a central portion between the first end portion and the second end portion.

A spring may be coupled to the central portion of the first jaw and the central portion of the second jaw and configured to force the first jaw and the second jaw together to compress the portion of the heart between the first jaw and the second jaw.

In aspects, a clip for a portion of a heart, the clip comprising: a first jaw extending from a first end portion to a second end portion and including a first cantilever beam extending from the second end portion towards the first end portion.

A second jaw may extend from a first end portion to a second end portion and including a second cantilever beam extending from the second end portion towards the first end portion, the first end portion of the second jaw being pivotally coupled to the first end portion of the first jaw such that a distance between the first cantilever beam and the second cantilever beam may be reduced to compress the portion of the heart between the first cantilever beam and the second cantilever beam.

In aspects, a method comprising: deploying a clip to close a portion of a heart.

The clip may include a first jaw extending from a first end portion to a second end portion and including a first cantilever beam extending from the second end portion towards the first end portion.

A second jaw may extend from a first end portion to a second end portion and including a second cantilever beam extending from the second end portion towards the first end portion, the first end portion of the second jaw being pivotally coupled to the first end portion of the first jaw such that a distance between the first cantilever beam and the second cantilever beam may be reduced to compress the portion of the heart between the first cantilever beam and the second cantilever beam.

In aspects, a clip for a portion of a heart, the clip comprising: a first jaw; a second jaw; and a four-link mechanism configured to move the first jaw relative to the second jaw to compress the portion of the heart between the first jaw and the second jaw.

In aspects, a method comprising: deploying a clip to close a portion of a heart, the clip including: a first jaw, a second jaw, and a four-link mechanism configured to move the first jaw relative to the second jaw to compress the portion of the heart between the first jaw and the second jaw.

In aspects, a clip for a portion of a heart, the clip comprising: a first jaw extending from a first end portion to a second end portion and including a central portion between the first end portion and the second end portion.

A second jaw may extend from a first end portion to a second end portion and including a central portion between the first end portion and the second end portion.

A spring may be coupled to the central portion of the first jaw and the central portion of the second jaw and configured to force the first jaw and the second jaw together to compress the portion of the heart between the first jaw and the second jaw.

In aspects, a method comprising: deploying a clip to close a portion of a heart, the clip including: a first jaw extending from a first end portion to a second end portion and including a central portion between the first end portion and the second end portion.

A second jaw may extend from a first end portion to a second end portion and including a central portion between the first end portion and the second end portion.

A spring may be coupled to the central portion of the first jaw and the central portion of the second jaw and configured to force the first jaw and the second jaw together to compress the portion of the heart between the first jaw and the second jaw.

In aspects, a clip for a portion of a heart, the clip comprising: a first jaw extending from a first end portion to a second end portion.

A second jaw may extend from a first end portion to a second end portion and extending along the first jaw to form a channel for receiving the portion of the heart.

A first spring may couple the first jaw to the second jaw and covering a first end of the channel at the first end portion of the first jaw and the first end portion of the second jaw, the first spring configured to force the first jaw towards the second jaw to compress the portion of the heart within the channel.

A second spring coupling the first jaw to the second jaw and covering a second end of the channel at the second end portion of the first jaw and the second end portion of the second jaw, the second spring configured to force the first jaw towards the second jaw to compress the portion of the heart within the channel.

In aspects, a method comprising: deploying a clip to close a portion of a heart.

The clip may include a first jaw extending from a first end portion to a second end portion.

A second jaw may extend from a first end portion to a second end portion and extending along the first jaw to form a channel for receiving the portion of the heart,

A first spring may couple the first jaw to the second jaw and covering a first end of the channel at the first end portion of the first jaw and the first end portion of the second jaw, the first spring configured to force the first jaw towards the second jaw to compress the portion of the heart within the channel.

A second spring may couple the first jaw to the second jaw and covering a second end of the channel at the second end portion of the first jaw and the second end portion of the second jaw, the second spring configured to force the first jaw towards the second jaw to compress the portion of the heart within the channel.

In aspects, a clip for a portion of a heart, the clip comprising: a first jaw extending from a first end portion to a second end portion.

A second jaw may extend from a first end portion to a second end portion and extending along the first jaw to form a channel for receiving the portion of the heart, and the second end portion of the second jaw being separable from the second end portion of the first jaw to form an axial opening for the channel.

A spring may be positioned at the first end portion of the first jaw and the first end portion of the second jaw and configured to force the first jaw and the second jaw together to compress the portion of the heart.

In aspects, a method comprising: deploying a clip to close a portion of a heart.

The clip may include a first jaw extending from a first end portion to a second end portion.

The clip may include a second jaw extending from a first end portion to a second end portion and extending along the first jaw to form a channel for receiving the portion of the heart, and the second end portion of the second jaw being separable from the second end portion of the first jaw to form an axial opening for the channel.

The clip may include a spring positioned at the first end portion of the first jaw and the first end portion of the second jaw and configured to force the first jaw and the second jaw together to compress the portion of the heart.

In aspects, a clip for a portion of a heart, the clip comprising: a first jaw extending from a first end portion to a second end portion.

A second jaw may extend from a first end portion to a second end portion.

A spring may couple the first jaw to the second jaw and configured to force the first jaw towards the second jaw to compress the portion of the heart between the first jaw and the second jaw, the spring being configured to slide relative to the second jaw to allow movement of the first jaw relative to the second jaw.

In aspects, a method comprising: deploying a clip to close a portion of a heart.

The clip may include a first jaw extending from a first end portion to a second end portion.

The clip may include a second jaw extending from a first end portion to a second end portion.

A spring may couple the first jaw to the second jaw and configured to force the first jaw towards the second jaw to compress the portion of the heart between the first jaw and the second jaw, the spring being configured to slide relative to the second jaw to allow movement of the first jaw relative to the second jaw.

In aspects, a clip for a portion of a heart, the clip comprising: a first jaw; a second jaw; and a slotted pin joint coupled to the first jaw and the second jaw and configured to allow the first jaw to move relative to the second jaw to compress the portion of the heart between the first jaw and the second jaw.

In aspects, a method comprising deploying a clip to close a portion of a heart, the clip including: a first jaw, a second jaw, and a slotted pin joint coupled to the first jaw and the second jaw and configured to allow the first jaw to move relative to the second jaw to compress the portion of the heart between the first jaw and the second jaw.

In aspects, a clip system for a left atrial appendage of a heart.

The clip system may comprise a first clip configured to be deployed proximate an ostium of the left atrial appendage to occlude a portion of the left atrial appendage.

The clip system may comprise a second clip configured to be deployed between the first clip and a wall of a left atrium of the heart to occlude a portion of the heart between the first clip and the wall of the left atrium; and a delivery apparatus configured to deploy one or more of the first clip or the second clip to the respective portions.

In aspects, a method comprising deploying a first clip proximate an ostium of a left atrial appendage to occlude a portion of the left atrial appendage; and deploying a second clip between the first clip and a wall of a left atrium of a heart to occlude a portion of the heart between the first clip and the wall of the left atrium.

In aspects, a clip for a portion of a heart, the clip comprising an elongate upper jaw extending along a first axis and having a first outer surface, the elongate upper jaw configured for the first outer surface to rotate about the first axis in a first rotational direction.

The clip comprising an elongate lower jaw extending along a second axis and having a second outer surface, the elongate lower jaw configured for the second outer surface to rotate about the second axis in a second rotational direction that is opposite the first rotational direction to draw a portion of the heart between the first outer surface and the second outer surface.

In aspects, a method comprising: deploying a clip to close a portion of a heart.

The clip may include: an elongate upper jaw extending along a first axis and having a first outer surface, the elongate upper jaw configured for the first outer surface to rotate about the first axis in a first rotational direction.

The clip may include an elongate lower jaw extending along a second axis and having a second outer surface, the elongate lower jaw configured for the second outer surface to rotate about the second axis in a second rotational direction that is opposite the first rotational direction to draw a portion of the heart between the first outer surface and the second outer surface.

In aspects, a clip for a portion of a heart, the clip comprising: a retainer body including: an upper receiver configured to be positioned on a first side of the portion of the heart and including a first pair of opposed tracks, a lower receiver configured to be positioned on a second side of the portion of the heart that is opposite the first side and including a second pair of opposed tracks, and a loop body coupling the upper receiver to the lower receiver.

The clip comprising an upper jaw configured to apply a compressive force to the first side of the portion of the heart, at least a portion of the upper jaw configured to be slidably engaged with the first pair of opposed tracks.

The clip comprising a lower jaw configured to apply a compressive force to the second side of the portion of the heart, at least a portion of the lower jaw configured to be slidably engaged with the second pair of opposed tracks.

In aspects, a method comprising deploying a clip to close a portion of a heart.

The clip including: a retainer body including: an upper receiver configured to be positioned on a first side of the portion of the heart and including a first pair of opposed tracks, a lower receiver configured to be positioned on a second side of the portion of the heart that is opposite the first side and including a second pair of opposed tracks, and a loop body coupling the upper receiver to the lower receiver.

An upper jaw configured to apply a compressive force to the first side of the portion of the heart, at least a portion of the upper jaw configured to be slidably engaged with the first pair of opposed tracks.

A lower jaw configured to apply a compressive force to the second side of the portion of the heart, at least a portion of the lower jaw configured to be slidably engaged with the second pair of opposed tracks.

In aspects, a clip for a portion of a heart, the clip comprising: an upper jaw forming a plate and including a first aperture and a first compressive surface configured to apply a compressive force to a first side of the portion of the heart.

The clip comprising a lower jaw forming a plate and including a second aperture and a second compressive surface configured to apply a compressive force to a second side of the portion of the heart.

The clip comprising a loop body configured to insert into the first aperture and the second aperture and configured to compress the upper jaw and the lower jaw towards each other.

In aspects, a method comprising: deploying a clip to close a portion of a heart.

The clip including: an upper jaw forming a plate and including a first aperture and a first compressive surface configured to apply a compressive force to a first side of the portion of the heart.

The clip including a lower jaw forming a plate and including a second aperture and a second compressive surface configured to apply a compressive force to a second side of the portion of the heart.

The clip including a loop body configured to insert into the first aperture and the second aperture and configured to compress the upper jaw and the lower jaw towards each other.

In aspects, a clip for a portion of a heart, the clip comprising: a first loop body including a first elongate arm, a second elongate arm, a first closed end, a first channel between the first elongate arm and the second elongate arm and a first opened end forming an opening for the first channel.

The clip comprising a second loop body including a third elongate arm, a fourth elongate arm, a second closed end, a second channel between the third elongate arm and the fourth elongate arm and a second opened end forming an opening for the second channel.

The second loop body configured to couple to the first loop body with the second opened end positioned proximate the first closed end and the second closed end positioned proximate the first opened end, the first elongate arm and the third elongate arm forming a first jaw for compressing a first side of the portion of the heart, and the second elongate arm and the fourth elongate arm forming a second jaw for compressing a second side of the portion of the heart.

In aspects, a method comprising: deploying a clip to close a portion of a heart.

The clip may include: a first loop body including a first elongate arm, a second elongate arm, a first closed end, a first channel between the first elongate arm and the second elongate arm and a first opened end forming an opening for the first channel.

The clip may include a second loop body including a third elongate arm, a fourth elongate arm, a second closed end, a second channel between the third elongate arm and the fourth elongate arm and a second opened end forming an opening for the second channel.

The second loop body may be configured to couple to the first loop body with the second opened end positioned proximate the first closed end and the second closed end positioned proximate the first opened end, the first elongate arm and the third elongate arm forming a first jaw for compressing a first side of the portion of the heart, and the second elongate arm and the fourth elongate arm forming a second jaw for compressing a second side of the portion of the heart.

In aspects, a method comprising: forming a fold of a first portion of a left atrial appendage of a heart with a second portion of the heart; and bonding the first portion to the second portion to maintain the fold.

In aspects, a system for bonding a first portion of a left atrial appendage of a heart to a second portion of the heart, the system comprising: an elongate applicator shaft; and an implant comprising a tip of the elongate applicator shaft and configured to release from the elongate applicator shaft, the implant including an outer surface having an adhesive configured to bond the first portion of the left atrial appendage of the heart to the second portion of the heart.

In aspects, a clip for a portion of a heart, the clip comprising: a first jaw extending from a first end portion to a second end portion along a length, the first jaw including a compression surface, an outer surface facing opposite the compression surface, a first side surface extending along the length and configured to face distally and being angled along the length, and a second side surface configured to face proximally and opposite the first side surface.

The clip comprising a second jaw extending from a first end portion to a second end portion along a length, the second jaw including a compression surface, an outer surface facing opposite the compression surface, a first side surface extending along the length of the second jaw and configured to face distally, and a second side surface configured to face proximally and opposite the first side surface of the second jaw.

The clip comprising at least one spring having a first end portion coupled to the first jaw and a second end portion coupled to the second jaw and a loop portion protruding proximally from the first end portion and the second end portion of the at least one spring, the at least one spring configured to force the first jaw and the second jaw together to compress the portion of the heart between the compression surface of the first jaw and the compression surface of the second jaw.

In aspects, a method comprising: deploying a clip to close a portion of a heart.

The clip including: a first jaw extending from a first end portion to a second end portion along a length, the first jaw including a compression surface, an outer surface facing opposite the compression surface, a first side surface extending along the length and configured to face distally and being angled along the length, and a second side surface configured to face proximally and opposite the first side surface.

The clip including a second jaw extending from a first end portion to a second end portion along a length, the second jaw including a compression surface, an outer surface facing opposite the compression surface, a first side surface extending along the length of the second jaw and configured to face distally, and a second side surface configured to face proximally and opposite the first side surface of the second jaw.

The clip including at least one spring having a first end portion coupled to the first jaw and a second end portion coupled to the second jaw and a loop portion protruding proximally from the first end portion and the second end portion of the at least one spring, the at least one spring configured to force the first jaw and the second jaw together to compress the portion of the heart between the compression surface of the first jaw and the compression surface of the second jaw.

In aspects, a clip for a portion of a heart, the clip comprising: a first jaw extending from a first end portion to a second end portion along a length, the first jaw including a compression surface, an outer surface facing opposite the compression surface, a first side surface extending along the length and configured to face distally and being angled along the length, and a second side surface configured to face proximally and opposite the first side surface.

The clip comprising a second jaw extending from a first end portion to a second end portion along a length, the second jaw including a compression surface and an outer surface facing opposite the compression surface.

The clip comprising at least one spring laterally offset proximally from the second side surface of the first jaw, the at least one spring configured to force the first jaw and the second jaw together to compress the portion of the heart between the compression surface of the first jaw and the compression surface of the second jaw.

In aspects, a method comprising: deploying a clip to close a portion of a heart, the clip including: a first jaw extending from a first end portion to a second end portion along a length, the first jaw including a compression surface, an outer surface facing opposite the compression surface, a first side surface extending along the length and configured to face distally and being angled along the length, and a second side surface configured to face proximally and opposite the first side surface.

The clip including a second jaw extending from a first end portion to a second end portion along a length, the second jaw including a compression surface and an outer surface facing opposite the compression surface.

The clip including at least one spring laterally offset proximally from the second side surface of the first jaw, the at least one spring configured to force the first jaw and the second jaw together to compress the portion of the heart between the compression surface of the first jaw and the compression surface of the second jaw.

In aspects, a clip for a portion of a heart, the clip comprising: a first jaw extending from a proximal end portion to a distal end portion.

A clip comprising a second jaw extending from a proximal end portion to a distal end portion.

The clip comprising a c-shaped spring having a first end and a second end and a loop portion that loops around the proximal end portion of the first jaw and the proximal end portion of the second jaw, the first end of the c-shaped spring configured to apply a compressive force to the distal end portion of the first jaw and the second end of the c-shaped spring configured to apply a compressive force to the distal end portion of the second jaw, the c-shaped spring configured to force the first jaw and the second jaw together.

In aspects, a method comprising: deploying a clip to close a portion of a heart, the clip including: a first jaw extending from a proximal end portion to a distal end portion.

The clip including a second jaw extending from a proximal end portion to a distal end portion.

The clip including a c-shaped spring having a first end and a second end and a loop portion that loops around the proximal end portion of the first jaw and the proximal end portion of the second jaw, the first end of the c-shaped spring configured to apply a compressive force to the distal end portion of the first jaw and the second end of the c-shaped spring configured to apply a compressive force to the distal end portion of the second jaw, the c-shaped spring configured to force the first jaw and the second jaw together.

### BRIEF DESCRIPTION OF THE DRAWINGS

FIG. 1 shows a cross sectional schematic view of a portion of a heart.

FIG. 2 illustrates a perspective view of a clip.

FIG. 3 illustrates a side view of the clip shown in FIG. 2.

FIG. 4 illustrates a top view of the clip shown in FIG. 2.

FIG. 5 illustrates a perspective view of the clip shown in FIG. 2 with a first jaw displaced from a second jaw.

FIGS. 6A-6C illustrate steps of a deployment of the clip shown in FIG. 2.

FIG. 7 illustrates a perspective view of the clip shown in FIG. 2 with a first jaw displaced from a second jaw.

FIG. 8 illustrates a perspective view of the clip shown in FIG. 2 flattened.

FIG. 9 illustrates a perspective view of a clip.

FIG. 10 illustrates a top view of the clip shown in FIG. 9.

FIG. 11 illustrates a perspective view of a clip.

FIG. 12 illustrates a top view of the clip shown in FIG. 11.

FIG. 13 illustrates a side view of the clip shown in FIG. 11.

FIG. 14 illustrates a perspective view of the clip shown in FIG. 11 with a first jaw displaced from a second jaw.

FIG. 15 illustrates a side view of the clip shown in FIG. 11 in the position shown in FIG. 14.

FIGS. 16A and 16B illustrate steps of a deployment of the clip shown in FIG. 11.

FIG. 17 illustrates a perspective view of a clip.

FIG. 18 illustrates a perspective view of the clip shown in FIG. 17 from an opposite side of the clip than shown in FIG. 17.

FIG. 19 illustrates a side view of the clip shown in FIG. 17.

FIG. 20 illustrates an end view of the clip shown in FIG. 17.

FIG. 21 illustrates a top view of the clip shown in FIG. 17.

FIG. 22 illustrates a perspective view of the clip shown in FIG. 17 with jaws shown in transparency.

FIG. 23 illustrates a cross sectional view of the clip shown in FIG. 17 along line A-A shown in FIG. 21.

FIGS. 24A and 24B illustrate a cross sectional view of the clip shown in FIG. 17 deployed to a LAA.

FIGS. 25A and 25B illustrate a side view of the clip shown in FIG. 17 deployed to a LAA.

FIG. 26 illustrates a perspective view of a clip.

FIG. 27 illustrates a perspective view of the clip shown in FIG. 26 from an opposite side of the clip than shown in FIG. 26.

FIG. 28 illustrates an end view of the clip shown in FIG. 26.

FIG. 29 illustrates an assembly view of the clip shown in FIG. 26.

FIG. 30 illustrates a perspective view of a clip.

FIG. 31 illustrates a perspective view of the clip shown in FIG. 30 from an opposite side of the clip than shown in FIG. 30.

FIG. 32 illustrates an end view of the clip shown in FIG. 30.

FIG. 33 illustrates a perspective view of a clip.

FIG. 34 illustrates a side view of a clip.

FIG. 35 illustrates a perspective view of a spring.

FIG. 36A illustrates a side view of a clip with jaws shown in transparency.

FIG. 36B illustrates a perspective view of the clip shown in FIG. 33 with the first jaw excluded from view.

FIG. 36C illustrates a cross sectional view of the clip shown in FIG. 33 in cross sectional view along line B-B.

FIG. 37 illustrates a perspective view of a clip.

FIG. 38 illustrates a side view of the clip shown in FIG. 37 from an opposite side of the clip than shown in FIG. 37.

FIG. 39 illustrates a top view of the clip shown in FIG. 37.

FIG. 40 illustrates an assembly view of the clip shown in FIG. 37.

FIG. 41 illustrates a cross sectional schematic view of the clip shown in FIG. 37 deployed to the left atrial appendage (LAA).

FIGS. 42A-42C illustrate steps of a deployment of the clip shown in FIG. 37.

FIG. 43 illustrates a perspective view of a clip.

FIG. 44 illustrates a side view of the clip shown in FIG. 43 in an opened state.

FIG. 45 illustrates a side view of a clip.

FIG. 46 illustrates an end view of the clip shown in FIG. 45.

FIG. 47 illustrates an end perspective view of the clip shown in FIG. 45 with the jaws bent.

FIG. 48 illustrates a top view of the clip shown in FIG. 45 with the jaws bent.

FIG. 49 illustrates a curvature measurement procedure being applied to a heart.

FIG. 50 illustrates a cross sectional schematic view of the clip shown in FIG. 45 deployed to a left atrial appendage (LAA).

FIG. 51 illustrates a side view of the clip shown in FIG. 45 in an opened state around a LAA.

FIG. 52 illustrates a side view of the clip shown in FIG. 45 moved to compress the LAA.

FIG. 53 illustrates a side view of the clip shown in FIG. 45 in a closed state.

FIG. 54 illustrates a side view of a delivery apparatus.

FIG. 55 illustrates a perspective view of an end of the delivery apparatus shown in FIG. 54.

FIG. 56 illustrates a side view of the delivery apparatus shown in FIG. 54.

FIG. 57 illustrates a perspective view of a clip.

FIG. 58 illustrates a perspective view of a clip.

FIG. 59 illustrates a perspective view of the clip shown in FIG. 58 from an opposite side of the clip than shown in FIG. 58.

FIG. 60 illustrates a side view of the clip shown in FIG. 58.

FIG. 61 illustrates an end view of the clip shown in FIG. 58.

FIG. 62 illustrates an assembly view of the clip shown in FIG. 58.

FIG. 63 illustrates a cross sectional schematic view of the clip shown in FIG. 58 deployed to the left atrial appendage (LAA).

FIGS. 64A-64C illustrate steps of a deployment of the clip shown in FIG. 58.

FIG. 65 illustrates a schematic view of a delivery apparatus.

FIGS. 66A and 66B illustrate a side view of a clip.

FIGS. 67A and 67B illustrate a side view of a clip.

FIG. 68 illustrates a perspective view of a clip.

FIGS. 69A and 69B illustrate the clip shown in FIG. 68 being deployed.

FIGS. 70A and 70B illustrate the clip shown in FIG. 68 being deployed.

FIG. 71 illustrates a perspective view of a clip.

FIG. 72 illustrates a perspective view of a clip.

FIG. 73 illustrates a side view of the clip shown in FIG. 72.

FIG. 74 illustrates a top view of the clip shown in FIG. 72.

FIG. 75 illustrates a perspective view of a clip.

FIG. 76 illustrates a side view of the clip shown in FIG. 75.

FIG. 77 illustrates a top view of the clip shown in FIG. 75.

FIG. 78 illustrates a perspective view of a clip.

FIG. 79 illustrates a perspective view of a clip.

FIG. 80 illustrates a side view of the clip shown in FIG. 79.

FIG. 81 illustrates a top view of the clip shown in FIG. 79.

FIG. 82 illustrates a side view of a clip.

FIG. 83 illustrates a perspective view of the clip shown in FIG. 82.

FIG. 84 illustrates a side view of a clip.

FIG. 85 illustrates a perspective view of a clip.

FIG. 86 illustrates a side view of a clip.

FIG. 87 illustrates a perspective view of the clip shown in FIG. 86.

FIG. 88 illustrates a side view of a clip.

FIG. 89 illustrates a perspective view of a clip shown in FIG. 88.

FIG. 90 illustrates a perspective view of a clip.

FIG. 91 illustrates a side view of the clip shown in FIG. 90 from an opposite side of the clip than shown in FIG. 90.

FIG. 92 illustrates a cross sectional schematic view of the clip shown in FIG. 90 deployed to a left atrial appendage (LAA).

FIGS. 93A-93C illustrate steps of a deployment of the clip shown in FIG. 90.

FIG. 94 illustrates a perspective view of a clip.

FIG. 95 illustrates a side view of the clip shown in FIG. 94 from an opposite side of the clip than shown in FIG. 94.

FIG. 96 illustrates a perspective view of a clip.

FIG. 97 illustrates a perspective view of a clip.

FIG. 98 illustrates a perspective view of the clip shown in FIG. 97 from an opposite side of the clip than shown in FIG. 97.

FIG. 99 illustrates a side view of the clip shown in FIG. 97.

FIG. 100 illustrates a top view of the clip shown in FIG. 97.

FIG. 101 illustrates an assembly view of the clip shown in FIG. 97.

FIG. 102 illustrates an assembly view of the clip shown in FIG. 97 at an opposite side of the clip than shown in FIG. 101.

FIGS. 103A and 103B illustrate steps of a deployment of the clip shown in FIG. 97.

FIGS. 104A and 104B illustrate steps of a deployment of the clip shown in FIG. 97.

FIG. 105 illustrates a perspective view of a clip.

FIG. 106 illustrates a perspective view of a portion of a jaw.

FIG. 107 illustrates a perspective view of a spring.

FIG. 108 illustrates a perspective view of a clip.

FIG. 109 illustrates a perspective view of the clip shown in FIG. 108 at an opposite side of the clip than shown in FIG. 108.

FIG. 110 illustrates a perspective view of a slide bar slid with respect to a jaw.

FIG. 11 illustrates a side view of a clip in an opened configuration.

FIG. 112 illustrates a side view of the clip shown in FIG. 11.

FIG. 113 illustrates a side view of the clip shown in FIG. 11 in a closed configuration.

FIG. 114 illustrates a side view of the clip shown in FIG. 11 closed upon a portion of a heart.

FIG. 115 illustrates a cross sectional view of the clip shown in FIG. 113 along line C-C.

FIG. 116 illustrates a cross sectional view of the clip shown in FIG. 113 along line D-D.

FIG. 117 illustrates a cross sectional schematic view of a left atrial appendage (LAA).

FIG. 118 illustrates a cross sectional schematic view of the clip shown in FIG. 11 deployed to a left atrial appendage (LAA).

FIG. 119 illustrates a side view of a clip in an opened configuration.

FIG. 120 illustrates a side view of the clip shown in FIG. 119 in a closed configuration.

FIG. 121 illustrates an end perspective view of the clip shown in FIG. 119 in an opened configuration.

FIG. 122 illustrates a side view of a clip.

FIG. 123 illustrates a top view of the clip shown in FIG. 122.

FIG. 124 illustrates an end view of the clip shown in FIG. 122 in the position shown in FIG. 125.

FIG. 125 illustrates a side view of the clip shown in FIG. 122 moved from the position shown in FIG. 122.

FIG. 126 illustrates a perspective view of a delivery apparatus that may be utilized in examples herein.

FIG. 127 illustrates a top view of a delivery head that may be utilized in examples herein.

FIG. 128 illustrates an end view of the delivery head shown in FIG. 127.

FIG. 129 illustrates a perspective view of the delivery head shown in FIG. 127 with clips coupled thereto.

FIG. 130 illustrates a side cross sectional view of a first clip deployed to a left atrial appendage (LAA).

FIG. 131 illustrates a top view of the first clip deployed to the LAA.

FIG. 132 illustrates a side cross sectional view of a second clip deployed between the first clip and a wall of the left atrium.

FIG. 133 illustrates a side view of a clip.

FIG. 134 illustrates a cross sectional view of the clip shown in FIG. 133 along line E-E.

FIG. 135 illustrates a cross sectional view of the clip shown in FIG. 133 along line F-F.

FIG. 136 illustrates a cross sectional view of the clip shown in FIG. 133 along line G-G.

FIG. 137 illustrates a side view of a clip for insertion into the clip shown in FIG. 133.

FIG. 138 illustrates a side cross sectional view of the clip shown in FIG. 133 deployed to a left atrial appendage (LAA).

FIG. 139 illustrates a side cross sectional view of the clip shown in FIG. 133 rotated.

FIG. 140 illustrates a side cross sectional view of a clip having axes eccentric with outer surfaces of jaws of the clip.

FIG. 141 illustrates a front view of a clip having closed ends.

FIG. 142 illustrates a top view of the clip shown in FIG. 141.

FIG. 143 illustrates a side view of a clip shown in FIG. 141.

FIG. 144 illustrates a perspective view of a clip.

FIG. 145 illustrates a cross sectional view of a retainer body of the clip shown in FIG. 144 along a mid-line.

FIG. 146 illustrates a cross sectional view of an upper jaw of the clip shown in FIG. 144 along a mid-line.

FIG. 147 illustrates a perspective view of the clip shown in FIG. 144.

FIG. 148 illustrates a side view of the clip shown in FIG. 144.

FIG. 149 illustrates a top schematic view of the clip shown in FIG. 144 being deployed to a left atrial appendage.

FIG. 150 illustrates a top schematic view of the clip shown in FIG. 144 being deployed to a left atrial appendage.

FIG. 151 illustrates a perspective view of jaws of a clip.

FIG. 152 illustrates a side view of a loop body for the clip shown in FIG. 151.

FIG. 153 illustrates a side view of the loop body shown in FIG. 152 and the jaws shown in FIG. 151.

FIG. 154 illustrates an end view of the clip shown in FIG. 153.

FIG. 155 illustrates a partial cross sectional view of the clip shown in FIG. 153.

FIG. 156 illustrates a top schematic view of the clip shown in FIG. 153 being deployed to a left atrial appendage.

FIG. 157 illustrates a top schematic view of the clip shown in FIG. 153 being deployed to a left atrial appendage.

FIG. 158 illustrates a front perspective view of a loop body of a clip.

FIG. 159 illustrates a rear perspective view of the loop body shown in FIG. 158.

FIG. 160 illustrates a side view of the loop body shown in FIG. 158.

FIG. 161 illustrates a perspective view of a loop body of a clip configured to couple to the loop body shown in FIG. 158.

FIG. 162 illustrates a side view of the loop body shown in FIG. 161.

FIG. 163 illustrates a side view of the loop body of the clip shown in FIG. 158 coupling with the loop body of the clip shown in FIG. 161.

FIG. 164 illustrates a side view of the loop body of the clip shown in FIG. 158 coupling with the loop body of the clip shown in FIG. 161.

FIG. 165 illustrates a perspective view of the loop body of the clip shown in FIG. 158 coupling with the loop body of the clip shown in FIG. 161.

FIG. 166 illustrates a side view of a loop body of a clip.

FIG. 167 illustrates a perspective view of the loop body shown in FIG. 166.

FIG. 168 illustrates engagement of a lock.

FIG. 169 illustrates a side view of a loop body of a clip.

FIG. 170 illustrates a side view of a loop body of a clip.

FIG. 171 illustrates a side view of loop bodies of a clip coupling to each other.

FIG. 172 illustrates a side view of loop bodies of a clip coupled to each other.

FIG. 173 illustrates a perspective view of a loop body applicator.

FIG. 174 illustrates a side view of the loop body shown in FIG. 158 coupled to the loop body applicator shown in FIG. 173.

FIG. 175 illustrates a side view of the loop body shown in FIG. 158 coupled to the loop body applicator shown in FIG. 173.

FIG. 176 illustrates a schematic view of the loop body shown in FIG. 158 extending over a left atrial appendage.

FIG. 177 illustrates a schematic view of the clip shown in FIG. 164 extending over a left atrial appendage.

FIG. 178 illustrates a perspective view of a clip.

FIG. 179 illustrates a side view of the clip shown in FIG. 178.

FIG. 180 illustrates a side view of a system according to examples herein.

FIG. 181 illustrates a cross sectional view of the system shown in FIG. 180.

FIG. 182 illustrates a cross sectional view of the system shown in FIG. 180 with the implant separated from the applicator shaft.

FIG. 183 illustrates a cross sectional view of a system according to examples herein.

FIGS. 184A-184F illustrate shapes of implants according to examples herein.

FIG. 185 illustrates a cross sectional schematic view of an implant approaching the left atrial appendage (LAA).

FIG. 186 illustrates a cross sectional schematic view of a portion of the LAA shown in FIG. 185 being folded.

FIG. 187 illustrates a cross sectional schematic view of an applicator shaft being withdrawn from the implant shown in FIG. 186.

FIG. 188 illustrates a cross sectional schematic view of the folded LAA shown in FIG. 187 being sutured.

FIG. 189 illustrates a perspective view of sutures closing tissue.

FIG. 190 illustrates a cross sectional schematic view of the folded LAA shown in FIG. 188 sutured.

FIG. 191 illustrates a cross sectional schematic view of a plurality of implants approaching the left atrial appendage (LAA).

FIG. 192 illustrates a cross sectional schematic view of a portion of the LAA shown in FIG. 191 being folded.

FIG. 193 illustrates a cross sectional schematic view along line H-H in FIG. 192 of the ostium of the LAA being closed.

FIG. 194 illustrates a cross sectional schematic view of a plurality of implants approaching the left atrial appendage (LAA).

FIG. 195 illustrates a cross sectional schematic view of a plurality of portions of the LAA shown in FIG. 194 being folded.

FIG. 196 illustrates a cross sectional schematic view of an implant approaching the left atrial appendage (LAA) that is grasped with a grasper.

FIG. 197 illustrates a cross sectional schematic view of an implant approaching the left atrial appendage (LAA) that is grasped with a grasper.

FIG. 198 illustrates a top view of a clip deployed to a left atrial appendage (LAA) of a heart.

FIG. 199 illustrates a front view of the clip shown in FIG. 198.

FIG. 200 illustrates a side view of the clip shown in FIG. 198.

FIG. 201 illustrates a cross sectional view of a pivot coupler of the clip shown in FIG. 198.

FIG. 202 illustrates a top view of the clip shown in FIG. 198 being deployed to a LAA of a heart.

FIG. 203 illustrates a side view of the clip shown in FIG. 198 retained by a delivery apparatus.

FIG. 204 illustrates a side view of the clip shown in FIG. 198 being deployed by a delivery apparatus.

FIG. 205 illustrates a side view of the clip shown in FIG. 198 deployed to a LAA.

FIG. 206 illustrates a cross sectional view of a compression surface of a clip.

FIG. 207 illustrates a rear view of a clip.

FIG. 208 illustrates a top view of the clip shown in FIG. 207 deployed to a left atrial appendage (LAA) of a heart.

FIG. 209 illustrates a rear view of a clip retained by a delivery apparatus.

FIG. 210 illustrates a rear view of the clip shown in FIG. 207 being deployed to a LAA.

FIG. 211 illustrates a rear view of the clip shown in FIG. 207 being deployed to a LAA.

FIG. 212 illustrates a rear view of the clip shown in FIG. 207 deployed to a LAA.

FIG. 213 illustrates a perspective view of a clip.

FIG. 214 illustrates a side view of the clip shown in FIG. 213.

FIG. 215 illustrates a top view of the clip shown in FIG. 213.

FIG. 216 illustrates an end view of the clip shown in FIG. 213.

FIG. 217 illustrates a perspective view of the first jaw shown in FIG. 213.

FIG. 218 illustrates a perspective view of the spring shown in FIG. 213.

FIG. 219 illustrates a side view of the clip shown in FIG. 213 with the jaws shown in transparency.

FIG. 220 illustrates a side view of a first jaw of the clip shown in FIG. 213 in an opened state.

FIG. 221 illustrates a side view of a first jaw of the clip shown in FIG. 213 in an opened state.

FIG. 222 illustrates a cross sectional view of a first jaw of a clip.

FIG. 223 illustrates a cross sectional view of a first jaw of a clip.

FIG. 224 illustrates a perspective view of a first jaw of a clip.

FIG. 225 illustrates a cross sectional view of the first jaw of the clip shown in FIG. 224.

### DETAILED DESCRIPTION

FIG. 1 illustrates a cross sectional view of a left atrium 10 and a left ventricle 12 of an individual's heart 14. The left atrium 10 is configured to fill with blood to pass into the left ventricle 12 via the mitral valve 16 during the cardiac cycle. The left atrial appendage (LAA) 18 protrudes from the outer wall 20 of the left atrium 10 and includes an ostium 22 and a cavity 24 extending from the ostium 22. A LAA wall 26 may surround the cavity 24 and may form an outer surface 28 of the LAA 18. The LAA 18 has a pouch shape extending from the left atrium 10. The cavity 24 may be configured to fill with blood, allowing the LAA to serve as a decompression chamber during systole and when pressure otherwise increases in the left atrium 10.

In certain individuals, blood may stagnate and form clots within the LAA 18. Clots or other undesired materials stemming from the LAA 18 may travel into the bloodstream, producing a variety of maladies including strokes. It may thus be beneficial to close the LAA 18, to reduce the possibility of clots or other undesired material from producing such maladies.

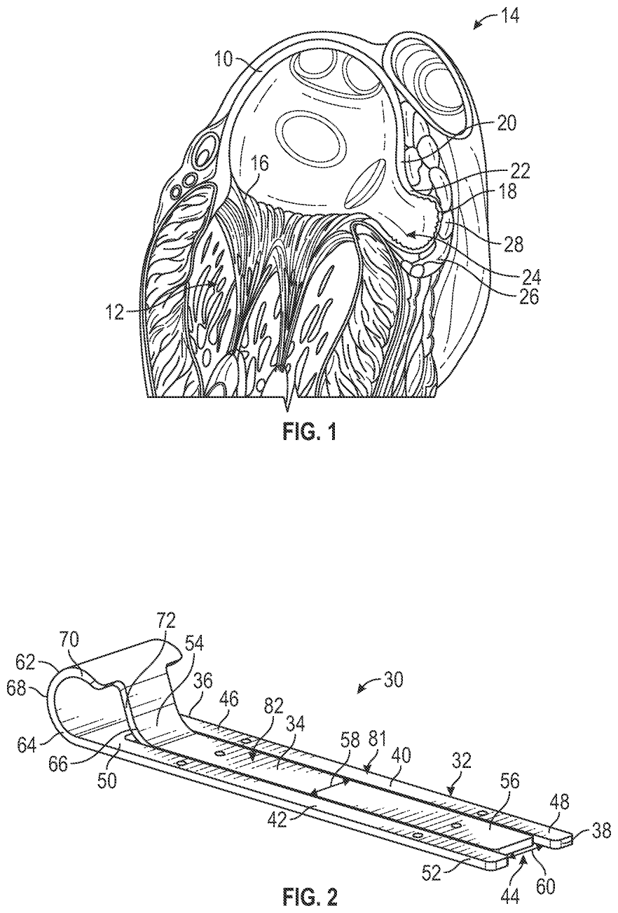

FIG. 2 illustrates an example of a clip 30 that may be utilized for a portion of a heart. The clip 30 may be configured to close a LAA 18, to reduce the possibility of clots or other undesired materials stemming from the LAA 18 from traveling into the bloodstream.

The clip 30 may include a first jaw 32 and a second jaw 34. The first jaw 32 may extend from a first end portion 36 to a second end portion 38. The first jaw 32 may include a first arm 40 and a second arm 42 separated from the first arm 40 with a gap 44. The first arm 40 and the second arm 42 may each be configured to apply a compressive force to a portion of the heart.

The first arm 40 may extend from a first end 46 to a second end 48 and may have an elongate shape. The first arm 40 may have a rectangular shape. The first arm 40 may have a rectangular cross section when viewed perpendicular to the longitudinal axis of the first arm 40.

The second arm 42 may extend from a first end 50 to a second end 52 and may have an elongate shape. The second arm 42 may have a rectangular shape. The second arm 42 may have a rectangular cross section when viewed perpendicular to the longitudinal axis of the second arm 42. The second arm 42 may extend parallel with the first arm 40, as shown in FIG. 2.

The arms 40, 42 of the first jaw 32 may each include an upper surface 81 and a lower surface facing opposite the upper surface 81.

The gap 44 may extend along the lengths of the first arm 40 and the second arm 42 and may extend from the first ends 46, 50 of the arms 40, 42 to the second ends 48, 52 of the arms 40, 42. The gap 44 may have a rectangular shape between the arms 40, 42.

The second jaw 34 may be positioned offset from the first arm 40 and the second arm 42 and aligned with the gap 44. The second jaw 34 may be configured to apply a compressive force to the portion of the heart in a direction towards the gap 44.

The second jaw 34 may extend from a first end portion 54 to a second end portion 56. The second jaw 34 may have an elongate shape, and may have a rectangular shape. The second jaw 34 may have a rectangular cross section when viewed perpendicular to the longitudinal axis of the second jaw 34. The second jaw 34 may be configured to pass into the gap 44 as shown in FIG. 2. The second jaw 34, for example, may have a width 58 that is at or less than the width 60 of the gap 44, to allow the second jaw 34 to fit within the gap 44 as shown in FIG. 2. The first jaw 32 and the second jaw 34 may extend in the same plane.

The second jaw 34 may include an upper surface 82 and a lower surface facing opposite the upper surface 82.

The first end portion 36 of the first jaw 32 may be coupled to the first end portion 54 of the second jaw 34. A spring 62 may be positioned at the first end portion 36 of the first jaw 32 and the first end portion 54 of the second jaw 34 and may couple the first jaw 32 to the second jaw 34. The spring 62 may be configured to force the first jaw 32 and the second jaw 34 together to compress the portion of the heart between the first jaw 32 and the second jaw 34. The spring 62 may comprise a hinge portion of the clip 30 that the second jaw 34 and/or first jaw 32 is configured to move relative to.

The spring 62, in examples, may comprise a loop of material coupling the first jaw 32 to the second jaw 34. The loop of material may include a first end portion 64 that is integral with the first end portion 36 of the first jaw 32 and a second end portion 66 that is integral with the first end portion 54 of the second jaw 34. The loop may include a first curved portion 68 that rises to an apex 70 of the loop that is offset from the plane of the first jaw 32 and the second jaw 34. The first curved portion 68 may be coupled to the first jaw 32 and extend from the first jaw 32 to the apex 70. The loop may include a second curved portion 72 that descends from the apex 70 of the loop towards the plane of the first jaw 32 and the second jaw 34. The second curved portion 72 may be coupled to the second jaw 34 and extend from the apex 70 to the second jaw 34.

In examples, the second end portion 66 of the loop may have a width 74 (marked in FIG. 4) that is less than the width 76 (marked in FIG. 4) of the first end portion 64. The width 74 of the second end portion 66 may match the width 58 of the second jaw 34 and the width 76 of the first end portion 64 may match a width 78 of the first jaw 32.

The arms 40, 42 of the first jaw 32 may extend in a forked configuration from the spring 62, with the second jaw 34 positioned between the arms 40, 42.

The clip 30 may comprise a unitary piece of material that is folded into the shape shown in FIG. 2. FIG. 8, for example, illustrates the clip 30 prior to folding, in which the unitary piece of material is cut out of a flat sheet of material in a desired shape. The material forming the second jaw 34 and the first jaw 32 are shown in a single plane in FIG. 8 prior to folding. The second jaw 34 is integral with the first jaw 32. The material may then be folded with the loop of the spring 62 formed between the first jaw 32 and the second jaw 34, and the second jaw 34 positioned between the arms 40, 42 as shown in FIG. 2.

The clip 30 may be shape set into the configuration shown in FIG. 2 in examples. The clip 30 may be made of a shape memory material that may be set into the configuration shown in FIG. 2. The shape memory material may comprise nitinol (NiTi alloy) or another form of shape memory material. The clip 30 may be configured to form into the shape shown in FIG. 2 upon deflection away from the shape shown in FIG. 2.

The clip 30 may be configured to move from an opened state to a closed state in which the portion of the heart is compressed by the first jaw 32 and second jaw 34. In the opened state, the second jaw 34 may be moved out of the plane of the first jaw 32, such that an axial opening 80 (marked in FIG. 5) for a space 79 between the first jaw 32 and the second jaw 34 is formed. The second end portion 38 of the first jaw 32 and the second end portion 56 of the second jaw 34 may form the axial opening 80 between the first jaw 32 and the second jaw 34. The loop of the spring 62 may close an opposite end of the space 79 between the first jaw 32 and the second jaw 34.

The clip 30 may be biased by the shape memory of the material of the clip 30 to return to a closed state, and towards the configuration shown in FIG. 2. As such, the second jaw 34 may apply a compressive force in a direction towards the gap 44 in the closed state to compress the portion of the heart within the space 79.

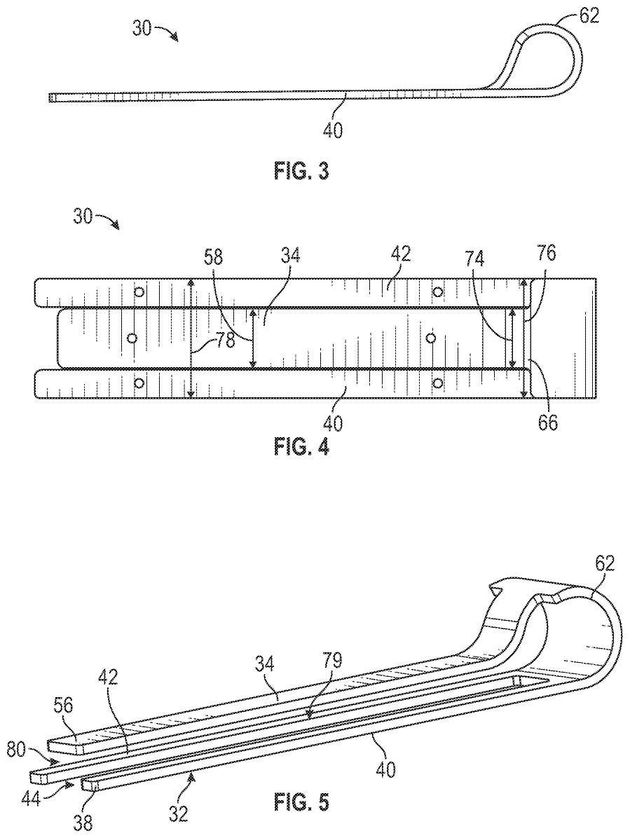

FIG. 3 illustrates a side view of the clip 30. FIG. 4 illustrates a top view of the clip 30.

FIG. 5 illustrates a perspective view of the clip 30 in the opened state. The second jaw 34 may be positioned offset from the plane of the first jaw 32, yet in a plane extending parallel with the first jaw 32.

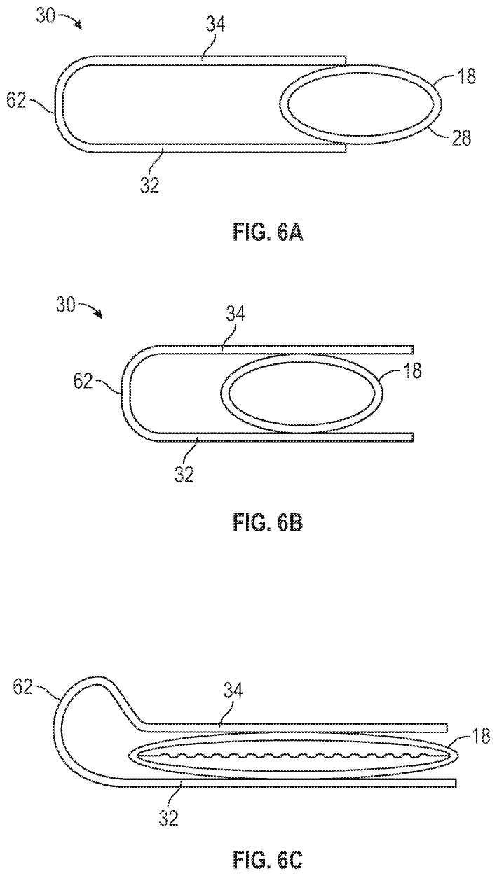

In operation, the clip 30 may be utilized to close the LAA 18. FIGS. 6A-6C illustrate a deployment sequence of the clip 30.

FIG. 6A illustrates the clip 30 may approach the LAA 18 in an opened state. Access may be provided to the LAA 18 in a variety of manners, including a transcatheter access or a surgical access such as a thoracotomy, mini thoracotomy, or other methods of access.

In examples, a delivery apparatus may be utilized to move the clip 30 towards the LAA 18, or in examples, a user (e.g., a medical technician such as a surgeon, or other form of medical technician) may manually place the clip 30. The clip 30 may be placed over the outer surface 28 of the LAA 18 in an opened state. A user may confirm a desired placement of the clip 30 over the LAA 18.

FIG. 6B illustrates the clip 30 positioned around the LAA 18 in an opened state. With the clip 30 in the desired position, the clip 30 may be moved to the closed state as shown in FIG. 6C. The LAA 18 may be compressed by the jaws 32, 34 of the clip 30 and closed. In examples, the clip 30 may utilize a shape memory in a closed state to move the clip 30 towards the closed state shown in FIG. 2 from an opened state.

The entire span of the LAA 18 may be closed as desired at the ostium or another portion of the LAA 18 may be closed.

In examples, if desired, the clip 30 may be opened and either removed or repositioned in a deployment procedure or another procedure. The clip 30 may be grasped and the force of the spring 62 may be overcome to open the clip 30.

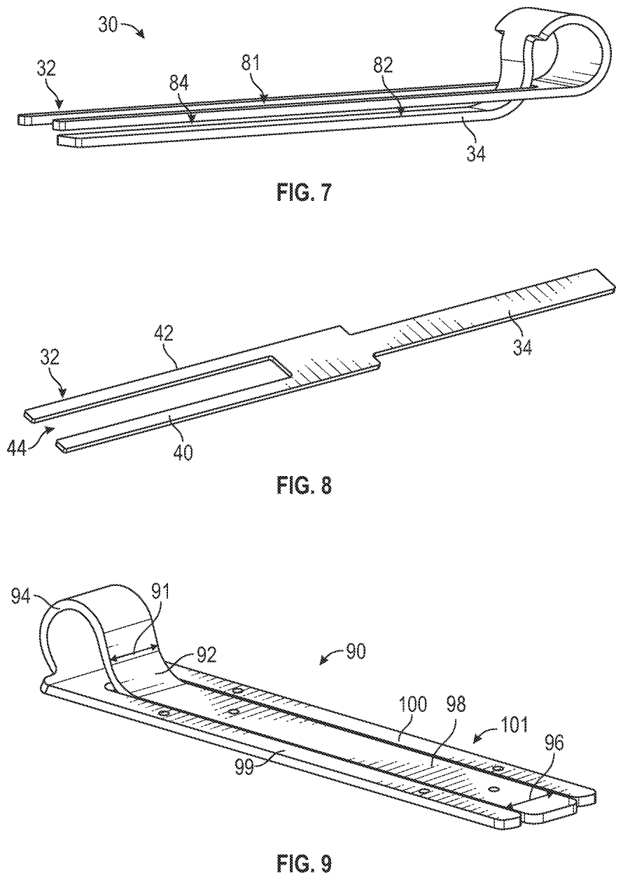

In examples, the clip 30 may operate to close the LAA 18 even if the tissue of the LAA 18 is positioned between an upper surface 82 of the second jaw 34 and a lower surface of the first jaw 32. FIG. 7 illustrates such a configuration, in which the LAA 18 may be positioned in the space 84 between the upper surface 82 of the second jaw 34 and the lower surface of the first jaw 32. The clip 30 may be configured to move towards the shape shown in FIG. 2 to apply a compressive force to the LAA 18.

In examples, the clip 30 may be shape set into the configuration shown in FIG. 7, with the second jaw 34 extending offset from the plane of the first jaw 32. The second jaw 34 may be positioned below the lower surface of the first jaw 32. The second jaw 34 may be moved above the upper surface 81 of the first jaw 32 in the opened state to allow the LAA 18 to be positioned between the upper surface 81 of the first jaw 32 and the lower surface of the second jaw 34. The clip 30 may be configured to move towards the shape shown in FIG. 7 to apply a compressive force to the LAA 18 in such a configuration.

The examples of the clip may be utilized to close the LAA 18, yet in examples other portions of a heart may be clipped or closed via use of the clip. In examples, other portions of a body, such as a tubular vessel or other portions of a body may be closed with the clip. Deployment may be via a delivery apparatus or via another method as desired.

Variations in the configuration of the clip may be provided as desired.

FIG. 9 illustrates an example of a clip 90 in which a width 91 of a first end portion 92 of the spring 94 is the width 96 of the second jaw 98. In such a configuration, the loop of the spring 94 may be displaced further with regard to the arms 99, 110 of the first jaw 101 because a greater proportion of the loop may pass through the gap between the arms 99, 100 than in the example shown in FIG. 2. The displacement between the first jaw 101 and the second jaw 98 accordingly may be greater than the example shown in FIG. 2.

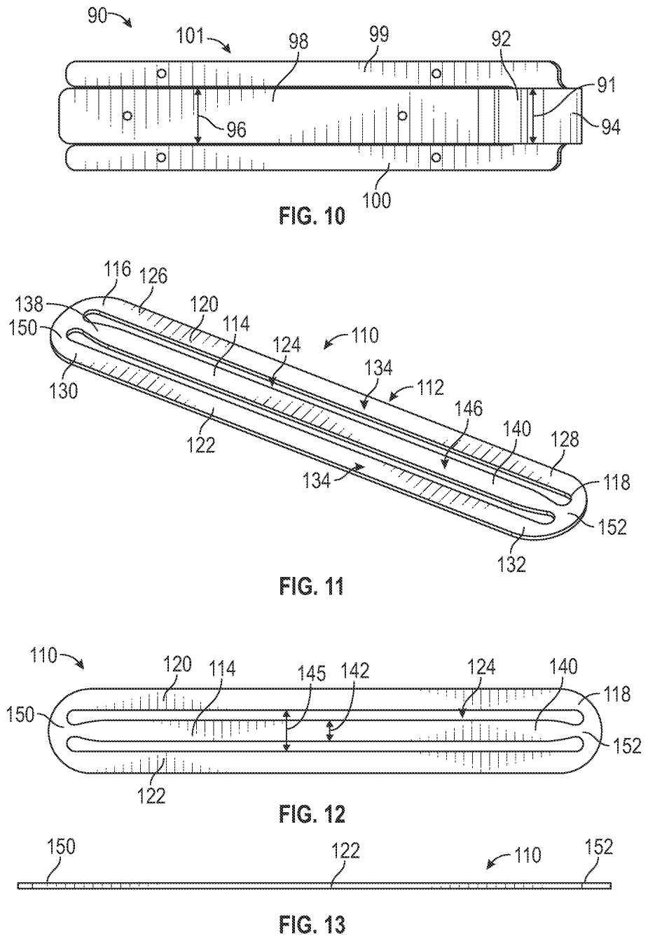

FIG. 10 illustrates a top view of the clip 90 shown in FIG. 9.

Variations in the configuration of the clip may be provided as desired.

FIGS. 11-15 illustrate an example of a clip 110 that may be utilized for a portion of a heart. The clip 110 may be configured to close a LAA 18, to reduce the possibility of clots or other undesired materials stemming from the LAA 18 from traveling into the bloodstream.

The clip 110 may include a first jaw 112 and a second jaw 114. The first jaw 112 may extend from a first end portion 116 to a second end portion 118. The first jaw 112 may include a first arm 120 and a second arm 122 separated from the first arm 120 with a gap 124. The first arm 120 and the second arm 122 may each be configured to apply a compressive force to a portion of the heart.

The first arm 120 may extend from a first end 126 to a second end 128 and may have an elongate shape. The first arm 120 may have a rectangular shape. The first arm 120 may have a rectangular cross section when viewed perpendicular to the longitudinal axis of the first arm 120.

The second arm 122 may extend from a first end 130 to a second end 132 and may have an elongate shape. The second arm 122 may have a rectangular shape. The second arm 122 may have a rectangular cross section when viewed perpendicular to the longitudinal axis of the second arm 122. The second arm 122 may extend parallel with the first arm 120, as shown in FIG. 11.

The arms 120, 122 of the first jaw 112 may each include an upper surface 134 and a lower surface facing opposite the upper surface 134.

The gap 124 may extend along the lengths of the first arm 120 and the second arm 122 and may extend from the first ends 126, 130 of the arms 120, 122 to the second ends 128, 132 of the arms 120, 122. The gap 124 may have a rectangular shape between the arms 120, 122.

The second jaw 114 may be positioned offset from the first arm 120 and the second arm 122 and aligned with the gap 124. The second jaw 114 may be configured to apply a compressive force to the portion of the heart in a direction towards the gap 124.

The second jaw 114 may extend from a first end portion 138 to a second end portion 140. The second jaw 114 may have an elongate shape, and may have a rectangular shape. The second jaw 114 may have a rectangular cross section when viewed perpendicular to the longitudinal axis of the second jaw 114. The second jaw 114 may be configured to pass into the gap 124 as shown in FIG. 11. The second jaw 114, for example, may have a width 142 (marked in FIG. 12) that is at or less than the width 145 of the gap 124 (as marked in FIG. 12), to allow the second jaw 114 to fit within the gap 124 as shown in FIG. 11. The first jaw 112 and the second jaw 114 may extend in the same plane.

The second jaw 114 may include an upper surface 146 and a lower surface facing opposite the upper surface 146.

The first end portion 116 of the first jaw 112 may be coupled to the first end portion 138 of the second jaw 98. A spring 150 may be positioned at the first end portion 116 of the first jaw 112 and the first end portion 138 of the second jaw 114 and may couple the first jaw 112 to the second jaw 114. The spring 150 may be configured to force the first jaw 112 and the second jaw 114 together to compress the portion of the heart between the first jaw 112 and the second jaw 114.

The spring 150 may comprise an end portion of the clip 110 that extends in the same plane as the first jaw 112 and the second jaw 114. The spring 150 may comprise a hinge portion of the clip 110 that the first jaw 112 and/or second jaw 114 is configured to move relative to. The spring 150 may have a flattened shape as shown in FIG. 11.

The second end portion 118 of the first jaw 112 may be coupled to the second end portion 140 of the second jaw 114. A spring 152 may be positioned at the second end portion 118 of the first jaw 112 and the second end portion 140 of the second jaw 114 and may couple the first jaw 112 to the second jaw 114. The spring 152 may be configured to force the first jaw 112 and the second jaw 114 together to compress the portion of the heart between the first jaw 112 and the second jaw 114.

The spring 152 may comprise an end portion of the clip 110 that is opposite the position of the spring 150. The spring 152 may extend in the same plane as the first jaw 112 and the second jaw 114. The spring 152 may comprise a hinge portion of the clip 110 that the first jaw 112 and/or second jaw 114 is configured to move relative to. The spring 152 may have a flattened shape as shown in FIG. 11.

The springs 150, 152 may enclose respective ends of the gap 124.

The clip 110 may comprise a unitary piece of material that is cut into the shape shown in FIG. 11. The material may be cut to produce a single plane of material as desired. The clip 110 may have a flattened shape. A flat unitary piece of material may be cut to produce the shape of the clip 110.

The clip 110 may be shape set into the configuration shown in FIG. 11 in examples. The clip 110 may be made of a shape memory material that may be set into the configuration shown in FIG. 11. The shape memory material may comprise nitinol (NiTi alloy) or another form of shape memory material. The clip 110 may be configured to move towards the shape shown in FIG. 11 upon deflection away from the shape shown in FIG. 11.

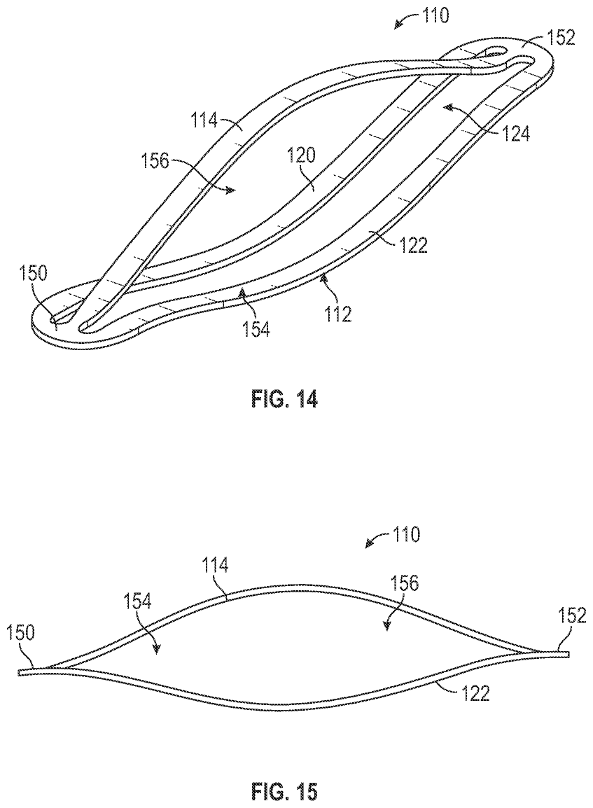

The clip 110 may be configured to move from an opened state to a closed state in which the portion of the heart is compressed by the first jaw 112 and second jaw 114. An opened state is shown in FIGS. 14 and 15 for example. In the opened state, the second jaw 114 may be moved out of the plane of the first jaw 112, such that an opening 154 (marked in FIGS. 14 and 15) for a space 156 between the first jaw 112 and the second jaw 114 is formed. The space 156 may be configured to receive a portion of a heart in a direction transverse to the axis of the first jaw 112 and the axis of the second jaw 114. The springs 150, 152 may enclose respective ends of the space 156.

The clip 110 may be biased by the shape memory of the material of the clip 110 to return to a closed state, and towards the configuration shown in FIG. 11. As such, the first second jaw 114 may apply a compressive force in a direction towards the gap 124 in the closed state to compress the portion of the heart within the space 156.

FIG. 12 illustrates a top view of the clip 110. FIG. 13 illustrates a side view of the clip 110.

FIG. 14 illustrates a perspective view of the clip 110 in the opened state. The second jaw 114 may be deflected away from the first jaw 112 to form a curved shape. The first jaw 112 may further form a curved shape. The jaws 112, 114 of the clip 110 may form an oval shape bounding the space 156. FIG. 15 illustrates a side view of the clip 110 in the opened state.

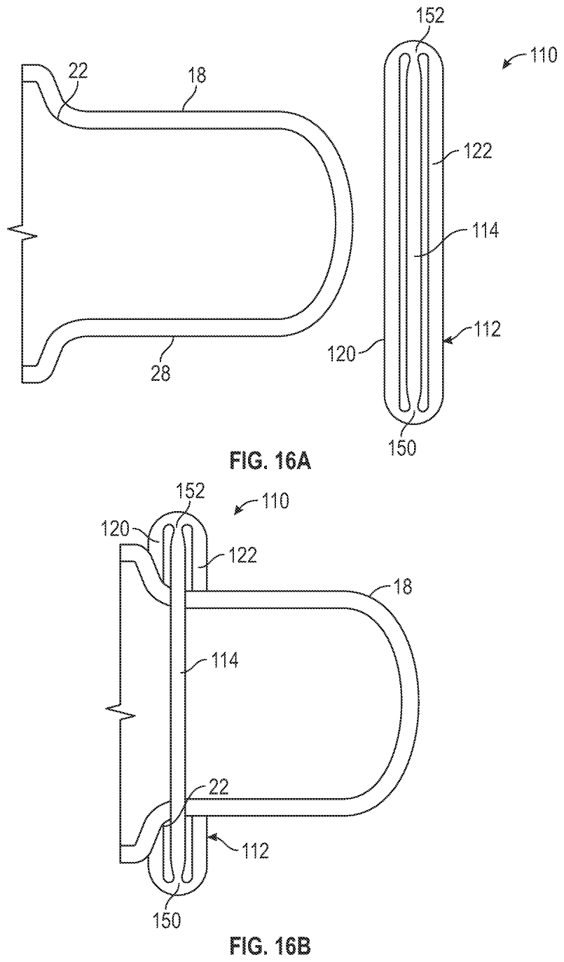

In operation, the clip 110 may be utilized to close the LAA 18. FIGS. 16A and 16B illustrate a deployment sequence of the clip 110.

FIG. 16A illustrates the clip 110 may approach the LAA 18 in an opened state. Access may be provided to the LAA 18 in a variety of manners, including a transcatheter access or a surgical access such as a thoracotomy, mini thoracotomy, or other methods of access.

In examples, a delivery apparatus may be utilized to move the clip 110 towards the LAA 18, or in examples, a user (e.g., a medical technician such as a surgeon, or other form of medical technician) may manually place the clip 110. The clip 110 may be placed over the outer surface 28 of the LAA 18 in an opened state. A user may confirm a desired placement of the clip 110 over the LAA 18.

The clip 110 may slide axially along the LAA 18, with the LAA 18 positioned within the space 156 shown in FIGS. 14 and 15 and the springs 150, 152 positioned on the sides of the LAA 18. The jaws 112, 114 may be positioned on opposite sides of the LAA 18.

With the clip 110 in the desired position, the clip 110 may be released and deployed to the LAA 18. The clip 110 may be moved to the closed state as shown in FIG. 16B. The LAA 18 may be compressed by the jaws 112, 114 of the clip 110 and closed.

The entire span of the LAA 18 may be closed as desired at the ostium or another portion of the LAA 18 may be closed.

In examples, if desired, the clip 110 may be opened and either removed or repositioned in a deployment procedure or another procedure. The clip 110 may be grasped and the force of the springs 150, 152 may be overcome to open the clip 110.

Various modifications of the clips disclosed herein may be provided. Various other methods of deployment and use of the clips may be provided as desired.

In examples, a cloth or other material may span the gap between the arms of the first jaw. The cloth or other material may allow for application of force to the portion of the heart by the cloth or other material.

In examples, the amount of force applied by the clips disclosed herein may be set by the shape set of the clips. The force applied by the clips may be tuned by the shape of the clip when set, and may be tuned by the material properties of the clips. A greater or lesser compressive force applied by the clip may be utilized as desired.

In examples, the clips may comprise a single body clip that applies the force to the portion of the heart. In examples, multiple bodies may be utilized to apply the force to the portion of the heart.

The clips as disclosed herein may be utilized to close the LAA or may be utilized to close another portion of a heart. In examples, the clips may be utilized to close other portions of a body, including other tubular vessels or other portions of a body. Deployment may be via a delivery apparatus or via another method as desired. Deployment may be via surgical methods and may be transcatheter or via non-invasive surgery in methods.

Variations in the clips and methods disclosed herein may be provided. Features across examples may be combined. Features may be excluded or added to in various examples disclosed herein. Combinations of features of examples may be provided. The features may be utilized solely or in methods disclosed herein, or in combination with other features disclosed herein.

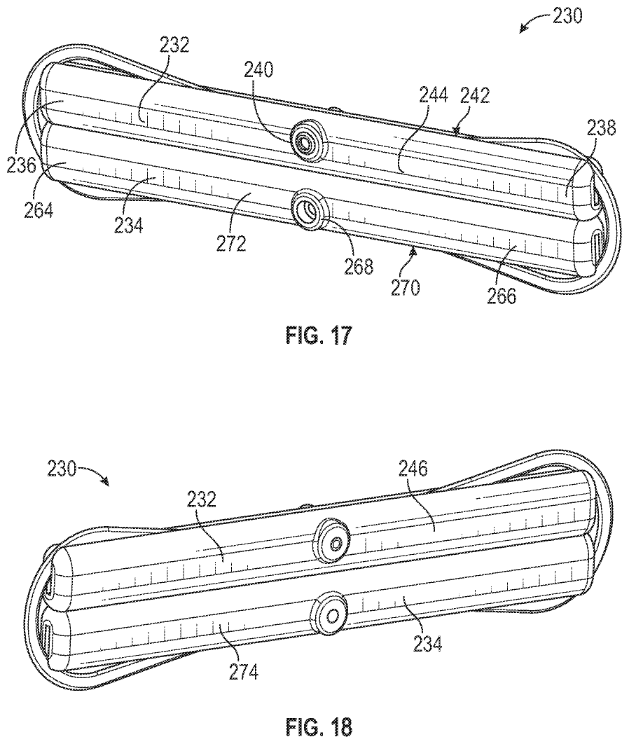

FIG. 17 illustrates an example of a clip 230 that may be utilized for a portion of a heart. The clip 230 may be configured to close a LAA 18, to reduce the possibility of clots or other undesired materials stemming from the LAA 18 from traveling into the bloodstream.

The clip 230 may include a first jaw 232 and a second jaw 234. The first jaw 232 may extend from a first end portion 236 to a second end portion 238 and may have an elongate shape. The first jaw 232 may be configured as an elongate beam. The first jaw 232 may have a central portion 240 between the first end portion 236 and the second end portion 238.

The first jaw 232 may include four sides, including an outer surface 242, a compression surface (facing opposite the outer surface 242 and towards the second jaw 234), and two side surfaces 244, 246 (with side surface 246 shown in FIG. 18) each extending between the compression surface and the outer surface 242.

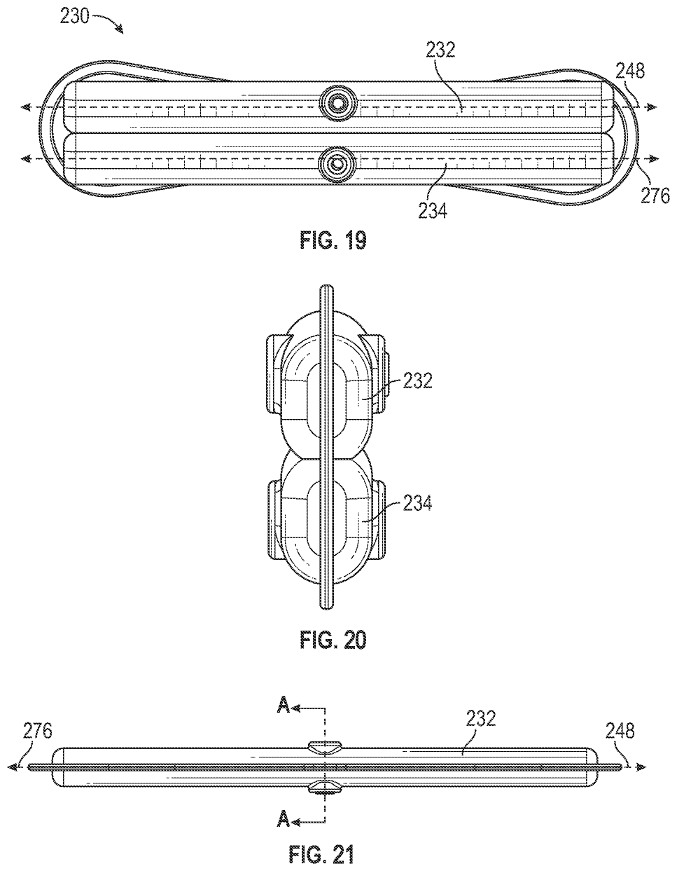

The first jaw 232 may extend along a longitudinal axis 248 (marked in FIGS. 19 and 21) and may have an oval cross section when viewed perpendicular to the longitudinal axis 248 (as visible in the end view of FIG. 20). The four sides of the first jaw 232 may each have flattened portions, and in examples, the first jaw 232 may be configured to have a rectangular shape as desired. The first jaw 232 may be rigid or may have an amount of flexibility in examples.

In examples, the first jaw 232 may include a channel 250 (marked in FIG. 23) that extends along the length of the first jaw 232 from the first end portion 236 to the second end portion 238. The channel 250 may include an interior wall 252 and side walls 254 that bound the channel 250. The portion of the channel 250 at the outer surface 242 of the first jaw 232 may be open for a spring 256 to pass through, and the portions of the channel 250 at the respective first end portion 236 and the second end portion 238 of the first jaw 232 may be open for the spring 256 to pass through.

FIG. 23 illustrates a cross sectional view of the first jaw 232 (and second jaw 234) at the center of the first jaw 232 and along line A-A in FIG. 21. A coupler 258 may be positioned within the channel 250 and between the side surfaces 244, 246 of the first jaw 232. The coupler 258 may be configured to couple to a spring 256. The coupler 258 may be positioned between the compression surface of the first jaw 232 and the outer surface 242 of the first jaw 232. The coupler 258 may be positioned centrally within the first jaw 232.

In examples, the coupler 258 may have a variety of forms. FIG. 23 illustrates the coupler 258 comprising a pin 260 extending through an aperture 262 in the spring 256. The coupler 258, for example, may allow loops of the spring 256 to pivot with respect to the pin 260. In examples, other forms of couplers such as clasps, or jaws may be utilized as desired.

Referring to FIG. 17, the second jaw 234 may extend from a first end portion 264 to a second end portion 266 and may have an elongate shape. The second jaw 234 may be configured as an elongate beam. The second jaw 234 may have a central portion 268 between the first end portion 264 and the second end portion 266.

The second jaw 234 may include four sides, including an outer surface 270, a compression surface (facing opposite the outer surface 270 and towards the first jaw 232), and two side surfaces 272, 274 (with side surface 274 shown in FIG. 18) each extending between the compression surface and the outer surface 270.

The second jaw 234 may extend along a longitudinal axis 276 (marked in FIGS. 19 and 21) and may have an oval cross section when viewed perpendicular to the longitudinal axis 276 (as visible in the end view of FIG. 20). The four sides of the second jaw 234 may each have flattened portions, and in examples, the second jaw 234 may be configured to have a rectangular shape as desired. The second jaw 234 may be rigid or may have an amount of flexibility in examples.

In examples, the second jaw 234 may include a channel 278 (marked in FIG. 23) that extends along the length of the second jaw 234 from the first end portion 264 to the second end portion 266. The channel 278 may include an interior wall 280 and side walls 282 that bound the channel 278. The portion of the channel 278 at the outer surface 270 of the second jaw 234 may be open for the spring 256 to pass through, and the portions of the channel 278 at the respective first end portion 264 and the second end portion 266 of the second jaw 234 may be open for the spring 256 to pass through.

FIG. 23 illustrates a cross sectional view of the second jaw 234 (and first jaw 232) at the center of the second jaw 234. A coupler 284 may be positioned within the channel 278 and between the side surfaces 272, 274 of the second jaw 234. The coupler 284 may be configured to couple to the spring 256. The coupler 284 may be positioned between the compression surface of the second jaw 234 and the outer surface 270 of the second jaw 234. The coupler 284 may be positioned centrally within the second jaw 234.

In examples, the coupler 284 may have a variety of forms. FIG. 23 illustrates the coupler 284 comprising a pin 286 extending through an aperture 288 in the spring 256. The coupler 284, for example, may allow loops of the spring 256 to pivot with respect to the pin 286. In examples, other forms of couplers such as clasps, or jaws may be utilized as desired.

The second jaw 234 may extend parallel with the first jaw 232. The first end portion 236 of the first jaw 232 may be aligned with the first end portion 264 of the second jaw 234, and the second end portion 238 of the first jaw 232 may be aligned with the second end portion 266 of the second jaw 234.

The clip 230 may be configured to move from an opened state to a closed state with the first jaw 232 moving relative to the second jaw 234 in a plane. A portion of the heart may be compressed between the first jaw 232 and the second jaw 234, and particularly between the compression surfaces of the first jaw 232 and the second jaw 234 when the first jaw 232 moves relatively towards the second jaw 234 in the plane.

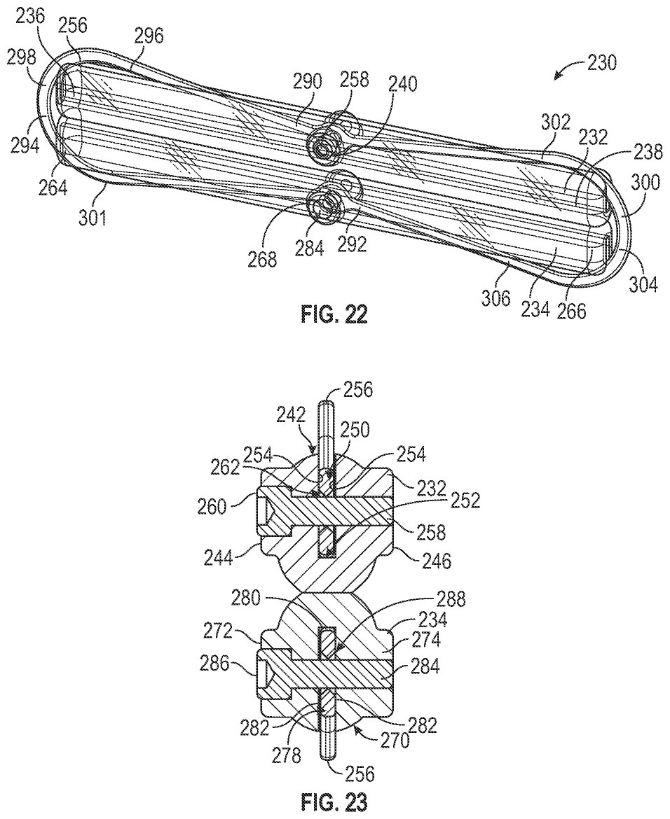

FIG. 22 illustrates a view of the clip 230 with the first jaw 232 and second jaw 234 shown in transparency and the spring 256 visible within the first jaw 232 and the second jaw 234. The spring 256 may be coupled to the central portion 240 of the first jaw 232 and the central portion 268 of the second jaw 234 and may be configured to force the first jaw 232 and the second jaw 234 together to compress the portion of the heart between the first jaw 232 and the second jaw 234.

The spring 256 may include a first central coupling portion 290 that couples to the coupler 258 of the first jaw 232. The spring 256 may include a second central coupling portion 292 that couples to the coupler 284 of the second jaw 234.

The spring 256 may include a first loop 294 that extends towards the first end portion 236 of the first jaw 232 and the first end portion 264 of the second jaw 234. The first loop 294 may couple the first central coupling portion 290 to the second central coupling portion 292. The first loop 294 may extend along the channel 250 of the first jaw 232 and the channel 278 of the second jaw 234. The first loop 294 may include a first straightened portion 296 that passes out of the channel 250 of the first jaw 232 and couples to a curved portion 298 that curves towards a second straightened portion 301 that passes out of the channel 278 of the second jaw 234. The curved portion 298 may protrude from the respective first end portions 236, 264 of the first jaw 232 and the second jaw 234.

The spring 256 may include a second loop 300 that extends towards the second end portion 238 of the first jaw 232 and the second end portion 266 of the second jaw 234. The second loop 300 may couple the first central coupling portion 290 to the second central coupling portion 292. The second loop 300 may extend along the channel 250 of the first jaw 232 and the channel 278 of the second jaw 234. The second loop 300 may include a first straightened portion 302 that passes out of the channel 250 of the first jaw 232 and couples to a curved portion 304 that curves towards a second straightened portion 306 that passes out of the channel 278 of the second jaw 234. The curved portion 304 may protrude from the respective second end portions 238, 266 of the first jaw 232 and the second jaw 234.

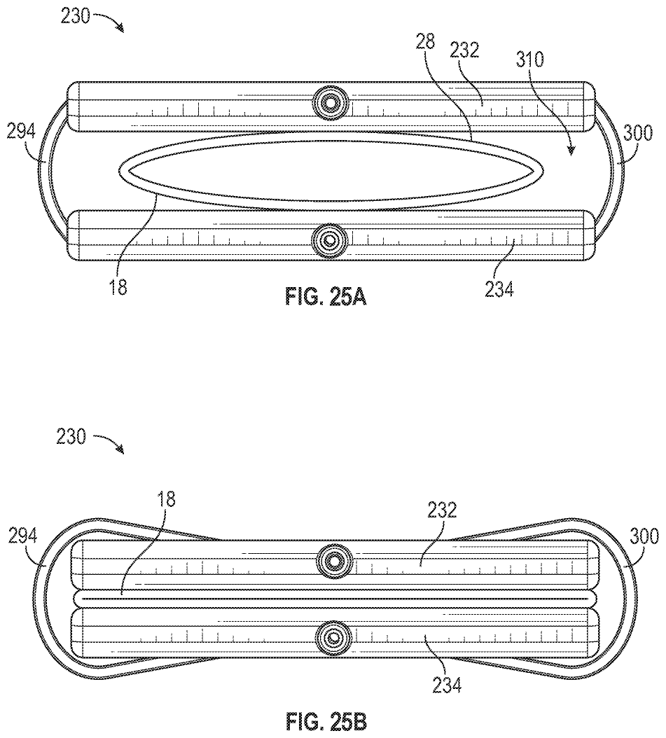

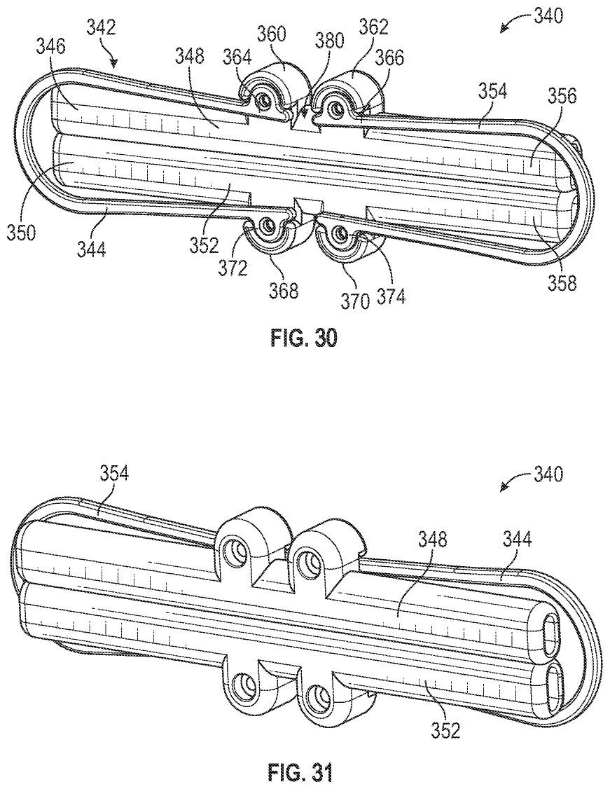

The spring 256 may comprise a continuous body in examples, or in examples may include multiple spring bodies (as shown in FIGS. 30-32) for example. The spring 256 may have a smaller diameter at the central coupling portions 290, 292 than at the curved portions 298, 304, thus forming a bowtie or hourglass shape with larger ends as shown in FIG. 22 for example. The spring 256 may be configured to deflect to accommodate the clip 230 being in an opened state. As such, the straightened portions 296, 301, 302, 306 may be configured to deflect further into the respective channels 250, 278 to allow the clip 230 to be positioned in an opened state (as shown in FIG. 25A for example).

The spring 256 may be positioned in the plane of movement of the first jaw 232 and second jaw 234 and configured to apply a central force to the first jaw 232 and the second jaw 234 to move the clip 230 to the closed state.

The spring 256 may allow the first jaw 232 and the second jaw 234 to move to an opened state (as shown in FIG. 25A for example) such that the clip 230 may be slid over the portion of the heart being compressed (e.g., the LAA 18) in a direction transverse to the axis 248 of the first jaw 232 and the axis 276 of the second jaw 234 (marked in FIG. 19). The portion of the heart may be positioned within the space 310 between the first jaw 232 and the second jaw 234 (as marked in FIG. 25A for example).

The first jaw 232, the second jaw 234 and the spring 256 may bound the space 310 between the first jaw 232 and the second jaw 234. For example, the first loop 294 may form a boundary of the space 310 (as marked in FIG. 25A for example), and the second loop 300 may form an opposite boundary of the space 310. The loops 294, 300 accordingly may reduce the possibility of tissue passing out of the space 310 during compression of the portion of the heart.

FIG. 18 illustrates a perspective view of the clip 230 on a side opposite the side shown in FIG. 17. FIG. 19 illustrates a side view of the clip 230. FIG. 20 illustrates an end view of the clip 230. FIG. 21 illustrates a top view of the clip 230. FIG. 22 illustrates a perspective view of the clip 230 with the jaws 232, 234 shown in transparency. FIG. 23 illustrates a cross sectional view of the clip 230 along line A-A in FIG. 21.

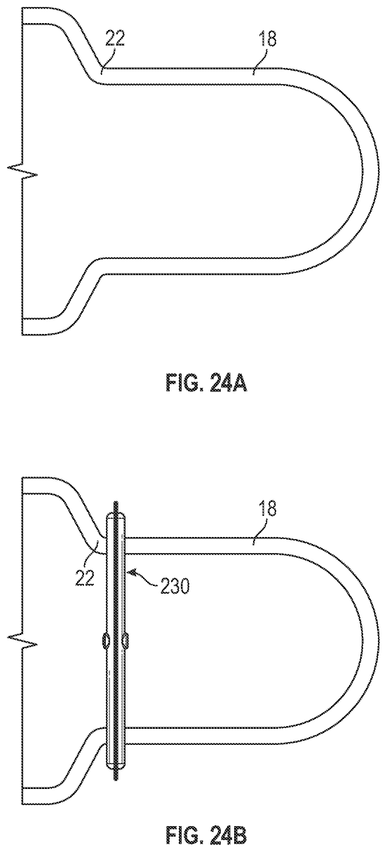

In operation, the clip 230 may be utilized to close the LAA 18. FIG. 24A illustrates a top schematic view of the LAA 18 prior to the clip 230 being applied. The clip 230 may be applied by the clip 230 being moved to the opened state and slid along the LAA 18 along the axis of the LAA 18. FIG. 24B illustrates the clip 230 deployed to the LAA 18 at the ostium 22 of the LAA 18.

FIG. 25A illustrates the clip 230 positioned around the LAA 18 in an opened state. With the clip 230 in the desired position, the clip 230 may be moved to the closed state as shown in FIG. 25B. The LAA 18 may be compressed by the compressive surfaces of the jaws 232, 234 of the clip 230 and closed. Access may be provided to the LAA 18 in a variety of manners, including a transcatheter access or a surgical access such as a thoracotomy, mini thoracotomy, or other methods of access.

In examples, a delivery apparatus may be utilized to move the clip 230 towards the LAA 18, or in examples, a user (e.g., a medical technician such as a surgeon, or other form of medical technician) may manually place the clip 230. The clip 230 may be placed over the outer surface 28 of the LAA 18 in an opened state. A user may confirm a desired placement of the clip 230 over the LAA 18.

The entire span of the LAA 18 may be closed as desired at the ostium (as shown in FIG. 24B) or another portion of the LAA 18 may be closed.

In examples, if desired, the clip 230 may be opened and either removed or repositioned in a deployment procedure or another procedure. The clip 230 may be grasped and the force of the spring 256 may be overcome to open the clip 230.

The clip 230 may be utilized to close the LAA 18, yet in examples other portions of a heart may be clipped or closed via use of the clip 230. In examples, other portions of a body, such as a tubular vessel or other portions of a body may be closed with the clip 230. Deployment may be via a delivery apparatus or via another method as desired.

Variations in the configuration of the clip 230 may be provided as desired.

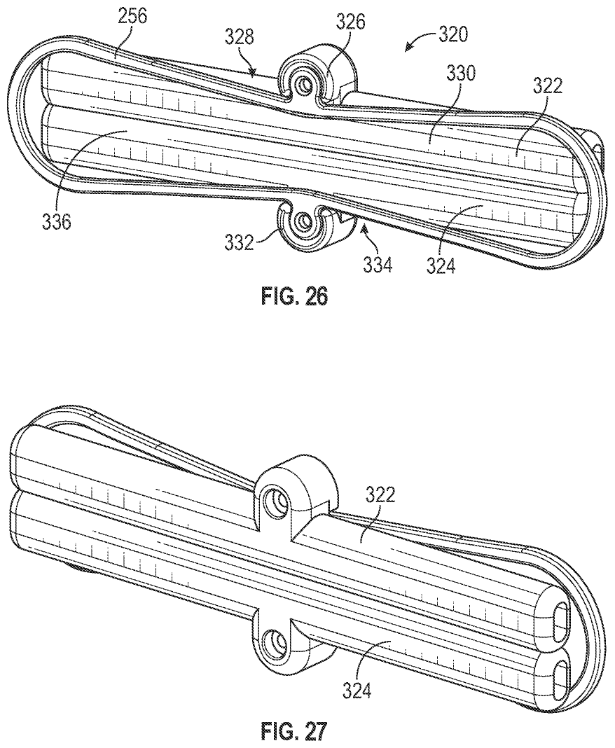

FIGS. 26-29, for example, illustrates an example of a clip 320 in which the spring 256 is positioned offset from a plane of the first jaw 322 and the second jaw 324. The first jaw 322, for example, may include a coupler 326 protruding outward from the outer surface 328 of the first jaw 322 and protruding from a side surface 330 of the first jaw 322. The coupler 326 may couple to the spring 256 outside of the first jaw 322. Similarly, the second jaw 324 may include a coupler 332 protruding outward from the outer surface 334 of the second jaw 324 and protruding from a side surface 336 of the second jaw 324. The coupler 332 may couple to the spring 256 outside of the second jaw 324. In such a configuration, the spring 256 may compress the jaws 322, 324 together, yet offset from the plane of movement of the jaws 322, 324. The spring 256 may extend in a plane that is parallel with the plane of movement of the jaws 322, 324.

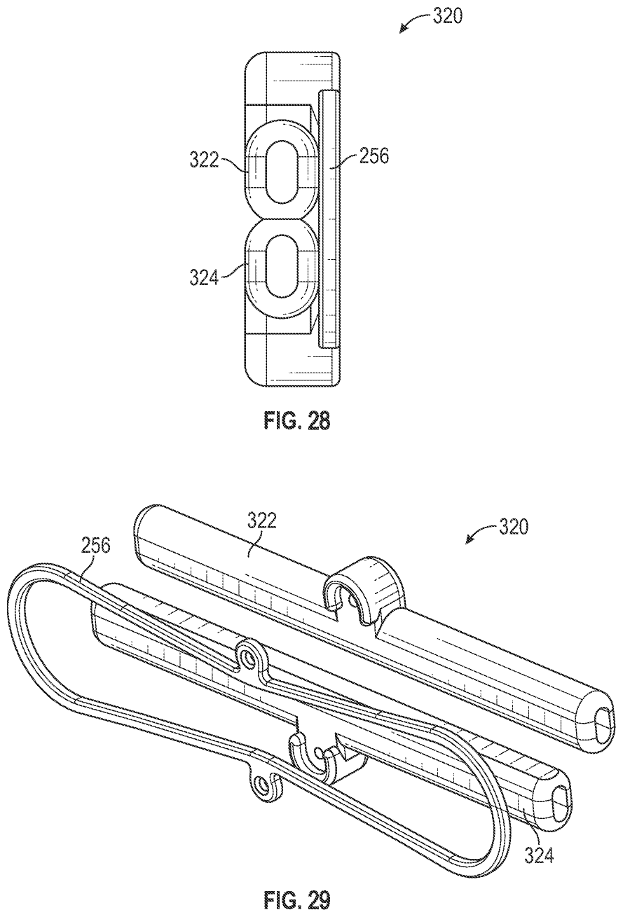

FIG. 27 illustrates a side perspective view of the clip 320 at an opposite side than shown in FIG. 26. FIG. 28 illustrates an end view of the clip 320 shown in FIG. 26 with the offset position of the spring 256 shown. FIG. 29 illustrates an assembly view of the clip 320.

FIGS. 30-32 illustrate a variation of the clip 320 shown in FIGS. 26-29, in which the clip 340 includes a spring 342 having a first spring body 344 extending towards the first end portion 346 of the first jaw 348 and the first end portion 350 of the second jaw 352, and a second spring body 354 extending towards the second end portion 356 of the first jaw 348 and the second end portion 358 of the second jaw 352. The first jaw 348 includes couplers 360, 362 for respective ends 364, 366 of the first spring body 344 and the second spring body 354, and the second jaw 352 includes couplers 368, 370 for ends 372, 374 of the first spring body 344 and the second spring body 354. A gap 380 is positioned between the first spring body 344 and the second spring body 354. The spring 342 may provide a similar function as disclosed herein of forcing the first jaw 348 relatively towards the second jaw 352 to compress a body therein. The spring 342 is offset from the plane of movement of the first jaw 348 and the second jaw 352 and may extend in a plane that is parallel with the plane of movement of the jaws 348, 352.

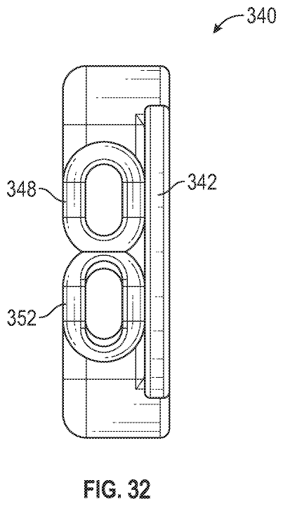

FIG. 31 illustrates a side perspective view of the clip 340 at an opposite side than shown in FIG. 30. FIG. 32 illustrates an end view of the clip 340 shown in FIG. 30 with the offset position of the spring 342 shown.

Variations in the configuration of the clips disclosed herein may be provided as desired.

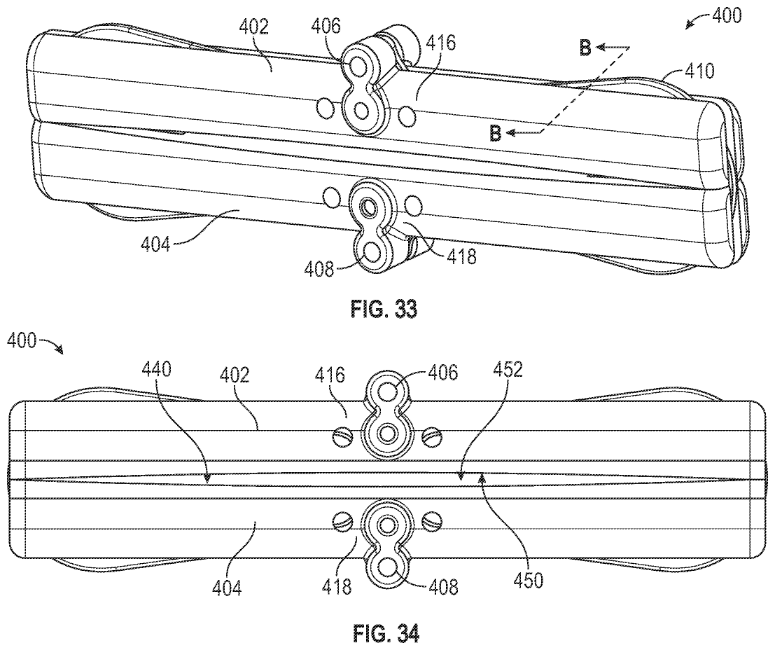

FIGS. 33-36C, for example, illustrate a clip 400 that comprises a variation of the clip 230 shown in FIGS. 17-25B. Features of the clip 230 shown in FIGS. 17-25B apply to the clip 400 shown in FIGS. 33-36C unless stated otherwise. The clip 400 may include a first jaw 402 and a second jaw 404 that may be configured similarly as the respective first jaw 232 and the second jaw 234 of the clip 230. The first jaw 402, however, may include an aperture 406 for receiving a portion of a delivery apparatus for the clip 400. The aperture 406, for example, may receive a protrusion of a delivery apparatus that may enter the aperture 406. The protrusion may comprise a jaw of a grasper of the delivery apparatus, or may have another configuration as desired.

The second jaw 404 may include an aperture 408 that may be for receiving a portion of a delivery apparatus for the clip 400. The aperture 408 of the second jaw 404 may be configured similarly as the aperture 406 of the first jaw 402, and may be configured to receive a protrusion of a delivery apparatus that may enter the aperture 408. The protrusions of the delivery apparatus may enter respective apertures 406, 408 of the first jaw 402 and second jaw 404 and may move the first jaw 402 and second jaw 404 away from each other during deployment as desired. The protrusions of the delivery apparatus may be closed or released to allow the clip 400 to close.

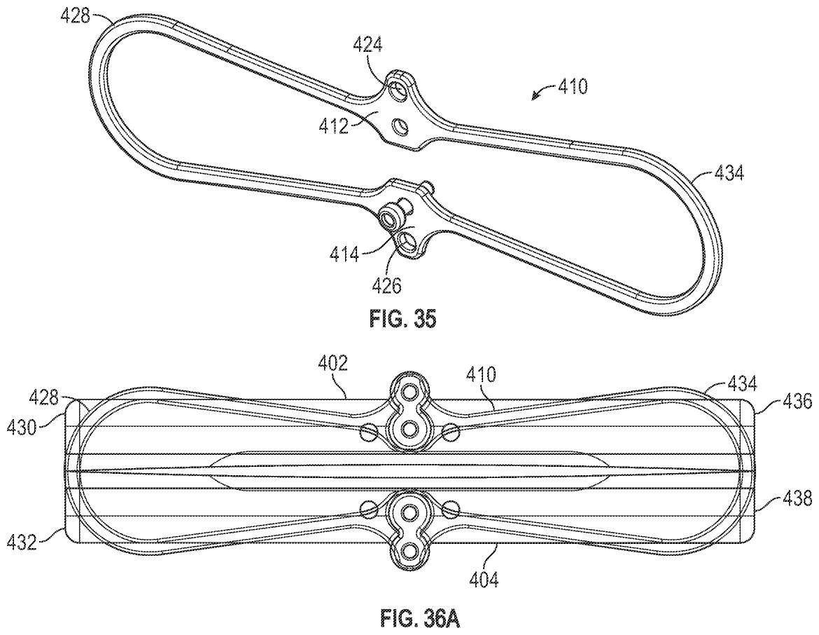

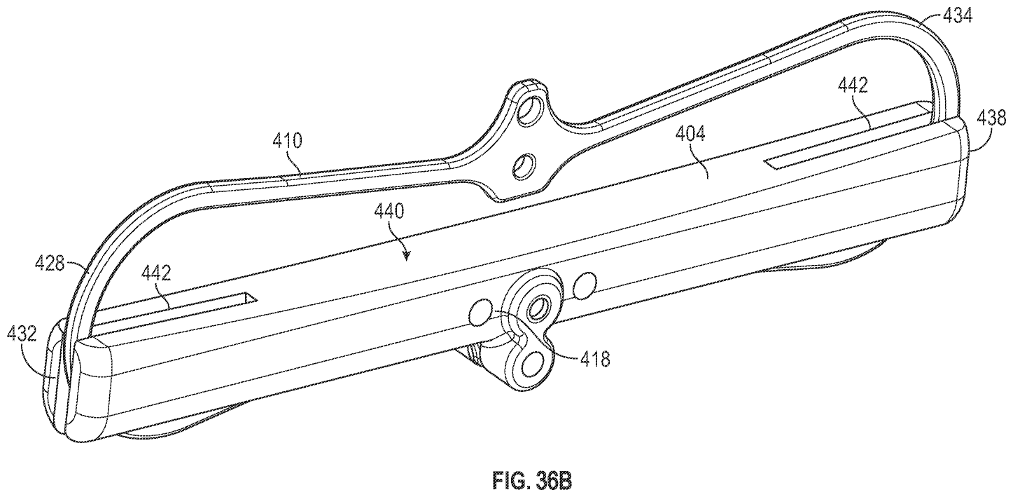

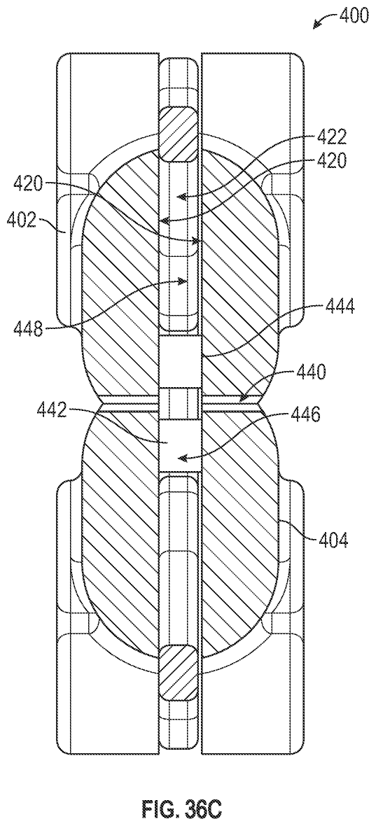

The spring 410 (more clearly shown in FIG. 35) may be configured similarly as the spring 256 of the clip 230 unless stated otherwise. The spring 410 may include a first central coupling portion 412 and a second central coupling portion 414. The first central coupling portion 412 may comprise a first flat plate portion that may be positioned at a central portion 416 (marked in FIG. 33) of the first jaw 402. The second central coupling portion 414 may comprise a second flat plate portion that may be positioned at a central portion 418 (marked in FIG. 33) of the second jaw 404. The flat plate portions of the spring 410 may contact the side walls 420 of the channel 422 (marked in FIG. 36C) to reduce the possibility of twisting of the spring 410 during opening or closing of the spring 410.

Referring to FIG. 35, the first flat plate portion of the first central coupling portion 412 may include an aperture 424 that may align with the aperture 406 of the first jaw 402. The second flat plate portion of the second central coupling portion 414 may include an aperture 426 that may align with the aperture 408 of the second jaw 404.

Referring to FIG. 36A, the first jaw 402 and the second jaw 404 are shown in transparency to show the relative position of the spring 410. The spring 410 may include a first loop 428 extending towards the first end portion 430 of the first jaw 402 and the first end portion 432 of the second jaw 404. The spring 410 may include a second loop 434 that may extend towards the second end portion 436 of the first jaw 402 and the second end portion 438 of the second jaw 404. The first end portion 430 of the first jaw 402 and the first end portion 432 of the second jaw 404 are each positioned at or protrude from the first loop 428. The second end portion 436 of the first jaw 402 and the second end portion 438 of the second jaw 404 are each positioned at or protrude from the second loop 434. As such, upon the clip 400 being opened, the loops 428, 434 may be positioned at or within the outer extent of the ends of the jaws 402, 404 to reduce the possibility of tissue being trapped or pinned between the ends of the jaws 402, 404 and the loops 428, 434 of the spring 410.

To facilitate movement of the spring 410 during opening of the clip 400, the compression surfaces of the first jaw 402 or the second jaw 404 may include one or more slots to allow the spring 410 to pass through. For example, FIG. 36B illustrates a view of the clip with the first jaw excluded from view. The compression surface 440 of the second jaw 404 may include slots 442 that may extend inward from the respective end portions 432, 438 of the second jaw 404 towards the central portion 418 of the second jaw 404. The first jaw 402 may include similar slots. The slots may allow the loops 428, 434 to deflect inward towards the center of the clip 400 upon opening of the clip 400. FIG. 36C, for example, illustrates a cross sectional view of the clip 400 along line B-B in FIG. 33. A slot 442 of the second jaw 404 and a slot 444 of the first jaw 402 may extend from the respective channels 446, 448 of the second jaw 404 and the first jaw 402.

Referring to FIG. 34, the compression surface 440 of the second jaw 404 may face towards a compression surface 450 of the first jaw 402. The compression surface 450 of the first jaw 402 may be curved away from the second jaw 404. The compression surface 440 of the second jaw 404 may be curved away from the first jaw 402. As such, a gap 452 may be positioned between the compression surfaces 440, 450 when the clip 400 is in a closed state. The gap 452 may have an ovoid shape or other shape based on the curvature of the respective compression surfaces 450, 440. The curvature of the compression surfaces 450, 440 may allow the jaws 402, 404 to accommodate a shape of the portion of the body being compressed.

Various other modifications of the clips disclosed herein may be provided. Various other methods of deployment and use of the clips may be provided as desired.

The clips as disclosed herein may be utilized to close the LAA or may be utilized to close another portion of a heart. In examples, the clips may be utilized to close other portions of a body, including other tubular vessels or other portions of a body. Deployment may be via a delivery apparatus or via another method as desired. Deployment may be via surgical methods and may be transcatheter or via non-invasive surgery in methods.

Variations in the clips and methods disclosed herein may be provided. Features across examples may be combined. Features may be excluded or added to in various examples disclosed herein. Combinations of features of examples may be provided. The features may be utilized solely or in methods disclosed herein, or in combination with other features disclosed herein.

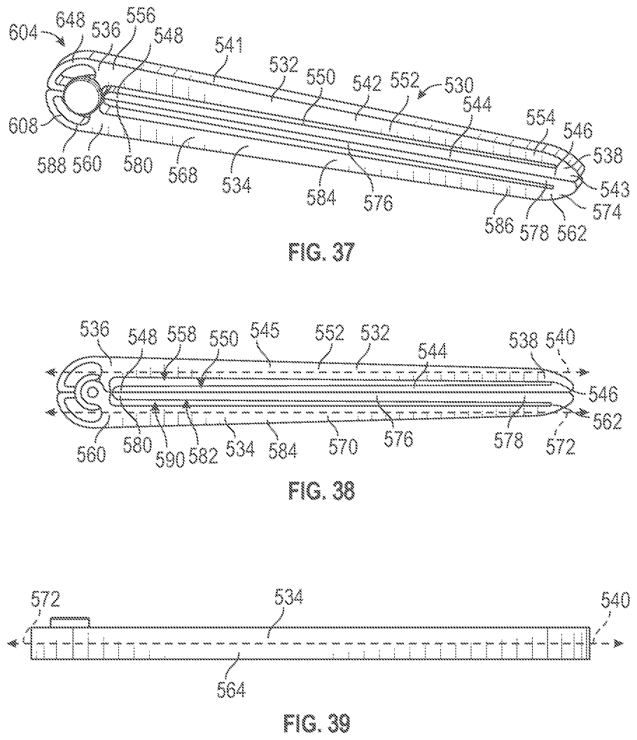

FIG. 37 illustrates an example of a clip 530 that may be utilized for a portion of a heart. The clip 530 may be configured to close a LAA 18, to reduce the possibility of clots or other undesired materials stemming from the LAA 18 from traveling into the bloodstream.

The clip 530 may include a first jaw 532 and a second jaw 534. The first jaw 532 may extend from a first end portion 536 to a second end portion 538 and may have an elongate shape. The first jaw 532 may include four sides, including an outer surface 541, a compression surface (opposite the outer surface 541), and two side surfaces 542, 545 (with the side surface 545 being marked in FIG. 38).

The first jaw 532 may extend along a longitudinal axis 540 (marked in FIGS. 38 and 39) and may taper downward in thickness from the first end portion 536 of the first jaw 532 to the second end portion 538 of the first jaw 532. The first jaw 532 may have a rectangular profile when viewed in cross section perpendicular to the longitudinal axis 540. The second end portion 538 of the first jaw 532 may comprise a tip 543 of the first jaw 532.

The first jaw 532 may include a first cantilever beam 544 extending from the second end portion 538 of the first jaw 532 towards the first end portion 536 of the first jaw 532. The first cantilever beam 544 may extend from the tip 543 of the first jaw 532 towards the first end portion 536 of the first jaw 532. The first cantilever beam 544 may include a first end 546 and a second end 548 and may taper downward in a direction from the first end 546 to the second end 548 of the first cantilever beam 544. A thickness of the first cantilever beam 544 may taper downward in a direction towards the first end portion 536 of the first jaw 532. The second end 548 of the first cantilever beam 544 may be free to deflect in a direction transverse to the longitudinal axis 540 of the first jaw 532. The first cantilever beam 544 may be unsupported along its length from the first end 546 to the second end 548 aside from its coupling to the tip 543 of the first jaw 532.

The first cantilever beam 544 may include the compression surface of the first jaw 532 and may include an outer surface 550 opposite the compression surface. The first cantilever beam 544 may have a rectangular profile when viewed in cross section perpendicular to the longitudinal axis 540 of the first jaw 532.

The first jaw 532 may include a first arm 552 positioned outward of the first cantilever beam 544 and extending from the first end portion 536 of the first jaw 532 to the second end portion 538 of the first jaw 532. The first arm 552 includes a first end 554 at the second end portion 538 of the first jaw 532 and a second end 556 at the first end portion 536 of the first jaw 532. The first arm 552 may extend from the second end 556 to the first end 554 and may taper downward in a direction from the second end 556 to the first end 554. The first end 554 of the first arm 552 may couple to the first end 546 of the first cantilever beam 544 at the tip 543 of the first jaw 532.

The first arm 552 may include the outer surface 541 of the first jaw 532 and may include an inner surface that faces opposite the outer surface 541 and towards the outer surface 550 of the first cantilever beam 544. The first arm 552 may include side surfaces that extend from the inner surface to the outer surface 541 of the first jaw 532, and may have a rectangular profile when viewed in cross section perpendicular to the longitudinal axis 540.

The first arm 552 may be spaced from the first cantilever beam 544 with a gap 558 (marked in FIG. 38). The thickness of the gap 558 may decrease in a direction from the first end portion 536 of the first jaw 532 towards the second end portion 538 of the first jaw 532. The gap 558 may be closed by the tip 543 of the first jaw 532 and may open into a space between the first jaw 532 and the second jaw 534 at the first end portion 536 of the first jaw 532. The gap 558 may be configured to receive the first cantilever beam 544 as the first cantilever beam 544 is deflected outward in a direction towards the first arm 552 during closure of the clip 530, for example.

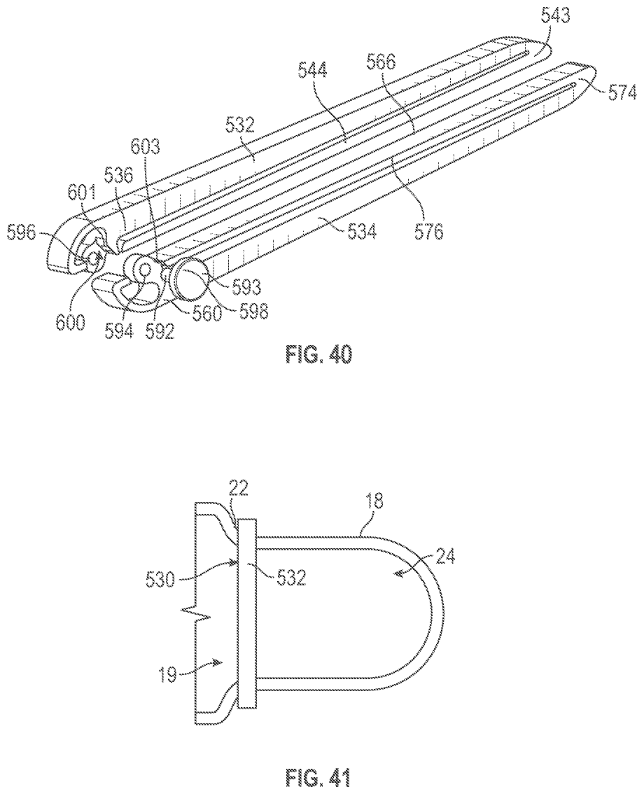

The second jaw 534 may be configured similarly as the first jaw 532 in examples. For example, the second jaw 534 may extend from a first end portion 560 to a second end portion 562 and may have an elongate shape. The second jaw 534 may include four sides, including an outer surface 564 (marked in FIG. 39), a compression surface 566 (opposite the outer surface 564 and marked in FIG. 40), and two side surfaces 568, 570 (marked in FIGS. 37 and 38).

The second jaw 534 may extend along a longitudinal axis 572 (marked in FIGS. 38 and 39) and may taper downward in thickness from the first end portion 560 of the second jaw 534 to the second end portion 562 of the second jaw 534. The second jaw 534 may have a rectangular profile when viewed in cross section perpendicular to the longitudinal axis 572. The second end portion 562 of the second jaw 534 may comprise a tip 574 of the second jaw 534.

The second jaw 534 may include a second cantilever beam 576 extending from the second end portion 562 of the second jaw 534 towards the first end portion 560 of the second jaw 534. The second cantilever beam 576 may extend from the tip 574 of the second jaw 534 towards the first end portion 560 of the second jaw 534. The second cantilever beam 576 may include a first end 578 and a second end 580 and may taper downward in a direction from the first end 578 to the second end 580 of the second cantilever beam 576. A thickness of the second cantilever beam 576 may taper downward in a direction towards the first end portion 560 of the second jaw 534. The second end 580 of the second cantilever beam 576 may be free to deflect in a direction transverse to the longitudinal axis 572 of the second jaw 534. The second cantilever beam 576 may be unsupported along its length from the first end 578 to the second end 580 aside from its coupling to the tip 574 of the second jaw 534.

The second cantilever beam 576 may include the compression surface 566 of the second jaw 534 and may include an outer surface 582 opposite the compression surface 566. The second cantilever beam 576 may have a rectangular profile when viewed in cross section perpendicular to the longitudinal axis 572 of the second jaw 534.

The second jaw 534 may include a second arm 584 positioned outward of the second cantilever beam 576 and extending from the first end portion 560 of the second jaw 534 to the second end portion 562 of the second jaw 534. The second arm 584 includes a first end 586 at the second end portion 562 of the second jaw 534 and a second end 588 at the first end portion 560 of the second jaw 534. The second arm 584 may extend from the second end 588 to the first end 586 and may taper downward in a direction from the second end 588 to the first end 586. The first end 586 of the second arm 584 may couple to the first end 578 of the second cantilever beam 576 at the tip 574 of the second jaw 534.

The second arm 584 may include the outer surface 564 of the second jaw 534 and may include an inner surface that faces opposite the outer surface 564 and towards the outer surface 582 of the second cantilever beam 576. The second arm 584 may include side surfaces that extend from the inner surface to the outer surface 564 of the second jaw 534, and may have a rectangular profile when viewed in cross section perpendicular to the longitudinal axis 572.

The second arm 584 may be spaced from the second cantilever beam 576 with a gap 590 (marked in FIG. 38). The thickness of the gap 590 may decrease in a direction from the first end portion 560 of the second jaw 534 towards the second end portion 562 of the second jaw 534 (more clearly visible in FIG. 38). The gap 590 may be configured to receive the second cantilever beam 576 as the second cantilever beam 576 is deflected outward in a direction towards the second arm 584 during closure of the clip 530, for example.

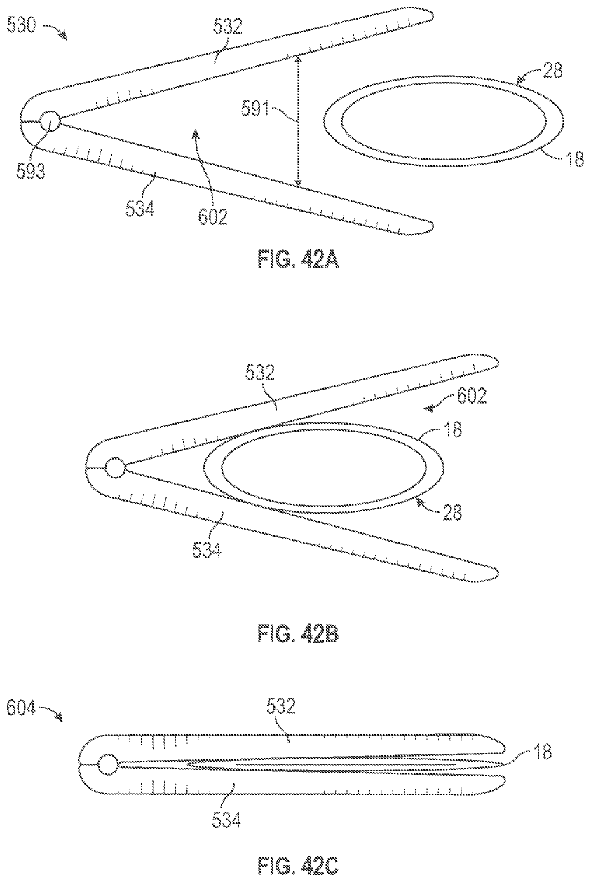

The first end portion 560 of the second jaw 534 may be pivotally coupled to the first end portion 536 of the first jaw 532 such that a distance 591 (as marked in FIG. 42A) between the first cantilever beam 544 and the second cantilever beam 576 may be reduced to compress a portion of a heart between the first cantilever beam 544 and the second cantilever beam 576. The pivotal coupling may be provided in a variety of manners, including an axle 592 (as marked in FIG. 40) that the first jaw 532 and second jaw 534 may pivot about, or a hinge, or a flexible joint between the first jaw 532 and the second jaw 534, among other manners. In examples, the pivotal coupling may be provided as a flexible joint between the first jaw 532 and the second jaw 534, with the first jaw 532 being a single body with the second jaw 534.

As shown in FIG. 40, the pivotal coupling may comprise an axle 592 of a pivot pin 593 that may pass through an aperture 594 in the first end portion 560 of the second jaw 534 and an aperture 596 in the first end portion 536 of the first jaw 532. The apertures 594, 596 may be adjacent to each other to allow the axle 592 to pass through. The pivot pin 593 may include a head 598 that may impede further movement of the axle 592 distally through the apertures 594, 596. In assembly, the first end portion 560 of the second jaw 534 may be inserted into a recess 600 of the first end portion 536 of the first jaw 532, and the pivot pin 593 may be passed through the apertures 594, 596. The first end portion 536 of the first jaw 532 may include a support surface 601 that may press against a stop surface 603 on the first end portion 560 of the second jaw 534 to impede closure of the clip 530 further than a desired point.

The first end portion 560 of the second jaw 534 may be pivotally coupled to the first end portion 536 of the first jaw 532 such that a distance between the tip 543 of the first jaw 532 and the tip 574 of the second jaw 534 varies. The second jaw 534 may pivot in an are relative to the first jaw 532 such that the clip 530 may move from an opened state (as shown in FIGS. 42A and 42B) to a closed state (as shown in FIG. 42C).

The compression surface of the first jaw 532 may be spaced from the compression surface 566 of the second jaw 534 in the opened state, such that a portion of the heart may be positioned between the compression surfaces. The tips 543, 574 of the first jaw 532 and the second jaw 534 may open such that an axial opening of a channel 602 (as marked in FIG. 42A) between the compression surfaces of the first jaw 532 and the second jaw 534 is formed or increased in size. A portion of a heart may be inserted into the axial opening.

The compression surfaces of the first jaw 532 and the second jaw 534 may be moved towards each other in the closed state. The first cantilever beam 544 and the second cantilever beam 576 may each deflect outward upon compression of the portion of the heart between the first cantilever beam 544 and the second cantilever beam 576.

Referring to FIG. 37, a spring 604 may be provided that may be configured to force the first jaw 532 towards the second jaw 534 and the first cantilever beam 544 towards the second cantilever beam 576. The spring 604 in examples may be positioned at the first end portion 536 of the first jaw 532 and at the first end portion 560 of the second jaw 534 in examples, although other positions may be utilized as desired. The spring 604 may include a single spring body or multiple spring bodies, with an upper spring body 606 and a lower spring body 608 shown in FIG. 37. The upper spring body 606 may be integral with the first jaw 532 in examples and the lower spring body 608 may be integral with the second jaw 534.

The spring 604 may comprise an arcuate body, as shown in FIG. 37. The arcuate body may be integral with one or more of the first jaw 532 or the second jaw 534 in examples. For example, the upper spring body 606 may comprise an arcuate body integral with the first jaw 532 and the lower spring body 608 may comprise an arcuate body integral with the second jaw 534. The arcuate body may protrude from the first end portion 536 of the first jaw 532 and the first end portion 560 of the second jaw 534 in a direction away from the respective second end portions 538, 562 of the first jaw 532 and second jaw 534. The upper spring body 606 and lower spring body 608 may form a curved or circular profile of an end of the clip 530.

The spring 604 may force the first jaw 532 towards the second jaw 534 due to pressure of the upper spring body 606 against the lower spring body 608 when the clip 530 is moved to the opened state (as marked in FIG. 42A). The spring may apply a force to the first jaw 532 and the second jaw 534 to rotate the first jaw 532 and the second jaw 534 to the closed state, for compression of the portion of the heart between the first jaw 532 and second jaw 534.

The configuration of the first jaw 532 and second jaw 534 may allow the force applied by the spring 604 to be more evenly distributed along the length of the compression surfaces of the first jaw 532 and the second jaw 534. For example, the coupling of the arms 552, 584 to the respective first and second cantilever beams 544, 576 at the tips 543, 574 of the jaws 532, 534 may allow for the force of the spring to be applied at the tips 543, 574 and also provided along the first and second cantilever beams 544, 576. The tapering of the first and second cantilever beams 544, 576 may be provided for a more even application of the force from the spring 604. Such a feature may reduce the possibility of the force from the spring 604 being great at the first end portions 536, 560 of the jaws 532, 534 and then significantly being reduced at the tips 543, 574, which may provide for incomplete closure of the portion of the heart at the tips 543, 574. The configuration of the jaws 532, 534 may allow for a more complete and even closure along the portion of the heart and along the entirety of the jaws 532, 534.

FIG. 38 illustrates a side view of an opposite side of the clip 530 than shown in FIG. 37. FIG. 39 illustrates a bottom view of the clip 530. FIG. 40 illustrates a perspective assembly view of the clip 530 illustrating the first jaw 532 separated from the second jaw 534 and the pivot pin 593 separated from both jaws 532, 534.

In operation, the clip 530 may be utilized to close the LAA 18. FIG. 41 illustrates a top schematic view of the clip 530 closing or occluding the LAA 18, and being positioned at the ostium 22 of the LAA 18. The clip 530 may span the ostium 22 to close the ostium 22 and reduce blood flow between the LAA 18 and the main chamber 19 of the left atrium 10.

FIGS. 42A-42C illustrate an exemplary deployment of the clip 530. Features of the clip 530 may be excluded from view in FIGS. 42A-42C. In FIG. 42A, the clip 530 may be placed in an opened state and moved towards the LAA 18 in a direction transverse to the main axis of the LAA 18. In examples, other approaches (e.g., along the main axis of the LAA 18) may be utilized as desired. Access may be provided to the LAA 18 in a variety of manners, including a transcatheter access or a surgical access such as a thoracotomy, mini thoracotomy, or other methods of access.

In FIG. 42B, the clip 530 may be placed over the outer surface 528 of the LAA 18. In examples, a delivery apparatus may be utilized to move the clip 530 towards the LAA 18, or in examples, a user (e.g., a medical technician such as a surgeon, or other form of medical technician) may manually place the clip 530. The clip 530 may be placed over the outer surface 28 of the LAA 18 in an opened state. A user may confirm a desired placement of the clip 530 over the LAA 18.

In FIG. 42C, the clip 530 may be moved to a closed state with the LAA 18 compressed between the first jaw 532 and the second jaw 534. The spring 604 may force the clip 530 to the closed state. The entire span of the LAA 18 may be closed as desired at the ostium (as shown in FIG. 41) or another portion of the LAA 18 may be closed.

In examples, if desired, the clip 530 may be opened and either removed or repositioned in a deployment procedure or another procedure. The clip 530 may be grasped and the force of the spring 604 may be overcome to open the clip 530.

The clip 530 may be utilized to close the LAA 18, yet in examples other portions of a heart may be clipped or closed via use of the clip 530. In examples, other portions of a body, such as a tubular vessel or other portions of a body may be closed with the clip 530. Deployment may be via a delivery apparatus or via another method as desired.

Variations in the configuration of the clip 530 may be provided as desired.

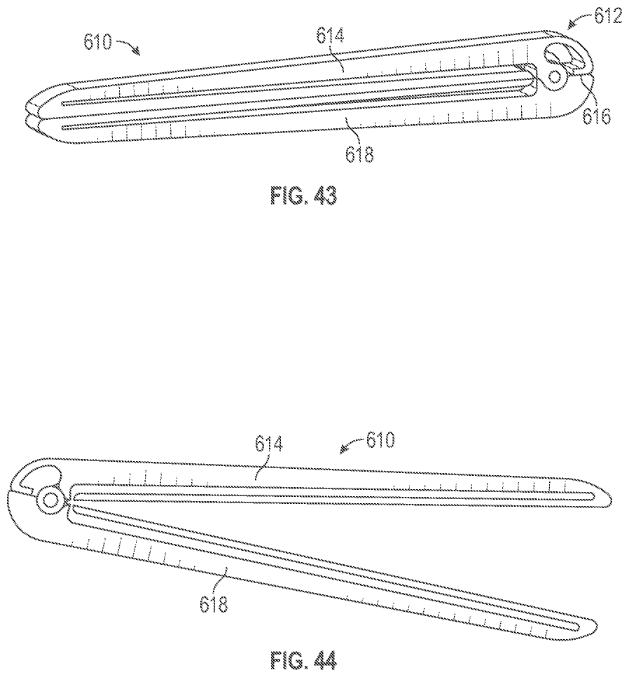

FIGS. 43-44, for example, illustrate a clip 610 configured similarly as the clip 530, yet including a spring 612 having only a single body. The spring 612 may be integral with the first jaw 614 and may abut a support surface 616 of the second jaw 618 that does not comprise a spring. The spring 612 may be configured to force the first jaw 614 and the second jaw 618 together in a similar manner as the spring 604. FIG. 44 illustrates the clip 610 in an opened state.

Various other modifications of the clips disclosed herein may be provided. Various other methods of deployment and use of the clips may be provided as desired.

The clips as disclosed herein may be utilized to close the LAA or may be utilized to close another portion of a heart. In examples, the clips may be utilized to close other portions of a body, including other tubular vessels or other portions of a body. Deployment may be via a delivery apparatus or via another method as desired. Deployment may be via surgical methods and may be transcatheter or via non-invasive surgery in methods.

Variations in the clips and methods disclosed herein may be provided. Features across examples may be combined. Features may be excluded or added to in various examples disclosed herein. Combinations of features of examples may be provided. The features may be utilized solely or in methods disclosed herein, or in combination with other features disclosed herein.

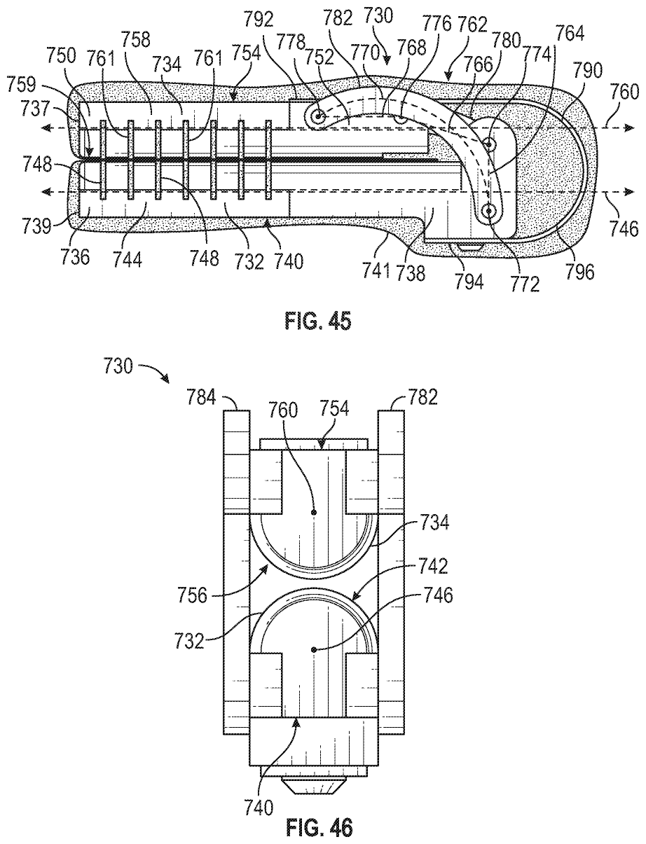

FIG. 45 illustrates an example of a clip 730 that may be utilized for a portion of a heart. The clip 730 may be configured to close a LAA 18, to reduce the possibility of clots or other undesired materials stemming from the LAA 18 from traveling into the bloodstream.

The clip 730 may include a first jaw 732 and a second jaw 734. The first jaw 732 may extend from a first end portion 736 to a second end portion 738 and may have an elongate shape. The first end portion 736 of the first jaw 732 may comprise a tip 739 of the first jaw 732 in examples. The first jaw 732 may be configured as an elongate beam.

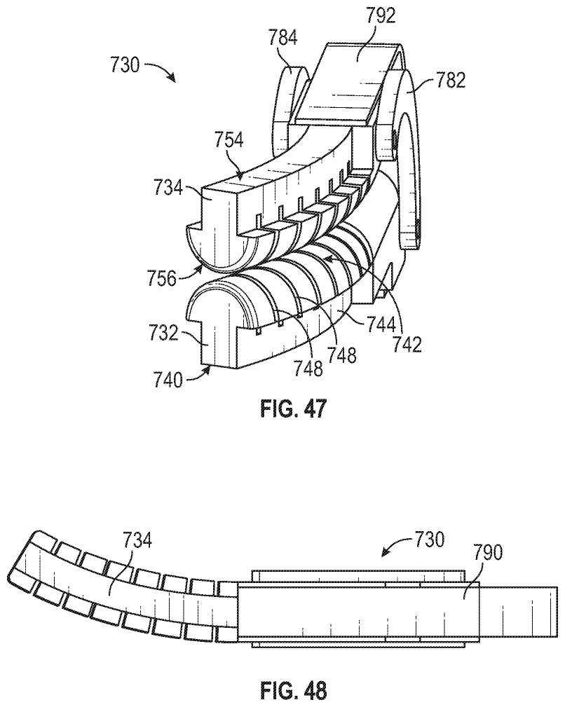

The first jaw 732 may include four sides, including an outer surface 740, a compression surface 742 (marked in FIG. 47 and facing opposite the outer surface 740 and towards the second jaw 734), and a first side surface 744 and a second side surface facing opposite the first side surface 744. The side surfaces each extend between the compression surface 742 and the outer surface 740.

The first jaw 732 may extend along a longitudinal axis 746 (marked in FIGS. 45 and 46). Referring to FIGS. 46 and 47, the compression surface 742 of the first jaw 732 may be wider than the outer surface 740 of the first jaw 732, and the first jaw 732 may have a cross sectional shape that is a “T” shape in examples, although other shapes may be utilized in examples as desired. The compression surface 742 may have a rounded shape and the outer surface 740 may have a flattened shape in examples as desired.

Referring to FIG. 45, in examples, the first jaw 732 may include a plurality of cuts 748. The cuts 748 may allow the first jaw 732 to be flexible and may allow the first jaw 732 to bend in a direction transverse to a plane of movement of the first jaw 732 and the second jaw 734, as shown in FIG. 47.

The second jaw 734 may extend from a first end portion 750 to a second end portion 752 and may have an elongate shape. The first end portion 750 of the second jaw 734 may comprise a tip 737 of the second jaw 734 in examples. The second jaw 734 may be configured as an elongate beam.

The second jaw 734 may include four sides, including an outer surface 754, a compression surface 756 (facing opposite the outer surface 754 and towards the first jaw 732), and a first side surface 758 and a second side surface facing opposite the first side surface 758. The side surfaces each extend between the compression surface 756 and the outer surface 754.

The second jaw 734 may extend along a longitudinal axis 760 (marked in FIGS. 45 and 46). Referring to FIGS. 45 and 46, the compression surface 756 of the second jaw 734 may be wider than the outer surface 754 of the second jaw 734, and the second jaw 734 may have a cross sectional shape that is a “T” shape in examples, although other shapes may be utilized in examples as desired. The compression surface 756 may have a rounded shape and the outer surface 754 may have a flattened shape in examples as desired.

Referring to FIG. 45, in examples, the second jaw 734 may include a plurality of cuts 761. The cuts 761 may allow the second jaw 734 to be flexible and may allow the second jaw 734 to bend in a direction transverse to a plane of movement of the first jaw 732 and the second jaw 734, as shown in FIG. 47.

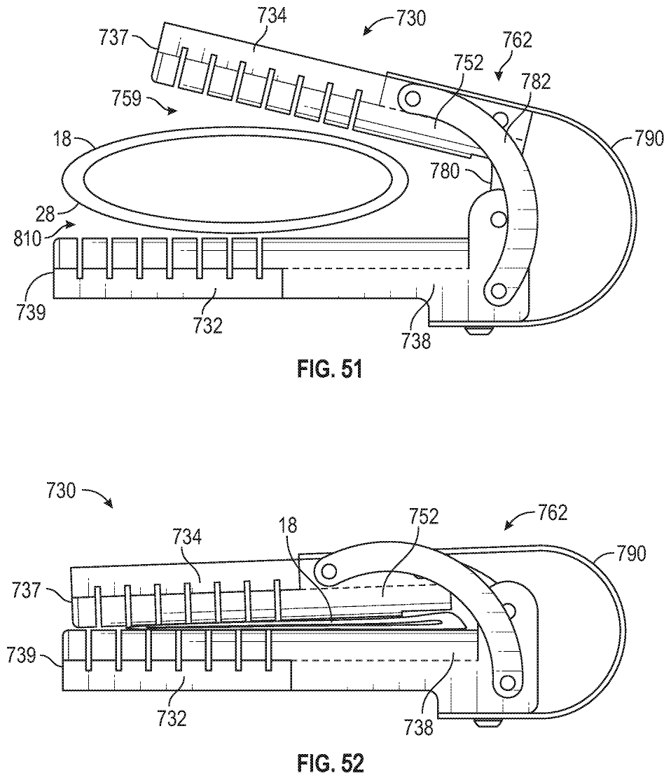

The second jaw 734 may be configured to extend parallel with the first jaw 732 and may be configured to compress a portion of a heart between the first jaw 732 and the second jaw 734. The portion of the heart may be compressed within a channel 759 (marked in FIG. 51) between the first jaw 732 and the second jaw 734. The second jaw 734 may be configured to move relative to the first jaw 732 in a plane.

A four-link mechanism 762 may be configured to move the first jaw 732 relative to the second jaw 734 to compress the portion of the heart between the first jaw 732 and the second jaw 734. The links of the four-link mechanism 762 may include a first link 764, a second link 766, a third link 768, and a fourth link 770. The first link 764 may extend from a first pivot 772 to a second pivot 774. The second link 766 may extend from the second pivot 774 to a third pivot 776. The third link 768 may extend from a third pivot 776 to a fourth pivot 778. The fourth link 770 may extend from the fourth pivot 778 to the first pivot 772.

The first link 764 may be the shortest link of the four-link mechanism 762. The first link 764 may comprise a portion of the first jaw 732 and may be positioned at the second end portion 738 of the first jaw 732. The first link 764 may extend transverse or perpendicular to the longitudinal axis 746 of the first jaw 732 as shown in FIG. 45. The first pivot 772 may be positioned at the second end portion 738 of the first jaw 732 and the second pivot 774 may be positioned at the second end portion 738 of the first jaw 732 along an axis transverse or perpendicular with the longitudinal axis 746 of the first jaw 732 in examples.

The second link 766 may be the second shortest link of the four-link mechanism 762. The second link 766 may comprise a first bar 780 that extends from the first jaw 732 to the second jaw 734. The first bar 780 may be positioned at the second end portion 738 of the first jaw 732 and the second end portion 752 of the second jaw 734. The first bar 780 may extend from the second end portion 738 of the first jaw 732 to the second end portion 752 of the second jaw 734 to form the second shortest link of the four-link mechanism 762. An end portion of the first bar 780 may couple to the second pivot 774 and an opposite end portion of the first bar 780 may couple to the third pivot 776. The first bar 780 may be configured to rotate about the second pivot 774 relative to the first link 764.

The third link 768 may be the third shortest link of the four-link mechanism 762. The third link 768 may comprise a portion of the second jaw 734 and may be positioned at the second end portion 752 of the second jaw 734. The third link 768 may extend parallel with the longitudinal axis 760 of the second jaw 734 in examples, as shown in FIG. 45. The third pivot 776 and the fourth pivot 778 may be positioned at the second end portion 752 of the second jaw 734. The third link 768 may be configured to rotate about the third pivot 776 relative to the second link 766.

The fourth link 770 may be the longest link of the four-link mechanism 762. The fourth link 770 may comprise a second bar 782 that extends from the second jaw 734 to the first jaw 732. The second bar 782 may be positioned at the second end portion 738 of the first jaw 732 and the second end portion 752 of the second jaw 734. The second bar 782 may extend from the second end portion 752 of the second jaw 734 to the second end portion 738 of the first jaw 732 to form the longest link of the four-link mechanism 762. An end portion of the second bar 782 may couple to the fourth pivot 778 and an opposite end portion of the second bar 782 may couple to the first pivot 772. The second bar 782 may be configured to rotate about the fourth pivot 778 relative to the third link 768 and may be configured to rotate about the first pivot 772 relative to the first link 764.

The second bar 782 may be curved and may be curved outward in a direction away from the channel 759 (marked in FIG. 51) of the clip 730. The second bar 782 may be positioned outward of the respective side surfaces 744, 758 of the first jaw 732 and the second jaw 734. Referring to FIG. 47, in examples, the fourth link 770 may include the second bar 782 and may include an opposing bar 784 on an opposite side of the first jaw 732 and the second jaw 734 that may be shaped similarly as the second bar 782. The jaws 732, 734 may be sandwiched between the second bar 782 and the opposing bar 784. In examples, the first bar 780 may be positioned inward of the first jaw 732 and the second jaw 734, and may be positioned inward of the second bar 782 and an opposing bar 784.

The four-link mechanism 762 may comprise a non-Grashof four-link mechanism 762 in examples. The four-link mechanism 762 may satisfy the non-Grashof condition of four-link mechanisms. The relative linkage lengths of the links may be 1 (first link 764), 1.25 (second link 766), 1.39 (third link 768) and 2.95 (fourth link 770) in examples. In examples, the linkage lengths may vary and may satisfy the non-Grashof condition of four-link mechanisms.

In examples, the four-link mechanism may comprise a Grashof four-link mechanism. In examples, other forms of four-link mechanisms and linkages may be utilized as desired.

The four-link mechanism 762 may be positioned at the second end portion 738 of the first jaw 732 and the second end portion 752 of the second jaw 734 and may close an end of the channel 759 (marked in FIG. 51) between the first jaw 732 and the second jaw 734. The first jaw 732 may extend from the pivot 772 between the longest fourth link 770 and the shortest first link 764 to the first end portion 736 of the first jaw 732. The second jaw 734 may extend from the pivot 778 between the longest fourth link 770 and the third shortest first link 768 to the first end portion 750 of the second jaw 734.

The four-link mechanism 762 may be configured to move to increase a distance between the first jaw 732 and the second jaw 734 and place the clip 730 in an opened state. The movement may be in a direction away from the first end portions 736, 750 of the respective first jaw 732 and the second jaw 734 in examples. The four-link mechanism 762 may be configured to move the first jaw 732 towards the second jaw 734 to compress the portion of the heart between the first jaw 732 and the second jaw 734 in a closed state.

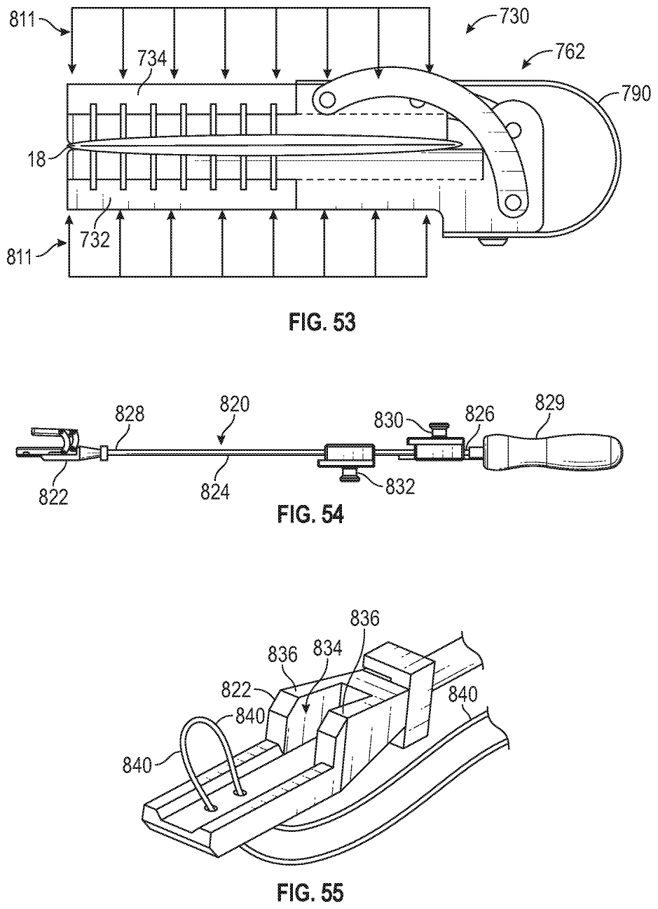

Referring to FIGS. 51 and 52, the movement of the four-link mechanism 762 may be such that the four-link mechanism 762 causes the first jaw 732 to open at an angle relative to the second jaw 734 and at a lateral displacement as shown in FIG. 51 in an opened state. Upon moving to a closed state the four-link mechanism 762 causes the tip 739 of the first jaw 732 and the tip 737 of the second jaw 734 to close prior to the second end portions 738, 752 of the first jaw 732 and the second jaw 734 respectively as shown in FIG. 52. As such, the closure of the clip 730 may position the first jaw 732 at an angle relative to the second jaw 734, with the four-link mechanism 762 fully closing with the first jaw 732 parallel with the second jaw 734 as shown in FIG. 53. The closing of the clip 730 may occur in a direction towards the respective second end portions 738, 752 of the first jaw 732 and the second jaw 734 to reduce the possibility of the portion of the heart extending outward from the tips 739, 737 of the jaws 732, 734.

The closing of the clip 730 shown in FIG. 53 may be reversible back to the opened state shown in FIG. 51, with the four-link mechanism 762 rotating in an opposite direction than the closure direction to open the clip 730.

Referring back to FIG. 45, a spring 790 may be configured to apply a compressive force to the four-link mechanism 762 to force the first jaw 732 towards the second jaw 734. The spring 790 may force the clip 730 towards the closed state. The spring 790 may be positioned at the second end portions 738, 752 of the first jaw 732 and second jaw 734. The spring 790 may include a first end 792 coupled to the first jaw 732 and may include a second end 794 coupled to the second jaw 734. A loop 796 may extend from the first end 792 to the second end 794 and may protrude from the second end portions 738, 752 of the first jaw 732 and second jaw 734. In examples, other configurations of springs may be utilized as desired.

In examples, a covering 741 may surround the clip 730. In examples, the covering 741 may have the form of a sheath that may be positioned around the clip 730 to provide an atraumatic outer surface for the clip 730. The sheath may conform to the shape of the clip 730 and may conform to the shape of a channel 759 (marked in FIG. 51) between the first jaw 732 and the second jaw 734. The sheath may be made of cloth or another material as desired. Other forms of coverings may be utilized in examples. In examples, a covering 741 may be excluded.

FIG. 46 illustrates an end view of the clip 730. FIG. 47 illustrates an end perspective view of the clip 730 showing a bending of the first jaw 732 and second jaw 734 in a direction transverse to the plane of movement of the first jaw 732 and the second jaw 734. FIG. 48 illustrates a top view of the clip 730 with the first jaw 732 and second jaw 734 bending in the direction transverse to the plane of movement of the first jaw 732 and the second jaw 734.

In operation, the clip 730 may be utilized to close the LAA 18 or another portion of a heart. A procedure may include gaining access to the heart 14. The access may be provided in a variety of manners, including a transcatheter access or a surgical access such as a thoracotomy, mini thoracotomy, or other methods of access. An open or mini sternotomy approach may be utilized as desired. A right-side approach may be utilized, for example, where direct visualization of the LAA 18 is compromised. Indirect visualization such as robotic visualization may be utilized as desired.

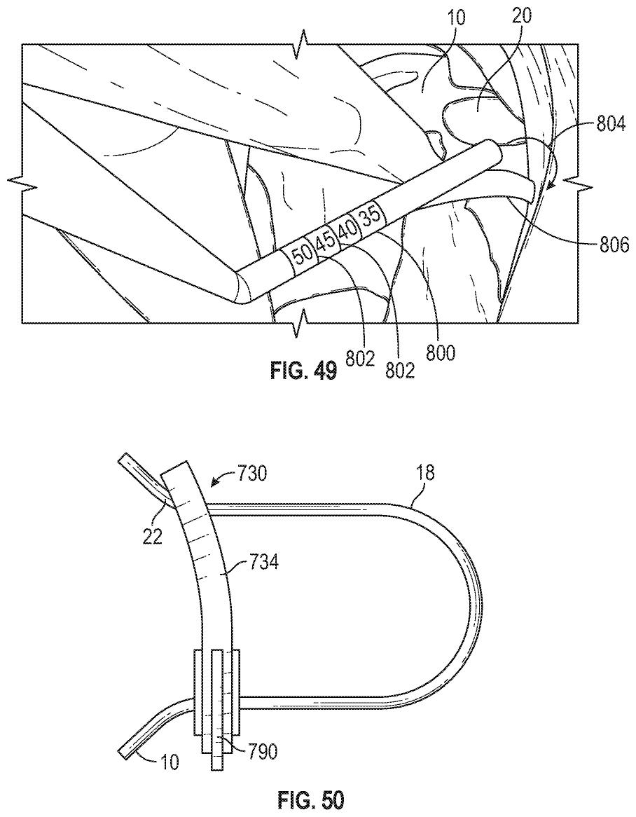

Referring to FIG. 49, in examples, a measurement of a length and/or contour of the outer wall 20 of the left atrium 10 at the base of the LAA 18 may be made. A measurement device 800 may be utilized to measure the length and/or the contour of the outer wall 20 of the left atrium 10 at the base of the LAA 18. The measurement device 800, for example, may include one or more length markers 802 that may be utilized to measure the length of the ostium 22 of the LAA 18 (as marked in FIG. 1 for example). The measurement of the length of the ostium 22 may be utilized to determine a depth that the LAA 18 may be positioned within the channel 759 (as shown in FIG. 51) between the jaws 732, 734 to compress the LAA 18 with the jaws 732, 734.

The measurement of the contour of the outer wall 20 of the left atrium 10 at the base of the LAA 18 may be utilized to determine an amount that the jaws 732, 734 may be bent (as shown in FIGS. 47 and 48) to contour to the outer wall 20 of the left atrium 10 at the base of the LAA 18. The measurement device 800, for example, may be made of a flexible or ductile material that may be curved to match the contour of the outer wall 20 of the left atrium 10 at the base of the LAA 18. Such a feature is shown in FIG. 49 via the curved arrow 804 and position of the end portion 806 of the measurement device 800. The end portion 806 of the measurement device 800 may remain in the curved position upon extraction from the patient's body and the jaws 732, 734 of the clip 730 may be bent to match the curvature.

Referring to FIG. 50, the clip 730 may be advanced towards the LAA 18 and the LAA 18 may be positioned between the jaws 732, 734 of the clip 730. As shown in FIG. 50, the jaws 732, 734 may have a curvature that was produced following the measurement step shown in FIG. 49. The clip 730 may be positioned at the ostium 22 of the LAA 18.

FIGS. 51-53 illustrate a closure of the clip 730 around the LAA 18. Referring to FIG. 51, the clip 730 may be advanced over the LAA 18 with the outer surface 28 of the LAA 18 positioned between the jaws 732, 734. The LAA 18 may pass through an axial opening 810 of the clip 730 that leads to the channel 759 between the jaws 732, 734.

As shown in FIG. 51, the clip 730 is in the opened state, with the four-link mechanism 762 moving the first jaw 732 away from the second jaw 734. The second jaw 734 may be positioned at an angle relative to the first jaw 732.

Referring to FIG. 52, the clip 730 may be moved towards a closed state, with the tip 739 of the first jaw 732 and the tip 737 of the second jaw 734 closing prior to the second end portions 738, 752 of the first jaw 732 and the second jaw 734 respectively. The spring 790 may force the first jaw 732 towards the second jaw 734. The closing of the clip 730 may occur in a direction towards the respective second end portions 738, 752 of the first jaw 732 and the second jaw 734 to reduce the possibility of the LAA 18 extending outward from the tips 739, 737 of the jaws 732, 734. The four-link mechanism 762 may bound an end of the channel 759 to reduce the possibility of the LAA 18 extending outward from the respective second end portions 738, 752 of the first jaw 732 and the second jaw 734.

The four-link mechanism 762 may continue to move the first jaw 732 towards the second jaw 734 to place the first jaw 732 parallel with the second jaw 734 as shown in FIG. 53. As represented in FIG. 53 by the arrows 811, a uniform force distribution may be provided along the first jaw 732 and the second jaw 734 along the lengths of the first jaw 732 and second jaw 734. The clip 730 may create a line of necrosis for the clip 730 along the ostium 22 of the LAA 18.

In examples, if desired, the clip 730 may be released or repositioned in a reverse operation of the four-link mechanism 762 than shown in FIGS. 51-53. The steps of the methods disclosed herein may be modified, substituted, excluded, or added to, as desired.

The clip 730 may be utilized to close the LAA 18, yet in examples other portions of a heart may be clipped or closed via use of the clip 730. In examples, other portions of a body, such as a tubular vessel or other portions of a body may be closed with the clip 730. Deployment may be via a delivery apparatus or via another method as desired. Deployment may be via surgical methods and may be transcatheter or via non-invasive surgery in methods.

FIG. 54, for example, illustrates an example of a delivery apparatus 820 that may be utilized to deploy or remove the clip 730 as desired. The delivery apparatus 820 may include a clip retainer 822 that may be utilized to retain the clip 730. The delivery apparatus 820 may include an elongate shaft 824 having a proximal end 826 and a distal end 828, with the clip retainer 822 positioned at the distal end 828 of the elongate shaft 824. The elongate shaft 824 may be configured to be flexible to allow for desired positioning if desired. In examples, a handle 829 may be positioned at the proximal end 826 of the elongate shaft 824 and may be configured for a user to grip to manipulate the clip retainer 822.

The delivery apparatus 820 may include a first control mechanism 830 that may be configured to control movement of the clip 730 between a closed state and an opened state. The delivery apparatus 820 in examples may include a second control mechanism 832 that may be configured to control release and capture of the clip 730 as desired.

Referring to FIG. 55, a perspective view of the clip retainer 822 is provided. The clip retainer 822 may have a variety of forms, and may include a channel 834 for retaining the clip 730. The first jaw 732 of the clip 730, for example, may fit into the channel 834 with the first end portion 736 of the first jaw 732 protruding distally from the channel 834 (as represented in FIG. 54 for example). Walls 836 of the channel 834 may reduce the possibility of the clip 730 undesirably rotating about the longitudinal axes 746, 760 of the jaws 732, 734 relative to the clip retainer 822 during deployment.

In examples, the second control mechanism 832 may include one or more tethers 840 that may couple to the clip 730 and may secure the clip 730 to the clip retainer 822. The one or more tethers 840 for example, may form a loop around a portion of the clip 730, such as the second end portion 738 of the first jaw 732. The loop may secure the clip 730 within the channel 834.

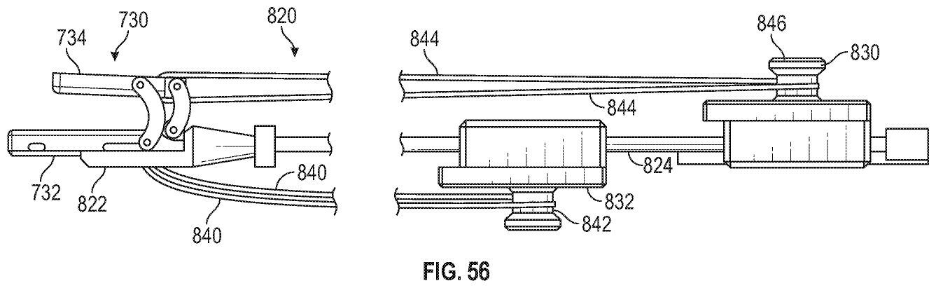

FIG. 56, for example, illustrates a side view of the delivery apparatus 820 showing the routing of the one or more tethers 840 of the second control mechanism 832. The one or more tethers 840 may be routed proximally from the clip retainer 822 to a securing point 842 on the delivery apparatus 820. The securing point 842 may be positioned on the elongate shaft 824 in examples and may be positioned to keep the one or more tethers 840 taut to secure the clip 730 to the clip retainer 822. The securing point 842 may comprise a knob or other form of securing point 842. In examples, the securing point 842 may be detachable from the delivery apparatus 820 or the one or more tethers 840 may otherwise be released from the delivery apparatus 820 to relieve the tension in the one or more tethers 840 and allow the clip 730 to decouple from the clip retainer 822. Thus, during deployment, upon clamping of the LAA 18 or other portion of the heart by the clip 730, the second control mechanism may be utilized to decouple the clip 730 from the clip retainer 822. The looped configuration of the one or more tethers 840 may allow an end of the tether 840 to be cut and then pulled through the clip 730 by pulling on the other end of the tether 840, to release the tether 840 from the clip 730.

The first control mechanism 830 may include one or more tethers 844 that may be looped around the second jaw 734 and configured to be pulled to overcome the force of the spring 790 (not shown in FIG. 56). The one or more tethers 844 may have end portions coupled to a securing point 846 that may be configured to slide relative to the delivery apparatus 820 and may slide relative to the elongate shaft 824. The securing point 846 may be configured to slide proximally to move the clip 730 to the opened state and may be configured to slide distally to allow the clip 730 to move to the closed state, with the relative tension in the one or more tethers 844 increased or decreased during movement. As such, during deployment, the one or more tethers 844 may hold the clip 730 in the opened state until the clip 730 is positioned as desired relative to the LAA 18 or other portion of the heart to be closed (as shown in FIG. 51 for example). The tension in the one or more tethers 844 may be released to allow the spring 790 to force the clip 730 to the closed state as shown in FIG. 53 for example. The looped configuration of the one or more tethers 844 may allow an end of the tether 844 to be cut and then pulled through the clip 730 by pulling on the other end of the tether 844, to release the tether 844 from the clip 730. If it is desired to reposition the clip 730, the tension in the tether 844 may be increased again to move the clip 730 to the opened state for repositioning.

Various forms of control mechanisms and other forms of delivery apparatuses may be utilized in examples as desired.

In examples, the spring 790 may comprise a leaf spring as shown herein, or may comprise another form of spring including a compression spring, a torsional spring, a screw, an external spring that is attached to the clip, or magnetic devices that function as a spring.

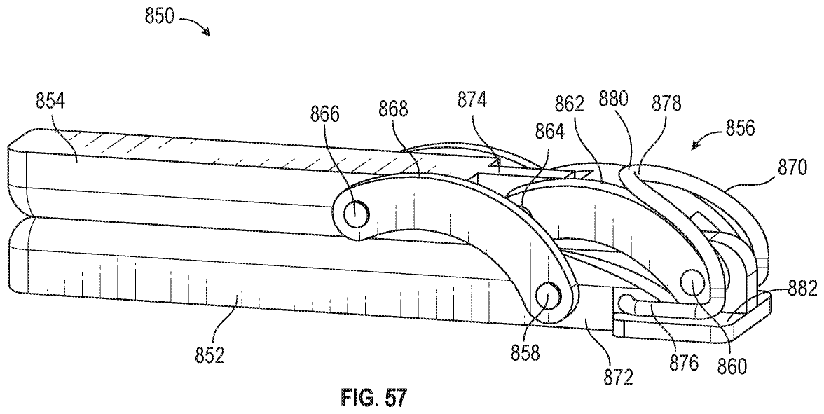

FIG. 57, for example, illustrates an example of a clip 850 including a first jaw 852 configured similarly as the first jaw 732 and a second jaw 854 configured similarly as the second jaw 734. The clip 850 may be configured similarly as the clip 730 and may include a four-link mechanism 856 configured similarly as the four-link mechanism 762 and configured to move the first jaw 852 relative to the second jaw 854 to compress a portion of a heart between the first jaw 852 and the second jaw 854 in a similar manner as the four-link mechanism 762.

The four-link mechanism 856 may include a first link between a first pivot 858 and a second pivot 860, a second link comprising a bar 862 between the second pivot 860 and a third pivot 864, a third link between the third pivot 864 and a fourth pivot 866, and a fourth link comprising a bar 868 between the fourth pivot 866 and the first pivot 858. The first pivot 858 and second pivot 860 may both be positioned on the first jaw 852 and the third pivot 864 and the fourth pivot 866 may be positioned on the second jaw 854.

The clip 850 may include a spring 870 configured to apply a compressive force to the four-link mechanism 856 to force the first jaw 852 towards the second jaw 854. The spring 870 may force the clip 850 towards the closed state. The spring 870 may be positioned at respective second end portions 872, 874 of the first jaw 852 and second jaw 854. The spring 870 may include a first end 876 coupled to the first jaw 852 and may include a second end 878 positioned along the bar 862 between the second pivot 860 and the third pivot 864.

The spring 870 may comprise a loop of material such as a loop of wire that extends from the first end 876 to an apex 880 of the spring 870 at the second end 878. The first end 876 of the spring 870 may be supported by a support surface 882 of the first jaw 852 that may extend perpendicular with a longitudinal axis of the first jaw 852. The spring 870 may extend to the second end 878 and may extend along an outer surface of the bar 862. The spring 870 may have a curvature that matches a curvature of the bar 862 in examples.

The clip 850 is shown in a closed state in FIG. 57. Upon movement of the clip 850 to the opened state, the bar 862 of the four-link mechanism 856 may press against the second end 878 of the spring 870. The second end 878 of the spring 870 may rotate with the bar 862 as the clip 850 is opened, with the first end 876 of the spring 870 held in position. The spring 870 may provide a force against the bar 862 to move the clip 850 towards the closed state as shown in FIG. 57. The second end 878 of the spring 870 may rotate relative to the first end 876 of the spring 870 in a direction opposite the opening direction to move the clip 850 towards the closed state.

In examples, other configurations of springs may be utilized as desired.

In examples, the linkage joints between the links may comprise revolute joints as disclosed herein. In examples, other forms of linkage joints may be utilized including living hinges or compliant material. In examples, alternative linkage lengths may be utilized. In examples, other methods may be utilized to allow the jaws 732, 734 to bend than use of the cuts 748, 761. For example, ductile material, or heat forming thermoplastic may be utilized. In examples, swappable jaws may be utilized, with various shapes available to a user upon deployment. Various other examples of the clips disclosed herein may be utilized.

Variations in the clips and methods disclosed herein may be provided. Features across examples may be combined. Features may be excluded or added to in various examples disclosed herein. Combinations of features of examples may be provided. The features may be utilized solely or in methods disclosed herein, or in combination with other features disclosed herein.

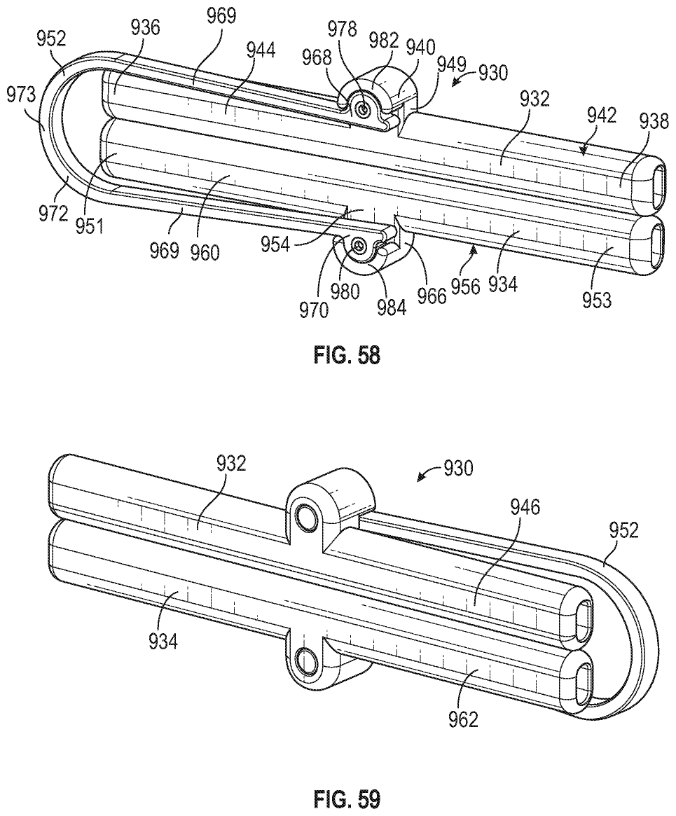

FIG. 58 illustrates an example of a clip 930 that may be utilized for a portion of a heart. The clip 930 may be configured to close a LAA 18, to reduce the possibility of clots or other undesired materials stemming from the LAA 18 from traveling into the bloodstream.

The clip 930 may include a first jaw 932 and a second jaw 934. The first jaw 932 may extend from a first end portion 936 to a second end portion 938 and may have an elongate shape. The first jaw 932 may be configured as an elongate beam. The first jaw 932 may be rigid or may have an amount of flexibility in examples. The first jaw 932 may have a central portion 940 between the first end portion 936 and the second end portion 938.

The first jaw 932 may include four sides, including an outer surface 942, a compression surface (facing opposite the outer surface 942 and towards the second jaw 934), and two side surfaces 944, 946 (with side surface 946 marked in FIG. 59) each extending between the compression surface and the outer surface 942.

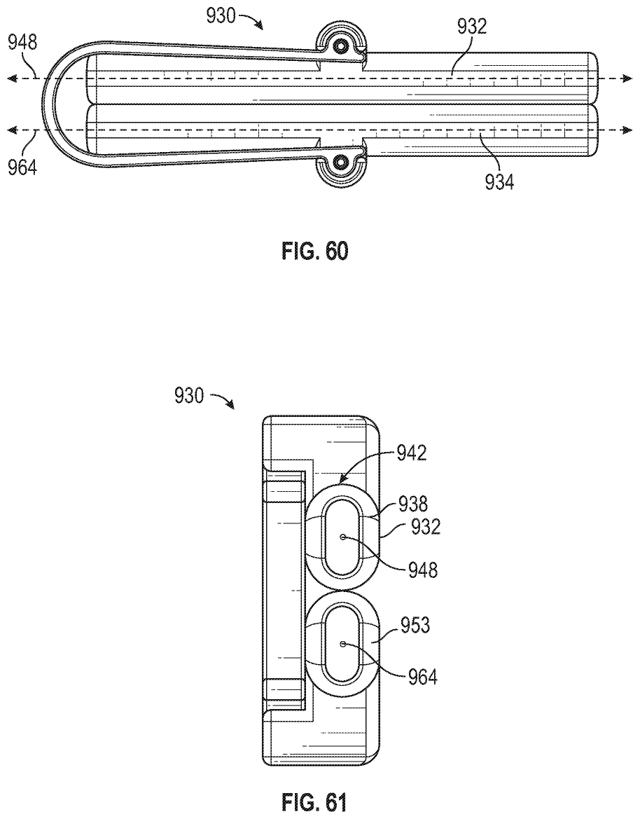

The first jaw 932 may extend along a longitudinal axis 948 (marked in FIGS. 60 and 61) and may have an oval cross section when viewed perpendicular to the longitudinal axis 948 (as visible in the end view of FIG. 61). The four sides of the first jaw 932 may each have flattened portions, and in examples, the first jaw 932 may be configured to have a rectangular shape as desired.

In examples, the first jaw 932 may have a hollow center (as visible in FIG. 61). The hollow center may extend longitudinally along the axis 948 from the first end portion 936 to the second end portion 938.

Referring to FIG. 58, the first jaw 932 may include a coupler 949. The coupler 949 may be configured to couple to a spring 952 and may protrude from the outer surface 942 of the first jaw 932. The coupler 949 may protrude from the side surface 944 of the first jaw 932. The coupler 949 may comprise a pivot coupler that the spring 952 may pivot about.

The second jaw 934 may extend from a first end portion 951 to a second end portion 953 and may have an elongate shape. The second jaw 934 may be configured as an elongate beam. The second jaw 934 may be rigid or may have an amount of flexibility in examples. The second jaw 934 may have a central portion 954 between the first end portion 951 and the second end portion 953.

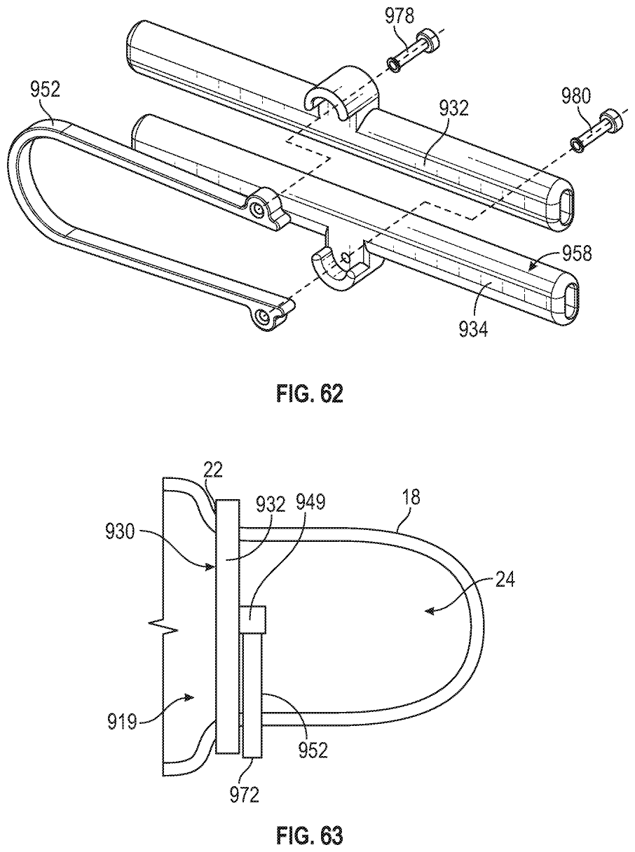

The second jaw 934 may include four sides, including an outer surface 956, a compression surface 958 (marked in FIG. 62 and facing opposite the outer surface 956 and towards the first jaw 932), and two side surfaces 960, 962 (with side surface 962 marked in FIG. 59) each extending between the compression surface 958 and the outer surface 956.

The second jaw 934 may extend parallel with the first jaw 932.

The second jaw 934 may extend along a longitudinal axis 964 (marked in FIGS. 60 and 61) and may have an oval cross section when viewed perpendicular to the longitudinal axis 964 (as visible in the end view of FIG. 61). The four sides of the second jaw 934 may each have flattened portions, and in examples, the second jaw 934 may be configured to have a rectangular shape as desired.

In examples, the second jaw 934 may have a hollow center (as visible in FIG. 61). The hollow center may extend longitudinally along the axis 964 from the first end portion 951 to the second end portion 953.

Referring to FIG. 58, the second jaw 934 may include a coupler 966. The coupler 966 may be configured to couple to the spring 952 and may protrude from the outer surface 956 of the second jaw 934. The coupler 966 may protrude from the side surface 960 of the second jaw 934. The coupler 966 may comprise a pivot coupler that the spring 952 may pivot about.

The spring 952 may be coupled to the central portion 940 of the first jaw 932 and the central portion 954 of the second jaw 934 and configured to force the first jaw 932 and the second jaw 934 together to compress a portion of the heart between the first jaw 932 and the second jaw 934. The spring 952, for example may have a “C” shape with a first end 968 and a second end 970 and a loop 972 coupled to the first end 968 and the second end 970. The loop 972 may include straight portions 969 coupled to a curved portion 973 forming the curve of the “C” shape. The first end 968 of the spring 952 may be coupled to the central portion 940 of the first jaw 932 and the second end 970 of the spring 952 may be coupled to the central portion 954 of the second jaw 934, and the loop 972 may extend towards the first end portion 936 of the first jaw 932 and the first end portion 951 of the second jaw 934.

The spring 952 may be positioned offset from a plane of movement of the first jaw 932 and the second jaw 934. The spring 952 may extend in a plane that is parallel with the plane of movement of the first jaw 932 and the second jaw 934. For example, the couplers 949, 966 may position the spring 952 offset from the side surfaces 944, 960 of the first jaw 932 and the second jaw 934 respectively, such that the spring 952 extends in a plane that is parallel yet offset from the plane of movement of the first jaw 932 and the second jaw 934. Such a feature may allow the side surfaces 946, 962 (marked in FIG. 59) to be positioned close to the ostium 22 of the LAA 18, without interference from the spring 952, as shown in FIG. 63 for example.

The spring 952 may be configured to allow the clip 930 to move from an opened state to a closed state, yet force the clip 930 towards the closed state. The spring 952 accordingly may provide a force that moves the compression surfaces of the first jaw 932 and second jaw 934 towards each other to compress a portion of the heart therein.

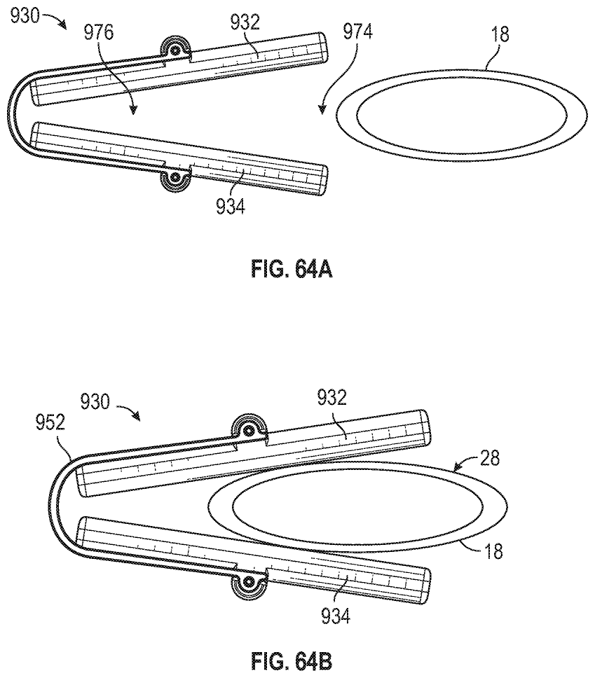

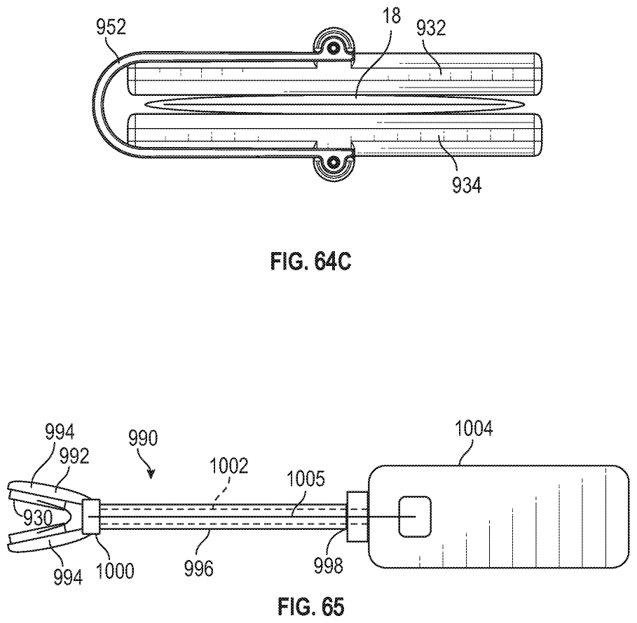

The spring 952 may have a “C” shape to allow the second end portions 938, 953 of the first jaw 932 and the second jaw 934 respectively to form an axial opening 974 (marked in FIG. 64A) for a space 976 (marked in FIG. 64A) between the first jaw 932 and the second jaw 934 for receiving the portion of the heart. The clip 930 may be positioned in an opened state and with the portion of the heart slid through the axial opening 974 and into the space 976. In examples, other methods of entry into the space 976 may be provided (e.g., along an axis of the LAA).

The loop 972 may form a boundary of the space 976 between the first jaw 932 and the second jaw 934. The loop 972 may protrude from the first end portion 936 of the first jaw 932 and the first end portion 951 of the second jaw 934. The loop 972 accordingly may prevent the clip 930 from sliding distally with respect to the LAA 18 upon deployment, and may prevent the tissue of the LAA 18 from protruding further than the loop 972 upon compression of the LAA 18.

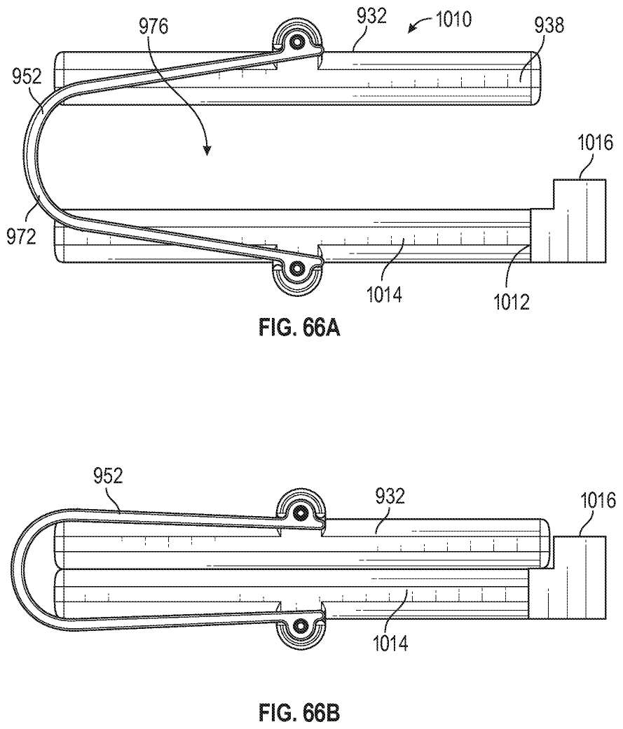

The spring 952 may be configured to pivotally couple to the couplers 949, 966. The pivotal coupling may be provided in a variety of manners, including each coupler 949, 966 including a respective axle 978, 980 (as shown in the assembly view of FIG. 62), or via hinges or a flexible joints, among other manners as desired. The pivoting of the spring 952 may allow the first jaw 932 and second jaw 934 to rotate during deployment between angled positions (as shown in FIG. 64A), and parallel positions (as shown in FIG. 64C) and FIG. 66A for example.

In examples, the first jaw 932 may include an outer covering 982 of the first end 968 of the spring 952. The second jaw 934 may include an outer covering 984 of the second end 970 of the spring 952.

FIG. 59 illustrates a side perspective view of the clip 930 opposite the side shown in FIG. 58. FIG. 60 illustrates a side view of the clip 930. FIG. 61 illustrates an end view of the clip 930 from the second end portions 938, 953 of the first jaw 932 and second jaw 934.

FIG. 62 illustrates an assembly view of the clip 930. The spring 952 may couple to the first jaw 932 and the second jaw 934 via axles 978, 971 passing through apertures in the respective first jaw 932 and second jaw 934.

In operation, the clip 930 may be utilized to close the LAA 18. FIG. 63 illustrates a top schematic view of the clip 930 closing or occluding the LAA 18, and being positioned at the ostium 22 of the LAA 18. The clip 930 may span the ostium 22 to close the ostium 22 and reduce blood flow between the LAA 18 and the main chamber 919 of the left atrium 10. The loop 972 of the spring 952 may be positioned at a side of the LAA 18.

FIGS. 64A-64C illustrate an exemplary deployment of the clip 930. Features of the clip 930 may be excluded from view in FIGS. 64A-64C. In FIG. 64A, the clip 930 may be placed in an opened state and moved towards the LAA 18 in a direction transverse to the main axis of the LAA 18. In examples, other approaches (e.g., along the main axis of the LAA 18) may be utilized as desired. Access may be provided to the LAA 18 in a variety of manners, including a transcatheter access or a surgical access such as a thoracotomy, mini thoracotomy, or other methods of access.

In FIG. 64B, the clip 930 may be placed over the outer surface 28 of the LAA 18. In examples, a delivery apparatus may be utilized to move the clip 930 towards the LAA 18 (as shown in FIG. 65), or in examples, a user (e.g., a medical technician such as a surgeon, or other form of medical technician) may manually place the clip 930. The clip 930 may be placed over the outer surface 28 of the LAA 18 in an opened state. A user may confirm a desired placement of the clip 930 over the LAA 18.

In FIG. 64C, the clip 930 may be moved to a closed state with the LAA 18 compressed between the first jaw 932 and the second jaw 934. The spring 952 may force the clip 930 to the closed state. The entire span of the LAA 18 may be closed as desired at the ostium (as shown in FIG. 63) or another portion of the LAA 18 may be closed.

In examples, if desired, the clip 930 may be opened and either removed or repositioned in a deployment procedure or another procedure. The clip 930 may be grasped and the force of the spring 952 may be overcome to open the clip 930.

The clip 930 may be utilized to close the LAA 18, yet in examples other portions of a heart may be clipped or closed via use of the clip 930. In examples, other portions of a body, such as a tubular vessel or other portions of a body may be closed with the clip 930. Deployment may be via a delivery apparatus or via another method as desired.

FIG. 65, for example, illustrates an example of a delivery apparatus 990 that may be utilized to deploy or remove the clip 930 as desired. The delivery apparatus 990 may include a clip retainer 992 that may be utilized to retain the clip 930. The clip retainer 992 may include arms 994 or another form of retainer that may hold the clip 930 in an opened state and may allow the clip 930 to be moved to the closed state at a desired time. The arms 994, for example, may be configured to pivot with respect to each other to allow the clip 930 to move to the closed state. The arms 994 may further be configured to reengage the clip 930 for removal or repositioning of the clip 930.

The delivery apparatus 990 may include an elongate shaft 996 having a proximal end 998 and a distal end 1000, with the clip retainer 992 positioned at the distal end 1000 of the elongate shaft 996. The elongate shaft 996 may be configured to be flexible to allow for desired positioning, and may be configured to be actively deflectable with a deflection mechanism 1002 for example. In examples, a handle 1004 may be positioned at the proximal end 998 of the elongate shaft 996 and may be configured for a user to grip to manipulate the clip retainer 992.

The delivery apparatus 990 may include a control mechanism 1005 that may allow for control of the clip retainer 992. For example, the control mechanism 1005 may be configured to allow the clip retainer 992 to deploy the clip 930 and may be utilized for removal or repositioning of the clip 930. The control mechanism 1005 and the deflection mechanism 1002 may be operable at the handle 1004 if desired.

In examples, other forms of delivery apparatuses may be utilized as desired.

Variations in the configuration of the clip 930 may be provided as desired.

FIG. 66A, for example, illustrates an example of a clip 1010 in which the second end portion 1012 of the second jaw 1014 includes a lip 1016 configured to extend over the second end portion 938 of the first jaw 932. The lip 1016 may be positioned opposite the loop 972 of the spring 952. The lip 1016 may overlap the second end portion 938 of the first jaw 932 to close the space 976 between the first jaw 932 and the second jaw 1014 when the clip 1010 is in the closed state. As such, a reduced possibility of LAA tissue protruding outward from the closed clip 1010 may result.

FIG. 66B illustrates an overlap of the lip 1016 with the first jaw 932 to reduce the possibility of LAA tissue protruding outward from the closed clip 1010.

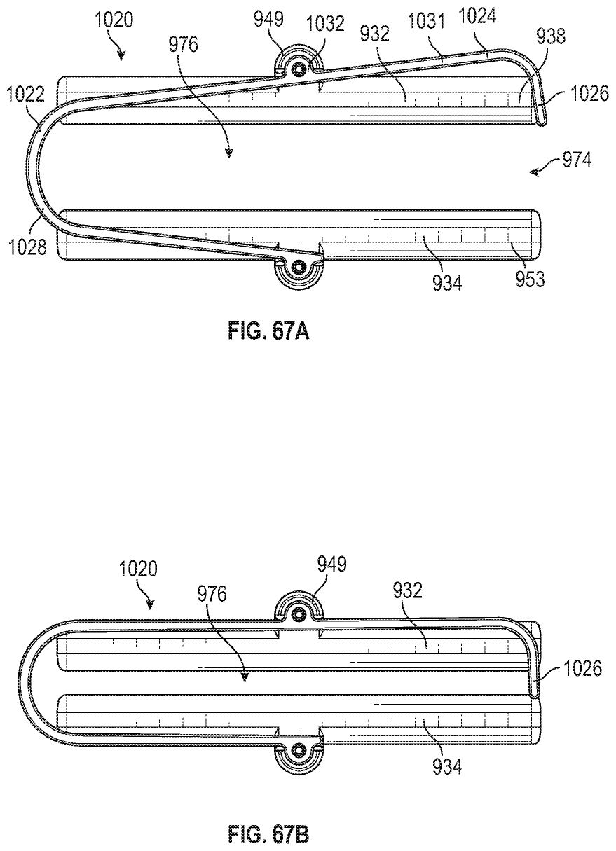

FIG. 67A illustrates an example of a clip 1020 in which the spring 1022 includes an end portion 1024 having a lip 1026 that is configured to close the space 976 between the first jaw 932 and the second jaw 934 opposite the position of the loop 1028 of the spring 1022. The lip 1026 may overhang the space 976 between the first jaw 932 and the second jaw 934 at the second end portions 938, 953 of the first jaw 932 and second jaw 934 to close the space 976. The spring 1022 may have a straight portion 1031 that extends towards the second end portion 938 of the first jaw 932, from a coupling portion 1032 that couples to the coupler 949. The straight portion 1031 may extend to the lip 1026.

FIG. 67A illustrates the clip 1020 in an opened state, with the lip 1026 pivoted out of the axial opening 974 of the clip 1020 to reduce the possibility of contact with the LAA 18 upon deployment of the clip 1020 and to increase the size of the opening 974. FIG. 67B illustrates the lip 1026 having rotated about the coupler 949 in the closed state to overhang the space 976.

Various other modifications of the clips disclosed herein may be provided. Various other methods of deployment and use of the clips may be provided as desired.

The clips as disclosed herein may be utilized to close the LAA or may be utilized to close another portion of a heart. In examples, the clips may be utilized to close other portions of a body, including other tubular vessels or other portions of a body. Deployment may be via a delivery apparatus or via another method as desired. Deployment may be via surgical methods and may be transcatheter or via non-invasive surgery in methods.

Variations in the clips and methods disclosed herein may be provided. Features across examples may be combined. Features may be excluded or added to in various examples disclosed herein. Combinations of features of examples may be provided. The features may be utilized solely or in methods disclosed herein, or in combination with other features disclosed herein.

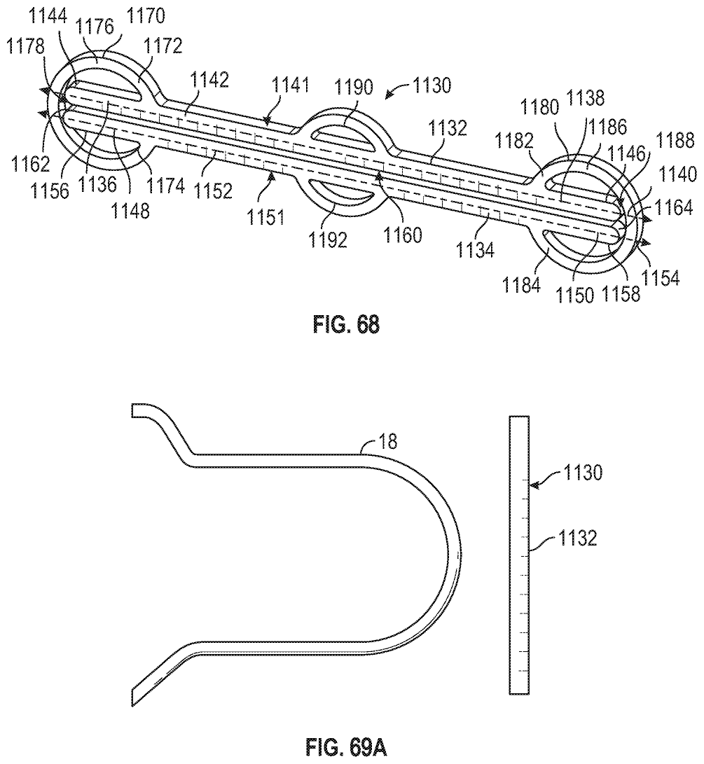

FIG. 68 illustrates an example of a clip 1130 that may be utilized for a portion of a heart. The clip 1130 may be configured to close a LAA 18, to reduce the possibility of clots or other undesired materials stemming from the LAA 18 from traveling into the bloodstream.

The clip 1130 may include a first jaw 1132 and a second jaw 1134. The first jaw 1132 may extend from a first end portion 1136 to a second end portion 1138 and may have an elongate shape. The first jaw 1132 may include four sides, including an outer surface 1141, a compression surface (opposite the outer surface 1141), and a first side surface 1142 and a second side surface facing opposite the first side surface 1142.

The first jaw 1132 may extend along a longitudinal axis 1140 and may be configured as a beam. The first end portion 1136 may comprise a tip 1144 of the first jaw 1132 and the second end portion 1138 may comprise an opposite tip 1146 of the first jaw 1132.

The second jaw 1134 may be configured similarly as the first jaw 1132 in examples. The second jaw 1134 may extend from a first end portion 1148 to a second end portion 1150 and may have an elongate shape. The second jaw 1134 may include four sides, including an outer surface 1151, a compression surface (opposite the outer surface 1151), and a first side surface 1152 and a second side surface facing opposite the first side surface 1152.

The second jaw 1134 may extend along a longitudinal axis 1154 and may be configured as a beam. The first end portion 1148 may comprise a tip 1156 of the second jaw 1134 and the second end portion 1150 may comprise an opposite tip 1158 of the second jaw 1134.

The second jaw 1134 may extend along the first jaw 1132 to form a channel 1160 for receiving a portion of the heart. The compressive surfaces of the first jaw 1132 and second jaw 1134 may face each other and may be positioned on opposite sides of the channel 1160. The channel 1160 may have a first end 1162 at the respective first end portions 1136, 1148 of the first jaw 1132 and the second jaw 1134 and may have a second end 1164 at the respective second end portions 1138, 1150 of the first jaw 1132 and the second jaw 1134.

A first spring 1170 may couple the first jaw 1132 to the second jaw 1134 and may cover the first end 1162 of the channel 1160 at the first end portion 1136 of the first jaw 1132 and the first end portion 1148 of the second jaw 1134. The first spring 1170 may be configured to force the first jaw 1132 towards the second jaw 1134 to compress the portion of the heart within the channel 1160. The first spring 1170 in examples may include a first end 1172 and a second end 1174 and a loop 1176 extending from the first end 1172 and the second end 1174.

The first end 1172 of the first spring 1170 may be coupled to the first jaw 1132 at the first end portion 1136 of the first jaw 1132. The second end 1174 of the first spring 1170 may be coupled to the second jaw 1134 at the first end portion 1148 of the second jaw 1134. The loop 1176 may protrude from the first end portion 1136 of the first jaw 1132 and the first end portion 1148 of the second jaw 1134. The loop 1176 may bound the first end 1162 of the channel 1160. The loop 1176 may have a circular shape in examples, with the first spring 1170 forming a “C” shape. In examples, the loop 1176 may have another shape or configuration as desired.

The first spring 1170 may be spaced from the first end 1162 of the channel 1160 with a gap 1178. The first spring 1170 accordingly may be spaced from the tips 1144, 1156 of the first jaw 1132 and the second jaw 1134 at the first end portions 1136, 1148.

A second spring 1180 may couple the first jaw 1132 to the second jaw 1134 and may cover the second end 1164 of the channel 1160 at the second end portion 1138 of the first jaw 1132 and the second end portion 1150 of the second jaw 1134. The second spring 1180 may be configured to force the first jaw 1132 towards the second jaw 1134 to compress the portion of the heart within the channel 1160. The second spring 1180 in examples may include a first end 1182 and a second end 1184 and a loop 1186 extending from the first end 1182 and the second end 1184.

The first end 1182 of the second spring 1180 may be coupled to the first jaw 1132 at the second end portion 1138 of the first jaw 1132. The second end 1184 of the second spring 1180 may be coupled to the second jaw 1134 at the second end portion 1150 of the second jaw 1134. The loop 1186 may protrude from the second end portion 1138 of the first jaw 1132 and the second end portion 1138 of the second jaw 1134. The loop 1186 may bound the second end 1164 of the channel 1160. The loop 1186 may have a circular shape in examples, with the second spring 1180 forming a “C” shape. In examples, the loop 1186 may have another shape or configuration as desired.

The second spring 1180 may be spaced from the second end 1164 of the channel 1160 with a gap 1188. The second spring 1180 accordingly may be spaced from the tips 1146, 1158 of the first jaw 1132 and the second jaw 1134 at the second end portions 1138, 1150.

The springs 1170, 1180 may force the compression surfaces of the first jaw 1132 and the second jaw 1134 towards each other to form a closed state for the clip 1130. The positioning of the springs 1170, 1180 may distribute the force applied by the springs 1170, 1180 towards the center of the first jaw 1132 and the second jaw 1134. A portion of a heart may be compressed within the channel 1160 between the first jaw 1132 and the second jaw 1134. In examples, the first jaw 1132 may be moved away for the second jaw 1134 to form an opened state for the clip 1130. In such a configuration the clip 1130 may be positioned over the portion of the heart to be compressed, and then the clip 1130 may be deployed to cause the clip 1130 to move to the closed state.

In examples, the first jaw 1132 may include a first aperture 1190 that may protrude from the outer surface 1141 of the first jaw 1132. The second jaw 1134 may include a second aperture 1192 that may protrude from the outer surface 1151 of the second jaw 1134. The apertures 1190, 1192 may each be configured to receive a device for applying an expansion force to the first spring 1170 and the second spring 1180 to move the clip 1130 to the opened state. The device, for example, may comprise jaws that enter the first aperture 1190 and the second aperture 1192 to open the clip 1130 during deployment. Other forms of devices may be utilized as desired.

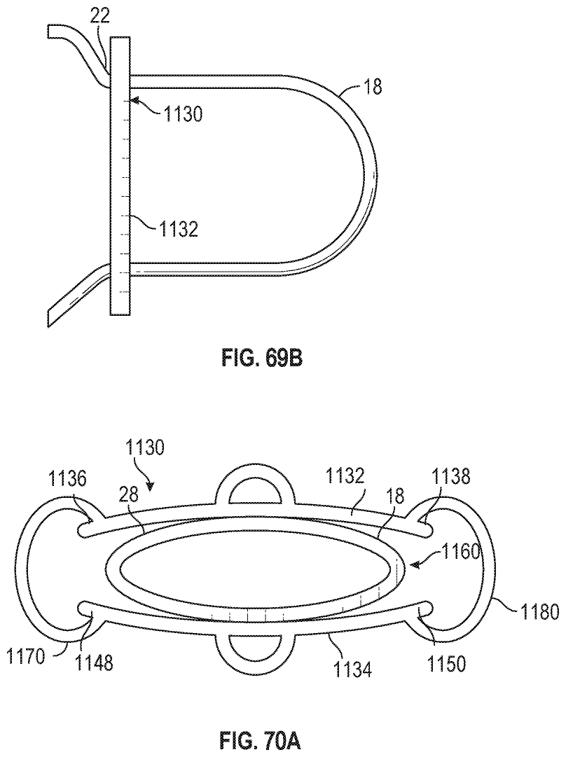

In operation, the clip 1130 may be utilized to close the LAA 18. FIG. 69A illustrates a top schematic view of the clip 1130 approaching the LAA 18. The LAA 18 may be passed through the channel 1160 between the first jaw 1132 and the second jaw 1134. FIG. 69B illustrates a top schematic view of the clip 1130 closing or occluding the LAA 18, and being positioned at the ostium 22 of the LAA 18. The clip 1130 may span the ostium 22 to close the ostium 22 and reduce blood flow between the LAA 18 and the main chamber of the left atrium 10.

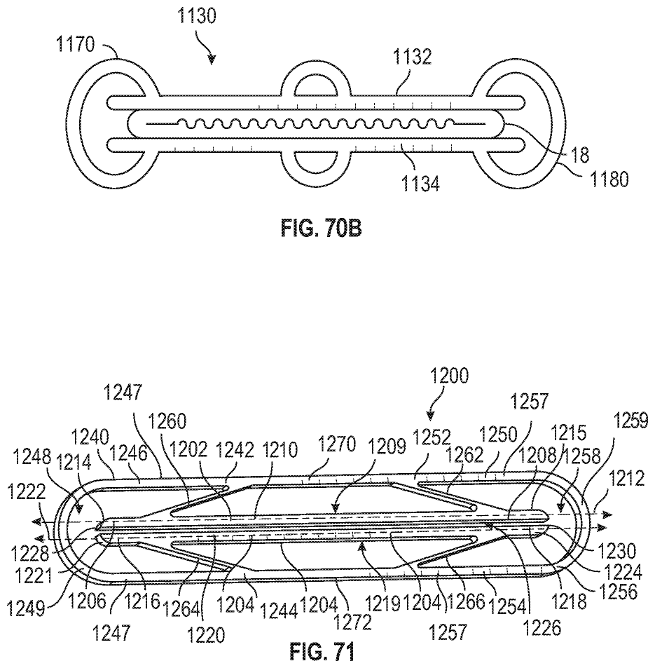

FIGS. 70A and 70B illustrate an exemplary deployment of the clip 1130. Features of the clip 1130 may be excluded from view in FIGS. 70A and 70B. In FIG. 70A, the clip 1130 may be placed in an opened state and moved towards the LAA 18 along the main axis of the LAA 18. Access may be provided to the LAA 18 in a variety of manners, including a transcatheter access or a surgical access such as a thoracotomy, mini thoracotomy, or other methods of access.

In examples, a delivery apparatus may be utilized to move the clip 1130 towards the LAA 18, or in examples, a user (e.g., a medical technician such as a surgeon, or other form of medical technician) may manually place the clip 1130. The clip 1130 may be placed over the outer surface 28 of the LAA 18 in an opened state. A user may confirm a desired placement of the clip 1130 over the LAA 18. In an opened state a central portion of the first jaw 1132 and a central portion of the second jaw 1134 may open to a greater extent than the end portions 1136, 1138, 1148, 1150 of the jaws 1132, 1134. The first jaw 1132 and second jaw 1134, for example, may be configured to bend outward at the central portions such the first jaw 1132 and second jaw 1134 form a curved shape.

In FIG. 70B, the clip 1130 may be moved to a closed state with the LAA 18 compressed between the first jaw 1132 and the second jaw 1134. The springs 1170, 1180 may force the clip 1130 to the closed state. The entire span of the LAA 18 may be closed as desired at the ostium (as shown in FIG. 70B) or another portion of the LAA 18 may be closed.

In examples, if desired, the clip 1130 may be opened and either removed or repositioned in a deployment procedure or another procedure. The clip 1130 may be grasped and the force of the springs 1170, 1180 may be overcome to open the clip 1130.

The clip 1130 may be utilized to close the LAA 18, yet in examples other portions of a heart may be clipped or closed via use of the clip 1130. In examples, other portions of a body, such as a tubular vessel or other portions of a body may be closed with the clip 1130. Deployment may be via a delivery apparatus or via another method as desired.

Variations in the configuration of the clip 1130 may be provided as desired.

FIG. 71 illustrates an example of a clip 1200 that may be utilized for a portion of a heart. The clip 1200 may be configured to close a LAA 18, to reduce the possibility of clots or other undesired materials stemming from the LAA 18 from traveling into the bloodstream.

The clip 1200 may include a first jaw 1202 and a second jaw 1204. The first jaw 1202 may extend from a first end portion 1206 to a second end portion 1208 and may have an elongate shape. The first jaw 1202 may include four sides, including an outer surface 1209, a compression surface (opposite the outer surface 1209), and a first side surface 1210 and a second side surface facing opposite the first side surface 1210.

The first jaw 1202 may extend along a longitudinal axis 1212 and may be configured as a beam. The first end portion 1206 may comprise a tip 1214 of the first jaw 1202 and the second end portion 1208 may comprise an opposite tip 1215 of the first jaw 1202.

The second jaw 1204 may be configured similarly as the first jaw 1202 in examples. The second jaw 1204 may extend from a first end portion 1216 to a second end portion 1218 and may have an elongate shape. The second jaw 1204 may include four sides, including an outer surface 1219, a compression surface (opposite the outer surface 1219), and a first side surface 1220 and a second side surface facing opposite the first side surface 1220.

The second jaw 1204 may extend along a longitudinal axis 1222 and may be configured as a beam. The first end portion 1216 may comprise a tip 1221 of the second jaw 1204 and the second end portion 1218 may comprise an opposite tip 1224 of the second jaw 1204.

The second jaw 1204 may extend along the first jaw 1202 to form a channel 1226 for receiving a portion of the heart. The compressive surfaces of the first jaw 1202 and second jaw 1204 may face each other and may be positioned on opposite sides of the channel 1226. The channel 1226 may have a first end 1228 at the respective first end portions 1206, 1216 of the first jaw 1202 and the second jaw 1204 and may have a second end 1230 at the respective second end portions 1208, 1218 of the first jaw 1202 and the second jaw 1204.

A first spring 1240 may couple the first jaw 1202 to the second jaw 1204 and may cover the first end 1228 of the channel 1226 at the first end portion 1206 of the first jaw 1202 and the first end portion 1216 of the second jaw 1204. The first spring 1240 may be configured to force the first jaw 1202 towards the second jaw 1204 to compress the portion of the heart within the channel 1226. The first spring 1240 in examples may include a first end 1242 and a second end 1244 and a loop 1246 extending from the first end 1242 and the second end 1244.

The first end 1242 of the first spring 1240 may be coupled to the first jaw 1202 and the second end 1244 of the first spring 1240 may be coupled to the second jaw 1204. The loop 1246 may protrude from the first end portion 1206 of the first jaw 1202 and the first end portion 1216 of the second jaw 1204. The loop 1246 may bound the first end 1228 of the channel 1226. The loop 1246 may include straight portions 1247 and a circular portion 1249, forming a “C” shape. In examples, the loop 1246 may have another shape or configuration as desired.

The first spring 1240 may be spaced from the first end 1228 of the channel 1226 with a gap 1248. The first spring 1240 accordingly may be spaced from the tips 1214, 1221 of the first jaw 1202 and the second jaw 1204 at the first end portions 1206, 1216.

A second spring 1250 may couple the first jaw 1202 to the second jaw 1204 and may cover the second end 1230 of the channel 1226 at the second end portion 1208 of the first jaw 1202 and the second end portion 1218 of the second jaw 1204. The second spring 1250 may be configured to force the first jaw 1202 towards the second jaw 1204 to compress the portion of the heart within the channel 1226. The second spring 1250 in examples may include a first end 1252 and a second end 1254 and a loop 1256 extending from the first end 1252 and the second end 1254.

The first end 1252 of the second spring 1250 may be coupled to the first jaw 1202. The loop 1256 may protrude from the second end portion 1208 of the first jaw 1202 and the second end portion 1218 of the second jaw 1204. The loop 1256 may bound the second end 1230 of the channel 1226. The loop 1256 may include straight portions 1257 and a circular portion 1259, forming a “C” shape. In examples, the loop 1256 may have another shape or configuration as desired.

The second spring 1250 may be spaced from the second end 1230 of the channel 1226 with a gap 1258. The second spring 1250 accordingly may be spaced from the tips 1215, 1224 of the first jaw 1202 and the second jaw 1204 at the second end portions 1208, 1218.

The springs 1240, 1250 may force the compression surfaces of the first jaw 1202 and the second jaw 1204 towards each other to form a closed state for the clip 1200. A portion of a heart may be compressed within the channel 1226 between the first jaw 1202 and the second jaw 1204. In examples, the first jaw 1202 may be moved away for the second jaw 1204 to form an opened state for the clip 1200. In such a configuration the clip 1200 may be positioned over the portion of the heart to be compressed, and then the clip 1200 may be deployed to cause the clip 1200 to move to the closed state.

In examples, a first deflectable arm 1260 may couple the first jaw 1202 to the first spring 1240 and a second deflectable arm 1262 may couple the first jaw 1202 to the second spring 1250. The first deflectable arm 1260 may be angled with respect to the first jaw 1202 and the second deflectable arm 1262 may be angled with respect to the first jaw 1202, in an opposite direction of angle as the first deflectable arm 1260. The deflectable arms 1260, 1262 may span a gap between the first jaw 1202 and the first spring 1240 and the second spring 1250 respectively.

In examples, a third deflectable arm 1264 and a fourth deflectable arm 1266 may be coupled to the second jaw 1204 and may provide a similar function as the first deflectable arm 1260 and the second deflectable arm 1262.

In examples, a first support arm 1270 may be positioned outward of the first jaw 1202 and may couple the first spring 1240 to the second spring 1250. Similarly, a second support arm 1272 may be positioned outward of the second jaw 1204 and may couple the first spring 1240 to the second spring 1250. In examples, the first spring 1240, the second spring 1250, the first support arm 1270, and the second support arm 1272 may form a continuous perimeter around the first jaw 1202 and the second jaw 1204.

The deflectable arms 1260, 1262, 1264, 1266 and the support arms 1270, 1272 may support the jaws 1202, 1204 during compression of the portion of the heart within the channel 1226.

In operation, the clip 1200 may be utilized to close the LAA 18 or another portion of a heart in a similar manner as the clip 1130.

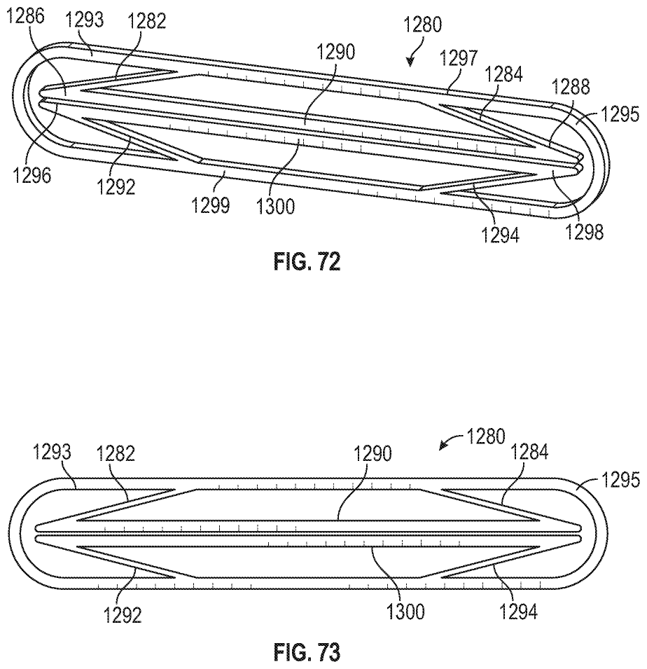

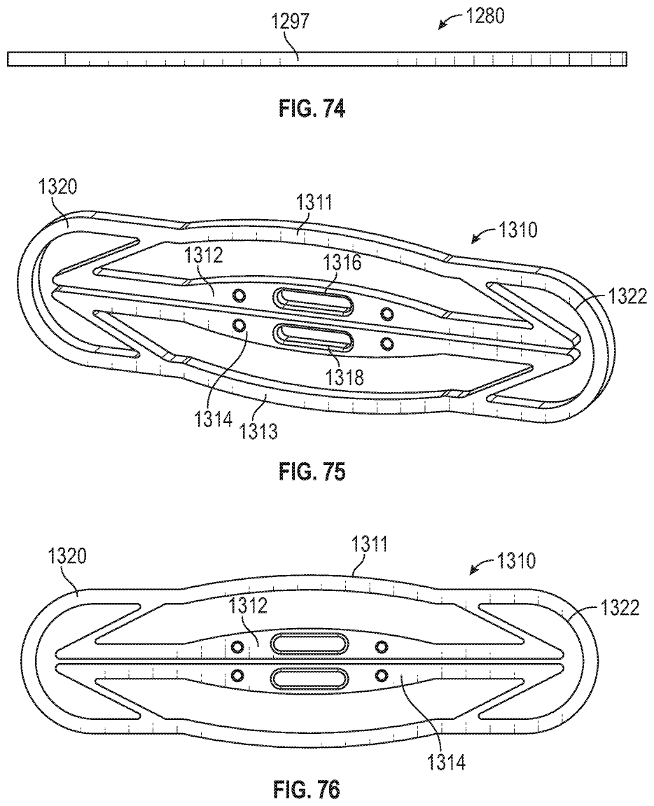

Various modification of the clip 1200 may be provided. FIGS. 72-74 illustrate a clip 1280 in which the first deflectable arm 1282 and the second deflectable arm 1284 are coupled to respective ends 1286, 1288 of the first jaw 1290. The third deflectable arm 1292 and the fourth deflectable arm 1294 are further coupled to respective ends 1296, 1298 of the second jaw 1300. The clip 1280 may include springs 1293, 1295 and support arms 1297, 1299. FIG. 73 illustrates a side view of the clip 1280 and FIG. 74 illustrates a top view.

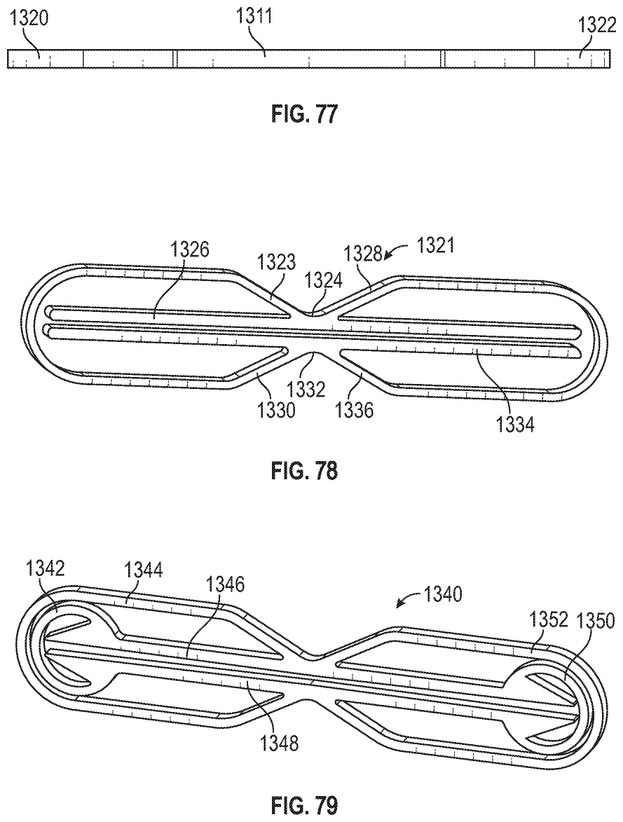

FIGS. 75-77 illustrate a clip 1310 in which the first jaw 1312 and the second jaw 1314 each include respective recesses 1316, 1318 for receiving a device for applying an expansion force to the springs 1320, 1322. The recess 1316, 1318 may pass through respective side surfaces of the first jaw 1312 and the second jaw 1314. The device, for example, may comprise jaws configured to move the jaws 1312, 1314 away from each other to expand the jaws 1312, 1314. The device may release the force against the jaws 1312, 1314 upon deployment. The clip 1310 may include support arms 1311, 1313.

FIG. 78 illustrates a clip 1321 in which the first deflectable arm 1323 is coupled to a central portion 1324 of the first jaw 1326, and a second deflectable arm 1328 is coupled to the central portion 1324 of the first jaw 1326. A third deflectable arm 1330 may be coupled to a central portion 1332 of the second jaw 1334, and a fourth deflectable arm 1336 is coupled to the central portion 1332 of the second jaw 1334.

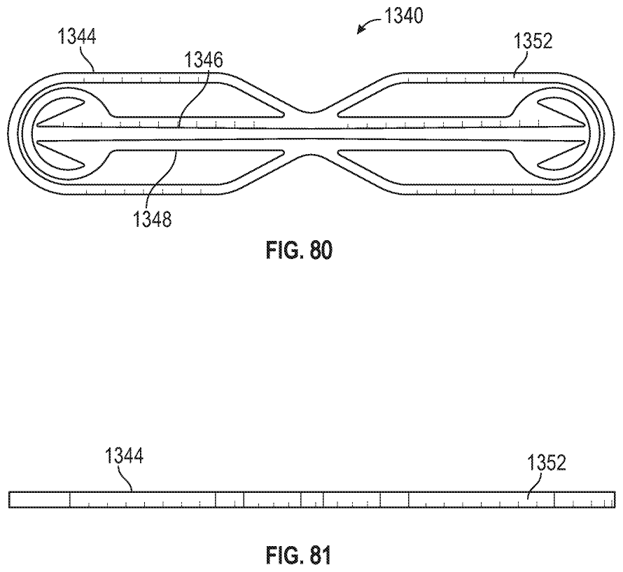

FIGS. 79-81 illustrate an example of a clip 1340 in which a third spring 1342 is positioned inward of the first spring 1344 and couples the first jaw 1346 to the second jaw 1348. In examples, the third spring 1342 may be configured as a loop at the ends of the first jaw 1346 and the second jaw 1348, similar to the spring 1170 shown in FIG. 68. A fourth spring 1350 may be positioned inward of the second spring 1352 and may couple the first jaw 1346 to the second jaw 1348. The fourth spring 1350 may be configured as a loop at the ends of the first jaw 1346 and the second jaw 1348, similar to the spring 1180 shown in FIG. 68. FIG. 80 illustrates a side view of the clip 1340 and FIG. 81 illustrates a top view.

In operation, the clips of FIGS. 71-81 may be utilized to close the LAA 18 or another portion of a heart in a similar manner as the clip 1130.

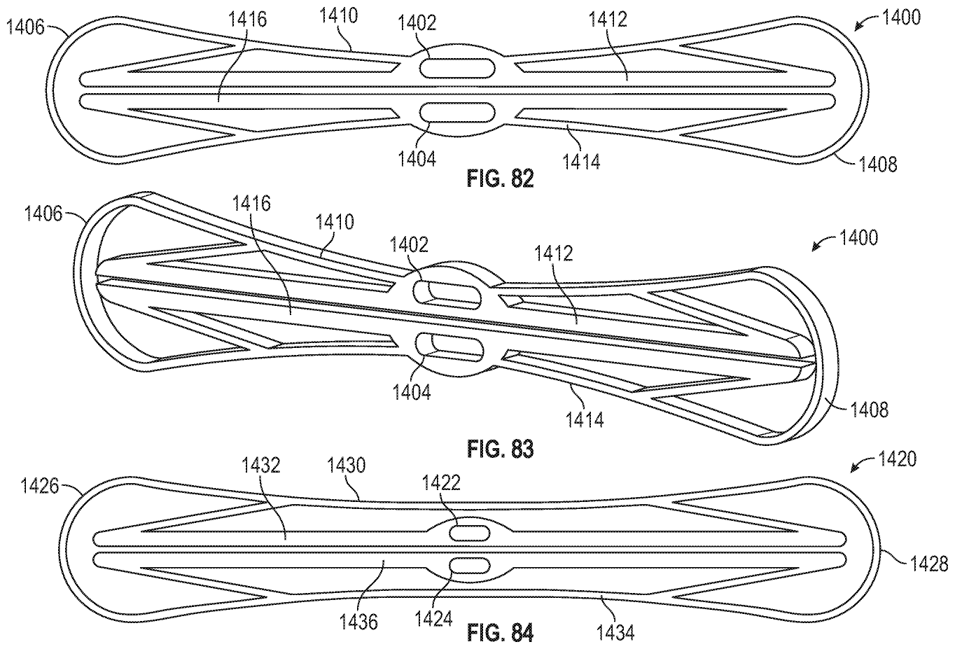

Various modification of the clips may be provided. FIGS. 82 and 83 illustrate a modification of the clip 1310 shown in FIGS. 75-77. Features of the clip 1310 shown in FIGS. 75-77 apply to the clip 1400 shown in FIGS. 82 and 83 unless stated otherwise. The clip 1400 of FIGS. 82 and 83 may include apertures 1402, 1404 for receiving a device for applying an expansion force to the springs 1406, 1408. A first support arm 1410 may be positioned outward of the first jaw 1412 and may couple the first spring 1406 to the second spring 1408 and may be coupled to the aperture 1402. Similarly, a second support arm 1414 may be positioned outward of the second jaw 1416 and may couple the first spring 1406 to the second spring 1408 and may be coupled to the aperture 1404. The apertures 1402, 1404 may comprise respective raised portions of the first jaw 1412 and the second jaw 1416. The apertures 1402, 1404 may comprise curved portions of the first jaw 1412 and the second jaw 1416.

The first support arm 1410 may be positioned outward of the first jaw 1412 and may be bowed towards the first jaw 1412. The second support arm 1414 may be positioned outward of the second jaw 1416 and may be bowed towards the second jaw 1416.

Various modifications of the clip 1400 may be provided. FIG. 84 illustrates a modification of the clip 1400 shown in FIGS. 82 and 83. Features of the clip 1400 shown in FIGS. 82 and 83 apply to the clip 1420 shown in FIG. 84 unless stated otherwise. The clip 1420 shown in FIG. 84 may include apertures 1422, 1424 for receiving a device for applying an expansion force to the springs 1426, 1428. A first support arm 1430 may be positioned outward of the first jaw 1432 and may couple the first spring 1426 to the second spring 1428 and may extend over the aperture 1422. Similarly, a second support arm 1434 may be positioned outward of the second jaw 1436 and may couple the first spring 1426 to the second spring 1428 and may be extend over the aperture 1424. The apertures 1422, 1424 may comprise respective raised portions of the first jaw 1432 and the second jaw 1436. The apertures 1422, 1424 may comprise curved portions of the first jaw 1432 and the second jaw 1436.

The first support arm 1430 may be positioned outward of the first jaw 1432 and may be bowed towards the first jaw 1432. The second support arm 1434 may be positioned outward of the second jaw 1436 and may be bowed towards the second jaw 1436.

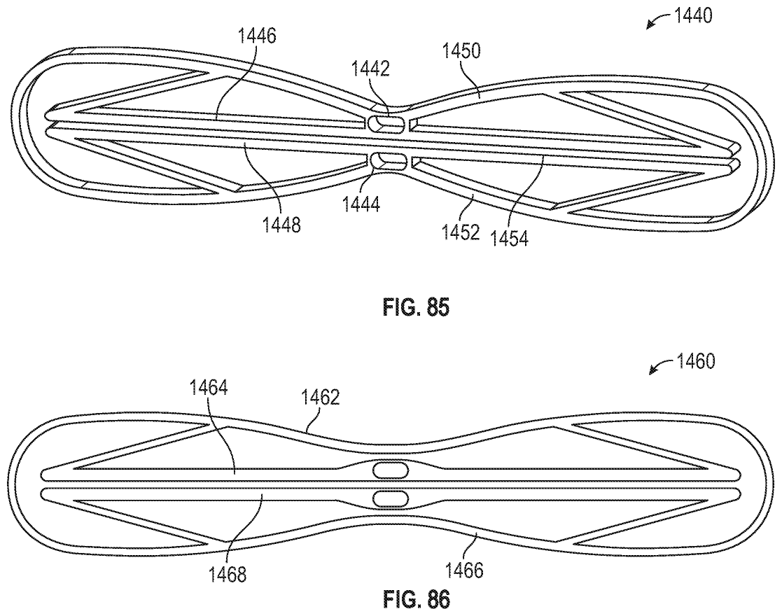

FIG. 85 illustrates a modification of the clip 1400 shown in FIGS. 82 and 83. Features of the clip 1400 shown in FIGS. 82 and 83 apply to the clip 1440 shown in FIG. 85 unless stated otherwise. The clip 1440 shown in FIG. 85 includes apertures 1442, 1444 comprising a raised portion of the first jaw 1446 and second jaw 1448 respectively. The first support arm 1450 may be bowed towards the first jaw 1446 and the second support arm 1452 may be bowed toward the second jaw 1454. The first support arm 1450 may be coupled to the aperture 1442 and the second support arm 1452 may be coupled to the aperture 1444.

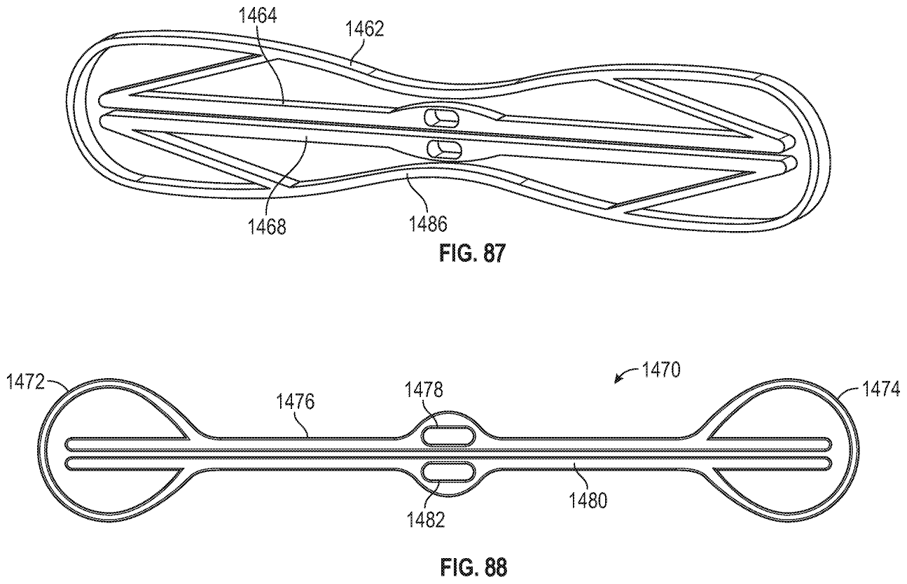

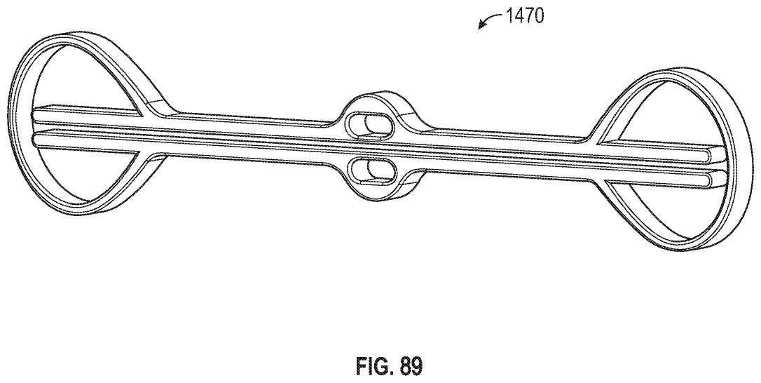

FIGS. 86 and 87 illustrate a modification of the clip 1420 shown in FIG. 84. Features of the clip 1420 shown in FIG. 84 apply to the clip 1460 shown in FIG. 86 unless stated otherwise. The clip 1460 may include a first support arm 1462 having an increased bow or curvature towards the first jaw 1464 than the first support arm 1430 shown in FIG. 84. The clip 1460 may also include a second support arm 1466 having an increased bow or curvature towards the second jaw 1468 than the second support arm 1434 shown in FIG. 84.

FIG. 88 illustrates a modification of the clip 1130 shown in FIG. 68. Features of the clip 1130 shown in FIG. 68 apply to the clip 1470 shown in FIG. 88 unless stated otherwise. The clip 1470 may include a first spring 1472 and a second spring 1474 each having a diameter greater than a combination of both the raised portion of the first jaw 1476 including a first aperture 1478 and the raised portion of the second jaw 1480 including a second aperture 1482.

The clips shown in FIGS. 68-88 may be covered with a soft material (e.g., polyester, for example, DACRON® PET or another form of soft material) that can be deployed to secure the closure of the portion of the body to be occluded. In examples, the clips may be coated with a material to produce a low-profile clip.

The clips may be cut from a single piece of material (e.g., nitinol or another form of shape memory material) in examples. The clips accordingly may be laser cut without welding or bonding in examples. The clips may be configured to seal a closure of the portion of the body to be occluded automatically upon release from a delivery apparatus.

In the clips of FIGS. 82-87, the clips may open in a first stage (e.g., 0 millimeters to 7 millimeters, or another range) at which the jaws (e.g., first jaw and second jaw) may open parallel with each other. The clips may then open in a second stage (e.g., 7 millimeters to 16 millimeters, or another distance) at which the jaws may form a football or ovoid shape. The design may allow for ease of placement over the portion of the body to be occluded and uniform compression across the portion of the body. The amount of compression needed to close the portion of the body may be varied by selecting the material thickness or other configuration of the clip as desired.

In the clip of FIG. 88, the clip may open with the jaws forming a football or ovoid shape.

The clips of FIGS. 68-88 may be recaptured by the delivery apparatus or another device for repositioning as desired.

Various other modifications of the clips disclosed herein may be provided. Various other methods of deployment and use of the clips may be provided as desired.

The clips as disclosed herein may be utilized to close the LAA or may be utilized to close another portion of a heart. In examples, the clips may be utilized to close other portions of a body, including other tubular vessels or other portions of a body. Deployment may be via a delivery apparatus or via another method as desired. Deployment may be via surgical methods and may be transcatheter or via non-invasive surgery in methods.

Variations in the clips and methods disclosed herein may be provided. Features across examples may be combined. Features may be excluded or added to in various examples disclosed herein. Combinations of features of examples may be provided. The features may be utilized solely or in methods disclosed herein, or in combination with other features disclosed herein.

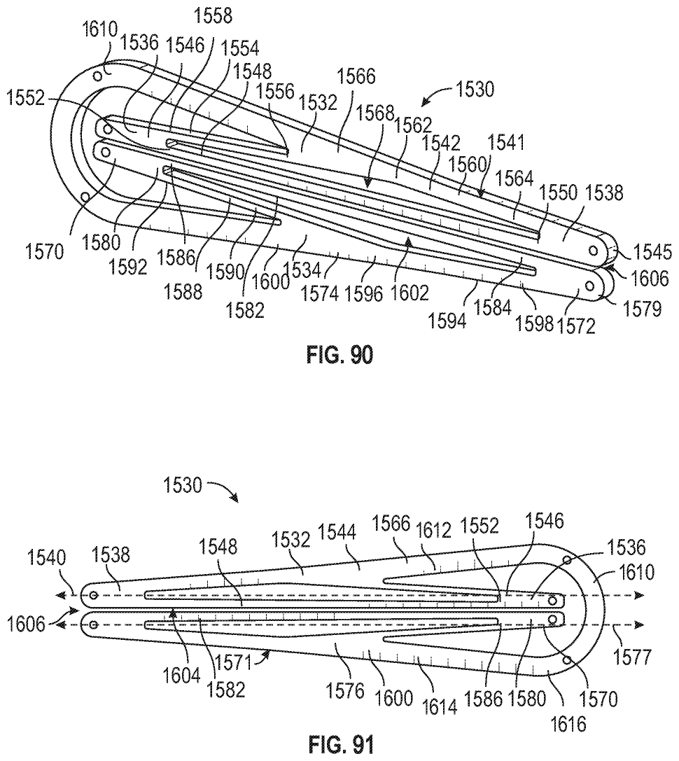

FIG. 90 illustrates an example of a clip 1530 that may be utilized for a portion of a heart. The clip 1530 may be configured to close a LAA 18, to reduce the possibility of clots or other undesired materials stemming from the LAA 18 from traveling into the bloodstream.

The clip 1530 may include a first jaw 1532 and a second jaw 1534. The first jaw 1532 may extend from a first end portion 1536 to a second end portion 1538 and may have an elongate shape. The first jaw 1532 may include four sides, including an outer surface 1541, a compression surface (opposite the outer surface 1541), and two side surfaces 1542, 1544 (with side surface 1544 shown in FIG. 91).

The first jaw 1532 may extend along a longitudinal axis 1540 (marked in FIG. 91) and may taper downward in a direction towards the second end portion 1538 of the first jaw 1532. The second end portion 1538 of the first jaw 1532 may comprise a tip 1545 of the first jaw 1532.

The first jaw 1532 may include a first cantilever beam 1546 extending from the second end portion 1538 of the first jaw 1532 towards the first end portion 1536 of the first jaw 1532. The first cantilever beam 1546 may extend from the tip 1545 of the first jaw 1532 towards the first end portion 1536 of the first jaw 1532. The first cantilever beam 1546 may include two arms, with a first arm 1548 coupled to the tip 1545 of the first jaw 1532 and forming a compressive surface of the first jaw 1532. The first arm 1548 may include a first end 1550 and a second end 1552. The first cantilever beam 1546 may include a second arm 1554 having a first end 1556 and a second end 1558. The second end 1558 of the second arm 1554 may be coupled to the second end 1552 of the first arm 1548.

The first jaw 1532 may include a third arm 1560 forming the outer surface 1541 of the first jaw 1532 and having a first end 1562 and a second end 1564. The third arm 1560 may be positioned outward of the first cantilever beam 1546. The second end 1564 of the third arm 1560 may be coupled to the first end 1550 of the first arm 1548 at the tip 1545 of the first jaw 1532. The third arm 1560 may be positioned at an angle relative to the first arm 1548. The first end 1556 of the second arm 1554 may be coupled to the first end 1562 of the third arm 1560. The second arm 1554 may extend at an angle relative to the first arm 1548.

The first end 1556 of the second arm 1554 may be coupled to the first end 1562 of the third arm 1560 at a central portion 1566 of the first jaw 1532. The central portion 1566 is between the first end portion 1536 and the second end portion 1538 of the first jaw 1532.

A gap 1568 may be positioned between the third arm 1560 and the first arm 1548, and the second arm 1554 and the first arm 1548. The gap 1568 may comprise a cut-out portion in the first jaw 1532.

The second jaw 1534 may be configured similarly as the first jaw 1532 in examples. The second jaw 1534 may extend from a first end portion 1570 to a second end portion 1572 and may have an elongate shape. The second jaw 1534 may include four sides, including an outer surface 1571, a compression surface (opposite the outer surface 1571), and two side surfaces 1574, 1576 (with side surface 1576 shown in FIG. 91).

The second jaw 1534 may extend along a longitudinal axis 1577 (marked in FIG. 91) and may taper downward in a direction towards the second end portion 1572 of the second jaw 1534. The second end portion 1572 of the second jaw 1534 may comprise a tip 1579 of the second jaw 1534.

The second jaw 1534 may include a second cantilever beam 1580 extending from the second end portion 1572 of the second jaw 1534 towards the first end portion 1570 of the second jaw 1534. The second cantilever beam 1580 may extend from the tip 1579 of the second jaw 1534 towards the first end portion 1570 of the second jaw 1534. The second cantilever beam 1580 may include two arms, with a first arm 1582 coupled to the tip 1579 of the second jaw 1534 and forming a compressive surface of the second jaw 1534. The first arm 1582 may include a first end 1584 and a second end 1586. The second cantilever beam 1580 may include a second arm 1588 having a first end 1590 and a second end 1592. The second end 1592 of the second arm 1588 may be coupled to the second end 1586 of the first arm 1582.

The second jaw 1534 may include a third arm 1594 forming the outer surface 1571 of the second jaw 1534 and having a first end 1596 and a second end 1598. The third arm 1594 may be positioned outward of the second cantilever beam 1580. The second end 1598 of the third arm 1594 may be coupled to the first end 1584 of the first arm 1582 at the tip 1579 of the second jaw 1534. The third arm 1594 may be positioned at an angle relative to the first arm 1582. The first end 1590 of the second arm 1588 may be coupled to the first end 1596 of the third arm 1594. The second arm 1588 may extend at an angle relative to the first arm 1582.

The first end 1590 of the second arm 1588 may be coupled to the first end 1596 of the third arm 1594 at a central portion 1600 of the second jaw 1534. The central portion 1600 is between the first end portion 1570 and the second end portion 1572 of the second jaw 1534.

A gap 1602 may be positioned between the third arm 1594 and the first arm 1582, and the second arm 1588 and the first arm 1582. The gap 1602 may comprise a cut-out portion in the second jaw 1534.

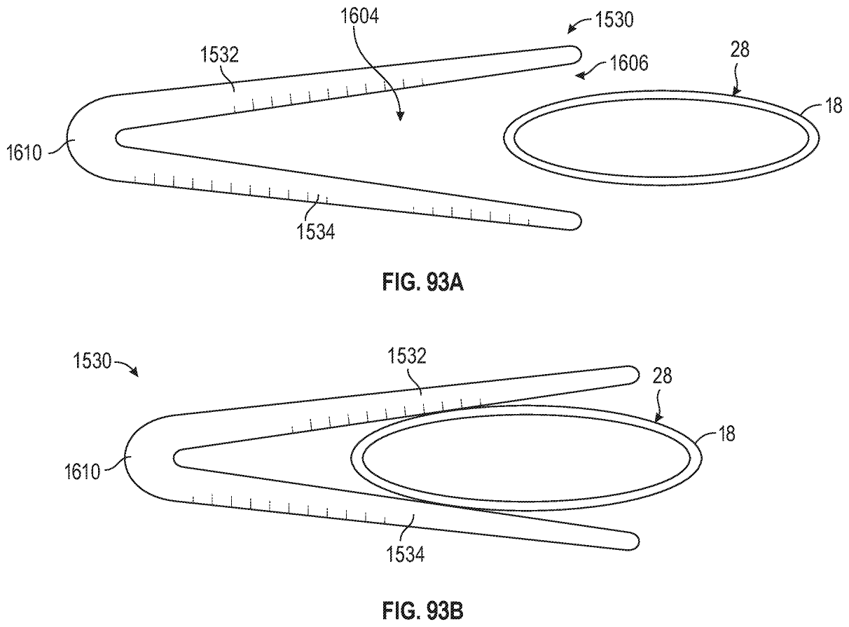

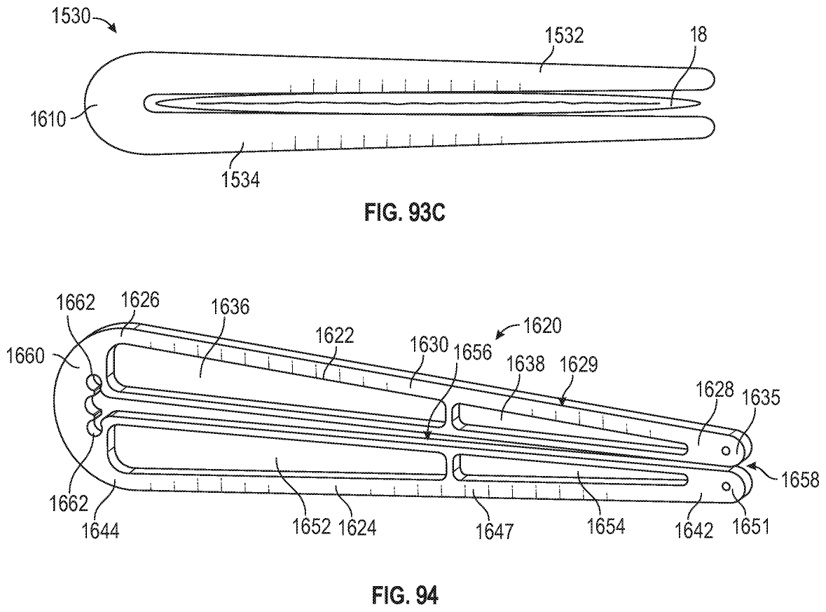

The second jaw 1534 may extend along the first jaw 1532 to form a channel 1604 (marked in FIGS. 91 and 93A) for receiving a portion of the heart. The compressive surfaces of the first jaw 1532 and second jaw 1534 may be positioned on opposite sides of the channel 1604.

The second end portion 1572 of the second jaw 1534 may be separable from the second end portion 1538 of the first jaw 1532 to form an axial opening 1606 for the channel 1604. The separation of the respective end portions 1538, 1578 of the jaws 1532, 1534 may allow a portion of the heart to be inserted into the channel 1604 through the axial opening 1606.

A spring 1610 may be positioned at the first end portion 1536 of the first jaw 1532 and the first end portion 1570 of the second jaw 1534 and configured to force the first jaw 1532 and the second jaw 1534 together to compress a portion of the heart. As shown in FIG. 90, the spring 1610 may comprise an arcuate body protruding outward from the first end portion 1536 of the first jaw 1532 and the first end portion 1570 of the second jaw 1534.

Referring to FIG. 91, the spring 1610 may include a first end 1612 and a second end 1614 and a loop 1616 coupling the first end 1612 to the second end 1614. The first end 1612 may be coupled to the central portion 1566 of the first jaw 1532 and the second end 1614 may be coupled to the central portion 1600 of the second jaw 1534. The loop 1616 may extend over the second end 1552 of the first arm 1548 of the first cantilever beam 1546 and the second end 1586 of the first arm 1582 of the second cantilever beam 1580. The loop 1616 may close an end of the channel 1604 between the first jaw 1532 and the second jaw 1534. The spring 1610 may be integral with the first jaw 1532 and the second jaw 1534, and in examples the clip 1530 may comprise a single body or single piece of material.

The first end portion 1536 of the first jaw 1532 may be pivotally coupled to the first end portion 1570 of the second jaw 1534 such that a distance between the first jaw 1532 and the second jaw 1534 may be increased to open the clip 1530 and reduced to compress a portion of a heart between the first jaw 1532 and the second jaw 1534. The pivotal coupling may be provided by the spring 1610 expanding to allow the first jaw 1532 and second jaw 1534 to pivot. The first jaw 1532 and second jaw 1534 may be pivotally coupled such that a distance between the tip 1545 of the first jaw 1532 and the tip 1579 of the second jaw 1534 varies. The second jaw 1534 may pivot in an are relative to the first jaw 1532 such that the clip 1530 may move from an opened state (as shown in FIGS. 93A and 93B) to a closed state (as shown in FIGS. 90, 91 and 93C).

The compression surfaces of the first jaw 1532 and the second jaw 1534 may be moved towards each other in the closed state. The first cantilever beam 1546 and the second cantilever beam 1580 may each deflect outward upon compression of the portion of the heart between the first cantilever beam 1546 and the second cantilever beam 1580. A deflection of the first cantilever beam 1546 and the second cantilever beam 1580 may increase the compression points at the second end portion 1538 of the first jaw 1532 and the second end portion 1572 of the second jaw 1534.

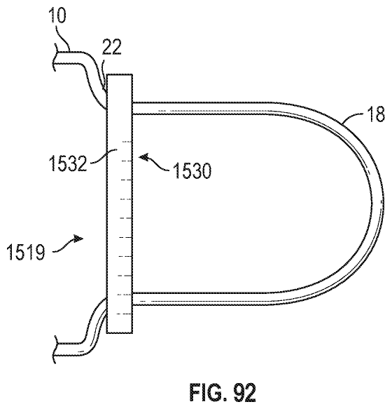

In operation, the clip 1530 may be utilized to close the LAA 18. FIG. 92 illustrates a top schematic view of the clip 1530 closing or occluding the LAA 18, and being positioned at the ostium 22 of the LAA 18. The clip 1530 may span the ostium 22 to close the ostium 22 and reduce blood flow between the LAA 18 and the main chamber 1519 of the left atrium 10.

FIGS. 93A-93C illustrate an exemplary deployment of the clip 1530. Features of the clip 1530 may be excluded from view in FIGS. 93A-93C. In FIG. 93A, the clip 1530 may be placed in an opened state and moved towards the LAA 18 in a direction transverse to the main axis of the LAA 18. In examples, other approaches (e.g., along the main axis of the LAA 18) may be utilized as desired. Access may be provided to the LAA 18 in a variety of manners, including a transcatheter access or a surgical access such as a thoracotomy, mini thoracotomy, or other methods of access.

In FIG. 93B, the clip 1530 may be placed over the outer surface 28 of the LAA 18. In examples, a delivery apparatus may be utilized to move the clip 1530 towards the LAA 18, or in examples, a user (e.g., a medical technician such as a surgeon, or other form of medical technician) may manually place the clip 1530. The clip 1530 may be placed over the outer surface 28 of the LAA 18 in an opened state. A user may confirm a desired placement of the clip 1530 over the LAA 18.

In FIG. 93C, the clip 1530 may be moved to a closed state with the LAA 18 compressed between the first jaw 1532 and the second jaw 1534. The spring 1610 may force the clip 1530 to the closed state. The entire span of the LAA 18 may be closed as desired at the ostium (as shown in FIG. 92) or another portion of the LAA 18 may be closed.

In examples, if desired, the clip 1530 may be opened and either removed or repositioned in a deployment procedure or another procedure. The clip 1530 may be grasped and the force of the spring 1610 may be overcome to open the clip 1530.

The clip 1530 may be utilized to close the LAA 18, yet in examples other portions of a heart may be clipped or closed via use of the clip 1530. In examples, other portions of a body, such as a tubular vessel or other portions of a body may be closed with the clip 1530. Deployment may be via a delivery apparatus or via another method as desired.

Variations in the configuration of the clip 1530 may be provided as desired.

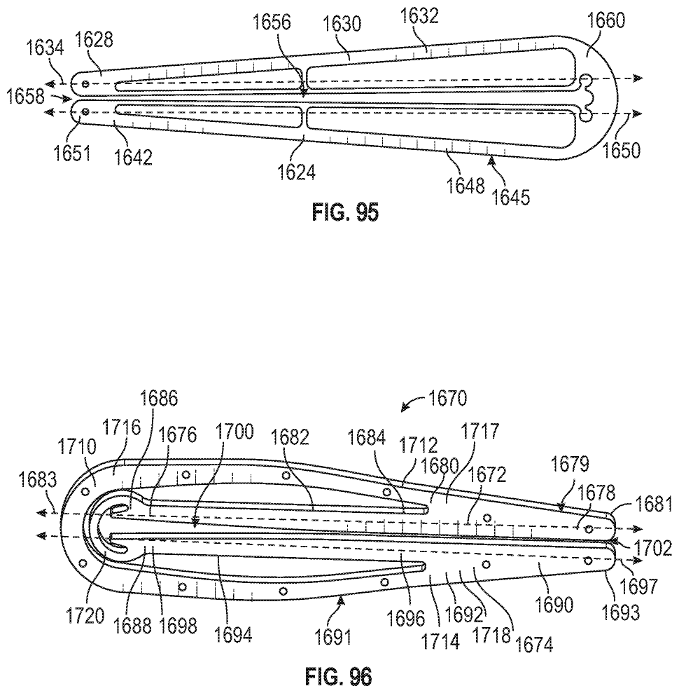

FIGS. 94 and 95 illustrates an example of a clip 1620 that may be utilized for a portion of a heart. The clip 1620 may be configured to close a LAA 18, to reduce the possibility of clots or other undesired materials stemming from the LAA 18 from traveling into the bloodstream.

The clip 1620 may include a first jaw 1622 and a second jaw 1624. The first jaw 1622 may extend from a first end portion 1626 to a second end portion 1628 and may have an elongate shape. The first jaw 1622 may include four sides, including an outer surface 1629, a compression surface (opposite the outer surface 1629), and two side surfaces 1630, 1632 (with side surface 1632 shown in FIG. 95).

The first jaw 1622 may extend along a longitudinal axis 1634 (marked in FIG. 95) and may taper downward in a direction towards the second end portion 1628 of the first jaw 1622. The second end portion 1628 of the first jaw 1622 may comprise a tip 1635 of the first jaw 1532.

The first jaw 1622 may include a plurality of cut out portions 1636, 1638.

The second jaw 1624 may be configured similarly as the first jaw 1622 in examples. The second jaw 1624 may extend from a first end portion 1644 to a second end portion 1642 and may have an elongate shape. The second jaw 1624 may include four sides, including an outer surface 1645, a compression surface (opposite the outer surface 1645), and two side surfaces 1647, 1648 (with side surface 1648 shown in FIG. 95).

The second jaw 1624 may extend along a longitudinal axis 1650 (marked in FIG. 95) and may taper downward in a direction towards the second end portion 1642 of the second jaw 1624. The second end portion 1642 of the second jaw 1624 may comprise a tip 1651 of the second jaw 1624.

The second jaw 1624 may include a plurality of cut out portions 1652, 1654.

The second jaw 1624 may extend along the first jaw 1622 to form a channel 1656 for receiving a portion of the heart. The compressive surfaces of the first jaw 1622 and second jaw 1624 may be positioned on opposite sides of the channel 1656.

The second end portion 1642 of the second jaw 1624 may be separable from the second end portion 1628 of the first jaw 1622 to form an axial opening 1658 for the channel 1656. The separation of the respective end portions 1628, 1642 of the jaws 1622, 1624 may allow a portion of the heart to be inserted into the channel 1656 through the axial opening 1658.

A spring 1660 may be positioned at the first end portion 1626 of the first jaw 1622 and the first end portion 1644 of the second jaw 1624 and configured to force the first jaw 1622 and the second jaw 1624 together to compress a portion of the heart. The spring 1660 may comprise an arcuate body protruding outward from the first end portion 1626 of the first jaw 1622 and the first end portion 1644 of the second jaw 1624. The spring 1660, in examples, may include stress relief holes 1662 to increase the width of the opening of the jaws 1622, 1624.

The spring 1660 may close an end of the channel 1656 between the first jaw 1622 and the second jaw 1624. The spring 1660 may be integral with the first jaw 1622 and the second jaw 1624, and in examples the clip 1620 may comprise a single body or single piece of material.

The first end portion 1626 of the first jaw 1622 may be pivotally coupled to the first end portion 1644 of the second jaw 1624 such that a distance between the first jaw 1622 and the second jaw 1624 may be reduced to compress a portion of a heart between the first jaw 1622 and the second jaw 1624. The pivotal coupling may be provided by the spring 1660 expanding to allow the first jaw 1622 and second jaw 1624 to pivot. The first jaw 1622 and second jaw 1624 may be pivotally coupled such that a distance between the tip 1635 of the first jaw 1622 and the tip 1651 of the second jaw 1624 varies. The second jaw 1624 may pivot in an arc relative to the first jaw 1622 such that the clip 1620 may move from an opened state to a closed state (as shown in FIGS. 94 and 95).

The compression surfaces of the first jaw 1622 and the second jaw 1624 may be moved towards each other in the closed state.

FIG. 95 illustrates a side view of an opposite side of the clip 1620 than shown in FIG. 94.

In operation, the clip 1620 may be utilized to close the LAA 18. The clip 1620 may span the ostium 22 to close the ostium 22 and reduce blood flow between the LAA 18 and the main chamber 1519 of the left atrium 10, in a similar manner as the clip 1530.

FIG. 96 illustrates an example of a clip 1670 that may be utilized for a portion of a heart. The clip 1670 may be configured to close a LAA 18, to reduce the possibility of clots or other undesired materials stemming from the LAA 18 from traveling into the bloodstream.

The clip 1670 may include a first jaw 1672 and a second jaw 1674. The first jaw 1672 may extend from a first end portion 1676 to a second end portion 1678 and may have an elongate shape. The first jaw 1672 may include four sides, including an outer surface 1679, a compression surface (opposite the outer surface 1679), and two side surfaces 1680 (with the other side surface facing opposite the side surface 1680).

The first jaw 1672 may extend along a longitudinal axis 1683 and may taper downward in a direction towards the second end portion 1678 of the first jaw 1672. The second end portion 1678 of the first jaw 1672 may comprise a tip 1681 of the first jaw 1672.

The first jaw 1672 may include a first arm 1682 extending from the second end portion 1678 of the first jaw 1672 towards the first end portion 1676 of the first jaw 1672. The first arm 1682 may form the compressive surface of the first jaw 1672 and may have a first end 1684 and a second end 1686.

The second jaw 1674 may be configured similarly as the first jaw 1672 in examples.

The second jaw 1674 may extend from a first end portion 1688 to a second end portion 1690 and may have an elongate shape. The second jaw 1674 may include four sides, including an outer surface 1691, a compression surface (opposite the outer surface 1691), and two side surfaces 1692 (with the other side surface facing opposite the side surface 1692).

The second jaw 1674 may extend along a longitudinal axis 1697 and may taper downward in a direction towards the second end portion 1690 of the second jaw 1674. The second end portion 1690 of the second jaw 1674 may comprise a tip 1693 of the second jaw 1674.

The second jaw 1674 may include a second arm 1694 extending from the second end portion 1690 of the second jaw 1674 towards the first end portion 1688 of the second jaw 1674. The second arm 1694 may form the compressive surface of the second jaw 1674 and may have a first end 1696 and a second end 1698.

The second jaw 1674 may extend along the first jaw 1672 to form a channel 1700 for receiving a portion of the heart. The compressive surfaces of the first jaw 1672 and second jaw 1674 may be positioned on opposite sides of the channel 1700.

The second end portion 1690 of the second jaw 1674 may be separable from the second end portion 1678 of the first jaw 1672 to form an axial opening 1702 for the channel 1700. The separation of the respective end portions 1690, 1678 of the jaws 1674, 1672 may allow a portion of the heart to be inserted into the channel 1700 through the axial opening 1702.

A spring 1710 may be positioned at the first end portion 1676 of the first jaw 1672 and the first end portion 1688 of the second jaw 1674 and configured to force the first jaw 1672 and the second jaw 1674 together to compress a portion of the heart. The spring 1710 may comprise an arcuate body protruding outward from first end portion 1676 of the first jaw 1672 and the first end portion 1688 of the second jaw 1674.

The spring 1710 may include a first end 1712 and a second end 1714 and a loop 1716 coupling the first end 1712 to the second end 1714. The first end 1712 may be coupled to a central portion 1717 of the first jaw 1672 and the second end 1714 may be coupled to the central portion 1718 of the second jaw 1674. The loop 1716 may extend over the second end 1686 of the first arm 1682 the second end 1698 of the second arm 1694. The spring 1710 may be integral with the first jaw 1672 and the second jaw 1674, and in examples the clip 1670 may comprise a single body or single piece of material.

The clip 1670 may include a spring 1720 coupled to the second end 1686 of the first arm 1682 and coupled to the second end 1698 of the second arm 1694. The spring 1720 may include a loop that may close an end of the channel 1700 between the first jaw 1672 and the second jaw 1674. The spring 1720 may comprise an arcuate body and may have a circular shape. The spring 1710 may extend around the spring 1720.

The first end portion 1676 of the first jaw 1672 may be pivotally coupled to the first end portion 1688 of the second jaw 1674 such that a distance between the first jaw 1672 and the second jaw 1674 may be increased to open the clip 1670 and reduced to compress a portion of a heart between the first jaw 1672 and the second jaw 1674. The pivotal coupling may be provided by both springs 1710, 1720 expanding to allow the first jaw 1672 and second jaw 1674 to pivot. The first jaw 1672 and second jaw 1674 may be pivotally coupled such that a distance between the tip 1681 of the first jaw 1672 and the tip 1693 of the second jaw 1674 varies. The second jaw 1674 may pivot in an are relative to the first jaw 1672 such that the clip 1670 may move from an opened state to a closed state.

The compression surfaces of the first jaw 1672 and the second jaw 1674 may be moved towards each other in the closed state. The arms 1682, 1694 may each deflect outward upon compression of the portion of the heart between the arms 1682, 1694. The deflection of the arms 1682, 1694 may increase the compression points at the second end portion 1678 of the first jaw 1672 and the second end portion 1690 of the second jaw 1674, to better secure the portion of the heart being compressed.

In operation, the clip 1670 may be utilized to close the LAA 18. The clip 1670 may span the ostium 22 to close the ostium 22 and reduce blood flow between the LAA 18 and the main chamber 1519 of the left atrium 10, in a similar manner as the clip 30.

Various other modifications of the clips disclosed herein may be provided. Various other methods of deployment and use of the clips may be provided as desired.

The examples of clips shown in FIGS. 90-96 may include a curvature. The curvature may match a curvature of a LAA in examples. The curvature of the LAA may be measured prior to or during a deployment operation and the jaws of the clip may have a curvature that matches the curvature of the LAA.

The clips as disclosed herein may be utilized to close the LAA or may be utilized to close another portion of a heart. In examples, the clips may be utilized to close other portions of a body, including other tubular vessels or other portions of a body. Deployment may be via a delivery apparatus or via another method as desired. Deployment may be via surgical methods and may be transcatheter or via non-invasive surgery in methods.

Variations in the clips and methods disclosed herein may be provided. Features across examples may be combined. Features may be excluded or added to in various examples disclosed herein. Combinations of features of examples may be provided. The features may be utilized solely or in methods disclosed herein, or in combination with other features disclosed herein.

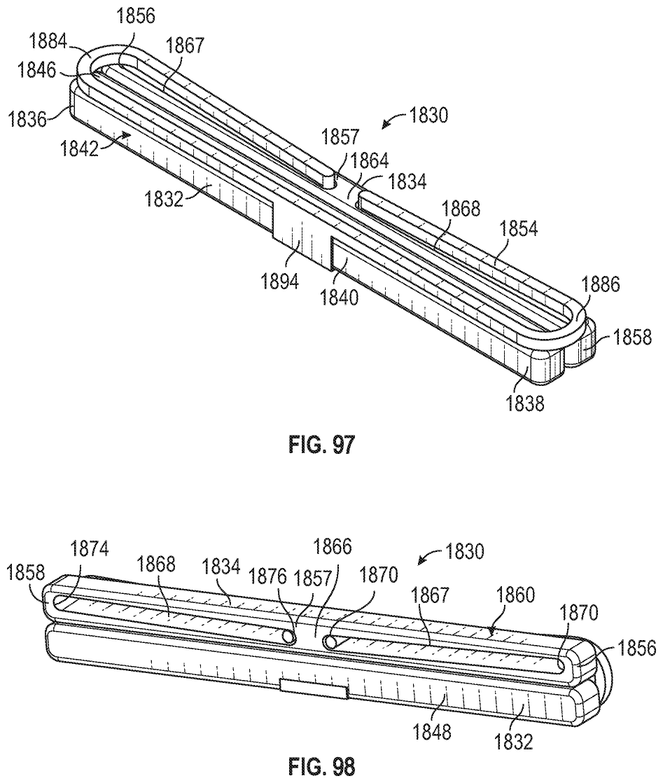

FIG. 97 illustrates an example of a clip 1830 that may be utilized for a portion of a heart. The clip 1830 may be configured to close a LAA 18, to reduce the possibility of clots or other undesired materials stemming from the LAA 18 from traveling into the bloodstream.

The clip 1830 may include a first jaw 1832 and a second jaw 1834. The first jaw 1832 may extend from a first end portion 1836 to a second end portion 1838 and may have an elongate shape. The first jaw 1832 may be configured as an elongate beam. The first jaw 1832 may have a central portion 1840 between the first end portion 1836 and the second end portion 1838.

The first jaw 1832 may include four sides, including an outer surface 1842, a compression surface 1844 (marked in FIG. 101 and facing opposite the outer surface 1842 and towards the second jaw 1834), and two side surfaces 1846, 1848 (marked in FIGS. 98 and 101) each extending between the compression surface 1844 and the outer surface 1842.

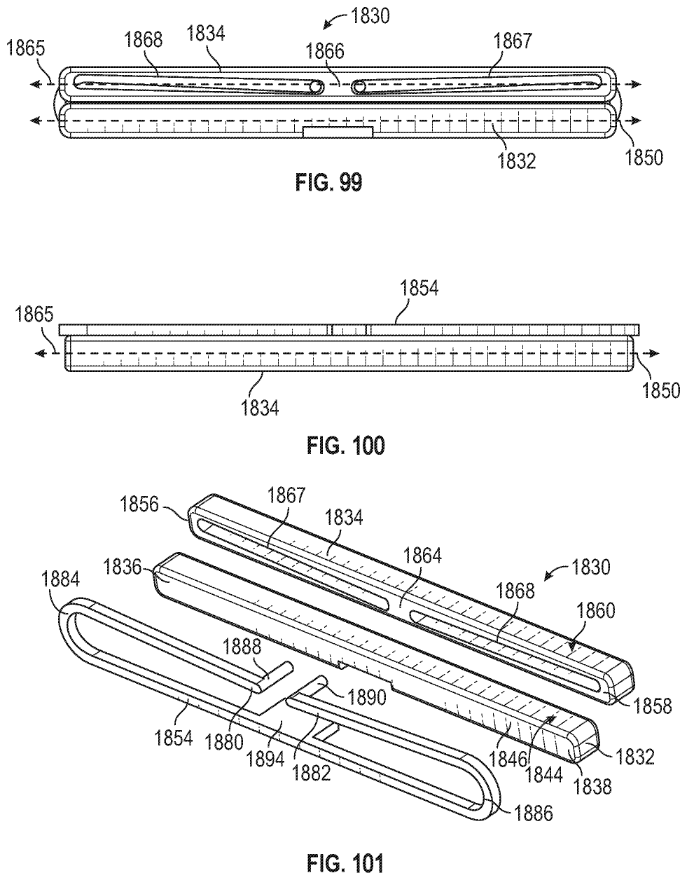

The first jaw 1832 may extend along a longitudinal axis 1850 (marked in FIGS. 99 and 100) and may have a rectangular cross section when viewed perpendicular to the longitudinal axis 1850. The first jaw 1832 may have another shape in examples as desired.

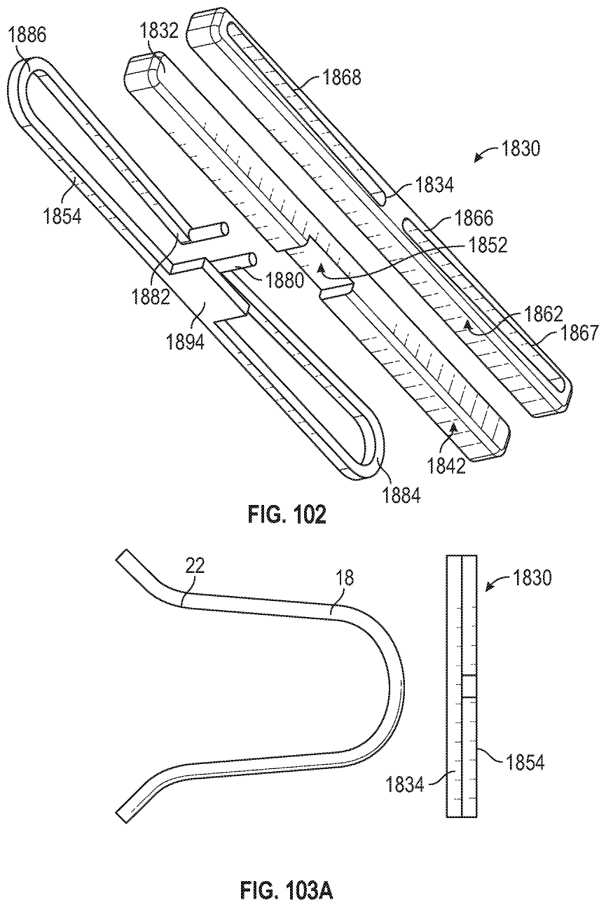

The first jaw 1832 may include a recess 1852 (marked in FIG. 102). The recess 1852 may be configured to couple to a portion of a spring 1854 in examples and may be positioned on the outer surface 1842 of the first jaw 1832.

Referring to FIG. 97, the second jaw 1834 may extend from a first end portion 1856 to a second end portion 1858 and may have an elongate shape. The second jaw 1834 may be configured as an elongate beam. The second jaw 1834 may have a central portion 1857 between the first end portion 1856 and the second end portion 1858.

The second jaw 1834 may include four sides, including an outer surface 1860 (marked in FIG. 98), a compression surface 1862 (marked in FIG. 102 and facing opposite the outer surface 1860 and towards the first jaw 1832), and two side surfaces 1864, 1866 (marked in FIGS. 101 and 102) each extending between the compression surface 1862 and the outer surface 1860.

The second jaw 1834 may extend parallel with the first jaw 1832.

The second jaw 1834 may extend along a longitudinal axis 1865 (marked in FIGS. 99 and 100) and may have rectangular cross section when viewed perpendicular to the longitudinal axis 1865. The second jaw 1834 may have another shape in examples as desired.

The second jaw 1834 may include a coupler for coupling to the spring 1854. The coupler may be in the form of one or more tracks 1867, 1868 extending along at least a portion of the second jaw 1834. The one or more tracks 1867, 1868 may be configured for the spring 1854 to slide along. The one or more tracks 1867, 1868 may be positioned on one or more of the side surfaces 1864, 1866 of the second jaw 1834. For example, FIG. 98 illustrates tracks 1867, 1868 on the side surface 1866. The tracks 1867, 1868 may pass through the second jaw 1834 from the side surface 1864 shown in FIG. 97 to the side surface 1866 shown in FIG. 98, or may pass through only a portion of the second jaw 1834.

In examples, the tracks 1867, 1868 may extend inward from the respective first end portion 1856 and the second end portion 1858 of the second jaw 1834 in a direction towards the central portion 1857 of the second jaw 1834. Referring to FIG. 98, the track 1867 may have a first end 1870 at the first end portion 1856 of the second jaw 1834 and a second end 1872 at the central portion 1857 of the second jaw 1834. The track 1868 may have a first end 1874 at the second end portion 1858 of the second jaw 1834 and a second end 1876 at the central portion 1857 of the second jaw 1834.

In examples, the tracks 1867, 1868 may be angled relative to the compression surface 1862 of the second jaw 1834. For example, the tracks 1867, 1868 may each be angled towards the compression surface 1862 of the second jaw 1834 in a direction from the respective end portion 1856, 1858 of the second jaw 1834 towards the central portion 1857. The tracks 1867, 1868 may each have a linear shape from the respective end portion 1856, 1858 of the second jaw 1834 towards the central portion 1857.

FIG. 101 illustrates an assembly view of the clip 1830. The spring 1854 may couple the first jaw 1832 to the second jaw 1834 and may be configured to force the first jaw 1832 towards the second jaw 1834 to compress the portion of the heart between the first jaw 1832 and the second jaw 1834. The spring 1854 may be configured to slide relative to the second jaw 1834 to allow movement of the first jaw 1832 relative to the second jaw 1834.

The spring 1854 may include a first end 1880 and a second end 1882 and may include a first loop 1884 and a second loop 1886. The first end 1880 may comprise a coupler 1888 that may be configured to couple to the second jaw 1834 and slide relative to the second jaw 1834. The coupler 1888, for example, may be positioned within the track 1867 of the second jaw 1834 and may be configured to slide within the track 1867. The coupler 1888 may comprise a pin configured to slide within the track 1867.

The second end 1882 may comprise a coupler 1890 that may be configured to couple to the second jaw 1834 and slide relative to the second jaw 1834. The coupler 1890, for example, may be positioned within the track 1868 of the second jaw 1834 and may be configured to slide within the track 1868. The coupler 1890 may comprise a pin configured to slide within the track 1868.

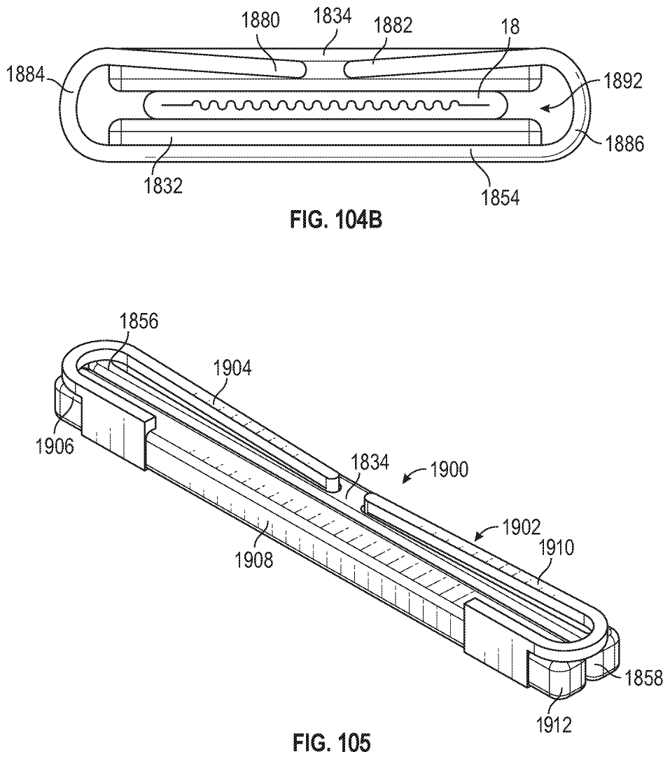

The first loop 1884 may extend from the first end 1880 towards the first end portion 1856 of the second jaw 1834 and the first end portion 1836 of the first jaw 1832. The second loop 1886 may extend from the second end 1882 towards the second end portion 1858 of the second jaw 1834 and the second end portion 1838 of the first jaw 1832. The first loop 1884 and the second loop 1886 may bound ends of a channel 1892 (marked in FIG. 104A) between the first jaw 1832 and the second jaw 1834.

The first loop 1884 and the second loop 1886 may each include ends that couple to a coupler 1894 of the spring 1854 that is configured to couple to the recess 1852 of the first jaw 1832. The spring 1854 may comprise a continuous body that forms the first loop 1884 extending towards the first end portion 1836 of the first jaw 1832 and the first end portion 1856 of the second jaw 1834 and the second loop 1886 extending towards the second end portion 1838 of the first jaw 1832 and the second end portion 1858 of the second jaw 1834.

Referring to FIG. 97, the spring 1854 may protrude from the side surface 1846 of the first jaw 1832 and the side surface 1864 of the second jaw 1834.

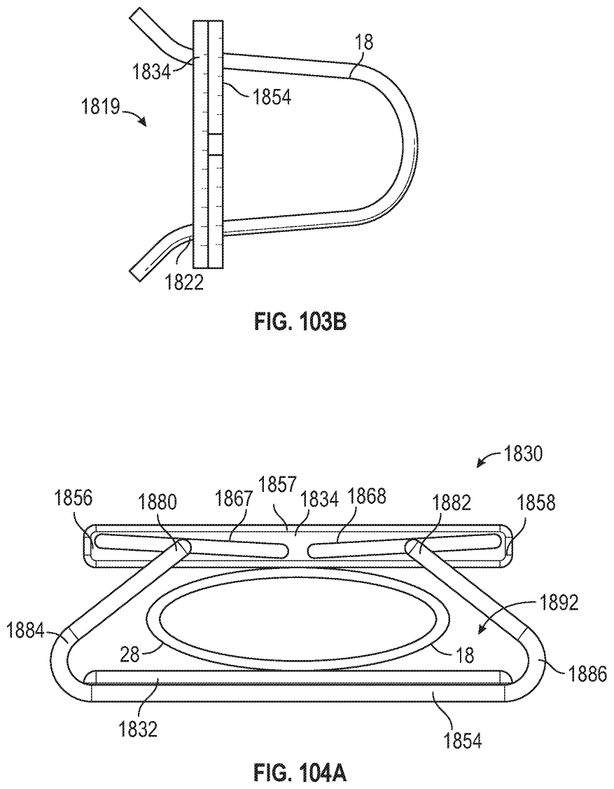

The spring 1854 may be positioned offset from a plane of movement of the first jaw 1832 and the second jaw 1834. The spring 1854 may extend in a plane that is parallel with the plane of movement of the first jaw 1832 and the second jaw 1834. Such a feature may allow the side surfaces 1848, 1866 (shown in FIG. 98) to be positioned close to the ostium 1822 of the LAA 18, without interference from the spring 1854, as shown in FIG. 103B for example.

The spring 1854 may be configured to force the clip 1830 towards a closed state. The spring 1854 may provide a force that moves the compression surfaces of the first jaw 1832 and second jaw 1834 towards each other to compress a portion of the heart therein.

A movement of the first jaw 1832 and the second jaw 1834 to an opened state may cause the spring 1854 to slide relative to the second jaw 1834. The sliding of the spring 1854 may allow the first jaw 1832 to move relative to the second jaw 1834 to allow the clip 1830 to move to the opened state (as shown in FIG. 104A for example). The sliding of the spring 1854 may allow the first jaw 1832 to move relative to the second jaw 1834 to allow the clip 1830 to move to the closed state (as shown in FIG. 104B for example).

FIG. 98 illustrates a side perspective view of the clip 1830 opposite the side shown in FIG. 97. FIG. 99 illustrates a side view of the clip 1830. FIG. 100 illustrates a top view of the clip 1830.

FIG. 101 illustrates an assembly view of the clip 1830. FIG. 102 illustrates an assembly view of the clip 1830 from a side opposite the side shown in FIG. 101.

In operation, the clip 1830 may be utilized to close the LAA 18. FIG. 103A illustrates a top schematic view of the clip 1830 being advanced towards the LAA 18. The clip 1830 may be slid axially over the LAA 18 with the LAA 18 passing into the channel 1892 (marked in FIG. 104A). FIG. 103B illustrates the clip 1830 in position at the ostium 22 of the LAA 18. The clip 1830 may span the ostium 22 to close the ostium 22 and reduce blood flow between the LAA 18 and the main chamber 1819 of the left atrium 10.

FIGS. 104A and 104B illustrate an exemplary deployment of the clip 1830. Features of the clip 1830 may be excluded from view in FIGS. 104A and 104B. In FIG. 104A, the clip 1830 may be placed in an opened state. The spring 1854 is shown to slide relative to the second jaw 1834, within the tracks 1867, 1868. The ends 1880, 1882 of the spring 1854 slide outward from the central portion 1857 of the second jaw 1834 towards the respective first end portion 1856 and second end portion 1858 of the second jaw 1834. The clip 1830 may be moved towards the LAA 18 in a direction transverse to the main axis of the LAA 18. In examples, other approaches (e.g., along the main axis of the LAA 18) may be utilized as desired. Access may be provided to the LAA 18 in a variety of manners, including a transcatheter access or a surgical access such as a thoracotomy, mini thoracotomy, or other methods of access.

The clip 1830 may be placed over the outer surface 28 of the LAA 18. In examples, a delivery apparatus may be utilized to move the clip 1830 towards the LAA 18, or in examples, a user (e.g., a medical technician such as a surgeon, or other form of medical technician) may manually place the clip 1830. The clip 1830 may be placed over the outer surface 28 of the LAA 18 in an opened state. A user may confirm a desired placement of the clip 1830 over the LAA 18.

In FIG. 104B, the clip 1830 may be moved to a closed state with the LAA 18 compressed between the first jaw 1832 and the second jaw 1834. The spring 1854 may force the clip 1830 to the closed state. The spring 1854 may slide relative to the second jaw 1834. The ends 1880, 1882 of the spring 1854 slide inward from respective first end portion 1856 and second end portion 1858 towards the central portion 1857 of the second jaw 1834. The ends 1880, 1882 may slide along the angled tracks 1867, 1868 towards the compression surface 1862 of the second jaw 1834. The entire span of the LAA 18 may be closed as desired at the ostium (as shown in FIG. 104B) or another portion of the LAA 18 may be closed.

In examples, if desired, the clip 1830 may be opened and either removed or repositioned in a deployment procedure or another procedure. The clip 1830 may be grasped and the force of the spring 1854 may be overcome to open the clip 1830.

The clip 1830 may be utilized to close the LAA 18, yet in examples other portions of a heart may be clipped or closed via use of the clip 1830. In examples, other portions of a body, such as a tubular vessel or other portions of a body may be closed with the clip 1830. Deployment may be via a delivery apparatus or via another method as desired.

In examples, other forms of delivery apparatuses may be utilized as desired.

Variations in the configuration of the clip 1830 may be provided as desired.

FIG. 105, for example, illustrates an example of a clip 1900 in which the spring 1902 includes a first spring body 1904 extending towards the first end portion 1906 of the first jaw 1908 and the first end portion 1856 of the second jaw 1834, and a second spring body 1910 extending towards the second end portion 1912 of the first jaw 1908 and the second end portion 1858 of the second jaw 1834. The spring 1902 accordingly may include at least two spring bodies.

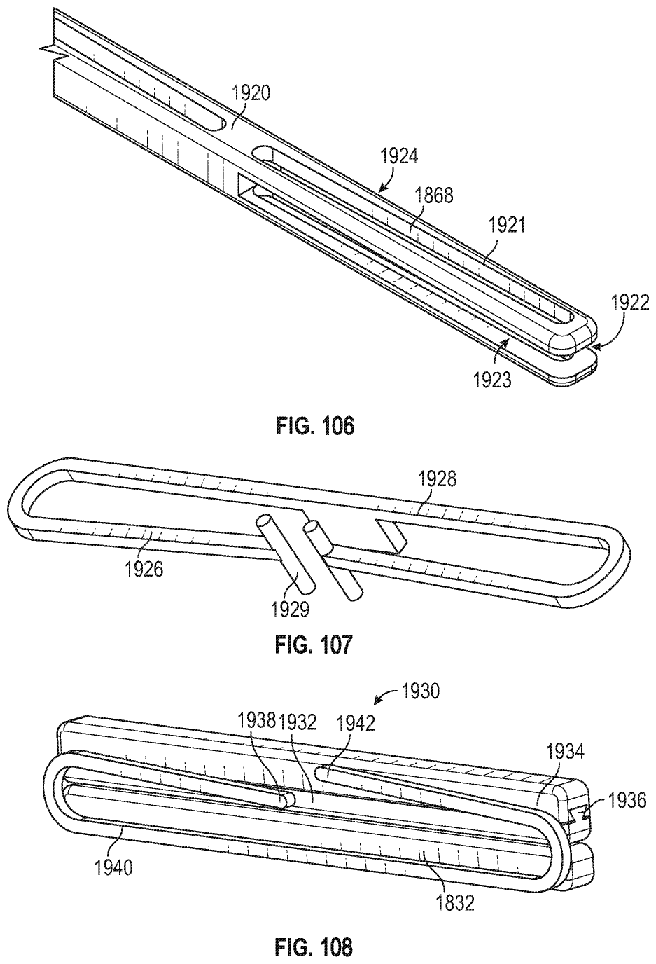

FIG. 106 illustrates an example in which the second jaw 1920 may include a track 1922 on the outer surface 1924 of the second jaw 1920. In such a configuration, a loop 1926 of the spring 1928 (as marked in FIG. 107) may pass through the track 1922 on the outer surface 1924. The loop 1926 shown in FIG. 107 may be positioned between the side surfaces 1921 (with a side surface being positioned opposite the side surface 1921 shown in FIG. 106) of the second jaw 1920 and may extend out of the track 1922 on the outer surface 1924. The loop 1926 may thus slide within a channel 1923 formed between the side surfaces of the second jaw 1920.

The spring 1928 may be positioned centrally with respect to the second jaw 1920 and configured to slide along the track 1922 on the outer surface 1924 of the second jaw 1920. The spring 1928 accordingly may extend in the same plane as the first jaw 1832 and the second jaw 1920 during movement of the first jaw 1832 and the second jaw 1920.

FIG. 107 illustrates a perspective view of a configuration of spring 1928 that may be utilized with the track 1922 shown in FIG. 106. The spring 1928 may include a coupler 1929 that may protrude outward through the track 1868 on the side surface 1921 of the second jaw 1920 to retain the spring 1928 within the track 1868 and allow for sliding movement along the track 1922 on the outer surface 1924. The track 1868 may remain angled as shown in FIG. 98 for example.

FIGS. 108-110 illustrate an example of a clip 1930 in which the second jaw 1932 is coupled to a slide bar 1934. The slide bar 1934 may be positioned outward of the second jaw 1932 in examples. The slide bar 1934 may be configured to slide relative to the second jaw 1932. The slide bar 1934 may slide along the length of the second jaw 1932 and parallel with the second jaw 1932. A slide coupler 1936 may be utilized, such as a dovetail joint or another form of slide coupler as desired.

The second jaw 1932 may be coupled to a first end 1938 of the spring 1940, and the slide bar 1934 may be coupled to a second end 1942 of the spring 1940. The slide bar 1934 and the spring 1940 may be configured to slide relative to the second jaw 1932 to allow movement of the first jaw 1832 relative to the second jaw 1932. The movement of the slide bar 1934 may be longitudinal with respect to the second jaw 1932. The second end 1942 of the spring 1940 may move longitudinally with the slide bar 1934. The longitudinal position of the second jaw 1932 and the first end 1938 of the spring may remain constant with respect to the first jaw 1832 during the movement of the slide bar 1934 or may vary during movement. The second end 1942 of the spring 1940 may slide in a direction away from the first end 1938 of the spring 1940 during an opening of the clip 1930, and may slide in a direction towards the first end 1938 of the spring 1940 during a closing of the clip 1930.

The movement of the slide bar 1934 may allow the first jaw 1832 to open relative to the second jaw 1932 in an opened state, and may allow the first jaw 1832 to be forced towards the second jaw 1932 by the spring 1940 to compress the portion of the heart between the first jaw 1832 and the second jaw 1932 in a closed state.

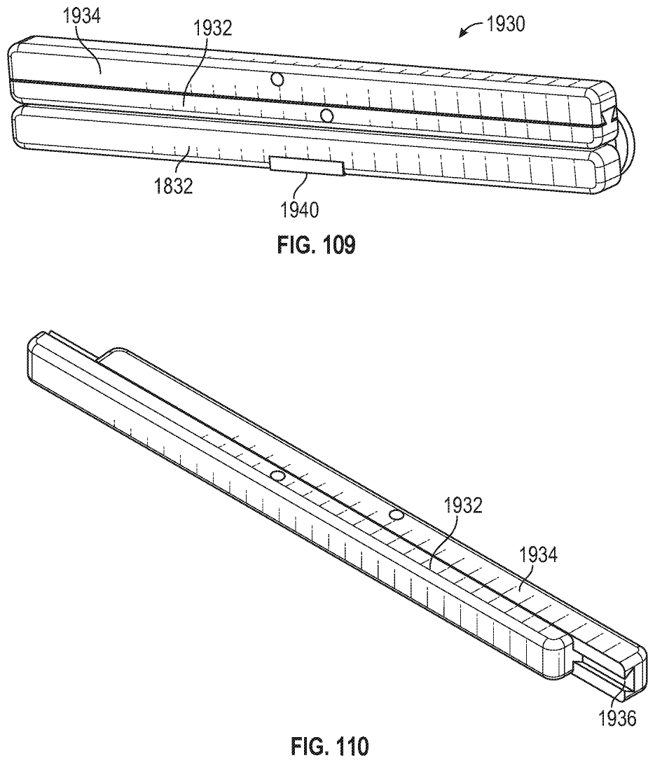

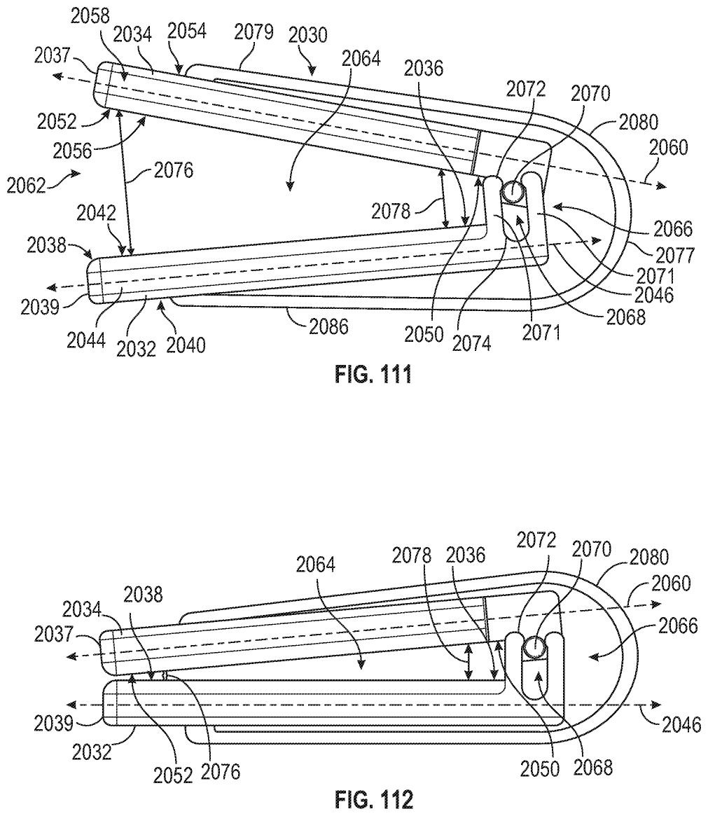

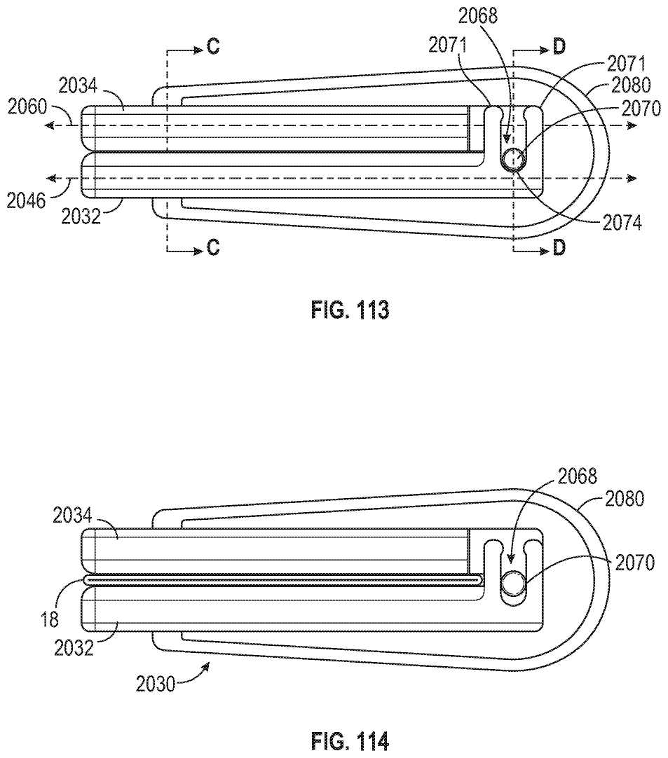

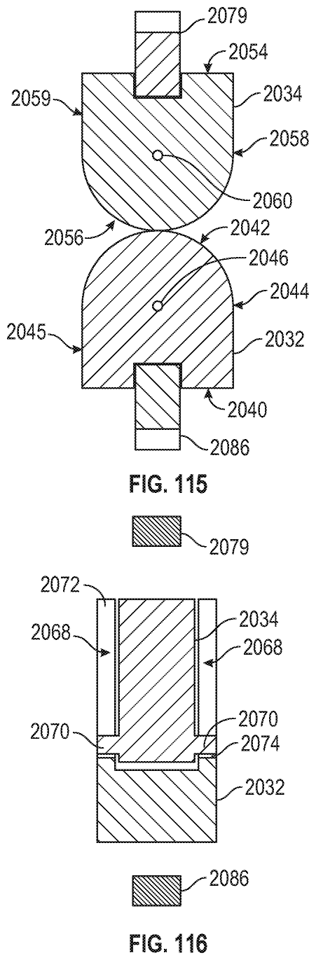

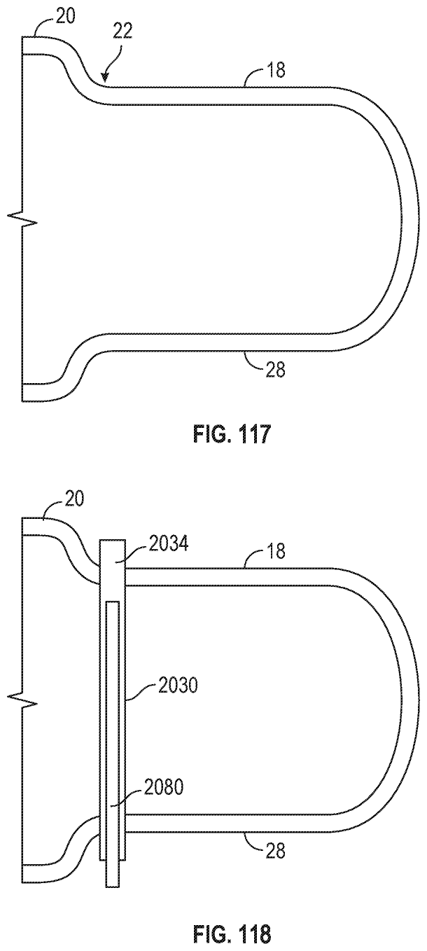

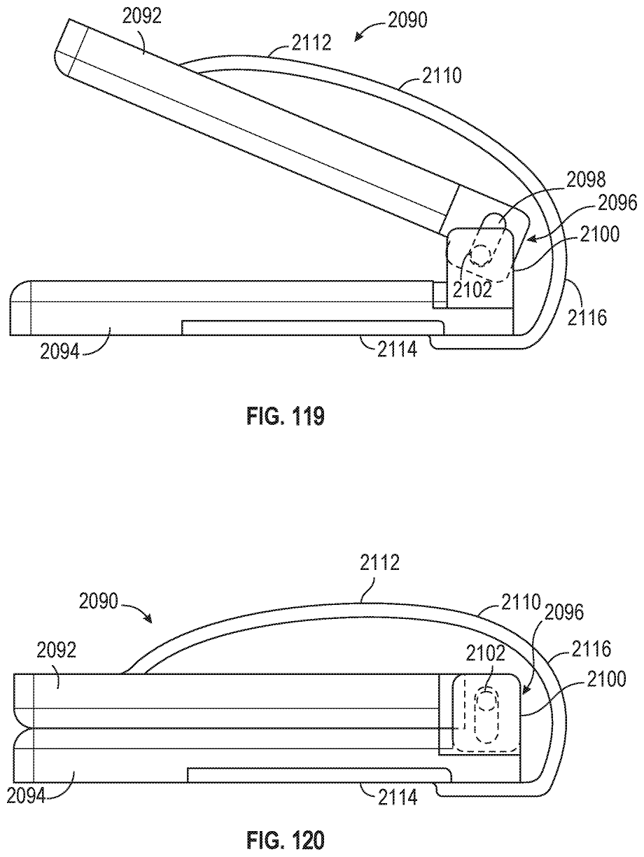

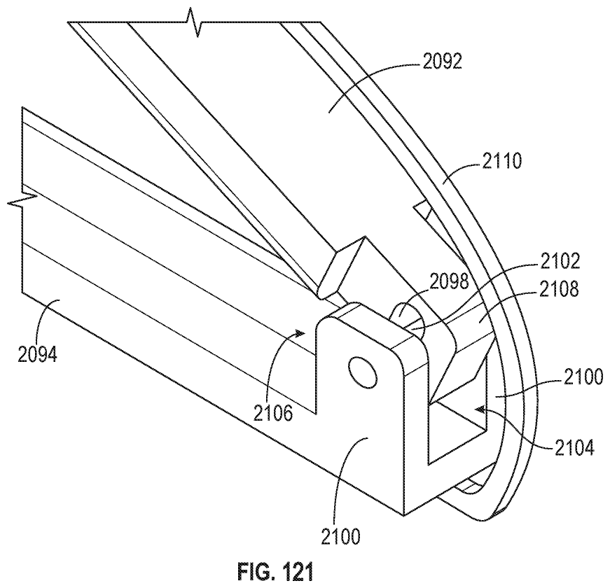

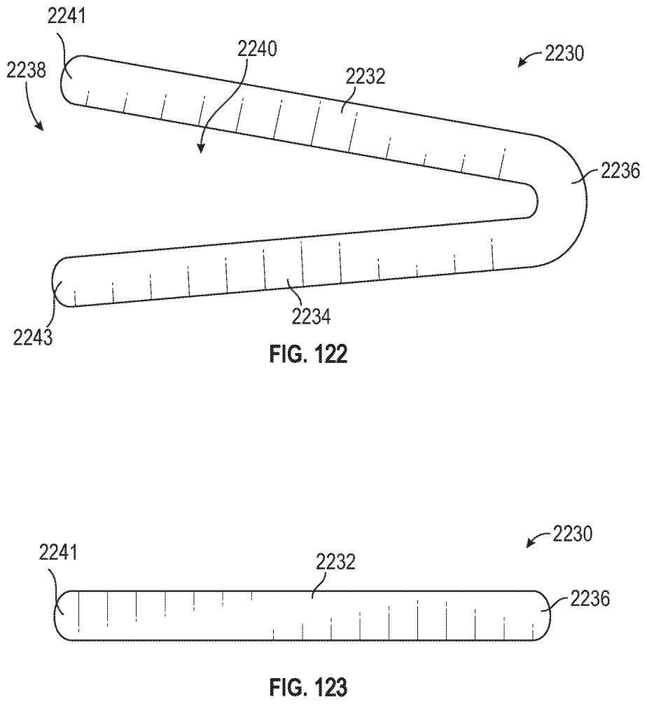

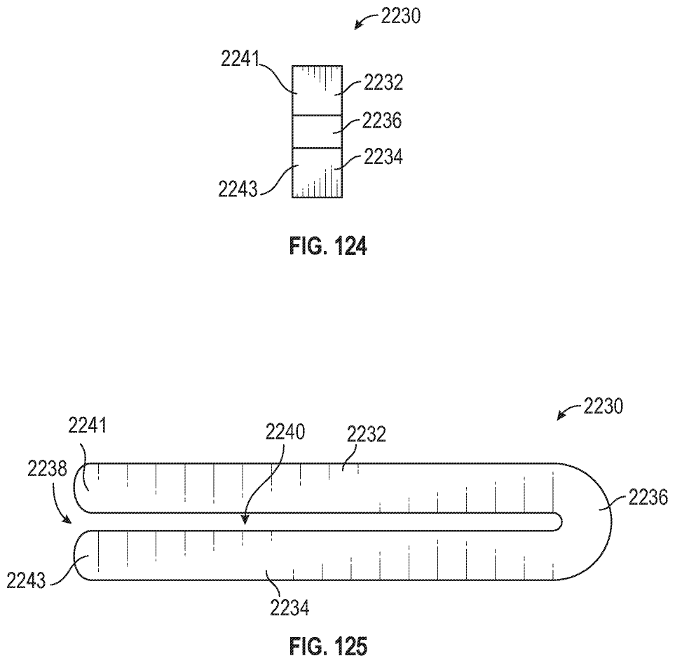

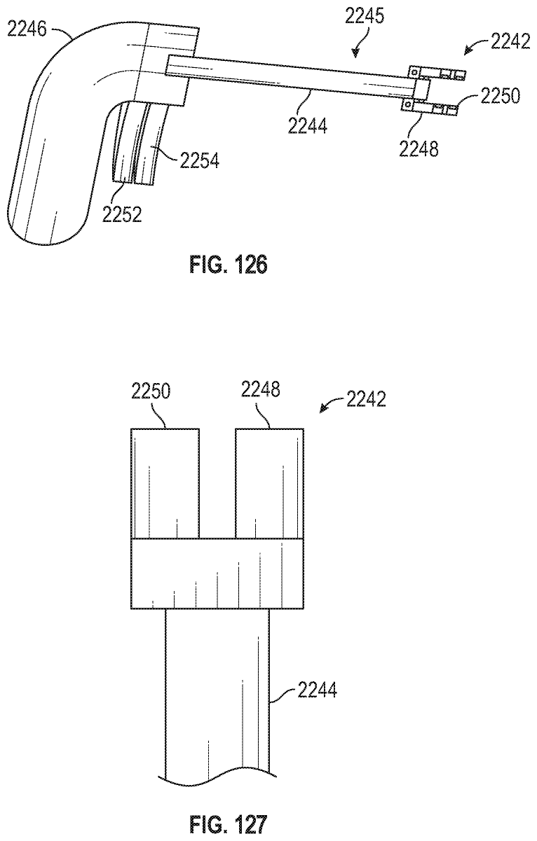

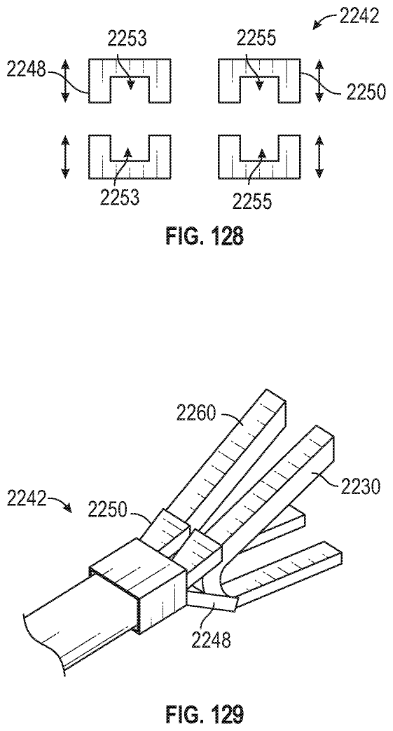

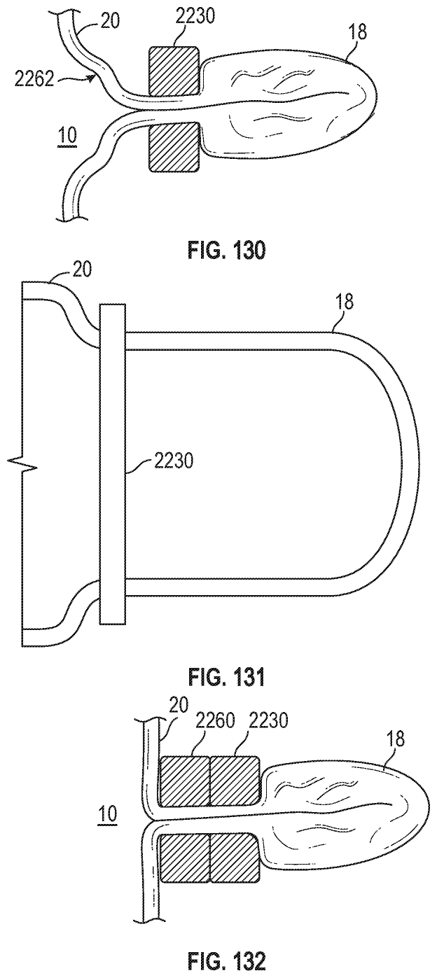

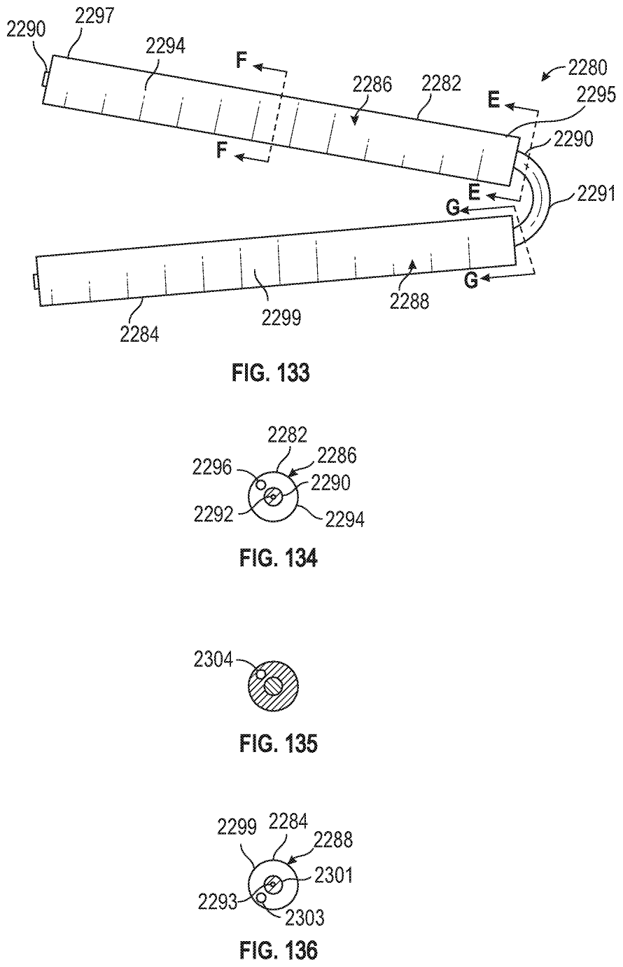

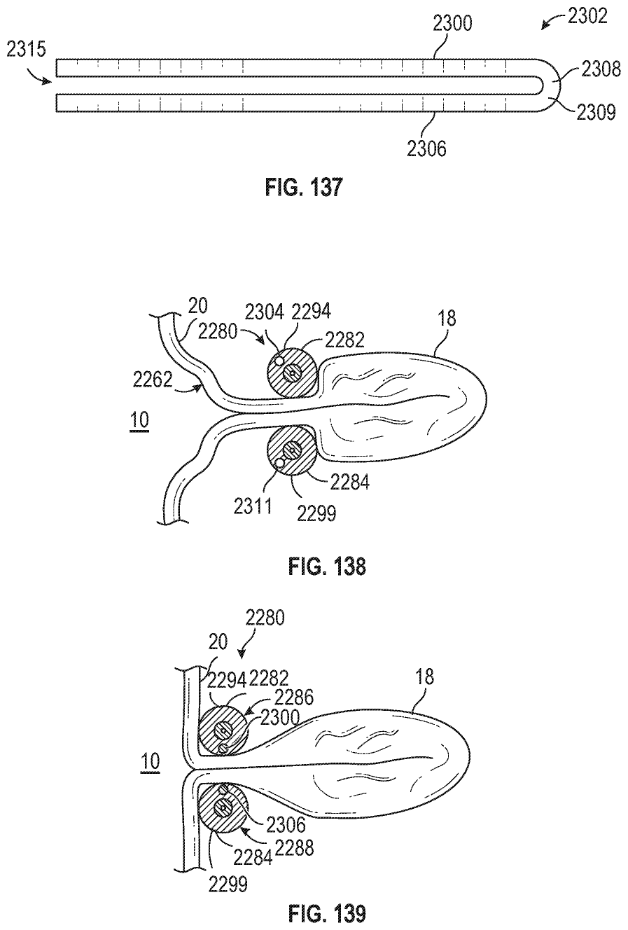

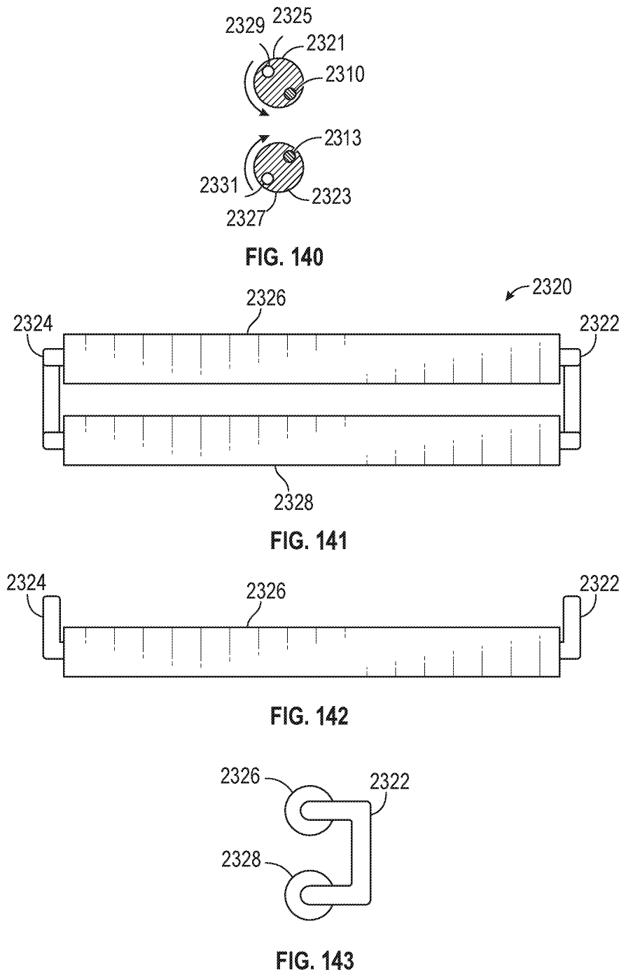

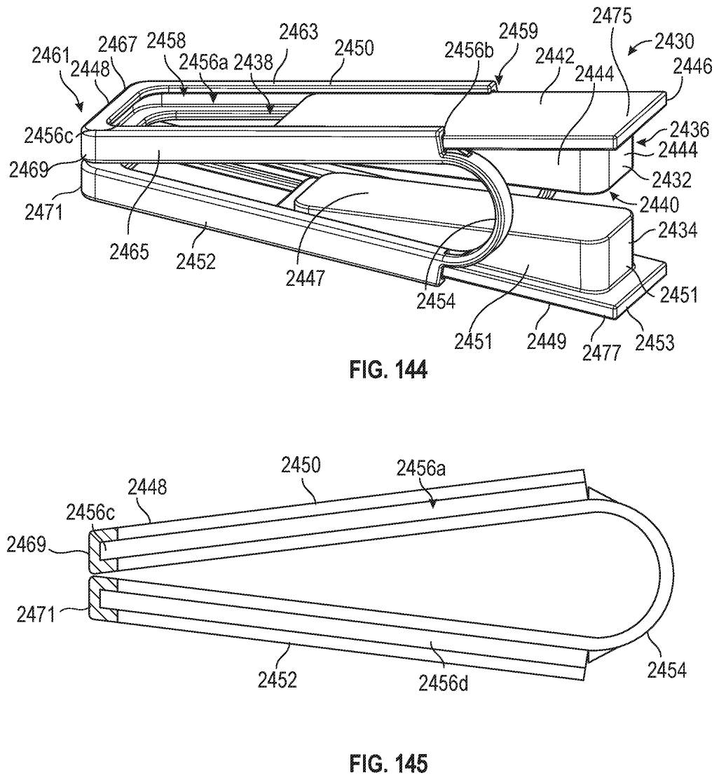

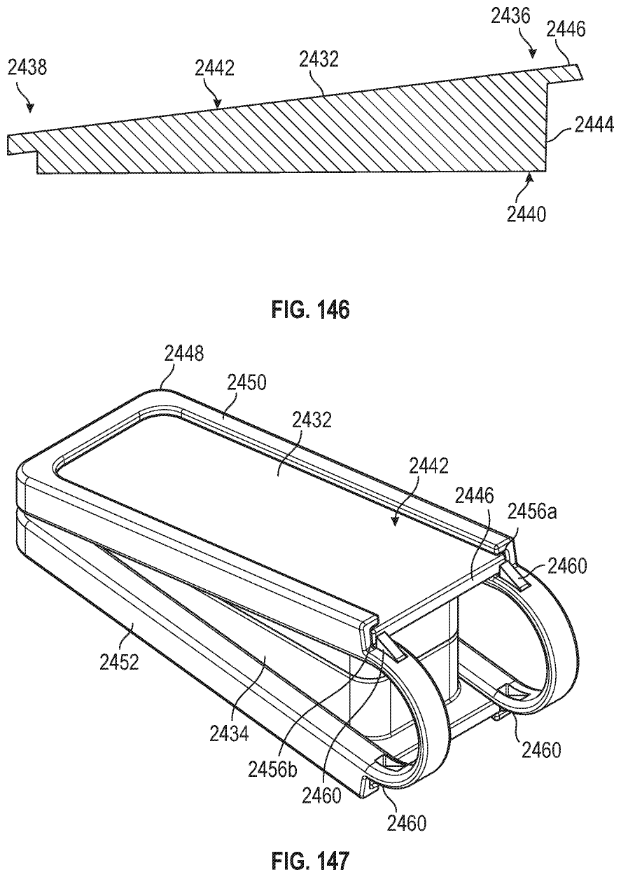

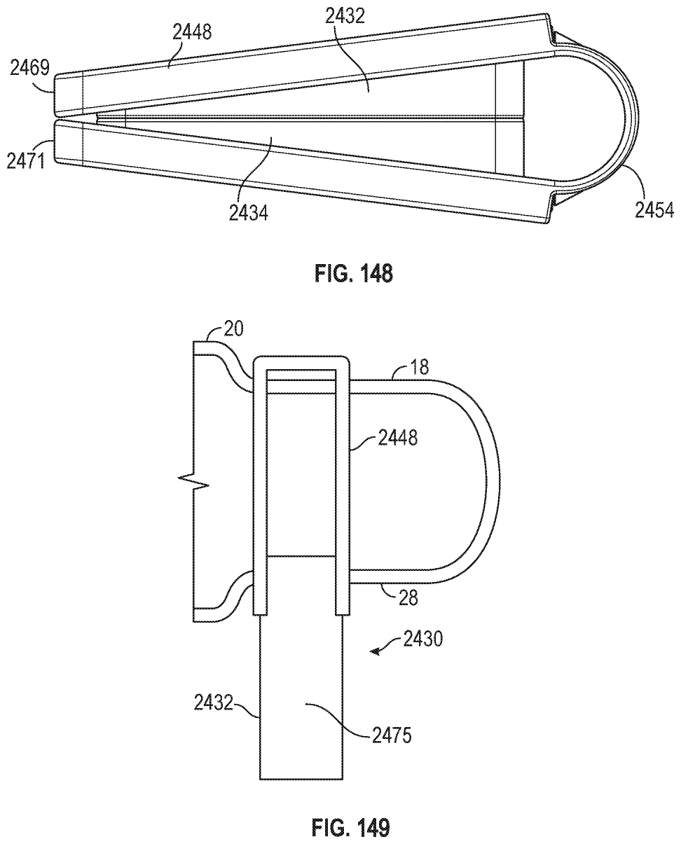

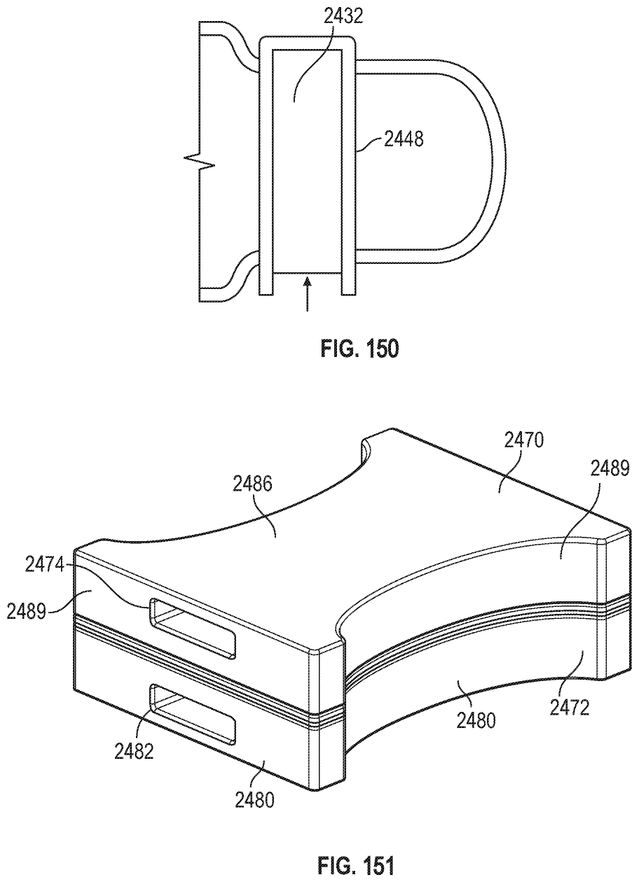

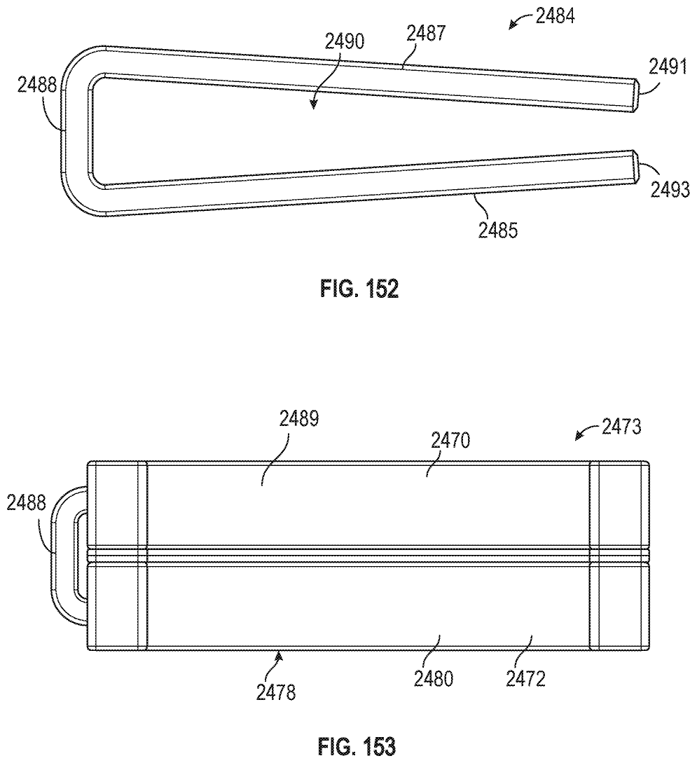

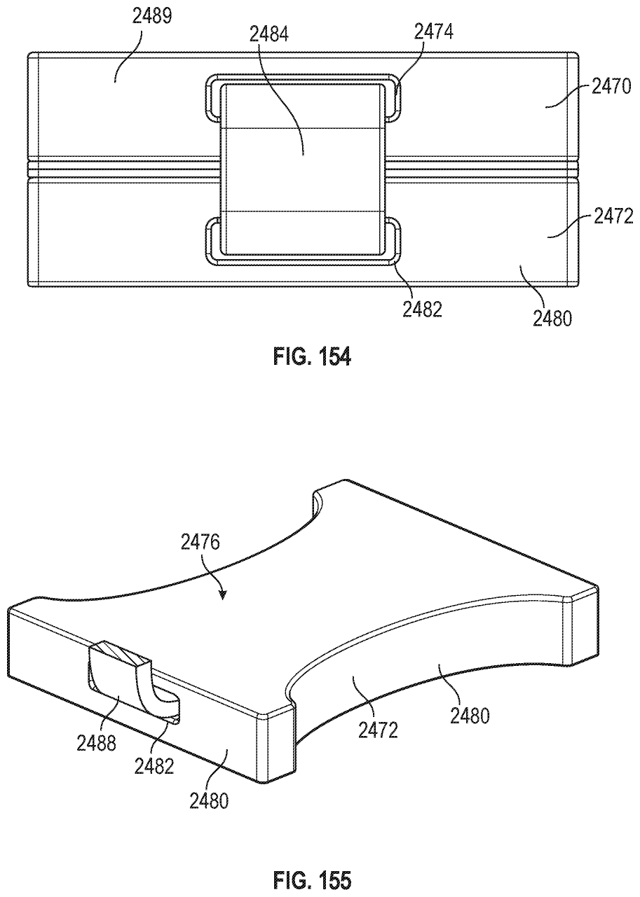

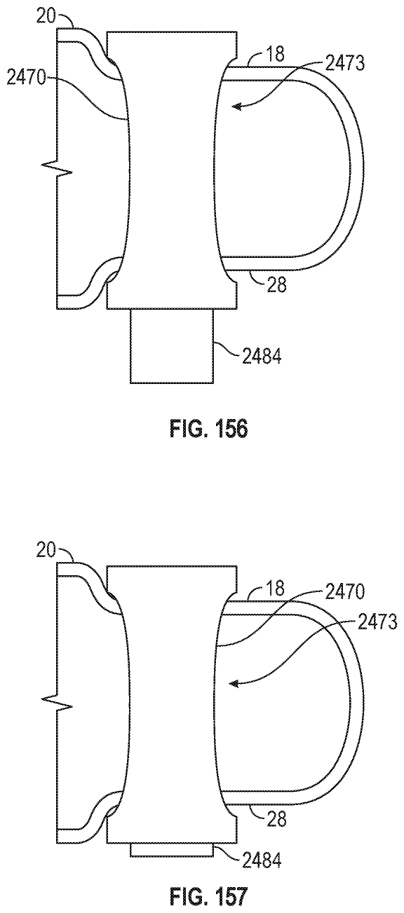

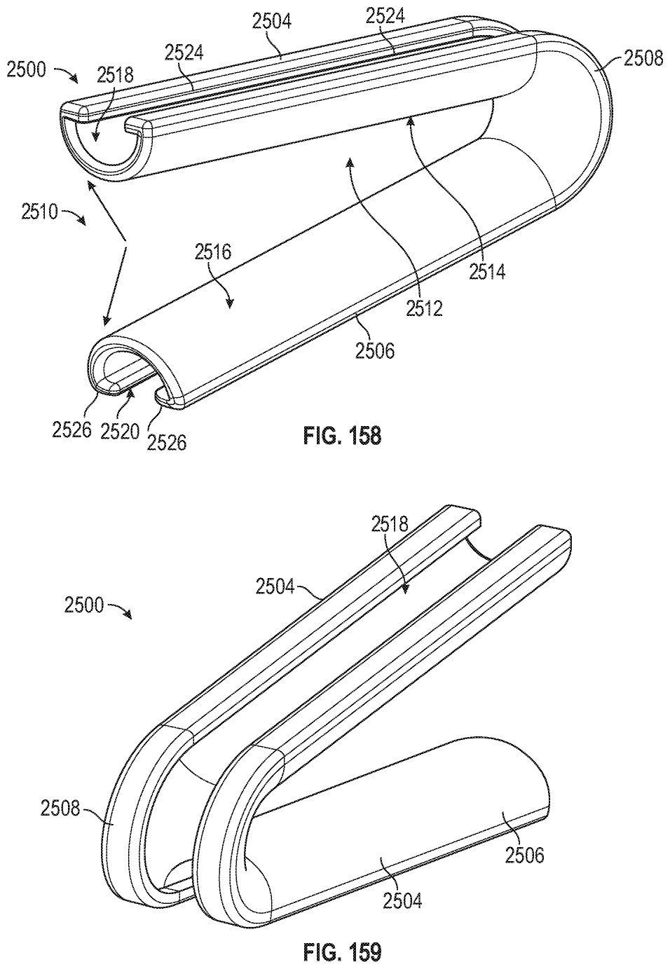

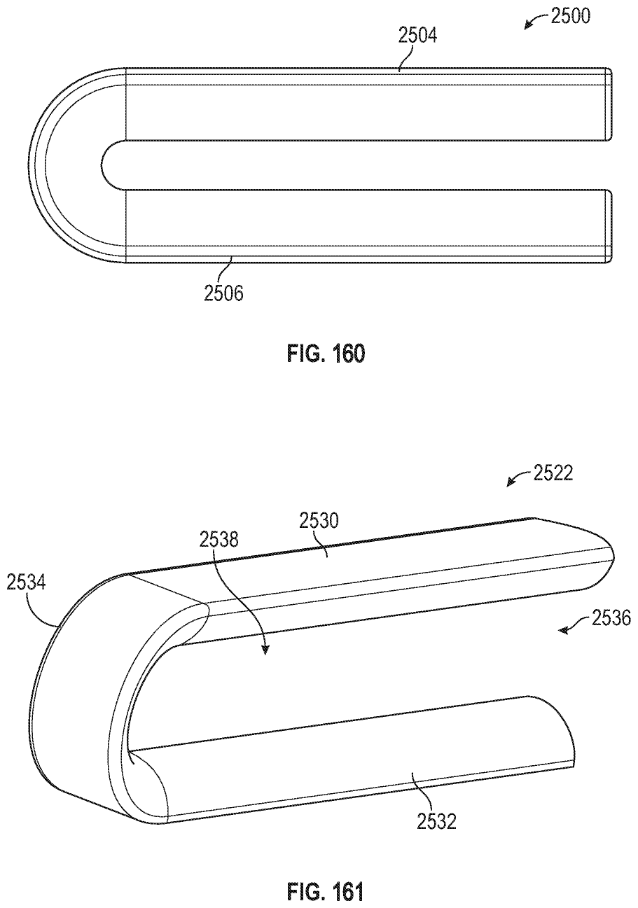

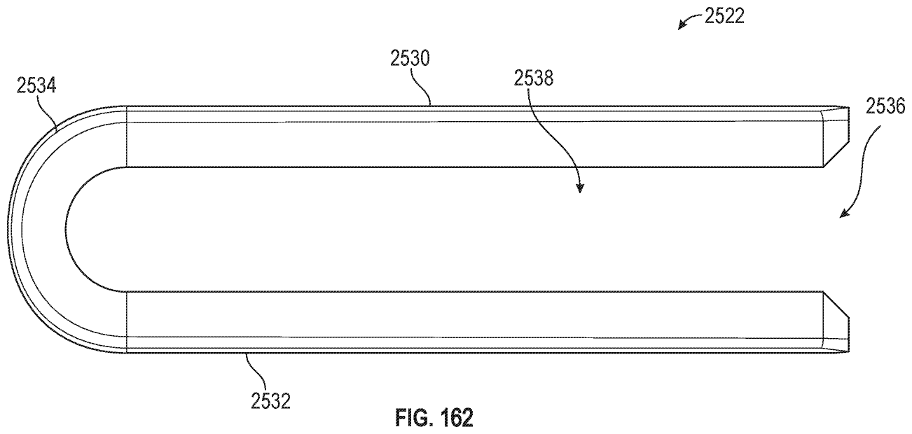

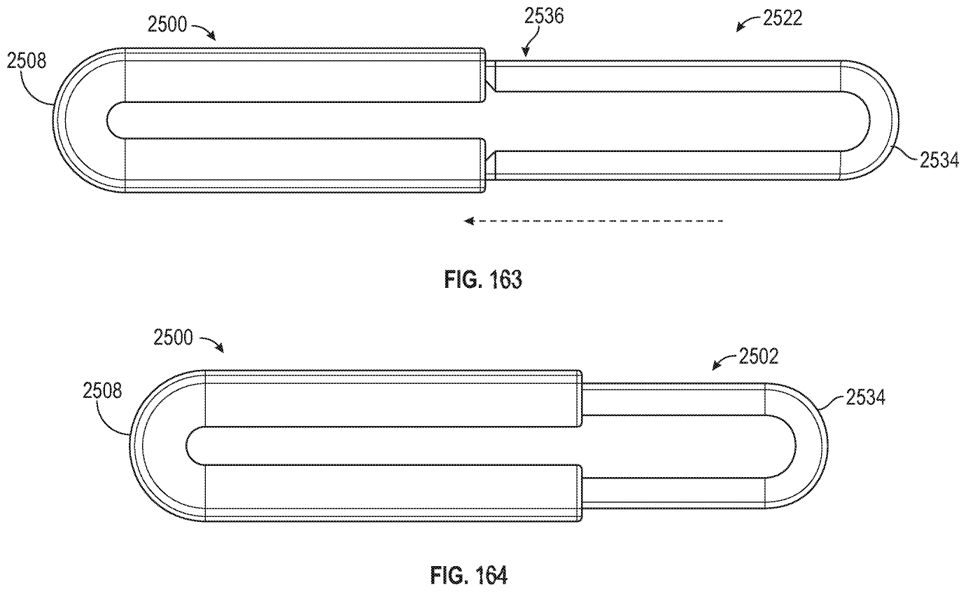

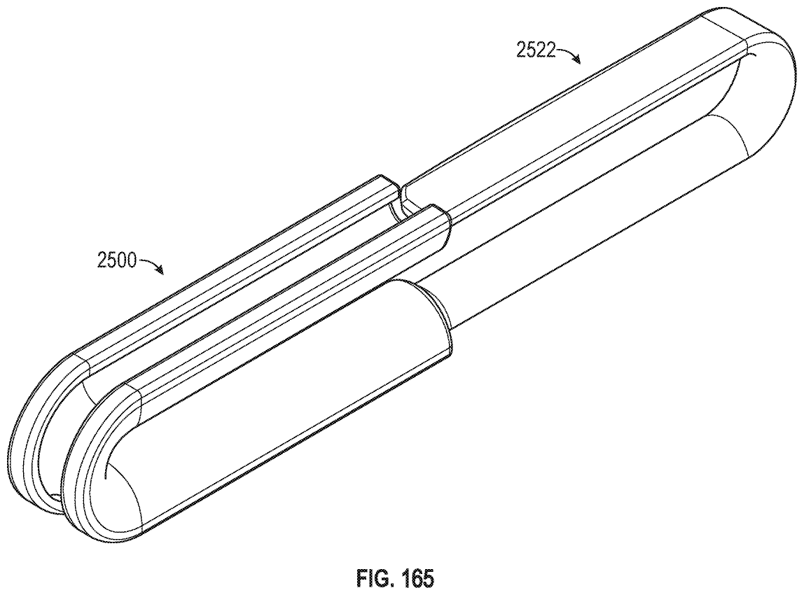

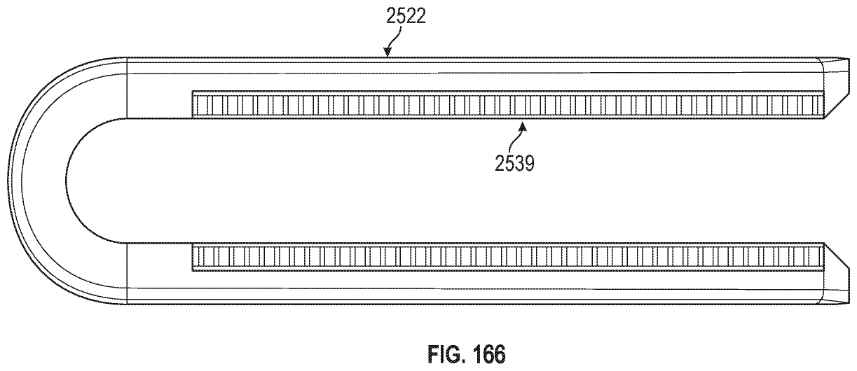

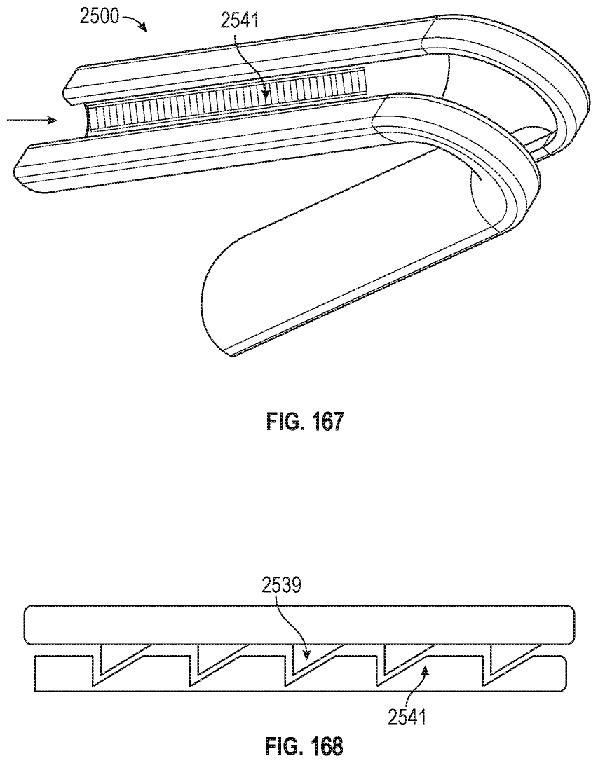

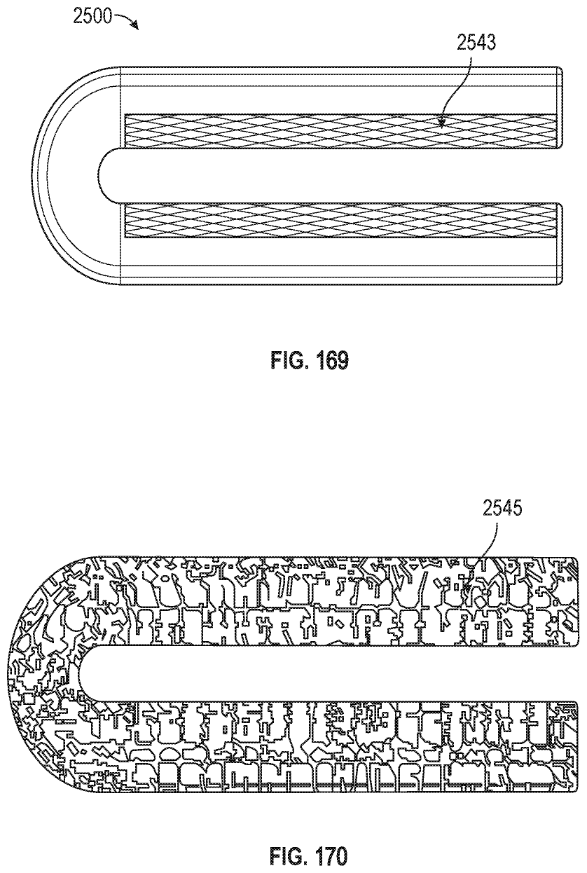

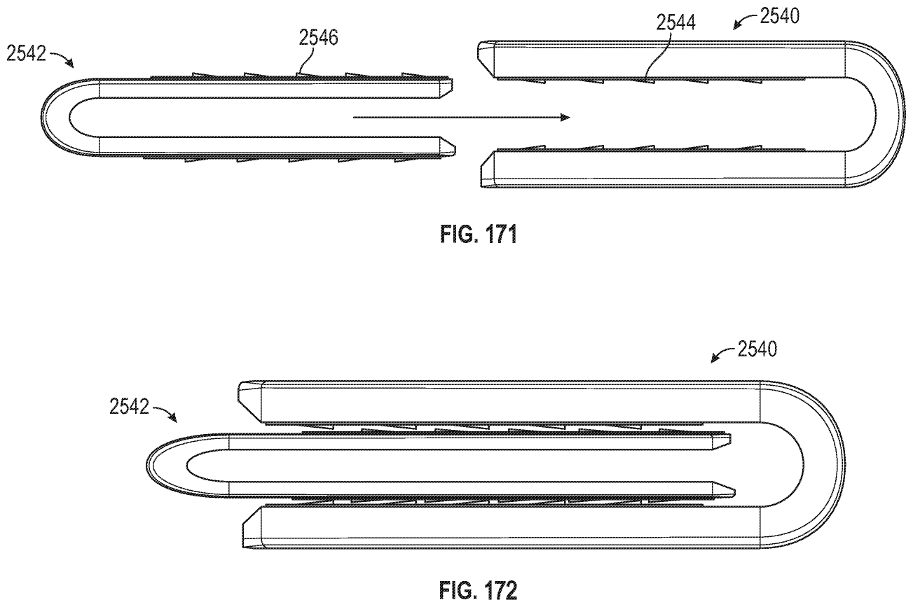

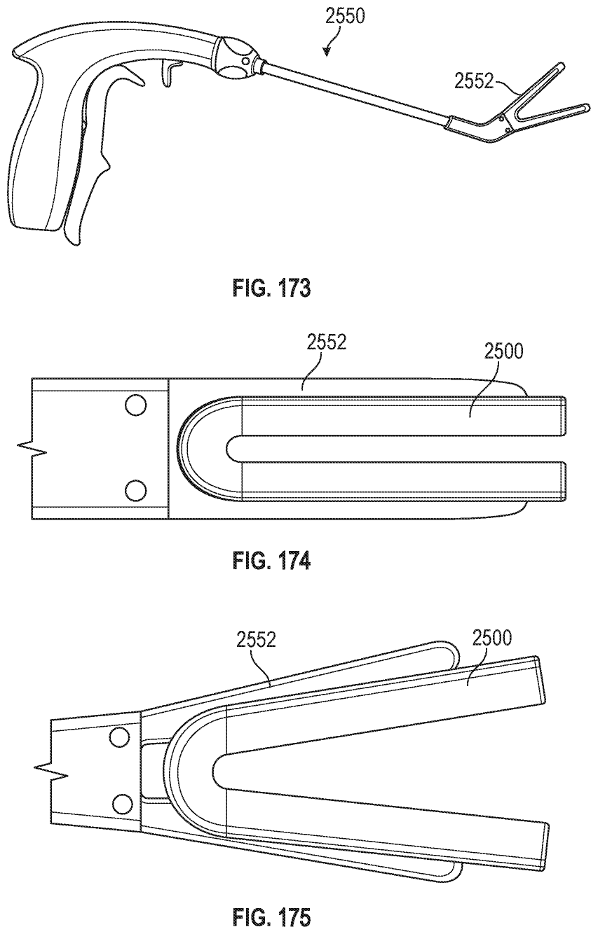

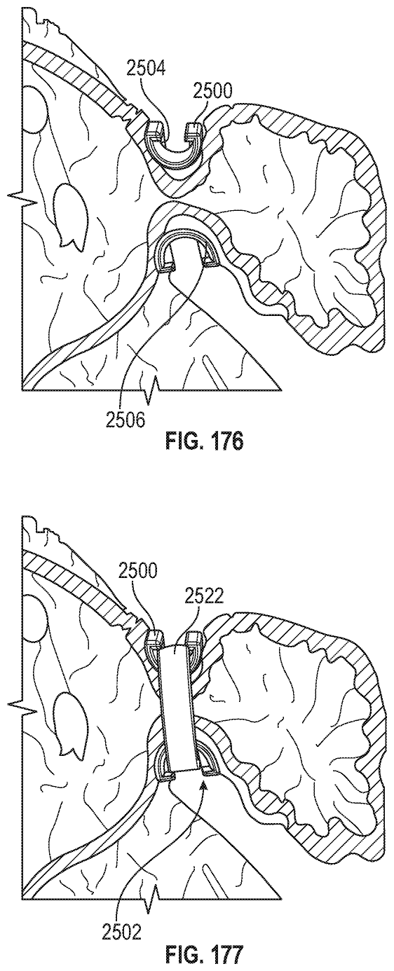

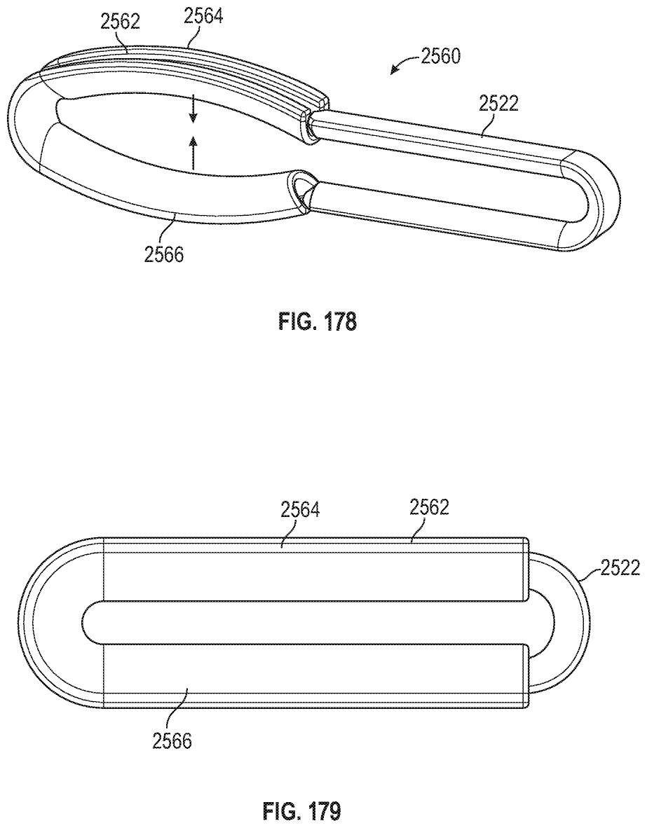

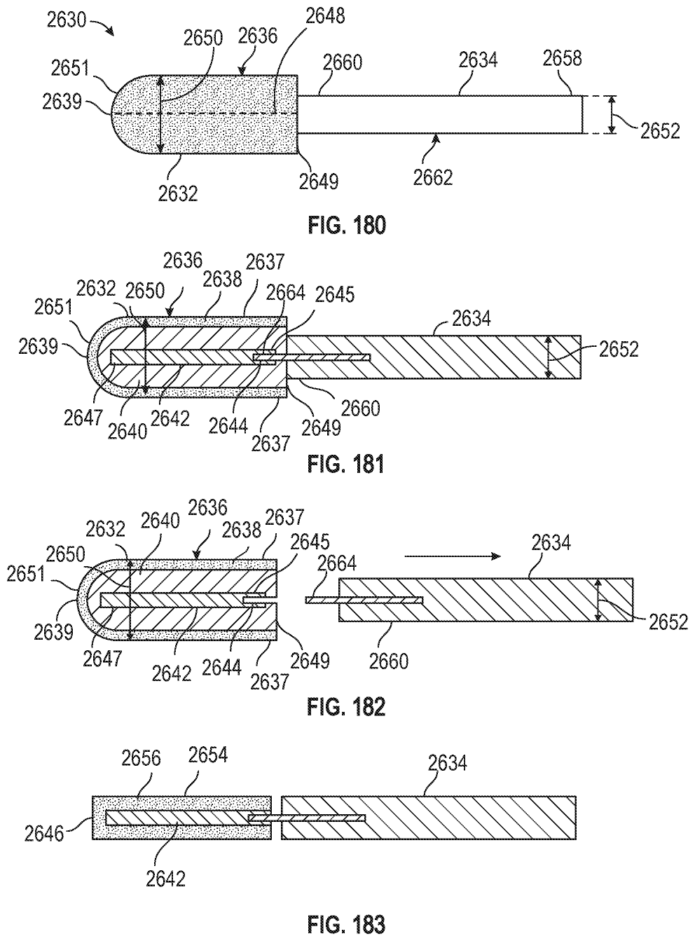

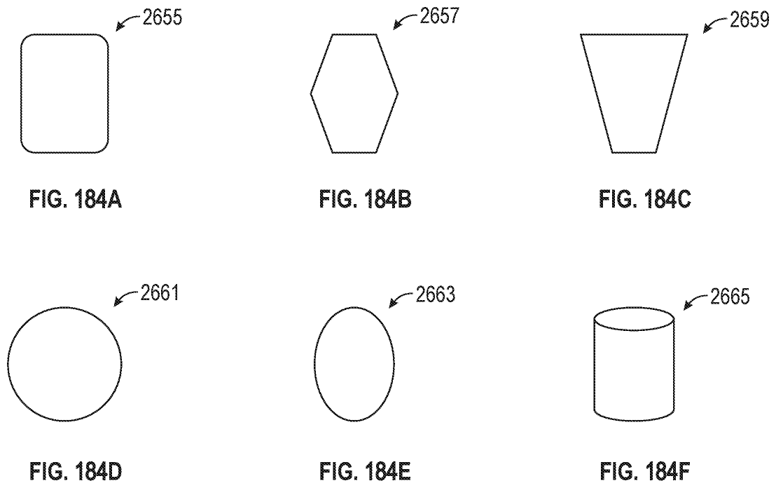

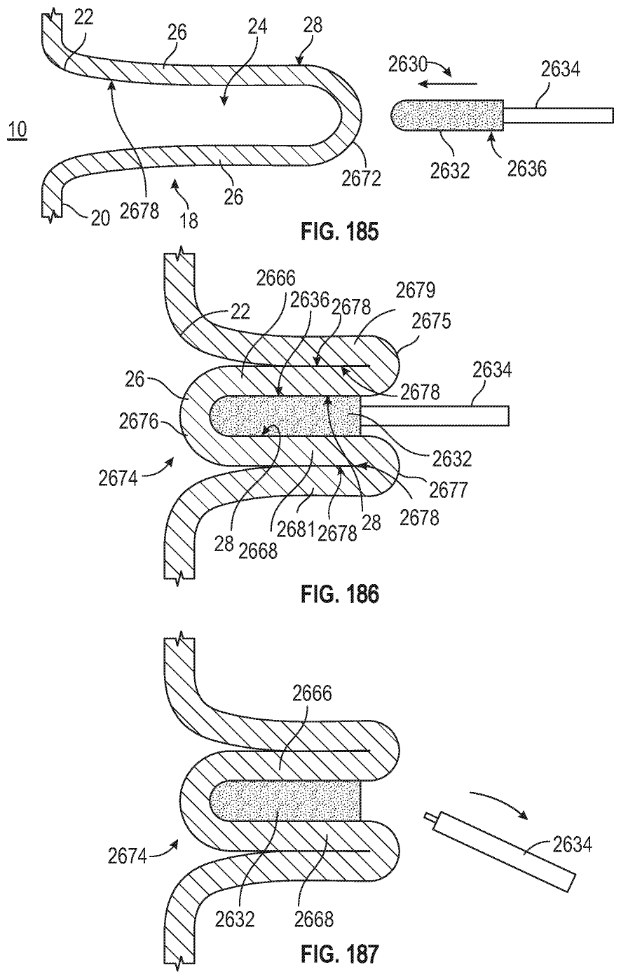

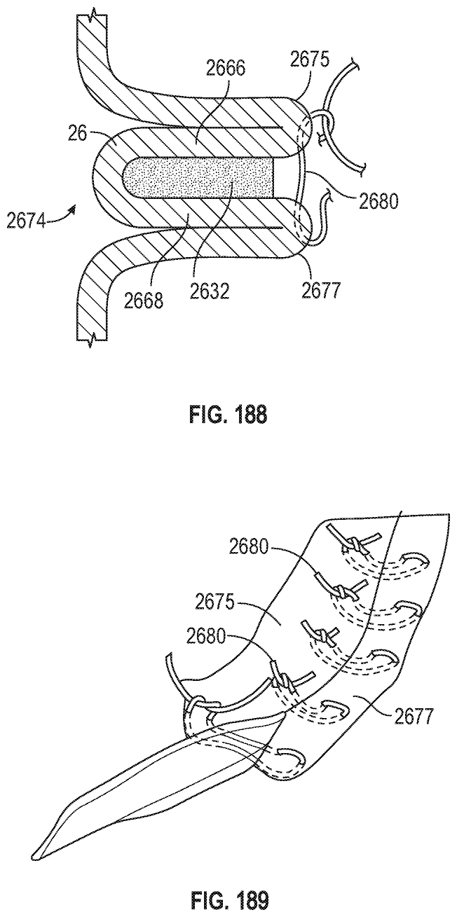

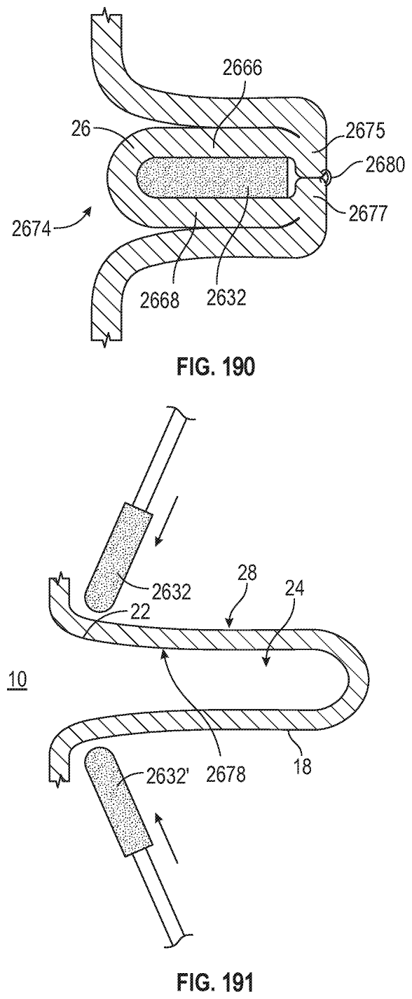

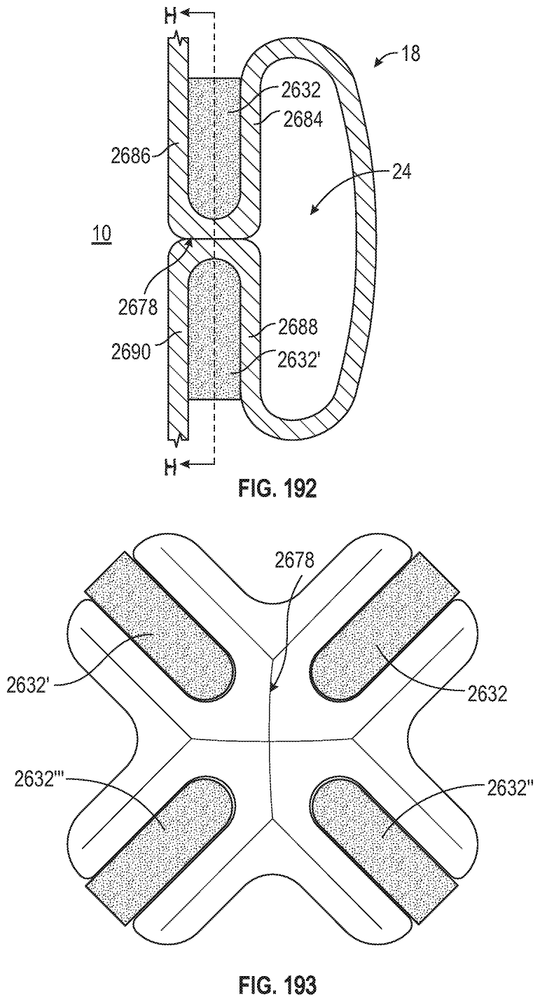

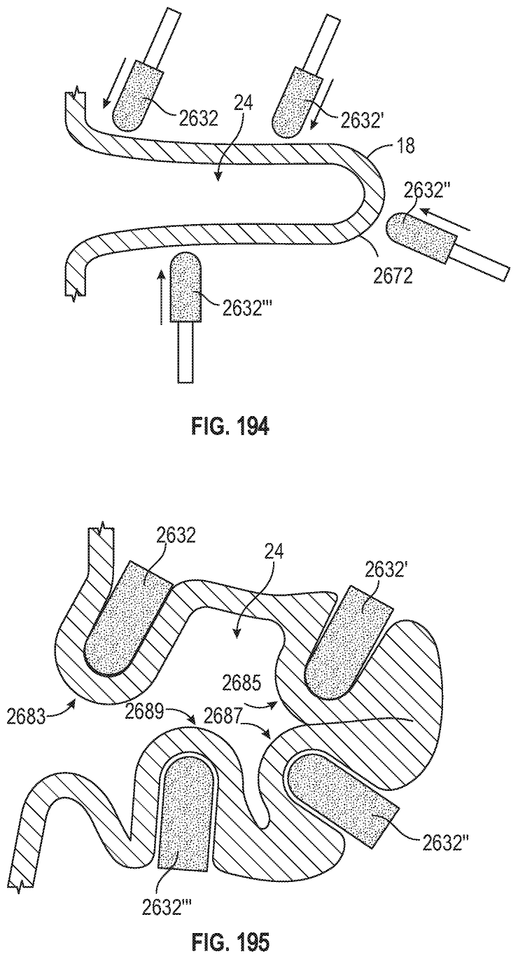

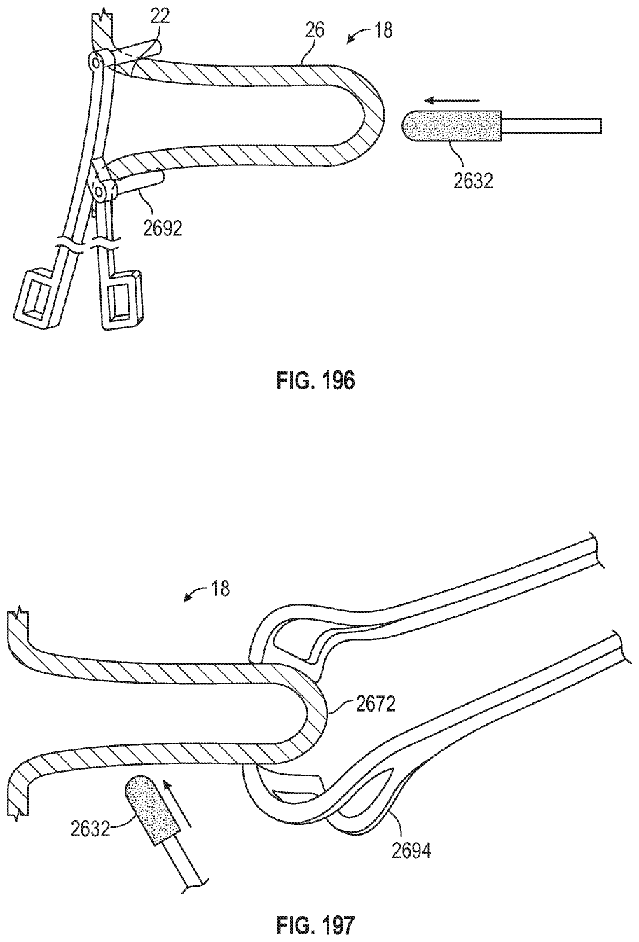

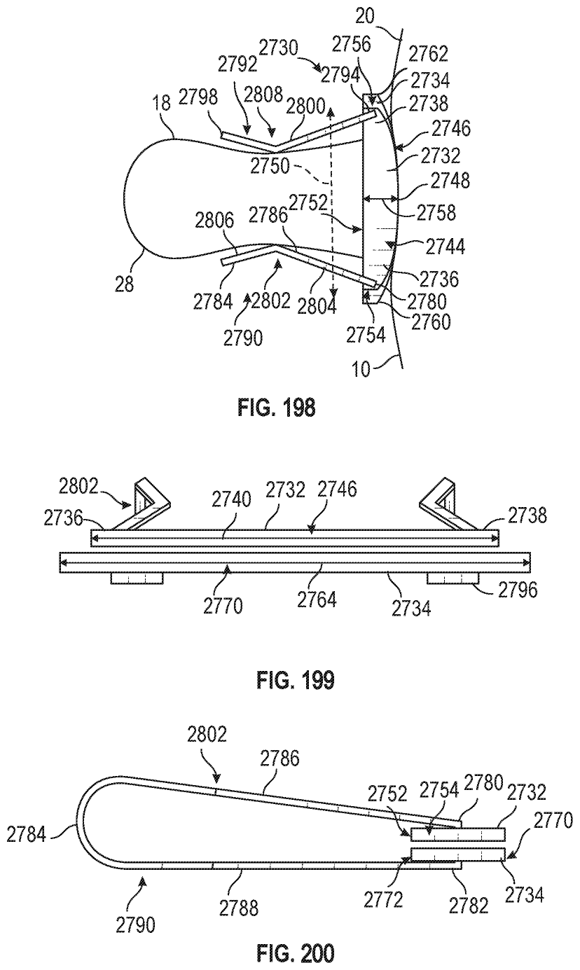

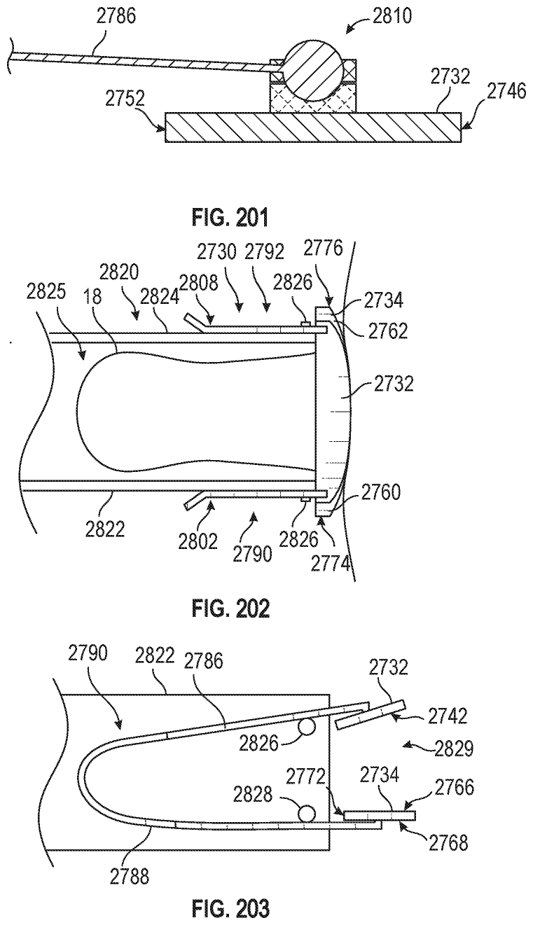

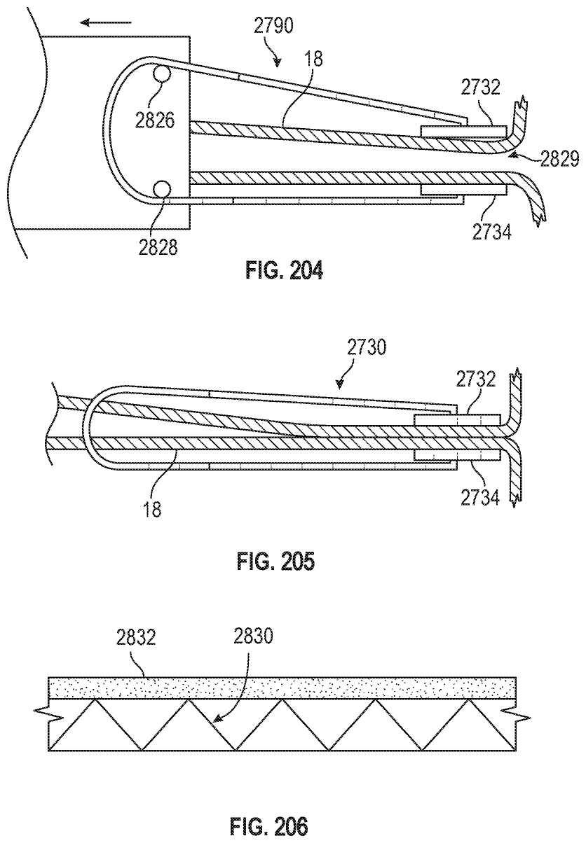

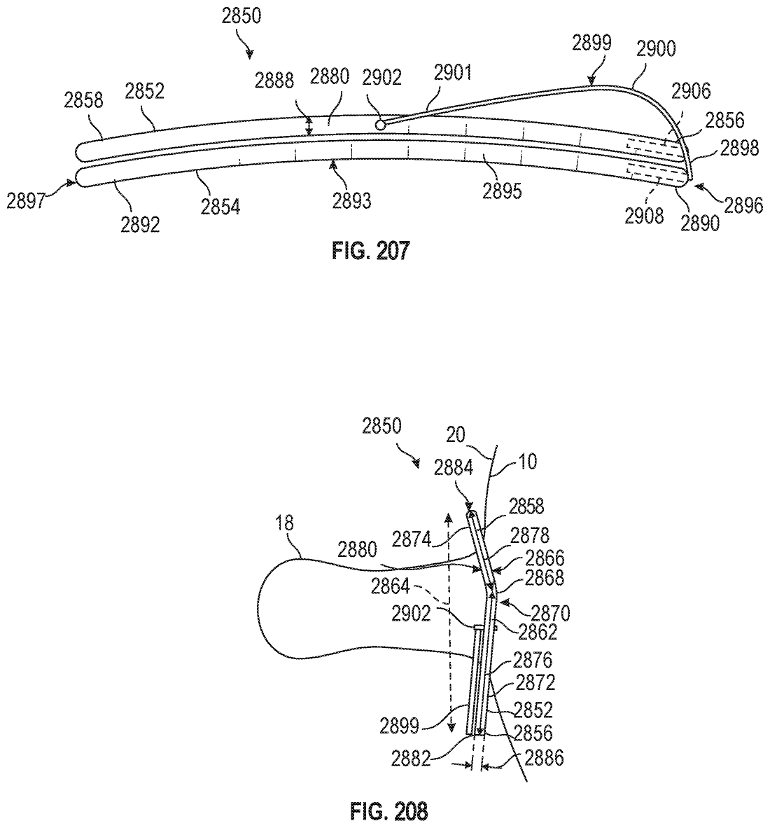

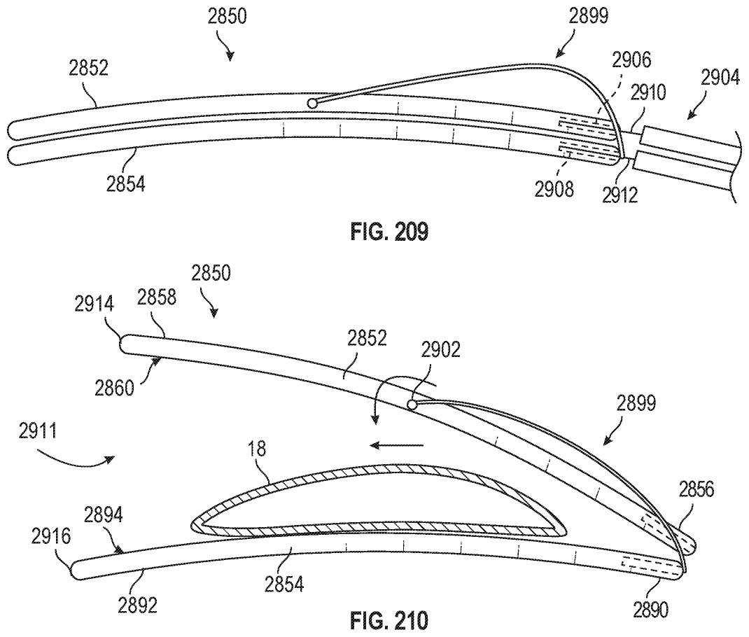

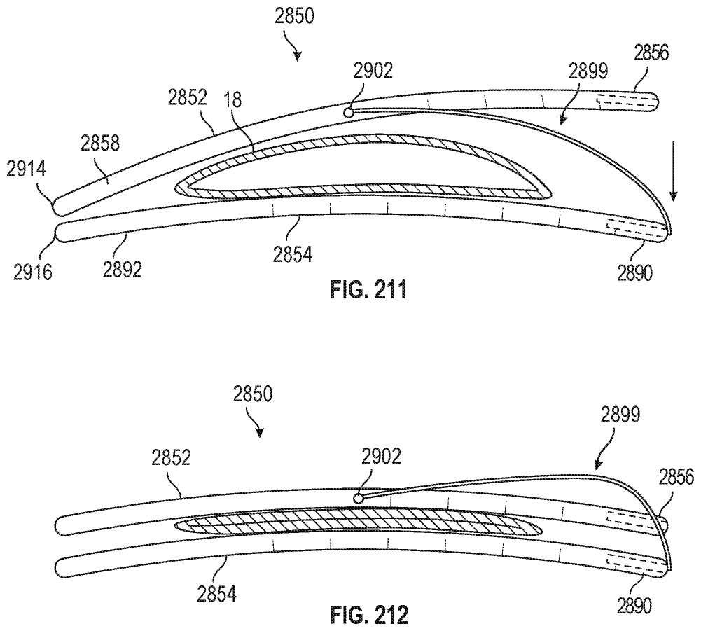

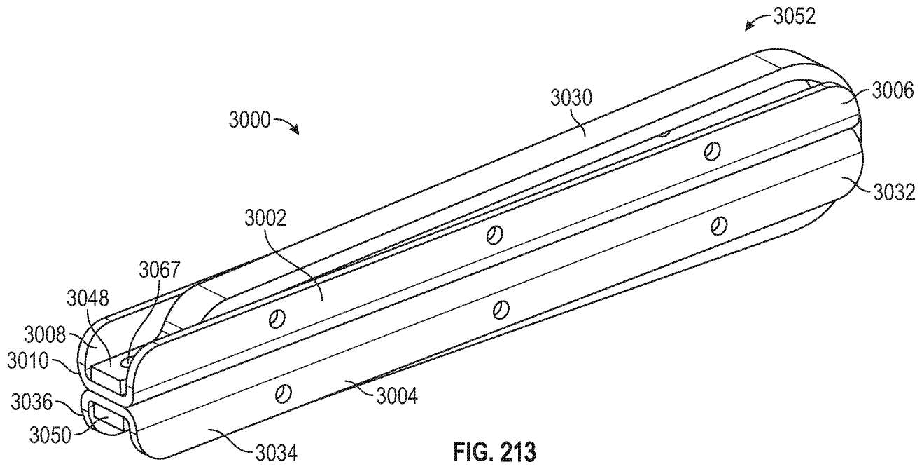

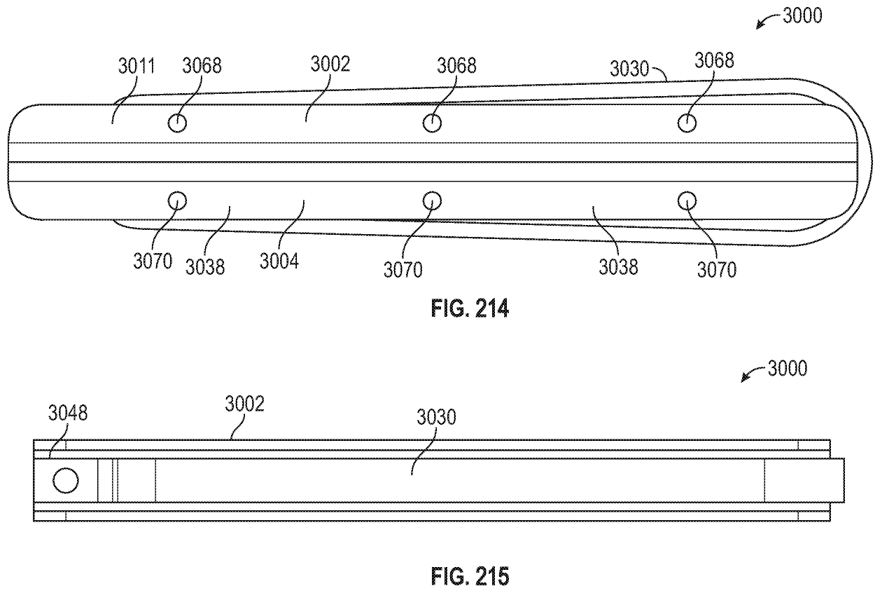

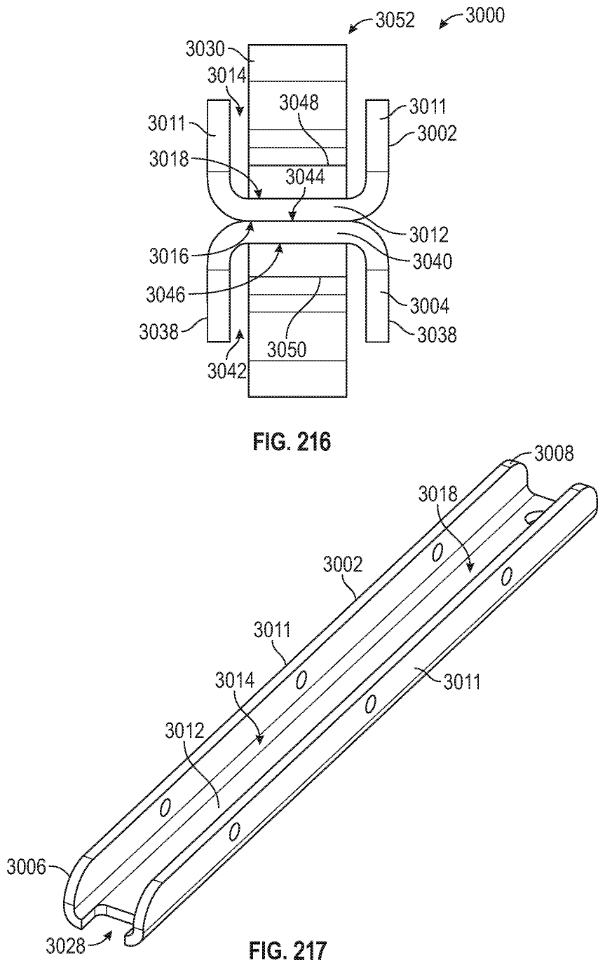

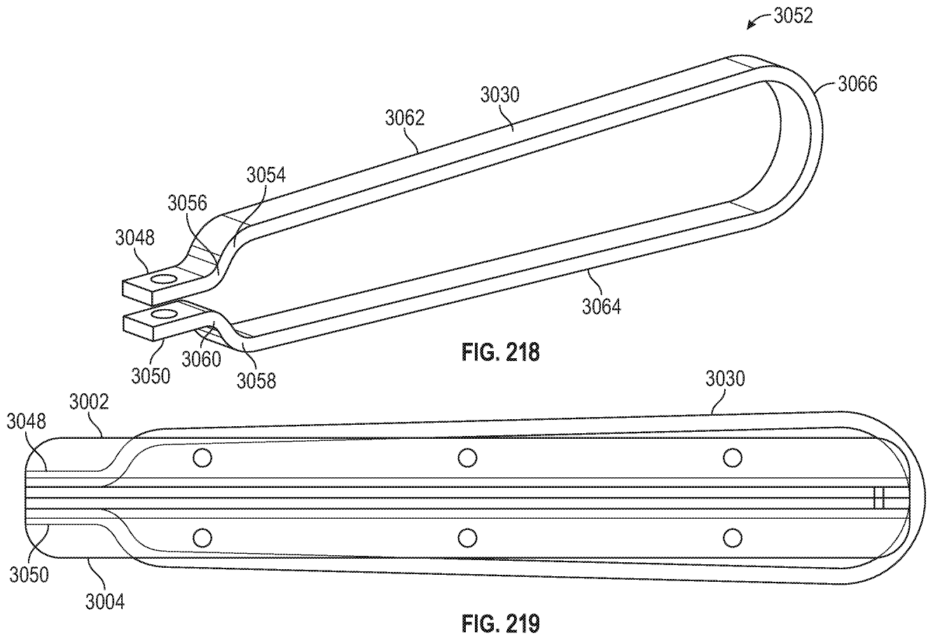

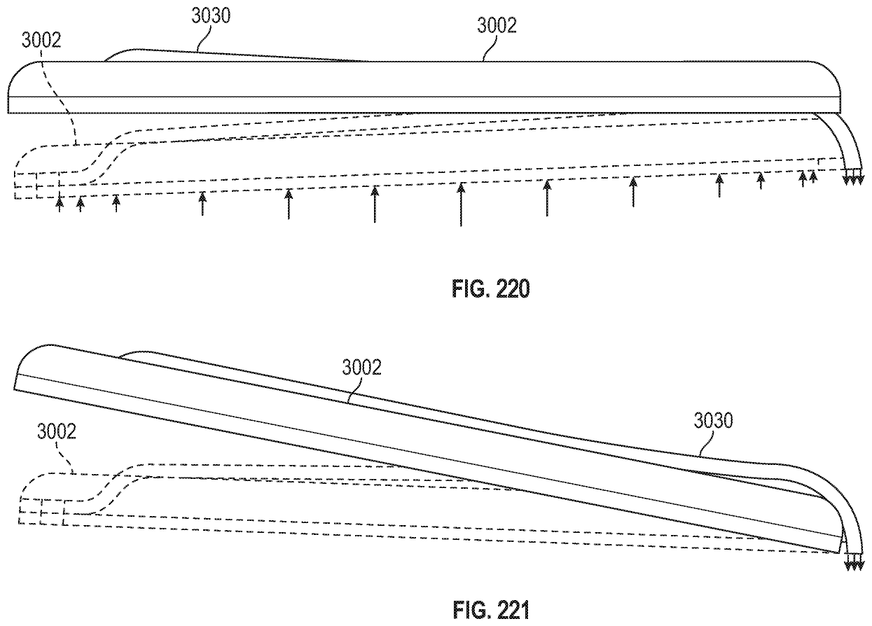

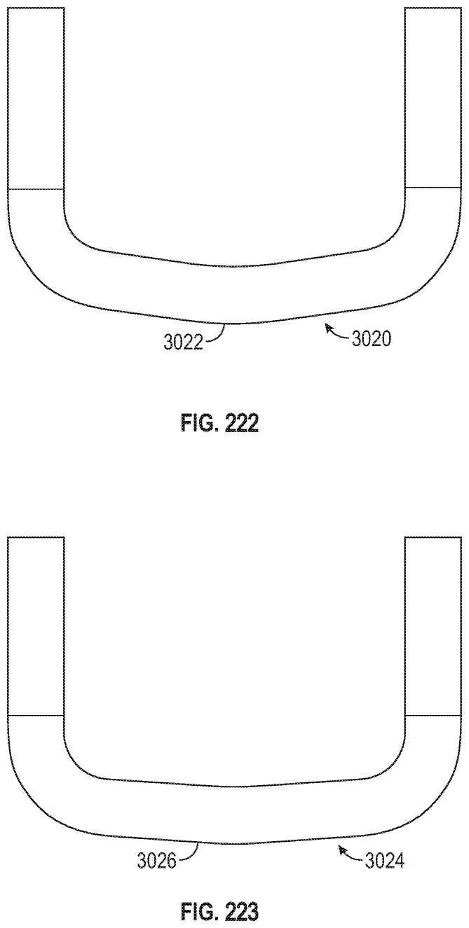

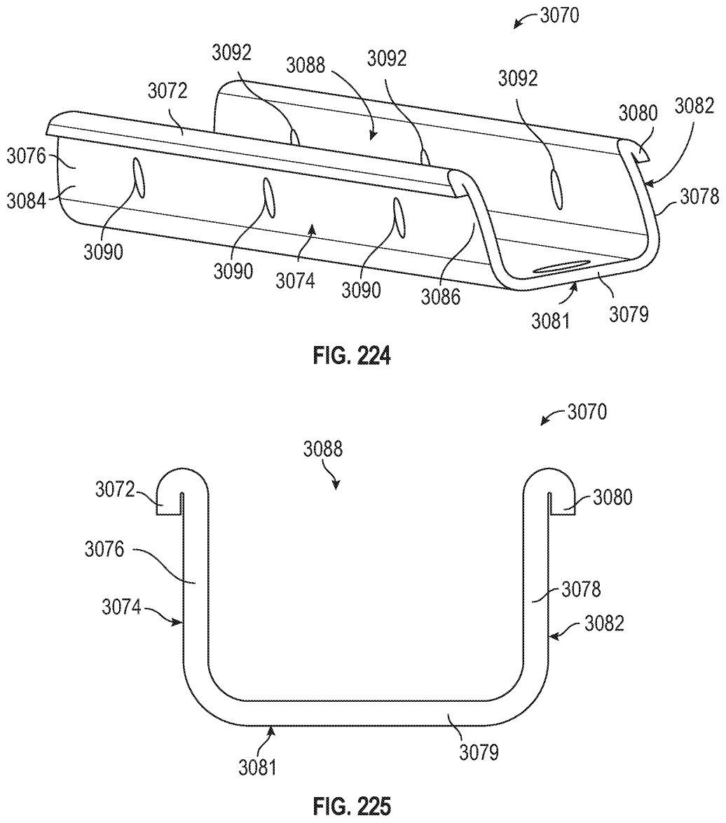

FIG. 109 illustrates a side perspective view of the clip 1930 at an opposite side than shown in FIG. 108. FIG. 110 illustrates the second jaw 1932 and the slide bar 1934 in isolated view, with the slide bar 1934 slid relative to the second jaw 1932.

Various other modifications of the clips disclosed herein may be provided. Various other methods of deployment and use of the clips may be provided as desired.

The clips as disclosed herein may be utilized to close the LAA or may be utilized to close another portion of a heart. In examples, the clips may be utilized to close other portions of a body, including other tubular vessels or other portions of a body. Deployment may be via a delivery apparatus or via another method as desired. Deployment may be via surgical methods and may be transcatheter or via non-invasive surgery in methods.

Variations in the clips and methods disclosed herein may be provided. Features across examples may be combined. Features may be excluded or added to in various examples disclosed herein. Combinations of features of examples may be provided. The features may be utilized solely or in methods disclosed herein, or in combination with other features disclosed herein.

FIG. 11 illustrates an example of a clip 2030 that may be utilized for a portion of a heart. The clip 2030 may be configured to close a LAA 18, to reduce the possibility of clots or other undesired materials stemming from the LAA 18 from traveling into the bloodstream.

The clip 2030 may include a first jaw 2032 and a second jaw 2034. The first jaw 2032 may extend from a proximal end portion 2036 to a distal end portion 2038 and may have an elongate shape. The distal end portion 2038 of the first jaw 2032 may comprise a distal tip 2039 of the first jaw 2032 in examples. The first jaw 2032 may be configured as an elongate beam or may have another configuration as desired.

The first jaw 2032 may include four sides, including an outer surface 2040, a compression surface 2042 (marked in FIG. 115 and facing opposite the outer surface 2040 and towards the second jaw 2034), and a first side surface 2044 and a second side surface 2045 (marked in FIG. 115) facing opposite the first side surface 2044. The side surfaces each extend between the compression surface 2042 and the outer surface 2040.

The first jaw 2032 may extend along a longitudinal axis 2046. Referring to FIGS. 11 and 115, the compression surface 2042 of the first jaw 2032 may have a rounded shape, although other shapes may be utilized as desired. For example, the compression surface 2042 may have a flat shape, and the outer surface 2040 may have a rounded shape, as desired.

The second jaw 2034 may extend from a proximal end portion 2050 to a distal end portion 2052 and may have an elongate shape. The distal end portion 2052 of the second jaw 2034 may comprise a distal tip 2037 of the second jaw 2034 in examples. The second jaw 2034 may be configured as an elongate beam or may have another configuration as desired.

The second jaw 2034 may include four sides, including an outer surface 2054, a compression surface 2056 (facing opposite the outer surface 2054 and towards the first jaw 2032), and a first side surface 2058 and a second side surface 2059 (marked in FIG. 115) facing opposite the first side surface 2058. The side surfaces each extend between the compression surface 2056 and the outer surface 2054.

The second jaw 2034 may extend along a longitudinal axis 2060. Referring to FIGS. 11 and 115, the compression surface 2056 of the second jaw 2034 may have a rounded shape, although other shapes may be utilized in examples as desired. For example, the compression surface 2056 may have a flat shape, and the outer surface 2054 may have a rounded shape, as desired.

The distal end portions 2038, 2052 of the respective first jaw 2032 and second jaw 2034 may define an opening 2062 for a channel 2064 for receiving an object to be compressed. The distal end portions 2038, 2052 may comprise an open end of the channel 2064 and the proximal end portions 2036, 2050 may comprise a closed end of the channel 2064. The object to be compressed (e.g., a portion of a heart such as a left atrial appendage (LAA)) may be inserted into the channel 2064 through the opening 2062 or the object may be slid into the channel 2064 transverse to the axes 2046, 2060 of the clip 2030.

A slotted pin joint 2066 may be coupled to the first jaw 2032 and the second jaw 2034 and may be configured to allow the first jaw 2032 to move relative to the second jaw 2034 to compress an object between the first jaw 2032 and the second jaw 2034. The slotted pin joint 2066, for example, may be positioned at the proximal end portions 2036, 2050 of the first jaw 2032 and the second jaw 2034. The slotted pin joint 2066 may comprise a pivot that one or more of the first jaw 2032 or the second jaw 2034 may rotate about to move between an opened configuration (as shown in FIG. 111) and a closed configuration (as shown in FIG. 113, for example). One or more of the first jaw 2032 or the second jaw 2034 may slide along the slotted pin joint 2066 to move between an opened configuration and a closed configuration.

The slotted pin joint 2066 may include a slot 2068 and a pin 2070. The slot 2068 may comprise a portion of the proximal end portion 2036 of the first jaw 2032 and may extend transverse to the axis 2046 of the first jaw 2032. For example, as shown in FIG. 111, the slot 2068 may extend perpendicular to the axis 2046 of the first jaw 2032. The slot 2068 may include arms 2071 extending perpendicular to the axis 2046, and extending above the compression surface 2042 of the first jaw 2032 such that the slot 2068 extends above the compression surface 2042 of the first jaw 2032. The arms 2071 may bound the slot 2068.

The pin 2070 may comprise a portion of the proximal end portion 2050 of the second jaw 2034. The pin 2070 may be configured to fit within the slot 2068 and slide within the slot 2068. The pin 2070 may be configured to rotate within the slot 2068.

Referring to FIG. 116, the pin 2070 may be configured to slide within the slot 2068 between an upper end 2072 of the slot 2068 and a lower end 2074 of the slot 2068. The pin 2070 may be positioned at the lower end 2074 of the slot 2068 when the clip 2030 is in a closed configuration (as shown in FIG. 113 for example). The pin 2070 may be positioned at the upper end 2072 of the slot 2068 when the clip 2030 is in an opened configuration (as shown in FIG. 11 for example).

Referring to FIG. 11, in an opened configuration, the pin 2070 may be slid along the slot 2068 towards the upper end 2072 of the slot 2068. Such a configuration may increase a distance between the first jaw 2032 and the second jaw 2034 and accordingly may increase a width of the channel 2064. The first jaw 2032 and the second jaw 2034 may be moved away from each other and may extend parallel with each other as desired (with the axes 2046, 2060 extending parallel with each other). Further, the pin 2070 may rotate within the slot 2068 to rotate the distal end portions 2038, 2052 away from each other. A resulting configuration is shown in FIG. 111. A distance 2076 between the distal end portions 2038, 2052 accordingly may be greater than the distance 2078 between the proximal end portions 2036, 2050. A width of the channel 2064 between the distal end portions 2038, 2052 may be greater than the width between the proximal end portions 2036, 2050. The first jaw 2032 may be angled with respect to the second jaw 2034 and the axes 2046, 2060 may be non-parallel and configured to intersect each other.

In an opened configuration as shown in FIG. 111, the wide opening 2062 at the distal end portions 2038, 2052 of the jaws 2032, 2034 may improve the ease in which an object, such as a portion of a heart, may be inserted into the channel 2064. The distal tips 2039, 2037 may be angled away from each other to improve the ease of entry into the channel 2064.

Upon moving the clip 2030 to a closed configuration, the pin 2070 may rotate within the slot 2068. The pin 2070 may rotate such that the distal tips 2039, 2037 are moved towards each other. FIG. 112, for example, illustrates such a configuration. The pin 2070 may remain at the upper end 2072 of the slot 2068. The distal end portions 2038, 2052 of the jaws 2032, 2034 may move towards each other to reduce the distance 2076 between the distal end portions 2038, 2052. As such, the slotted pin joint 2066 may be configured to allow the first jaw 2032 to angle relative to the second jaw 2034 such that the channel 2064 has a width (corresponding to distance 2078) at the proximal end portions 2036, 2050 of the jaws 2032, 2034 that is greater than a width (corresponding to distance 2076) at the distal end portions 2038, 2052 of the jaws 2032, 2034. The axes 2046, 2060 may be non-parallel and configured to intersect each other.

In a configuration as shown in FIG. 112, the slotted pin joint 2066 is configured to allow the distal end portions 2038, 2052 of the jaws 2032, 2034 to close prior to the proximal end portions 2036, 2050 of the jaws 2032, 2034. Such a feature may reduce a possibility of an object being compressed, such as a portion of a heart, from escaping distally from the distal end portions 2038, 2052 and not being compressed between the jaws 2032, 2034.

Referring to FIG. 113, in the closed configuration, the pin 2070 slides along the slot 2068 towards the lower end 2074 of the slot 2068 to reduce the distance between the first jaw 2032 and the second jaw 2034. The distance 2078 at the proximal end portions 2036, 2050 is shown to be reduced. The jaws 2032, 2034 may extend parallel with each other, with the axes 2046, 2060 extending parallel with each other. The pin 2070 may rotate within the slot 2068 to allow the jaws 2032, 2034 to extend parallel with each other with the pin 2070 positioned at the lower end 2074 of the slot 2068.

A movement of the jaws 2032, 2034 from the position shown in FIG. 112 to the position shown in FIG. 113 may allow the compression of the object within the channel 2064 to occur in a direction from the distal end portions 2038, 2052 of the jaws 2032, 2034 towards the proximal end portions 2036, 2050. The direction of compression may further reduce the possibility of the object being compressed from escaping distally from the distal end portions 2038, 2052 of the jaws 2032, 2034. The distal tips 2039, 2037 of the jaws 2032, 2034 may close prior to the jaws 2032, 2034 at the closed end of the channel 2064. The proximal end portions 2036, 2050 may comprise the closed end of the channel 2064 that may retain the object within the channel 2064. The arms 2071, for example, may bound the proximal end of the channel 2064.

FIG. 114 illustrates the clip 2030 in a closed configuration, with a left atrial appendage 18 positioned between the jaws 2032, 2034 of the clip 2030. The clip 2030 may be moved from the closed configuration back to an opened configuration as shown in FIG. 111, for example, via a reverse operation than shown in FIGS. 111-113.

In examples, a spring 2080 may be provided that may be configured to force the first jaw 2032 towards the second jaw 2034. Referring to FIG. 11, for example, the spring 2080 may include an upper distal portion 2079, a proximal portion 2077, and a lower distal portion 2086. The upper distal portion 2079 may extend over the outer surface 2054 of the second jaw 2034 and may apply a force to the second jaw 2034 at the distal end portion 2052 or other portion of the second jaw 2034 as desired. The lower distal portion 2086 may extend over the outer surface 2040 of the first jaw 2032 and may apply a force to the first jaw 2032 at the distal end portion 2038 or other portion of the first jaw 2032 as desired. The proximal portion 2077 may comprise a bend portion that extends over the proximal end portions 2036, 2050 of the jaws 2032, 2034 and couples the upper distal portion 2079 to the lower distal portion 2086. The bend portion may have a curved loop shape, and the upper distal portion 2079 and lower distal portion 2086 may each comprise straightened arms, or may have another shape as desired. The spring 2080 may have a “C” shape or another shape as desired.

The spring 2080 may be made of a variety of materials including a metal such as cobalt-chromium, titanium, or stainless steel, among other forms of metal. The material may comprise a shape memory material such as nitinol or another form of shape memory material as desired. The material may have another form, such as a plastic or ceramic material in examples, or another form of material as desired.

In examples, the spring 2080 may provide the force that moves the clip 2030 from the opened configuration shown in FIG. 11 to the closed configuration shown in FIG. 113. In examples, a delivery system may be utilized to control movement of the clip 2030 from the opened configuration shown in FIG. 111 to the closed configuration shown in FIG. 113. In examples, a combination of a delivery system and the spring 2080 may control movement of the clip 2030 from the opened configuration to the closed configuration.

FIG. 115 illustrates a cross sectional view of a portion of the clip 2030 along line C-C in FIG. 113. FIG. 116 illustrates a cross sectional view of a portion of the clip 2030 along line D-D in FIG. 113.

In operation, the clip 2030 may be utilized to close the LAA 18 or another portion of a heart. A procedure may include gaining access to the heart 14. The access may be provided in a variety of manners, including a transcatheter access or a surgical access such as a thoracotomy, mini thoracotomy, or other methods of access. An open or mini sternotomy approach may be utilized as desired. A right-side approach may be utilized, for example, where direct visualization of the LAA 18 is compromised. Indirect visualization such as robotic visualization may be utilized as desired.

FIGS. 117 and 118 illustrate a closure of the clip 2030 around the LAA 18. FIG. 117 illustrates a cross sectional schematic view of a LAA 18. Referring to FIG. 118, the clip 2030 may be advanced over the LAA 18 with the outer surface 28 of the LAA 18 positioned between the jaws 2032, 2034. The LAA 18 may pass through the opening 2062 of the clip 2030 marked in FIG. 11 that leads to the channel 2064 between the jaws 2032, 2034. The clip 2030 may be positioned at the ostium 22 or other portion of the LAA 18.

The clip 2030 may be moved to the closed configuration upon the LAA 18. In examples, if desired, the clip 2030 may be released or repositioned in a reverse operation of the closure steps shown in FIGS. 111-113. The steps of the methods disclosed herein may be modified, substituted, excluded, or added to, as desired.

The clip 2030 may be utilized to close the LAA 18, yet in examples other portions of a heart may be clipped or closed via use of the clip 2030. In examples, other portions of a body, such as a tubular vessel or other portions of a body may be closed with the clip 2030. Deployment may be via a delivery apparatus or via another method as desired. Deployment may be via surgical methods and may be transcatheter or via non-invasive surgery in methods.

Variations in the clips and methods disclosed herein may be provided. FIGS. 119-121, for example illustrate a variation of a clip 2090 that may be configured similarly as the clip 2030. The clip 2090, for example, may include a first jaw 2092 and a second jaw 2094 and a slotted pin joint 2096 coupled to the first jaw 2092 and the second jaw 2094 and configured to allow the first jaw 2092 to move relative to the second jaw 2094 to compress a portion of a heart between the first jaw 2092 and the second jaw 2094.

The slotted pin joint 2096 may operate in a similar manner as the slotted pin joint 2066 shown in FIGS. 111-116. The slotted pin joint 2096, however, may be configured with the slot 2098 positioned interior of arms 2100 that support the pin 2102. FIG. 121, for example, illustrates a perspective end view of the clip 2090 showing the position of the slot 2098. A groove 2104 may be positioned at the proximal end portion 2106 of the second jaw 2094 between the arms 2100. The groove 2104 may receive the proximal end portion 2108 of the first jaw 2092 and may reduce the possibility of the jaws 2092, 2094 twisting laterally relative to each other undesirably.

Referring to FIG. 119, the spring 2110 may include an upper distal portion 2112 that may extend over and be raised above the first jaw 2092, and may include a lower distal portion 2114 that may be in contact with the second jaw 2094. The lower distal portion 2114 may extend parallel with the second jaw 2094. The spring 2110 may include a bend portion 2116 that couples the upper distal portion 2112 to the lower distal portion 2114. The spring 2110 may otherwise operate in a similar manner as the spring 2080 shown in FIGS. 111-114, and the features disclosed in regard to the clip 2030 may be applied to the operation of the clip 2090.

Variations in the clips and methods disclosed herein may be provided. Features across examples may be combined. Features may be excluded or added to in various examples disclosed herein. Combinations of features of examples may be provided. The features may be utilized solely or in methods disclosed herein, or in combination with other features disclosed herein.

Further, in examples, it may be beneficial to close the LAA 18 proximate the ostium 22, to reduce the possibility of pits or divots of the LAA 18 at the ostium 22 releasing undesired materials into the bloodstream.

FIG. 122 illustrates an example of a clip 2230 that may be utilized with systems herein. The clip 2230 may be for occluding a portion of a heart, which may be portion of the LAA 18. The clip 2230 may comprise a loop body and may include a first elongate jaw 2232, a second elongate jaw 2234, a closed end 2236, and an opened end 2238 opposite the closed end 2236. A channel 2240 may be between the first elongate jaw 2232 and the second elongate jaw 2234 for receiving the portion of the heart to be compressed or occluded. The first elongate jaw 2232, the second elongate jaw 2234, and the closed end 2236 may bound a channel 2240 for receiving the portion of the heart to be compressed or occluded. The opened end 2238 may comprise an opening for the channel 2240.

The clip 2230 may comprise a V-clip in examples, although other forms of clips may be utilized in examples.

The clip 2230 may have an outer covering that may be compliant or atraumatic, such as a cloth covering comprising an outer surface of the clip 2230. An outer surface of the clip 2230 may be configured to provide tissue adhesion in examples. Other forms of clips may be utilized.

FIG. 123 illustrates a top view of the clip 2230.

The clip 2230 may be configured to move between opened and closed positions. The clip 2230 is shown in FIG. 122 in an opened position. In such a position, the clip 2230 may be positioned over the portion of the heart to be compressed or occluded with the portion inserted through the opened end 2238. The tips 2241, 2243 of the respective first elongate jaw 2232 and the second elongate jaw 2234 may be moved away from each other in the opened position and may be moved towards each other in the closed position. The jaws 2232, 2234 of the clip 2230 may pivot about the closed end 2236 to move between the opened position and the closed position.

FIG. 124 illustrates an end view of the clip 2230 in a closed position. FIG. 125 illustrates a side view of the clip 2230 in a closed position. The clip 2230 may be closed to compress or occlude a portion of the heart between the jaws 2232, 2234.

FIG. 126 illustrates a perspective view of a delivery apparatus 2245 that may be utilized in examples herein. The delivery apparatus 2245 may include a deployment head 2242 and may include other features such as an elongate delivery shaft 2244 and a handle 2246 at a proximate end of the delivery shaft 2244. The deployment head 2242 may include retainers 2248, 2250 for retaining respective clips and moving respective clips between opened and/or closed positions.

A control mechanism may be utilized to close and/or open the clips. The control mechanism, for example, may include the retainers 2248, 2250 and may include a control actuator such as triggers 2252, 2254 for controlling the retainers 2248, 2250. In examples, the retainers 2248, 2250 may be independently controlled to separately control actuation of the retainers 2248, 2250 and the respective clips coupled to the retainers 2248, 2250. For example, each trigger 2252, 2254 may be independently operated to independently control a respective retainer 2248, 2250. In examples, other forms of control mechanisms may be utilized.

FIG. 127 illustrates a top view of the deployment head 2242. The retainers 2248, 2250 are shown to extend parallel with each other such that clips retained by the retainers 2248, 2250 may extend parallel with each other and may be adjacent to each other. The deployment head 2242 may be configured to retain the first clip 2230 parallel with the second clip 2260. The retainers 2248, 2250 may retain the clips proximate to each other at a desired distance to place the clips proximate to each other upon deployment.

FIG. 128 illustrates an end view of the deployment head 2242. The retainers 2248, 2250 may be movable to close and/or open the clips coupled thereto as desired. The retainers 2248, 2250 for example may comprise movable arms that may extend over the clips and may open and close away and towards each other respective to open and close the clips. The moveable arms may include channels 2253, 2255 that the clips may slide out of upon deployment.

A system as utilized herein may utilize a plurality of clips, or at least two clips. Each clip may each be configured similarly as the clip 2230 shown in FIG. 122, or may have other configurations as desired. The clip 2230 shown in FIG. 122, for example, may comprise a first clip. A second clip 2260 may be utilized that may be similar to the first clip 2230 or may have another configuration in examples. The clips 2230, 2260 may be held by the retainers 2248, 2250 parallel with each other and for adjacent deployment, as shown in FIG. 129 for example.

Upon deployment, the clips may be deployed to the heart in sequence. A clip system may be utilized. Referring to FIG. 130, the first clip 2230 may be deployed proximate an ostium of the LAA to occlude a portion of the LAA. The first clip 2230 may occlude the LAA. FIG. 131, for example, illustrates the clip 2230 occluding the LAA. Notably, a portion of the interior wall 2262 of the left atrium 10 may not be compressed by the clip 2230 and other pits and divots of the interior surface of the LAA may be uncompressed as well. Such pits and divots may be capable of releasing undesired materials or emboli into the blood stream.

As such, the second clip 2260 may be deployed between the position of the first clip 2230 and the wall 20 of the left atrium 10 to occlude a portion of the heart between the first clip 2230 and the wall 20 of the left atrium 10. The delivery apparatus 2245 may be configured to deploy one or more of the first clip 2230 and/or the second clip 2260 to the respective portions.

Referring to FIG. 132, the second clip 2260 is shown deployed adjacent to the first clip 2230 and parallel with the first clip 2230. The clips 2260, 2230 may be positioned at the neck of the LAA.

The second clip 2260 may draw a portion of the outer wall 20 and the interior wall 2262 of the left atrium 10 into the portion of the heart occluded by the second clip 2260. LAA tissue and left atrial tissue may be stretched and pulled into the compressed portion of the second clip 2260. The leading edge of the ostium may be drawn into the compressed portion of the second clip 2260. The second clip 2260 may draw pits or divots of the LAA into the portion of the heart occluded by the second clip 2260. As such, the possibility for pits or divots of the LAA remaining exposed to the left atrium chamber may be reduced and the possibility for emboli undesirably releasing into the bloodstream may be reduced. Pits or divots may be compressed by the clip 2260 to reduce the possibility of leakages at these areas. The combination of the first clip 2230 and the second clip 2260 may reduce the possibility of bleeding or other maladies entering the bloodstream. Irregularities common to the opening of the ostium may be addressed.

In examples, the first clip 2230 and the second clip 2260 may be deployed sequentially. For example, deployment of the second clip 2260 may follow deployment of the first clip 2230. The first clip 2230 may serve to stabilize and position the second clip 2260 upon deployment of the second clip 2260. The second clip 2260 may serve as a redundant closure line of the LAA to prevent residual leaks. An improved closure line may result. The delivery apparatus 2245 may be utilized to sequentially and independently deploy the first clip 2230 and the second clip 2260.

In examples, the first clip 2230 may be applied and not yet released from the delivery apparatus 2245 until the second clip 2260 is applied. The first clip 2230 and second clip 2260 may each be released and readjusted for desired occlusion.

Variations in the clips or the deployment method may be provided as desired.

The features of FIGS. 122-132 may be utilized solely or in combination with any other example disclosed herein.

Other forms of clips may be utilized in examples.

FIG. 133, for example, illustrates a clip 2280 including a first jaw or elongate upper jaw 2282 and a second jaw or elongate lower jaw 2284. The elongate upper jaw 2282 may extend along a first axis 2292 (marked in FIG. 134). The elongate upper jaw 2282 may include an outer surface 2286 that may be configured to rotate about the first axis 2292 in a first rotational direction.

A connecting portion 2291 of the clip 2280 may comprise a loop body that may couple the elongate upper jaw 2282 to the elongate lower jaw 2284. The jaws 2282, 2284 may be configured to pivot about the connecting portion 2291 to move from an opened position (as shown in FIG. 133) to a closed position (with the jaws 2282, 2284 parallel with each other).

Referring to FIG. 134, the upper jaw 2282 may include a first axle 2290 extending along the first axis 2292. The axle 2290 may comprise an elongate axle coincident with the first axis 2292. A roller 2294 may surround the first axle 2290 and may be configured to rotate about the first axle 2290. The outer surface 2286 may rotate about the first axle 2290. The roller 2294 may comprise a cylinder extending over the first axle 2290 or may have another configuration in examples. As shown in FIG. 133, the roller 2294 may have a proximal portion 2295 and a distal portion 2297.

Referring to FIG. 134, in examples, the roller 2294 may include an opening 2296 that may be configured to receive an elongate arm 2300 of a clip body 2302 as shown in FIG. 137 for example. The opening 2296 may comprise an entry to a channel 2304 that extends along the length of the roller 2294, as shown in FIG. 135 for example. The channel 2304 may extend along the length of the roller 2294 and parallel with the axis 2292.

The roller 2294 may comprise a silicone body or other soft material (e.g., polyetheretherketone (PEEK)). The axle 2290 may be made of metal or other supporting material. The roller 2294 may be covered with a textile to provide grip as desired. Thromboresistant material may be utilized as desired, or material to provide cell overgrowth.

The lower jaw 2284 may be configured similarly as the upper jaw 2282, as shown in FIG. 136. The lower jaw 2284 may extend along a second axis 2293 and may have a second outer surface 2288. The lower jaw 2284 may be configured for the second outer surface 2288 to rotate about the second axis 2293 in a second rotational direction that is opposite the first rotational direction. The lower jaw 2284 may include a roller 2299 and an axle 2301 similar to the upper jaw 2282. The lower jaw 2284 may include an opening 2303 that leads to a channel 2311 (marked in FIG. 138) configured similarly as the respective opening 2296 and channel 2304 of the upper jaw 2282.

The openings and channels of the respective upper jaw 2282 and lower jaw 2284 may be configured to rotate positions about the axes 2292, 2293 and the axles 2290, 2301.

FIG. 137 illustrates a side view of a clip body 2302 that may be inserted into the clip 2280. The clip body 2302 may be configured to be inserted into the upper jaw 2282 and the lower jaw 2284 to cause the outer surfaces 2286, 2288 to rotate. The clip body 2302 may include the first elongate arm 2300 and a second elongate arm 2306 extending parallel with the first elongate arm 2300. A connecting portion 2308 may be bent or curved and may connect the elongate arms 2300, 2306. The clip body 2302 may comprise a loop body include a closed end 2309 and an opened end 2315. The clip body 2302 may have a “U” shape. The clip body 2302 may comprise a spring configured to draw the elongate arms 2300, 2306 towards each other when deflected away from each other.

The clip body 2302 may be inserted into the openings 2296, 2303 of the rollers 2294, 2299 and may apply a compressive force to the rollers 2294, 2299 to rotate the rollers about the axles 2290, 2301. The first elongate arm 2300 may extend into the upper jaw 2282 and the second elongate arm 2306 may extend into the lower jaw 2284.

FIG. 138, for example, illustrates an exemplary deployment. The clip 2280 may be deployed to the LAA proximate the ostium of the LAA. The clip 2280 is shown in an opened position in FIG. 133 and may be closed such that the rollers 2294, 2299 extend parallel with each other in a closed position. The rollers 2294, 2299 may pivot about the connecting portion 2291 of the clip 2280 to move to a closed position.

The channels 2304, 2311 may be positioned distal from each other. The clip body 2302 may then be inserted into the clip 2280 to cause the rollers 2294, 2299 to rotate, as shown in FIG. 139 for example. The channels 2304, 2311 may be drawn proximally towards each other as shown in FIG. 139 (with the arms 2300, 2306 inserted into the channels 2304, 2311). Portions of the heart, particularly the inner wall of the left atrium or the LAA, may be drawn between the outer surfaces 2286, 2288 of the clip 2280. Portions of the heart, particularly the inner wall of the left atrium or the LAA, may be drawn through the rollers 2294, 2299 to reduce the possibility of emboli releasing from any pits or divots of the LAA. Pits or divots of a LAA may be drawn into a portion of the heart occluded by the clip 2280, in a similar manner as discussed in regard to FIG. 132. Enhanced sealing of the LAA may result.

The clip body 2302 may further provide an additional compression force for the clip 2280.

FIG. 140 illustrates a variation in which axles 2310, 2313 of a clip may be eccentric with respect to the outer surface 2321, 2323 of the respective rollers 2325, 2327. As such, an inward motion of the rollers 2325, 2327 towards each other may occur to further occlude the LAA. The rollers 2325, 2327 may include respective channels 2329, 2331 for receiving arms of a clip, in a similar manner as the channels 2304, 2311. Various shapes of rollers 2325, 2327 may be utilized including circular shapes, ovoid shapes, or other shapes (e.g., triangular, hexagonal, etc.).

In examples, the clip 2280 may be a clip having an open end, or may be a clip with closed ends. A clip having an open end may be configured to pivot opened or closed in a similar manner as shown in FIGS. 122 and 125.

FIG. 141 illustrates a variation of a clip 2320 having closed ends 2322, 2324. The clip 2320 may be slid along the length of the LAA and placed in position, with the rollers 2326, 2328 configured to roll against the LAA. The closed ends 2322, 2324 may comprise loops that couple the rollers 2326, 2328 together. The closed ends 2322, 2324 may be coupled to axles for the rollers 2326, 2328 to roll around, in a similar manner as the axles 2290, 2301.

FIG. 142 illustrates a top view of the clip 2320. FIG. 143 illustrates a side view of the clip 2320.

The features of FIGS. 133-143 may be utilized solely or in combination with any other example disclosed herein.

Variations in the clips and methods disclosed herein may be provided. Features across examples may be combined. Features may be excluded or added to in various examples disclosed herein. Combinations of features of examples may be provided. The features may be utilized solely or in methods disclosed herein, or in combination with other features disclosed herein.

FIG. 144 illustrates an example of a clip 2430 that may be utilized for a portion of a heart. The clip 2430 may be configured to close a LAA 18, to reduce the possibility of clots or other undesired materials stemming from the LAA 18 from traveling into the bloodstream. The clip 2430 may comprise a V-clip in examples or may have another configuration.

The clip 2430 may include a first jaw or upper jaw 2432 and a second jaw or lower jaw 2434. The upper jaw 2432 may be configured to apply a compressive force to a first side of the portion of the heart to be compressed. The lower jaw 2434 may be configured to apply a compressive force to a second side of the portion of the heart to be compressed.

The upper jaw 2432 may extend from a proximal portion 2436 to a distal portion 2438. The proximal portion 2436 may comprise a proximal end of the upper jaw 2432 and the distal portion 2438 may comprise a distal end of the upper jaw 2432.

The upper jaw 2432, in examples, may have a compressive surface 2440 and an upper surface 2442 facing opposite the compressive surface 2440. The compressive surface 2440 may be configured to apply the compressive force to the first side of the portion of the heart.

The upper jaw 2432 may include one or more side surfaces 2444 positioned between the compressive surface 2440 and the upper surface 2442. In examples, the side surfaces 2444 may include a flange 2446 that may protrude from the side surfaces 2444 and extend circumferentially about the upper jaw 2432. The flange 2446 may form an outer perimeter of the one or more side surfaces 2444. The flange 2446 may be positioned at the upper surface 2442 of the upper jaw 2432.

In examples, the upper jaw 2432 may have a wedge shape as shown in FIGS. 144 and 146. FIG. 146, for example, illustrates a cross sectional view of the upper jaw 2432 along a mid-line. The upper surface 2442 is shown to be at an acute angle with respect to the compressive surface 2440, with the thickness of the distal portion 2438 of the upper jaw 2432 being less than the proximal portion 2436 of the upper jaw 2432.

The upper jaw 2432, in examples, may be compliant. The upper jaw 2432, in examples, may comprise a material such as a polymer that may be configured to have some pliability or flexibility yet have sufficient strength to apply a compressive force to a portion of a heart. The material may comprise a silicone molded with a rigid implantable material in examples, or another material.

The second or lower jaw 2434 may be configured similarly as the first or upper jaw 2432. The lower jaw 2434 may be positioned to oppose the upper jaw 2432 and may be positioned on an opposite side of the portion of the heart to be compressed (e.g., an opposite side of a left atrial appendage (LAA)). The lower jaw 2434 may include a compressive surface 2447 that may be configured to apply a compressive force to the second side of the portion of the heart to be compressed. The compressive surfaces 2440, 2447 of the upper jaw 2432 and the lower jaw 2434 may comprise flat surfaces that may extend parallel with each other. The lower surface 2449 of the lower jaw 2434 may extend at an angle with respect to the compressive surface 2447, similar to the angle between the upper surface 2442 and the compressive surface 2440.

FIG. 146, for example, illustrates the upper surface 2442 extending at an angle with respect to the respective compressive surface 2440.

The lower surface 2449 of the lower jaw 2434 may face opposite the compressive surface 2447 and side surfaces 2451 extending between the compressive surface 2447 and the lower surface 2449. A flange 2453 may be positioned at the lower surface 2449 of the lower jaw 2434 and may be configured similarly as the flange 2446. The lower jaw 2434 may comprise a duplicate and an inversion of the upper jaw 2432 in examples. In examples, the lower jaw 2434 may have a different configuration than the upper jaw 2432.

Referring to FIG. 144, the clip 2430 may include a retainer body 2448. The retainer body 2448 may include an upper receiver 2450 configured to be positioned on a first side of the portion of the heart to be compressed, and may include a lower receiver 2452 configured to be positioned on a second side of the portion of the heart to be compressed that is opposite the first side. A loop body 2454 may couple the upper receiver 2450 to the lower receiver 2452.

The retainer body 2448 may comprise a frame in examples. The retainer body 2448 may be made a spring material. In examples, the retainer body 2448 may comprise a shape memory material such as nitinol, or another form of shape memory material in examples. Other materials, such as plastic may be utilized. A plastic material may be compounded with powder silicone to make the parts atraumatic. The retainer body 2448 may be more rigid than the material of the jaws.

The upper receiver 2450 may include a pair of opposed tracks 2456a, b that are configured to slidably engage with the upper jaw 2432. The pair of opposed tracks 2456a, b, for example, may receive the flange 2446 of the upper jaw 2432.

The upper receiver 2450 may include a proximal portion 2459 and a distal portion 2461.

The upper receiver 2450 may comprise a frame having a “U” shape surrounding a central cavity 2458. The “U” shape may extend in a plane that is perpendicular to the plane of opening and closing of the clip 2430. The upper receiver 2450, for example, may include a first elongate arm 2463 and a second elongate arm 2465 both extending from the proximal portion 2459 of the upper receiver 2450 to the distal portion 2461 of the upper receiver 2450. The first elongate arm 2463 and the second elongate arm 2465 may both extend parallel and on opposite sides of the central cavity 2458. The “U” shape may extend around the outer perimeter of the side surfaces of the upper jaw 2432. The “U” shape may include an opening at the proximal portion 2459 of the upper receiver 2450 for the side surfaces 2444 of the upper jaw 2432 to pass through (as shown in FIG. 2).

A connecting portion 2467 may connect the first elongate arm 2463 and the second elongate arm 2465 at the distal portion 2461 of the upper receiver 2450. The connecting portion 2467 may include a track 2456c that may connect the tracks 2456a, 2456b and may be positioned at the distal portion 2461 of the upper receiver 2450.

The pair of opposed tracks 2456a, b may each comprise a channel for the flange 2446 to insert into and slide along. The track 2456c may comprise a channel that is continuous with the channels of the tracks 2456a, b. In examples, the tracks 2456a, b, c may have other forms. For example, the tracks may comprise protrusions configured to insert into a portion of the upper jaw 2432 for the upper jaw 2432 to slide along.

The channels of the pair of opposed tracks 2456a, b may have openings at the proximal portion 2459 of the upper receiver 2450 for the flange 2446 to be inserted into. The upper jaw 2432 may be configured to slide along the pair of opposed tracks 2456a, b in a direction from the proximal portion 2459 of the upper receiver 2450 towards the distal portion 2461 of the upper receiver 2450.

In examples, one or more locks 2460 (marked in FIG. 147) may be positioned at the proximal portion 2459 of the upper receiver 2450. The one or more locks 2460 may lock the upper jaw 2432 to the upper receiver 2450 upon full insertion into the upper receiver 2450. The one or more locks 2460 may be configured to lock at least a portion of the upper jaw 2432 within the first pair of opposed tracks 2456a, b and lock at a least a portion of the lower jaw 2434 within the pair of opposed tracks of the lower receiver 2452. The locks 2460 may comprise protrusions that may contact a proximal end of the flange 2446 to lock the upper jaw 2432 within the upper receiver 2450.

The lower receiver 2452 may have a similar construction as the upper receiver 2450, and may be configured to receive the lower jaw 2434. The lower receiver 2452 may comprise a duplicate and an inversion of the upper receiver 2450 in examples. The lower receiver 2452 may include a pair of opposed tracks (a track 2456d is shown in FIG. 145), which may be similar to the pair of opposed tracks 2456a, b. In examples, the lower receiver 2452 may have a different configuration than the upper receiver 2450.

The loop body 2454 may be positioned at a proximal portion of the upper receiver 2450 and the lower receiver 2452. The loop body 2454 may have a curved shape and may be configured to be positioned at a side of the portion of the heart being compressed. The curved shape may extend in a proximal direction to accommodate a shape of the portion of the heart being compressed. The loop body 2454, in examples, may comprise a spring body configured to compress the upper receiver 2450 and lower receiver 2452 towards each other and accordingly compress the upper jaw 2432 and lower jaw 2434 towards each other The loop body 2454 may be configured to angle the upper receiver 2450 and lower receiver 2452 towards each other such that the distal tips 2469, 2471 of the upper receiver 2450 and lower receiver 2452 are closer to each other than the proximal ends of the upper receiver 2450 and lower receiver 2452 at which the loop body 2454 is positioned.

FIG. 145 illustrates a side cross sectional view of the retainer body 2448 showing the track 2456c. A track 2456d of the lower receiver 2452 is shown in FIG. 145. FIG. 146 illustrates a side cross sectional view of the upper jaw 2432.

The upper jaw 2432 and lower jaw 2434, in examples, may be slid distally along the retainer body 2448 to seat within the retainer body 2448. FIG. 147, for example, illustrates a perspective view of the upper jaw 2432 and the lower jaw 2434 slid distally within the upper receiver 2450 and the lower receiver 2452. At least a portion of the upper jaw 2432 is configured to be slidably engaged with the first pair of opposed tracks 2456a, b of the upper receiver 2450 and at least a portion of the lower jaw 2434 is configured to be slidably engaged with the second pair of opposed tracks of the lower receiver 2452. The locks 2460 may lock the upper jaw 2432 and the lower jaw 2434 to the retainer body 2448.

FIG. 148, for example, illustrates a side view of the upper jaw 2432 and the lower jaw 2434 seated within the retainer body 2448.

In examples, upon deployment, the upper jaw 2432 and the lower jaw 2434 may be in a retracted position, as shown in FIG. 144, for example, relative to the retainer body 2448. In such a position, the upper jaw 2432 and the lower jaw 2434 may be retracted proximally such a force may be applied to the proximally protruding portions 2475, 2477 of the upper jaw 2432 and the lower jaw 2434, respectively, to open the clip 2430. The protruding portions of the upper jaw 2432 and lower jaw 2434, for example, may increase a leverage force to open the clip 2430.

FIG. 149, for example, illustrates the clip 2430 being advanced to the portion of the heart to be compressed, which is the LAA in this example. The upper jaw 2432 and the lower jaw 2434 are positioned proximally, to increase a leverage force to open the clip 2430 and advance the clip over the LAA surface. Proximally protruding portions 2475, 2477 (marked in FIG. 144) of the jaws 2432, 2434 may be contacted to open the clip 2430 and hold the clip 2430 open. With the retainer body 2448 placed in the desired position over the LAA surface, the upper jaw 2432 and the lower jaw 2434 may be slid distally as represented in FIG. 150 to compress and occlude the LAA. The upper jaw 2432 and lower jaw 2434 may be advanced to a position as shown in FIG. 147.

The deployment of the clip 2430 may allow the tips 2469, 2471 of the clip 2430 to close first, due to the angled configuration of the retainer body 2448. The tips 2469, 2471 of the clip 2430 may close to occlude the LAA. The compliant material of the jaws 2432, 2434 may reduce trauma to the surface of the LAA.

In examples, the upper jaw 2432 or the lower jaw 2434 may be retracted from the retainer body 2448 to reduce a force upon the LAA for repositioning of the clip 2430 as desired. The clip 2430 may be removed for adjustment as desired. The clip 2430 may comprise a low-profile clip that may allow for reduced bulkiness of the clip 2430.

In examples, the upper jaw 2432 and the lower jaw 2434 may be in a position as shown in FIG. 147 upon deployment. Such a configuration may be utilized for a deployment in a direction perpendicular to the ostium of the LAA. Such a configuration may further be utilized for a deployment in a direction parallel to the LAA as shown in FIGS. 149 and 150.

The clip 2430 may be covered with material such as a polymer to allow for sealing with the occlusion site. A material such as silicone may be utilized. Other forms of material, such as soft material, may cover the clip 2430.

The features of the examples of FIGS. 144-150 may be utilized solely or in combination with any other example disclosed herein.

FIG. 151 illustrates a view of a first jaw or upper jaw 2470 and a second jaw or lower jaw 2472 that may be utilized with a clip 2473 (marked in FIG. 153). The lower jaw 2472 may form a plate having a compressive surface 2476 (marked in FIG. 155) and a lower surface 2478 (marked in FIG. 153) facing opposite the compressive surface 2476. The compressive surface 2476 may be configured to apply a compressive force to a side of the portion of the heart. The clip 2473 may comprise a V-clip in examples or may have another configuration.

The compressive surface 2476 may include a layer of padding in examples. For example, a layer of silicone padding may be provided as desired, or other materials.

The lower jaw 2472 may include one or more side surfaces 2480 extending between the compressive surface 2476 and the lower surface 2478. One of the side surfaces 2480 may include an aperture 2482. The aperture 2482 may be for receiving a second portion 2485 of a loop body 2484 shown in FIG. 152 for example.

The upper jaw 2470 may be configured similarly as the lower jaw 2472. The upper jaw 2470, for example, may form a plate. The upper jaw 2470 may include a compressive surface configured to apply a compressive force to a side of a portion of the heart and may include an upper surface 2486 facing opposite the compressive surface. The compressive surface may include a layer of padding in examples. For example, a layer of silicone padding may be provided as desired, or other materials.

The upper jaw 2470 may include one or more side surfaces 2489 positioned between the compressive surface and the upper surface 2486. The upper jaw 2470 may include an aperture 2474 on a side surface 2489 for receiving a first portion 2487 of the loop body 2484 shown in FIG. 152 for example.

The compressive surfaces of the upper jaw 2470 and lower jaw 2472 may both be flat surfaces and may extend parallel with each other. The compressive surfaces may include a soft material such as silicone if desired. The upper jaw 2470 and lower jaw 2472 may each have a rectangular shape when viewed from the side and from a top view, or may have another shape as desired.

The apertures 2474, 2482 may each form entrances to an elongate channel for receiving the respective first portion 2487 and second portion 2485 of the loop body 2484 shown in FIG. 152. The apertures 2474, 2482 and channels may each extend parallel with each other and may be aligned with each other in a plane.

FIG. 152 illustrates a side view of the loop body 2484. The loop body 2484 may be configured to insert into the apertures 2474, 2482 and configured to compress the upper jaw 2470 and the lower jaw 2472 towards each other. The loop body 2484 may have a “U” shape. The loop body 2484 may include a first portion 2487 in the form of an elongate arm, and a second portion 2485 in the form of an elongate arm. The first portion 2487 may be configured to insert into the aperture 2474 and the second portion 2485 may be configured to insert into the aperture 2482. The first portion 2487 may be configured to be slid along the aperture 2474, and the second portion 2485 may be configured to be slid along the aperture 2482.

The first portion 2487 and the second portion 2485 may be connected with a connecting portion 2488 extending between the proximal portions of the first portion 2487 and the second portion 2485. In examples, the loop body 2484 may be deflected inward towards a central channel 2490. The first portion 2487 and second portion 2485 may be angled towards each other such that the tips 2491, 2493 of the respective first portion 2487 and second portion 2485 are closer to each other than the portions 2487, 2485 at the connecting portion 2488. The loop body 2484 accordingly may be configured to press the upper jaw 2470 and lower jaw 2472 towards each other due to the force applied by the loop body 2484. The loop body 2484 may include compression points to provide the compressive force. The loop body 2484 may be spring biased to compress the upper jaw 2470 and lower jaw 2472 towards each other.

The loop body 2484 may be made from a spring material. In examples, the loop body 2484 may comprise a shape memory material such as nitinol, or another form of shape memory material in examples. Other materials, such as plastic may be utilized. A plastic material may be compounded with powder silicone to make the parts atraumatic.

FIG. 153 illustrates the loop body 2484 having been inserted into the apertures 2474, 2482. FIG. 154 illustrates an end view of the loop body 2484 positioned within the apertures 2474, 2482.

FIG. 155 illustrates a partial cross sectional view of the loop body 2484 within the aperture 2482.

Prior to deployment, the loop body 2484 may be retracted from the apertures 2474, 2482 fully or partially. FIG. 156, for example, illustrates the clip 2473 approaching the portion of the heart to be compressed (e.g., the LAA), with the loop body 2484 partially retracted from the apertures 2474, 2482. As such, the loop body 2484 may apply a reduced compressive force to the jaws 2470, 2472 to allow the clip 2473 to open.

Upon the jaws 2470, 2472 being placed in a desired position relative to the LAA, the loop body 2484 may be slid distally along the apertures 2474, 2482 to apply a greater compressive force to the jaws 2470, 2472. FIG. 157, for example, illustrates the loop body 2484 advanced and the jaws 2470, 2472 applying the compressive force to the LAA.

In examples, the loop body 2484 may be retracted from the apertures 2474, 2482 for repositioning of the clip 2473.

In examples, the loop body 2484 may be fully advanced distally into the apertures 2474, 2482 during deployment of the clip 2473. Such a configuration may be utilized for a deployment in a direction perpendicular to the ostium of the LAA. Such a configuration may further be utilized for a deployment in a direction parallel to the LAA as shown in FIGS. 156 and 157.

The clip 2473 may be coated with a soft material such as silicone if desired. Other soft material may comprise coatings or coverings of the clip 2473.

The features of the examples of FIGS. 151-157 may be utilized solely or in combination with any other example disclosed herein.

FIG. 158 illustrates an example of a loop body 2500 that may be utilized with a clip 2502 as shown in FIG. 164. The loop body 2500 may include a first elongate arm 2504 and a second elongate arm 2506, a first closed end 2508 and a first opened end 2510. A first channel 2512 may be positioned between the first elongate arm 2504 and the second elongate arm 2506. The first opened end 2510 may form an opening for the first channel 2512.

The first elongate arm 2504 and the second elongate arm 2506 may each include a compressive surface 2514, 2516 for applying a compressive force to a portion of the heart to be compressed or occluded (e.g., the LAA). The compressive surfaces 2514, 2516 may face each other and may face the first channel 2512 that may receive the portion of the heart to be compressed or occluded.

The first elongate arm 2504 and the second elongate arm 2506 may each include a respective receiving portion 2518, 2520 for receiving a portion of a second loop body 2522 shown in FIG. 161 for example. The receiving portions 2518, 2520 may each comprise elongate cavities or channels for receiving elongate arms of the second loop body 2522. The first elongate arm 2504 may include an elongate cavity for receiving a third elongate arm 2530 shown in FIG. 161. The second elongate arm 2506 may include an elongate cavity for receiving a fourth elongate arm 2532. Each elongate cavity may include lips 2524, 2526 for retaining the elongate arms of the second loop 2522 within the respective cavity. The lips 2524, 2526 may be positioned at an end of the elongate cavity.

The first closed end 2508 may comprise a bend in the loop that may couple the proximal ends of the first elongate arm 2504 and the second elongate arm 2506 to each other. The first closed end 2508 may be integral with the first elongate arm 2504 and the second elongate arm 2506 or may comprise a hinge or other pivotal structure configured to allow the first elongate arm 2504 and the second elongate arm 2506 to open away from each other or close towards each other.

FIG. 159 illustrates a rear perspective view of the loop body 2500. The receiving portion 2518 comprising an elongate channel may extend along the first closed end 2508 and may continuously join to the receiving portion 2520 (marked in FIG. 158) of the second elongate arm 2506. An elongate channel of the receiving portion 2518, for example, may be continuous with an elongate channel of the receiving portion 2520.

The loop body 2500 is shown in an opened configuration in FIGS. 158 and 159, yet may be biased towards a closed configuration as shown in FIG. 160. The loop body 2500 may be configured with a bias toward the closed configuration, yet a force may move the loop body 2500 to an opened configuration as shown in FIGS. 158 and 159 for example. The loop body 2500, for example, may comprise a spring body (e.g., a U-shaped metal clip) having a spring bias to hold its shape. Upon being opened, the loop body 2500 may return to a resting shape pinching and applying pressure to the LAA tissue to occlude the LAA lumen.

FIG. 161 illustrates a rear perspective view of a second loop body 2522 that may couple to the first loop body 2500. The second loop body 2522 may include a third elongate arm 2530, a fourth elongate arm 2532, a second closed end 2534, and a second opened end 2536. The third elongate arm 2530 and fourth elongate arm 2532 may bound a second channel 2538 between the third elongate arm 2530 and the fourth elongate arm 2532. The second opened end 2536 may form an opening for the second channel 2538.

FIG. 162 illustrates a side view of the second loop body 2522.

The second loop body 2522 may comprise a securing body that may serve to couple to the first loop body 2500 to secure the first loop body 2500 to the portion of the heart to be compressed or occluded. The second loop body 2522 may be inserted into the receiving portions 2518, 2520 of the first loop body 2500 to secure the first loop body 2500 in position.

Referring to FIG. 163 for example, the second loop body 2522 may be inserted into the first loop body 2500 with the respective closed ends 2508, 2534 positioned distal from each other. The second loop body 2522 may be configured to be slid along the first loop body 2500 in a direction from the first opened end 2510 of the first loop body 2500 towards the first closed end 2508 to couple to the first loop body 2500. The loop bodies 2500, 2522 may couple to form a ring or closed loop extending around the portion of the heart to be compressed. FIG. 165, for example, illustrates the second loop body 2522 being inserted into the first loop body 2500.

FIG. 164 illustrates the second loop body 2522 continue to be advanced into the first loop body 2500. The second loop body 2522 may couple to the first loop body 2500 with the second opened end 2536 of the second loop body 2522 positioned proximate the first closed end 2508 of the first loop body 2500, and the second closed end 2534 of the second loop body 2522 positioned proximate the first opened end 2510 of the first loop body 2500. The closed ends 2508, 2534 accordingly may positioned at opposite ends of the ring or closed loop from each other. The closed ends 2508, 2534 may bound the portion of the heart positioned within the channel 2512.

The elongate arms 2504, 2530 may extend parallel with each other, and the elongate arms 2506, 2532 may extend parallel with each other. The elongate arms 2504, 2530 together may form a first jaw for compressing a first side of a portion of the heart, and the elongate arms 2506, 2532 together may form a second jaw for compressing a second side of a portion of the heart. The first elongate arm 2504 may be positioned closer to a side of the portion of the heart to be compressed than the third elongate arm 2530. The second elongate arm 2506 similarly may be positioned closer to an opposite side of the portion of the heart to be compressed than the fourth elongate arm 2532.

In examples, one or more locks may be utilized to lock the first loop body 2500 to the second loop body 2522. FIG. 166, for example, illustrates a locking surface 2539 positioned on one or more arms of the second loop body 2522. The locking surface 2539 may be configured to lock with a corresponding locking surface 2541 on the first loop body 2500 (as shown in FIG. 167 for example).

FIG. 168 illustrates that the locking surfaces 2539, 2541 may comprise ratcheting surfaces that may lock the loop bodies 2500, 2522 in position. As such, the loop bodies 2500, 2522 may be advanced towards each other, yet not retracted from each other.

Various modifications may be provided. FIG. 169, for example, illustrates a gripping surface 2543 that may be applied to the first loop body 2500. The gripping surface 2543 may be configured to enhance the securement of the first loop body 2500 to the portion of the heart.

In examples, a cloth or compliant surface may be provided for the first loop body 2500. FIG. 170, for example, illustrates a compliant, compressible surface 2545 that may be provided on an outer surface of the first loop body 2500. A compliant surface may include polytetrafluoroethylene (PTFE) or polyethylene terephthalate (PET). In examples, a surface may be provided that may promote tissue growth with the clip.

FIG. 171 illustrates a variation in which a second loop body 2540 may extend over a first loop body 2542. The second loop body 2540 may include one or more locks 2544 on an interior surface of the second loop body 2540 that may engage with one or more locks 2546 on an exterior surface of the first loop body 2542. The locks 2544, 2546 may engage each other as shown in FIG. 172, with the first loop body 2542 forming an interior surface for contacting the portion of the heart to be compressed.

FIGS. 173-179 illustrate an exemplary deployment sequence. A delivery apparatus 2550 as shown in FIG. 173 may be utilized to grip the first loop body 2500. The delivery apparatus 2550, for example, may include an engagement portion 2552 that may couple to the first loop body 2500 and may be configured to move the first loop body 2500 to an opened configuration. FIG. 174 illustrates the first loop body 2500 in a closed configuration, and the engagement portion 2552 may move the first loop body 2500 to the opened configuration as shown in FIG. 175.

Referring to FIG. 176, the first loop body 2500 may approach the LAA and may be positioned at the ostium of the LAA. The elongate arms 2504, 2506 may be positioned on opposite sides of the LAA. An initial amount of occlusive force may be provided by the first loop body 2500.

In examples, a delivery apparatus may then approach and provide the second loop body 2522 in an opposite direction as shown in FIG. 177. The clip 2502 may be secured in position upon the LAA. The second loop body 2522 may increase the locking force. The clip 2502 may be released and repositioned as desired.

The delivery procedure may be performed around the epicardial surface of the left atrial appendage (LAA) and may extend over the outside edges of the LAA. The procedure may be performed under direct visualization and the closure may be assessed by TEE imaging if desired.

The arms of the clip may apply symmetrical compression to the sides of the LAA. Parallel compression planes may apply a symmetrical pressure over the entire area of contact. Improved apposition of LAA tissue may result.

Variations in the clip may be provided. FIG. 178, for example, illustrates a variation in which the clip 2560 includes a first loop body 2562 having elongate arms 2564, 2566 that are both curved. The curved elongate arms 2564, 2566 may aid in positioning the first loop body 2562 upon the left atrial appendage (LAA) during deployment. The curved elongate arms 2564, 2566 may be deflected upon the second loop body 2522 being inserted into the first loop body 2562 as shown in FIG. 179.

Other variations may be provided as desired.

The features of the examples of FIGS. 158-179 may be utilized solely or in combination with any other example disclosed herein.

Examples of clips disclosed herein may reduce the use of other methods of securement such as sutures for the clips. The clips may be self-closing and securing in examples. The clips may not need to be crimped in some examples.

Variations in the clips and methods disclosed herein may be provided. Features across examples may be combined. Features may be excluded or added to in various examples disclosed herein. Combinations of features of examples may be provided. The features may be utilized solely or in methods disclosed herein, or in combination with other features disclosed herein.

FIG. 180 illustrates an example of a system 2630 that may be utilized with examples herein. The system 2630 may include an implant 2632 that may be coupled to an applicator device 2634. In examples, the system 2630 may be for bonding a first portion of a left atrial appendage of a heart to a second portion of the heart.

The implant 2632 may be configured to bond portions of a heart together. The implant 2632, for example, may comprise an adhesive body that may be configured to adhere the portions of the heart together. The adhesive body may comprise an adhesive pad in examples.

The implant 2632 may be made of a biocompatible material for implantation within the patient's body. The implant 2632 may be configured to remain implanted within the patient's body upon deployment.

The implant 2632 may include an outer surface 2636 that may comprise an adhesive surface. The adhesive surface may be configured to bond a first portion of the LAA to a second portion of a heart. The outer surface 2636, for example, may comprise a biocompatible adhesive that may bond with an outer surface of the LAA or other portion of the heart. The outer surface 2636 may be configured to be pressed against the LAA to bond the first portion of the LAA to the second portion of the heart in examples. The adhesive may comprise a quick curing adhesive in examples, or another form of adhesive. The adhesive may be configured to permanently bond with the tissue in examples, or may comprise a releasable bond.

Referring to the cross sectional view of FIG. 181, a layer 2638 of the adhesive may comprise the outer surface 2636 of the implant 2632. The layer 2638, for example, may surround an inner body 2640 of the implant 2632. The inner body 2640 may surround a central shaft 2642 in examples.

In examples, the outer surface 2636 may comprise a porous body that may be impregnated or otherwise provided with the adhesive. The outer surface 2636, for example, may comprise a sponge pad or other form of porous body for retaining an adhesive. In an example as shown in FIGS. 181 and 182, a layer comprising the outer surface 2636 may be wrapped around the inner body 2640 and may form the side surfaces 2637 and the tip 2639 of the implant 2632, among other portions.

The inner body 2640 may support the outer surface 2636. The inner body 2640, in examples, may comprise a compliant body. The inner body 2640 may be atraumatic to the tissue of the patient's heart and may provide a cushion for the outer surface 2636 in examples. In examples, the inner body 2640 may be made of a porous material, or may be made of a non-porous material as desired.

The central shaft 2642 may comprise a shaft for supporting the inner body 2640 and the outer surface 2636. The central shaft 2642 may include a proximal end portion 2645 and a distal end portion 2647. The central shaft 2642 may be flexible in examples or may be rigid as desired.

In examples, a coupler 2644 may be configured to couple the implant 2632 to the applicator device 2634. The coupler 2644 may be positioned on the implant 2632. The coupler 2644, in examples, may comprise a portion of the central shaft 2642. The coupler 2644 may be positioned on the proximal end portion 2645 of the central shaft 2642, for example, or may have another location as desired.

The implant 2632 may have an elongate shape. For example, the implant 2632 may have a cylindrical shape and may include a tip 2639 that may have a rounded shape as shown in FIGS. 180-182 or may comprise a tip 2646 have a flattened shape as shown in FIG. 183 for example. Side surfaces 2637 of the implant 2632 may have a flattened shape or another shape (e.g., rounded) as desired. The implant 2632 may extend along a central axis 2648 (marked in FIG. 180) and the central shaft 2642 may extend along the central axis 2648. The implant may include a proximal end portion 2649 and a distal end portion 2651 including the tip 2639.

The diameter 2650 of the implant 2632 may be greater than a diameter 2652 of the applicator device 2634, or may be at or lesser than a diameter 2652 of the applicator device 2634. FIG. 183, for example, illustrates an example of an implant 2654 having a same diameter as the diameter of the applicator device 2634.

In the example of FIG. 183, a layer 2656 of adhesive material may be positioned directly upon the central shaft 2642. An inner body 2640 between the layer 2656 and the central shaft 2642 may be excluded in examples.

Referring to FIG. 180, the applicator device 2634 may comprise an elongate applicator shaft. The elongate applicator shaft may include a proximal end portion 2658 and a distal end portion 2660 and a length therebetween. The elongate applicator shaft may be configured to be grasped and manipulated to orient the implant 2632 into a desired position. For example, an outer surface 2662 of the elongate applicator shaft may be configured to be grasped by a user's hand or by graspers to be manipulated into a desired position.

The implant 2632 may comprise a tip of the applicator device 2634. The implant 2632 may be configured to release from the applicator device 2634 in examples.

Referring to FIG. 181, the applicator device 2634 may include a body 2664 configured to couple with the coupler 2644. The body 2664, for example, may comprise a shaft and may be positioned at the distal end portion 2660 of the applicator device 2634. The body 2664 may be configured to insert into the coupler 2644 of the implant 2632 for example. The coupler 2644 may comprise an aperture for example. Other forms of couplers and bodies may be utilized. For example, the coupler 2644 may be configured to insert into a portion of the applicator device 2634 in examples. The coupler 2644 may comprise other forms such as adhesives, hooks, latches, pins, magnets, among others, as desired.

In examples, the coupler 2644 may be configured to allow the implant 2632 to release from the applicator device 2634. In examples, the applicator device 2634 may be withdrawn from the implant 2632 to allow for such release. For example, referring to FIG. 182, upon the adhesive of the implant 2632 bonding to tissue of a patient's heart, the applicator device 2634 may be withdrawn or retracted proximally to release from the implant 2632 with the implant 2632 remaining bonded in position. The implant 2632 may be configured to release from the applicator device 2634 with the applicator device 2634 being retracted from the LAA 2618. The body 2664 may be withdrawn from the coupler 2644 to release from the implant 2632. Other forms of release may be utilized in examples. For example, releasable adhesives, hooks, latches, pins, or magnets, among others, may be utilized as desired.

The implant may be provided in a variety of shapes. FIGS. 184A-184F for example illustrate exemplary shapes that may be provided for the implant, although others may be provided as desired. FIGS. 184A-184E illustrate side views of an implant and FIG. 184F illustrates a perspective view of an implant. FIG. 184A illustrates a cylindrical shape with rounded edges for an implant 2655. FIG. 184B illustrates a hexagonal shape for the implant 2657. FIG. 184C illustrates a trapezoidal shape or a conical frustum shape for the implant 2659. FIG. 184D illustrates a spherical shape for the implant 2661. FIG. 184E illustrates an ovoid shape for the implant 2663. FIG. 184F illustrates a cylindrical shape for the implant 2665. Combinations of shapes may be provided for the implant. Further, the cross sectional profile of the implant may be circular, or may have a variety of shapes including the shapes disclosed herein, among others.

In examples, the implants disclosed herein may comprise spacers that may space portions of a wall of an LAA from each other. In examples, the implants may be relatively thin and may comprise one or more sheets of material that may be implanted as desired. For example, sheets of adhesive material or other forms of implants may be utilized. Various other forms of implants may be utilized as desired.

FIG. 185 illustrates a step in a method that may be utilized herein. The left atrial appendage (LAA) 18 is shown to protrude from the outer wall 20 of the left atrium 10. The inner chamber or cavity 24 has a volume.

According to examples herein, a fold of a first portion 2666 (marked in FIG. 186) of the LAA 18 may be formed with a second portion 2668 (marked in FIG. 186) of the heart, which may comprise a second portion 2668 of the LAA 18. The first portion 2666 and the second portion 2668, in examples, may each comprise a portion of a wall 26 of the LAA 18 that may surround the inner chamber or cavity 24.

In examples, the first portion 2666 and the second portion 2668 may be folded utilizing the system 2630. For example, referring to FIG. 185, the applicator device 2634 may be advanced distally towards the LAA 18 to correspondingly move the implant 2632 towards the LAA 18. The implant 2632 may contact the outer surface 28 of the LAA 18 and may press the wall 26 of the LAA 18 inward towards the inner chamber 24 to form the fold 2674 (marked in FIG. 186). The outer surface 2636 of the implant 2632 may be configured to be pressed against the LAA 18 to fold the portions 2666, 2668. The wall 26 of the LAA 18 may cave inward and the portions 2666, 2668 may move closer to other portions of the heart.

FIG. 185, for example, illustrates the implant 2632 advanced such that the implant 2632 contacts the apex 2672 of the LAA 18 and pushes the apex 2672 inwards towards the inner chamber or cavity 24.

The movement of the implant 2632 may provide a variety of results for the LAA 18. For example, the fold 2674 (marked in FIG. 186) may be formed such that the inner chamber 24 has its volume reduced and may be eliminated as shown in FIG. 186 for example. Further, the fold 2674 may be formed such that the wall 26 of the LAA 18 may occlude the LAA 18 at the ostium 22 as shown in FIG. 186 for example. As such, a portion of the LAA 18 may occlude the ostium 22. In examples, the wall 26 of the LAA 18 may be pushed into the ostium 22 to occlude the ostium 22. In examples, the wall 26 of the LAA 18 may be pushed through the ostium 22 and may protrude into the left atrium 10.

FIG. 186 illustrates a resulting fold 2674 of the LAA 18. The apex 2676 of the fold 2674 may comprise a distal end portion of the fold 2674 and may join the first portion 2666 and the second portion 2668 that extend proximally from the apex 2676. The apex 2676 of the fold 2674 may be positioned at the ostium 22 as shown in FIG. 186 for example. Other configurations may result in examples.

The fold 2674 may further result in apexes 2675, 2677 at respective proximal end portions of the first portion 2666 and second portion 2668. The apex 2675 may join the first portion 2666 to an adjacent wall portion 2679 of the heart, and the apex 2677 may join the second portion 2668 to an adjacent wall portion 2681 of the heart. The adjacent wall portions 2679, 2681 may be portions of the LAA 18.

In examples, the interior surface 2678 of the wall 26 of the LAA 18 may be in contact at the folded portions of the wall 26. For example, the interior surface 2678 of the first portion 2666 may be in contact with the interior surface 2678 of the adjacent wall portion 2679. Further, the interior surface 2678 of the second portion 2668 may be in contact with the interior surface 2678 of the adjacent wall portion 2681. The surfaces may coapt with each other. Such contact may reduce the possibility of blood clots or other material releasing from the interior surfaces 2678 of the wall 26 into the blood stream. The portions 2666, 2679 may comprise layers of the wall 26 in contact with each other, and the portions 2668, 2681 may comprise layers of the wall 26 in contact with each other. In examples, the interior surfaces 2678 of adjacent portions may be spaced from each other.

The fold 2674 may be formed such that an outer surface 28 of the first portion 2666 is faced with an outer surface 28 of the second portion 2668. As shown in FIG. 186, the implant 2632 may be positioned between the outer surface 28 of the first portion 2666 and the outer surface 28 of the second portion 2668. The outer surface 28 of the LAA 18 may be in contact with the implant 2632. The implant 2632 may be positioned between the portions 2666, 2668 of the LAA 18 and may form a spacer between the portions 2666, 2668 of the LAA 18. The volume of the implant 2632 may aid to fill space that was previously occupied by all or a portion of the inner chamber 24. The diameter and length of the implant 2632 may be configured to fill a desired amount of space that was previously occupied by the inner chamber 24.

The outer surface 28 of the LAA 18 may be in contact with an adhesive (e.g., an adhesive outer surface 2636) of the implant 2632 and thus may be bonded to the implant 2632. The implant 2632 may bond the first portion 2666 to the second portion 2668 to maintain the fold 2674. The first portion 2666 may be bonded to the second portion 2668 with the adhesive to maintain the fold 2674.

With the implant 2632 bonded to the first portion 2666 and the second portion 2668, the applicator device 2634 may be withdrawn from the implant 2632. FIG. 187, for example, illustrates a configuration with the first portion 2666 being bonded to the second portion 2668 to maintain the fold 2674. The implant 2632 may be released from the applicator device 2634. The implant 2632 remains adhered to the first portion 2666 and the second portion 2668.

In such a configuration, additional securement of the fold 2674 may be provided if desired. FIG. 188, for example, illustrates that bonding in the form of sutures 2680 may be provided at proximal end portions of the wall 26, which may include the apexes 2675, 2677. The sutures 2680 may provide additional bonding of the portions 2666, 2668 to each other to maintain the fold 2674. FIG. 189, for example, illustrates a line of sutures 2680 that may bond the proximal end portions of the first portion 2666 and second portion 2668 of the wall 2626 to each other.

FIG. 190 illustrates the portions 2666, 2668 bonded to each other with sutures 2680 to maintain the fold 2674. The implant 2632 may be positioned within a pocket between the portions 2666, 2668 and the sutures 2680.

In examples, the implant 2632 may be utilized to maintain the fold 2674, or the sutures 2680 may be utilized to maintain the fold 2674 alternatively or in combination. For example, the implant 2632 may be excluded in examples and other methods may be utilized to fold the portions 2666, 2668. A device may fold the portions 2666, 2668 without use of an implant. The sutures 2680 may be utilized to bond the first portion 2666 to the second portion 2668 to maintain the fold 2674 without use of the implant 2632 in examples. In examples, the use of the sutures 2680 may be excluded.

In examples, other portions of the LAA may be folded. FIGS. 191-193 illustrate an example in which portions of the LAA proximate the ostium 22 may be folded. The ostium 22 may be occluded by the folding of portions of the LAA in examples and/or the volume of the inner chamber 24 may be reduced by the folding of the portions. Multiple implants 2632, 2632′, 2632″, 2632′″ may be utilized as desired, or a single implant 2632 may be utilized in examples. The implants 2632′, 2632″, 2632′″ may each be configured similarly as each other.

FIG. 191, for example, illustrates implants 2632, 2632′ approaching the ostium 22 of the LAA 18, which may be at an angle that is transverse to the longitudinal axis of the LAA 18. The implants 2632, 2632′ may press against the outer surface 28 in a direction towards the inner chamber 24.

FIG. 192, for example, illustrates a fold of the portions 2684, 2686 being formed by the implant 2632, and a fold of the portions 2688, 2690 being formed by the implant 2632′. The portion 2684 may comprise a wall of the LAA 18 and the portion 2686 may comprise a wall of the left atrium 10 of the heart (e.g., the outer wall 20 of the left atrium). Similarly, the portion 2688 may comprise a wall of the LAA 18 and the portion 2690 may comprise a wall of the left atrium 10 of the heart (e.g., the outer wall 20 of the left atrium). The LAA 18 at the ostium 22 is shown to be occluded. The volume of the inner chamber 24 has reduced. Multiple folds of the LAA 18 may be provided to occlude the ostium 22 and reduce the volume of the inner chamber 24.

Other implants may be utilized as desired. FIG. 193, for example, illustrates a cross sectional view along line H-H in FIG. 192. Other implants 2632″, 2632′″ may be utilized to occlude the ostium and may be directed towards the center of the LAA 18. The implants 2632, 2632′, 2632″, 2632′″ may each bond portions of the heart, including the LAA, to each other to maintain respective folds of the LAA. The implants 2632, 2632′, 2632″, 2632′ may extend radially inward towards the inner chamber 24 of the LAA 18. The portions of the heart may be bonded with the adhesive to maintain respective folds of the LAA. In examples, sutures or other forms of bonding may be utilized in combination with an adhesive or as an alternative to the adhesive as disclosed herein.

In examples, the ostium 22 may be fully occluded as shown in FIG. 193 or may be partially occluded as desired.

Multiple folds of the LAA 18 may be formed to reduce the volume of the inner chamber 24. In examples, multiple implants may fold other portions of the LAA 18. FIGS. 194 and 195 illustrate an example in which multiple implants 2632, 2632′, 2632″, 2632′″ may form folds of portions of the LAA 18 to reduce a volume of the inner chamber 24. The implants 2632, 2632′, 2632″, 2632′″ may each be pressed inwards towards the inner chamber 24 to create respective folds 2683, 2685, 2687, 2689, as shown in FIG. 195 for example. The implants 2632, 2632′, 2632″, 2632′″ may each be pressed inward at side walls of the LAA 18 or at the apex 2672 of the LAA 18 as desired. The implants 2632, 2632′, 2632″, 2632′″ may each bond portions of the heart, including the LAA, to each other to maintain respective folds 2683, 2685, 2687, 2689 of the LAA. The portions of the heart may be bonded with the adhesive to maintain respective folds of the LAA. In examples, sutures or other forms of bonding may be utilized in combination with an adhesive or as an alternative to the adhesive as disclosed herein. The volume of the inner chamber 24 shown in FIG. 195, for example, has reduced to reduce the possibility of blood stagnating within the inner chamber 24. A variety of other positions of implants may be utilized in examples.

Other directions of folds may be utilized in examples herein. For example, in examples tissue may be grasped and pulled proximally to form a fold according to methods herein. Combinations of tissue pressed into the inner chamber 24 and pulled away from the inner chamber 24 in examples may be utilized.

In examples, graspers may be utilized in combination with systems, apparatuses, and methods herein. FIG. 196, for example, illustrates a grasper 2692 utilized to grasp the LAA 18 at the ostium 22. The grasper 2692 may aid with the positioning of the implant 2632 relative to the LAA 18 upon advancement of the implant 2632. For example, upon the implant 2632 being inserted in a direction towards the ostium 22 (as shown in FIG. 186 for example), the grasper 2692 may be positioned at the ostium 22 for an identification of when the ostium 22 has been occluded. For example, a user may feel via pressure upon the grasper 2692 when the ostium 22 is occluded by the wall 26 of the LAA 18.

FIG. 197 illustrates an example in which a grasper 2694 may grasp proximate an apex 2672 of the LAA 18. Such a position may aid in the positioning of the implant 2632 relative to the LAA 18 upon advancement of the implant 2632.

The methods disclosed herein may be utilized under a visualization system. For example, ultrasound or fluoroscopy or other forms of visualization may be utilized as desired.

In examples, the implants disclosed herein may have a variety of forms. The systems, apparatuses, and methods disclosed herein may result in a reduced possibility of clots or other undesired materials from entering into the bloodstream. The tissue of the LAA 18 may remain viable and may receive blood flow from the heart. Such a feature may be a benefit over LAA occlusive methods that may sever or cut off blood flow to the LAA, and may produce a residual stump. A reduced possibility of adverse necrosis may occur.

Further, a variety of different shapes of a LAA may be accounted for according to methods herein. For example, a user may be able to view the shape of the LAA and determine an appropriate placement of folds according to methods herein to provide a desired treatment to the LAA.

The systems, apparatuses, and methods disclosed herein may be utilized with a LAA or may be utilized with other portions of a heart or of a body as desired.

Examples of adhesives that may be utilized may include a combination of glutaraldehyde and concentrate bovine albumin; or purified bovine serum albumin and polyaldehyde which rapidly cross link with tissue or graft material to form a strong and stable bond; or biocompatible polyethylene glycols that combine with dilute hydrogen chloride solution to rapidly form a covalently bonded hydrogel and adhere to tissue; or a formulation of polyethelene and human serum albumin to form a strong flexible seal; among other forms of adhesives. Combinations of adhesives may be utilized as desired. In examples, an adhesive, or sutures, or another form of bonding may be utilized without use of an implant. For example, a liquid adhesive or other form of adhesive may be directly applied to a portion of a heart (including the LAA) without use of an implant.

Variations in the clips and methods disclosed herein may be provided. Features across examples may be combined. Features may be excluded or added to in various examples disclosed herein. Combinations of features of examples may be provided. The features may be utilized solely or in methods disclosed herein, or in combination with other features disclosed herein.

FIG. 198 illustrates an example of a clip 2730 that may be utilized for a portion of a heart. The clip 2730 is shown in FIG. 198 closing or occluding the LAA 18 at the ostium of the LAA 18. The clip 2730 may be configured to close the LAA 18 to reduce the possibility of clots or other undesired materials stemming from the LAA 18 from traveling into the bloodstream.

The clip 2730 may include a first jaw 2732 and a second jaw 2734. The first jaw 2732 may extend from a first end portion 2736 to a second end portion 2738 along a length 2740 (marked in FIG. 199). The first jaw 2732 may be configured as an elongate beam.

The first jaw 2732 may include a compression surface 2742 (marked in FIG. 203) and an outer surface 2744 (marked in FIG. 198) facing opposite the compression surface 2742. The compression surface 2742 may face downward in the FIG. 198 view and the outer surface 2744 may face upward. The compression surface 2742 may be configured to apply a compressive force to the portion of the heart (including the LAA 18). The compression surface 2742 may have a flat profile (as shown in FIG. 203 for example) or may have a varied shape as desired. The outer surface 2744 may have a flat profile or a varied shape as desired.

The first jaw 2732 may include a first side surface 2746 (marked in FIGS. 198 and 199) that extends along the length 2740 and is configured to face distally. The first side surface 2746 may be configured to face towards a wall of the heart, including the outer wall 20 of the left atrium 10 as shown in FIG. 198 for example. The first side surface 2746 may extend vertically and join the compression surface 2742 to the outer surface 2744 in examples.

The first side surface 2746 may be angled along the length 2740. For example, as shown in FIG. 198, the first side surface 2746 may be non-linear and may be curved along the length 2740. The curvature may be convex in the distal direction and may be towards the wall of the heart, including the outer wall 20 of the left atrium 10.

The first side surface 2746 may have portions having varied angulation or curvature. For example, referring to FIG. 198, the first side surface 2746 at a central portion 2748 of the first jaw 2732 positioned between the first end portion 2736 and the second end portion 2738 may have a first curvature. The first side surface 2746 at the first end portion 2736 may have a second curvature that differs from the first curvature at the central portion 2748. For example, the first curvature at the central portion 2748 may be less than the second curvature at the first end portion 2736 as shown in FIG. 198. As such, the first side surface 2746 at the central portion 2748 may be flatter than the first side surface 2746 at the first end portion 2736. Similarly, the first side surface 2746 at the central portion 2748 may have a lesser curvature than the first side surface 2746 at the second end portion 2738. In examples, the first side surface 2746 at the central portion 2748 may have a lesser curvature than either or both of the first end portion 2736 and the second end portion 2738.

The curvature of the first side surface 2746 may be cylindrical and may curve about an axis that passes through a plane of closure 2750 of the clip 2730 or a plane of the closure line of the clip 2730. The curvature of the first side surface 2746 may have a varied radius of curvature to provide for different portions of the first jaw 2732 having different curvatures. The first side surface 2746 may bow outward in a distal direction.

The first side surface 2746 at the central portion 2748 may protrude distally relative to the first side surface 2746 at first end portion 2736 or the second end portion 2738. As shown in FIG. 198, the first side surface 2746 at the central portion 2748 may protrude distally relative to the first side surface 2746 at both the first end portion 2736 and the second end portion 2738.

The angle of the first side surface 2746 may provide benefits including improved positioning of the clip 2730 and an improved closure line of the clip 2730. For example, the angle may reduce the possibility of a stump that may be produced upon deployment of the clip 2730. The stump may comprise a portion of the LAA 18 that is not occluded by the clip 2730 and in which blood may pool undesirably. Further, a straighter or flatter central portion 2748 may reduce the amount of the left atrium 10 wall 20 or tissue that is compressed by the clip 2730.

In addition, the angle of the end portions 2736, 2738 may provide varied shapes of the clip 2730 facing the left atrium 10. For example, a user (e.g., a surgeon or other medical technician) may slide the clip 2730 longitudinally along the length 2740 in positioning the clip 2730 to provide a desired profile of the first side surface 2746 facing the left atrium 10. Such a feature may reduce the possibility of “dog ears” or portions of the LAA forming an undesired pocket during closure of the LAA. Varied shapes of the LAA 18, the ostium, and the left atrium 10 may be accommodated.

The first side surface 2746 may have a flat or linear shape in a vertical direction or along an axis that passes through a plane of closure 2750 of the clip 2730. In examples, the first side surface 2746 may be angled in a vertical direction or along an axis that passes through a plane of closure 2750 of the clip 2730.

In examples, the first side surface 2746 may have a variety of other shapes as desired. For example, the first side surface 2746 may include bends or angles with vertices (e.g., a trapezoidal shape), or another shape as desired. Portions of the first side surface 2746 may be flat or linear if desired.

The clip 2730 may include a second side surface 2752 that may be configured to face proximally and opposite the first side surface 2746. The second side surface 2752 may face away from a wall of the heart, including the outer wall 20 of the left atrium 10 as shown in FIG. 198 for example. The second side surface 2752 may extend vertically and join the compression surface 2742 to the outer surface 2744 in examples. The clip 2730 may further have end surfaces 2754, 2756 positioned at the respective first end portion 2736 and second end portion 2738 of the clip 2730. The end surfaces 2754, 2756 may join the compression surface 2742 to the outer surface 2744 at the end portions 2736, 2738.

The second side surface 2752 may comprise a flat or linear surface as shown in FIG. 198 or may have another shape as desired. A flat or linear surface may allow the thickness 2758 of the first jaw 2732 to vary along the length 2740 of the first jaw 2732. In examples, the second side surface 2752 may be angled and may follow a contour of the first side surface 2746 in examples.

The second jaw 2734 may be configured similarly as the first jaw 2732 in examples. For example, the second jaw 2734 may extend from a first end portion 2760 to a second end portion 2762 along a length 2764 (marked in FIG. 199). The second jaw 2734 may include a compression surface 2766 and an outer surface 2768 (marked in FIG. 203) facing opposite the compression surface 2766. The second jaw 2734 may include a first side surface 2770 (marked in FIG. 199) extending along the length 2764 of the second jaw 2734 and configured to face distally, similar to the first side surface 2746 of the first jaw 2732. The second jaw 2734 may include a second side surface 2772 (marked in FIG. 200) configured to face proximally and opposite the first side surface 2770 of the second jaw 2734. The second jaw 2734 may include end surfaces 2774, 2776 (marked in FIG. 202) at respective first and second end portions 2760, 2762 of the second jaw 2734.

The first side surface 2770 of the second jaw 2734 may be angled along the length 2764 of the second jaw 2734. In examples, the first side surface 2770 of the second jaw 2734 may be angled in a similar manner as the first side surface 2746 of the first jaw 2732. For example, the first side surface 2770 of the second jaw 2734 may follow the contour of the first side surface 2746 of the first jaw 2732. As shown in FIG. 200, the distal protrusion of the first side surface 2770 of the second jaw 2734 may be same as the first side surface 2746 of the first jaw 2732. In examples, the first side surface 2770 of the second jaw 2734 may have a different profile as desired.

The second side surface 2772 of the second jaw 2734 may be configured similarly as the second side surface 2752 of the first jaw 2732. The second side surface 2772 may have a same profile as the second side surface 2752 of the first jaw 2732 or may have a different profile as desired.

The length 2764 of the second jaw 2734 may be greater than the length 2740 of the first jaw 2732. For example, as shown in FIG. 198, the end portions 2760, 2762 of the second jaw 2734 may protrude from the respective end portions 2736, 2738 of the first jaw 2732 along the length of the first jaw 2732. Such a feature may beneficially allow for visualization of the end portions 2760, 2762 of the second jaw 2734 during a deployment procedure when viewed by a user in a downward direction. The presence of any tissue protruding from the end portions 2736, 2738 of the first jaw 2732 may be determined during the deployment procedure. As shown in FIG. 198, the length 2740 of the first jaw 2732 being less than the length 2764 of the second jaw 2734 may produce end portions 2736, 2738 of the first jaw 2732 that taper proximally relative to the end portions 2760, 2762 of the second jaw 2734. In examples, the length 2764 of the second jaw 2734 may be the same as or less than the length of the first jaw 2732.

At least one spring may be configured to force the first jaw 2732 and the second jaw 2734 together to compress the portion of the heart between the compression surface 2742 of the first jaw 2732 and the compression surface 2766 of the second jaw 2734.

The at least one spring may include a first end portion 2780 coupled to the first jaw 2732 and a second end portion 2782 (marked in FIG. 200) coupled to the second jaw 2734 and a loop portion 2784 that protrudes proximally from the first end portion 2780 and the second end portion 2782 of the at least one spring. Referring to FIG. 200, the at least one spring may include an upper arm 2786 that extends proximally from the first end portion 2780 to the loop portion 2784, and may include a lower arm 2788 that extends proximally from the second end portion 2782 to the loop portion 2784. The upper arm 2786 may be aligned with the lower arm 2788 in a vertical plane extending transverse to the length 2740 of the first jaw 2732 or may be otherwise oriented as desired.

The at least one spring may include a first spring 2790 and a second spring 2792. The first spring 2790 may include the first end portion 2780, the second end portion 2782, and the loop portion 2784. The second spring 2792 may include a first end portion 2794 coupled to the first jaw 2732, a second end portion 2796 (marked in FIG. 199) coupled to the second jaw 2734, and a loop portion 2798 that protrudes proximally from the first end portion 2794 and the second end portion 2796. The second spring 2792 may be configured similarly as the first spring 2790 and features of the second spring 2792 may provide similar function as the first spring 2790. The second spring 2792, for example, may include an upper arm 2800 configured similarly as the upper arm 2786 of the first spring 2790 and may include a lower arm configured similarly as the lower arm 2788 of the first spring 2790.

The springs 2790, 2792 may be positioned proximal of the jaws 2732, 2734 to improve visualization of the closure line of the clip 2730. Improved deployment of the clip 2730 may result.

In examples, the first end portion 2780 of the first spring 2790 may be coupled to the first end portion 2736 of the first jaw 2732 and the second end portion 2782 of the first spring 2790 may be coupled to the first end portion 2760 of the second jaw 2734. The first end portion 2794 of the second spring 2792 may be coupled to the second end portion 2738 of the first jaw 2734 and the second end portion 2796 of the second spring 2792 may be coupled to the second end portion 2762 of the second jaw 2734. The first spring 2790 and the second spring 2792 may each be biased to apply a compressive force to the first and second jaws 2732, 2734. The loop portion 2784 of the first spring 2790 may compress the upper arm 2786 and the lower arm 2788 towards each other, and the loop portion 2798 may compress the upper arm 2800 and the lower arm of the second spring 2792 towards each other.

The first spring 2790 may include an angled portion 2802 that may be angled towards the second spring 2792. The angled portion 2802 may comprise a portion of the upper arm 2786 and the lower arm 2788 of the first spring 2790. The angled portion 2802 may produce a distal portion 2804 of the upper arm 2786 that angles towards the second spring 2792 in a proximal direction, and a proximal portion 2806 of the upper arm 2786 that angles away from the second spring 2792 in the proximal direction. The distal portion 2804 and proximal portion 2806 of the upper arm 2786 may each be straight or linear with the angled portion 2802 forming a bend in the upper arm 2786. The lower arm 2788 of the first spring 2790 may include a similar configuration.

The second spring 2792 may include an angled portion 2808 that may be angled towards the first spring 2790. The angled portion 2808 may be configured similarly as the angled portion 2802 of the first spring 2790 yet inverted to be angled towards the first spring 2790.

The first spring 2790 and the second spring 2792 together may form an hourglass shape, as shown in FIG. 198. The angled portion 2802 of the first spring 2790 and the angled portion 2808 of the second spring 2792 may each be biased towards each other to apply a compressive force towards each other. The compressive force may be applied to a delivery apparatus that may be positioned between the angled portions 2802, 2808 and may retain the clip 2730 to the delivery apparatus in examples. The angled portions 2802, 2808 may form a waist that may bear upon the delivery apparatus and serve as a contact point with the delivery apparatus. Other configurations of the springs 2790, 2792 may be utilized in examples as desired.

Referring to FIG. 201, in examples, the at least one spring may couple to the first jaw 2732 with a pivot coupler 2810. The pivot coupler 2810 for example, may comprise a hinge or may comprise a ball joint (as shown in FIG. 201), or may comprise another form of pivot coupler as desired. The pivot coupler 2810 may allow the first jaw 2732 to pivot relative to the at least one spring to vary the plane that the first jaw 2732 extends in. FIG. 203, for example, illustrates the first jaw 2732 angled with the compression surface 2742 facing distally and FIG. 204 illustrates the first jaw 2732 angled proximally from the angle shown in FIG. 203. The first jaw 2732 may float relative to the second jaw 2734. Multiple degrees of freedom (e.g., three degrees of freedom for a ball joint) or planes of movement may be provided be a pivot coupler 2810 such as a ball joint. A pivot coupler 2810 may be utilized at the coupling of the first spring 2790 to the first jaw 2732 and may be utilized at the coupling of the second spring 2792 to the first jaw 2732.

In examples, a pivot coupler may couple the second end portion 2782 of the first spring 2790 to the second jaw 2734. In examples, one or more pivot couplers may couple the second spring 2792 to the first jaw 2732 or to the second jaw 2734 or both jaws 2732, 2734. In examples, a static coupling may be utilized for one jaw (e.g., the second jaw 2734) while the other jaw (e.g., the first jaw 2732) may be configured to pivot relative to the first spring 2790 and the second spring 2792. In examples, static couplings may be utilized for both jaws.

In examples, the first jaw 2732 being pivotal and the second jaw 2734 being static may increase the simplicity of the deployment procedure. The second jaw 2734 may be held in position against the outer surface of the LAA and the first jaw 2732 may pivot to accommodate the shape of the LAA. Other configurations may be utilized.

FIGS. 202-205 illustrate an exemplary deployment sequence of the clip 2730 to close a portion of a heart. The deployment sequence may be varied (e.g., steps added, substituted, removed, modified) as desired. Referring to FIG. 202, the clip 2730 may be retained by a delivery apparatus 2820 that may include a first body portion 2822 and a second body portion 2824. In examples, the first body portion 2822 and the second body portion 2824 may have a channel 2825 therebetween that may allow the portion of the heart to be compressed (e.g., the LAA 18) to be positioned in. The body portions 2822, 2824 may be rigid to support a compressive force applied by the springs 2790, 2792 to the body portions 2822, 2824. The delivery apparatus 2820 may comprise a pair of tongs that may retain the clip 2730 or may have another form as desired. The body portions 2822, 2824 may comprise the arms of the tongs that may be rectangular in shape or may have another shape in examples.

The delivery apparatus 2820 may include one or more protrusions 2826, 2828 that may protrude from the body portions 2822, 2824 to contact the springs 2790, 2792. The protrusions 2826, 2828 may hold the springs 2790, 2792 in an opened configuration and may be released from the springs 2790, 2792 to allow the springs 2790, 2792 to move to a closed configuration. For example, referring to FIG. 203, an upper protrusion 2826 may contact the upper arm 2786 of the first spring 2790 and a lower protrusion 2828 may contact the lower arm 2788 of the first spring 2790. The protrusions 2826, 2828 may hold the first spring 2790 in an opened configuration. Protrusions 2826, 2828 on the second body portion 2824 may hold the second spring 2792 in an opened configuration. In examples, the protrusions 2826, 2828 may be withdrawn (e.g., moved proximally) from the first spring 2790 and second spring 2792 to allow the first spring 2790 and second spring 2792 to close. In examples, the protrusions 2826, 2828 may be mechanically retracted into the respective body portions 2822, 2824 to release from the springs 2790, 2792. In examples, protrusions 2826, 2828 may engage the angled portions 2802, 2808 or the waist of the springs 2790, 2792.

Referring to FIG. 202, the angled portions 2802, 2808 of the respective first spring 2790 and the second spring 2792 may apply a compressive force to the respective body portions 2822, 2824 of the delivery apparatus 2820. Such compressive force may retain the clip 2730 to the delivery apparatus 2820 during a deployment procedure. As such, in examples, a simplified retention method between the clip 2730 and the delivery apparatus 2820 may be provided. In examples, the delivery apparatus 2820 may engage only the springs 2790, 2792 to allow the first jaw 2732 or second jaw 2734 or both to freely pivot during a deployment procedure due to one or more pivot couplers with the springs 2790, 2792.

The clip 2730 may be positioned in an opened configuration and advanced to the deployment location of the clip 2730. For example, referring to FIG. 203, the springs 2790, 2792 of the clip 2730 may be held in an opened configuration, producing a channel 2829 between the compression surfaces 2742, 2766 of the respective jaws 2732, 2734. The portion of the heart to be compressed may be positioned within the channel 2829.

The delivery apparatus 2820 may be positioned proximal of the jaws 2732, 2734 to improve visualization of the closure line of the clip 2730.

The delivery apparatus 2820 may be positioned in a desired location to position the clip 2730 as desired relative to the portion of the heart to be compressed (e.g., the LAA 18). The delivery apparatus 2820 may be advanced distally or retracted proximally, or may be slid longitudinally along the length of the clip 2730, to produce a desired deployment orientation of the clip 2730.

The delivery apparatus 2820 may be retracted or otherwise released from the clip 2730 as shown in FIG. 204 for example. FIG. 204 may illustrate a partially closed state or adjustment state that may hold the tissue while permitting repositioning to provide a desired closure line. The protrusions 2826, 2828 may be moved relative to the springs 2790, 2792 to reduce the size of the channel 2829 and move the jaws 2732, 2734 towards each other. The jaws 2732, 2734 may be positioned at the ostium of the LAA 18 or at another location as desired.

The delivery apparatus 2820 may be fully withdrawn as shown in FIG. 205 to allow the jaws 2732, 2734 to move to a closed configuration and close the portion of the heart (e.g., the LAA 18). The jaws 2732, 2734 may close in the plane of closure 2750 of the clip 2730 (marked in FIG. 198).

In examples, the clip 2730 may be recaptured for removal or repositioning as desired. For example, the delivery apparatus 2820 may recapture the clip 2730 to move the jaws 2732, 2734 from the closed configuration. The clip 2730 may be removed or repositioned as desired.

Variations in the clip 2730 may be provided in examples.

FIG. 206 illustrates a grip surface 2830 that may be provided for one or more of the compression surfaces 2742, 2766 of the first jaw 2732 or second jaw 2734. The grip surface 2830 may include a plurality of teeth or may have another configuration as desired. In examples, the grip surface 2830 may be covered with a compliant layer 2832 that may reduce the possibility of puncture by the grip surface 2830 of the portion of the heart being closed. The compliant layer 2832 may comprise expanded polytetrafluoroethylene (ePTFE) or silicone or another material as desired. An elastomeric surface or porous surface may be provided in examples. In examples, the grip surface 2830 may be excluded and the compliant layer 2832 may be utilized solely. In examples, tissue may be lubricated to reduce friction (e.g., flushed with saline or another material).

The features of the clip 2730 disclosed herein may be utilized solely or in combination with any other example disclosed herein.

FIG. 207 illustrates an example of a clip 2850 that may be utilized for a portion of a heart. The clip 2850 may be configured to close the LAA 18 to reduce the possibility of clots or other undesired materials stemming from the LAA 18 from traveling into the bloodstream.

The clip 2850 may include a first jaw 2852 and a second jaw 2854. The first jaw 2852 may extend from a first end portion 2856 to a second end portion 2858 along a length. The first jaw 2852 may be configured as an elongate beam.

The first jaw 2852 may include a compression surface 2860 (marked in FIG. 210) and an outer surface 2862 (marked in FIG. 208) facing opposite the compression surface 2860. The compression surface 2860 may face downward in the FIG. 208 view and the outer surface 2862 may face upward. The compression surface 2860 may be configured to apply a compressive force to the portion of the heart (including the LAA 18). The compression surface 2860 may have a curved profile (as shown in FIG. 207 for example) that may be a concave profile along the length of the first jaw 2852 or may have another shape as desired (e.g., linear or flat). The first jaw 2852 may be bowed upward in a plane of closure 2864 of the clip 2850 (marked in FIG. 208) or may have another shape as desired. The outer surface 2862 may have a curved or convex profile that may match the profile of the compression surface 2860 or may have another shape as desired (e.g., linear or flat).

The first jaw 2852 may include a first side surface 2866 (marked in FIG. 208) that extends along the length of the first jaw 2852 and is configured to face distally. The first side surface 2866 may be configured to face towards a wall of the heart, including the outer wall 20 of the left atrium 10 as shown in FIG. 208 for example. The first side surface 2866 may extend vertically and join the compression surface 2860 to the outer surface 2862 in examples.

The first side surface 2866 may be angled along the length of the first jaw 2852. For example, as shown in FIG. 208, the first side surface 2866 may be non-linear and may include a bend 2868. The bend 2868 may be positioned at a central portion 2870 of the first jaw 2852 between the first end portion 2856 and the second end portion 2858. The bend 2868 may form a first elongate portion 2872 and a second elongate portion 2874 of the first jaw 2852, with the bend 2868 positioned between the first elongate portion 2872 and the second elongate portion 2874. The bend 2868 may angle the first elongate portion 2872 relative to the second elongate portion 2874. The first elongate portion 2872 may be straight or linear and the second elongate portion 2874 may be straight or linear in examples.

The angle of the bend 2868 may be set as desired. For example, the angle may be between 140 degrees and 170 degrees in examples. In examples, the angle may be between 150 to 160 degrees in examples. Other angles may be provided as desired.

In examples, the bend 2868 may be malleable and may have an angle that may be adjusted. For example, a user may adjust the degree of the bend 2868 prior to or during a deployment procedure to provide a desired fit at the deployment location. In examples, the bend 2868 may comprise a hinge with an angle that may be adjusted as desired.

In examples, the bend 2868 may be angled in an opposite direction than shown in FIG. 208. For example, the bend 2868 may angle the second elongate portion 2874 proximally as shown in FIG. 208 or may angle the second elongate portion 2874 distally (e.g., with the second end portion 2858 angled towards the left atrium) as desired.

The first elongate portion 2872 may have a length 2876 and the second elongate portion 2874 may have a length 2878 that is less than the length 2876 of the first elongate portion 2872. As such, the elongate portions 2872, 2874 may have unequal lengths in examples. The length 2878 of the second elongate portion 2874 may comprise 80% or less of the length of the first elongate portion 2872. In examples, the length 2878 of the second elongate portion 2874 may comprise 60% or less of the length of the first elongate portion 2872. In examples, the length 2878 of the second elongate portion 2874 may comprise 40% or less of the length of the first elongate portion 2872. In examples, the length 2878 of the second elongate portion 2874 may comprise 20% or less of the length of the first elongate portion 2872. In examples, the elongate portions 2872, 2874 may have equal lengths.

The angle of the first side surface 2866 may provide benefits including improved positioning of the clip 2850 and an improved closure line of the clip 2850. For example, the angle between the first elongate portion 2872 and the second elongate portion 2874 may provide for improved contour to the shape of the heart, including reducing the amount of the left atrium 10 wall 20 or tissue that is compressed by the clip 2730. The angle may reduce the possibility of a stump that may be produced upon deployment of the clip 2850.

A user (e.g., a surgeon or other medical technician) may slide the clip 2850 longitudinally along the length in positioning the clip 2850 to provide a desired profile of the first side surface 2866 facing the left atrium 10. Varied shapes of the LAA 18 and the left atrium 10 may be accommodated.

The first side surface 2866 may have a flat or linear shape in a vertical direction or along an axis that passes through a plane of closure 2864 of the clip 2850. In examples, the first side surface 2866 may be angled in a vertical direction or along an axis that passes through a plane of closure 2864 of the clip 2850.

In examples, the first side surface 2866 may have a variety of other shapes as desired. For example, the first side surface 2866 may include curved portions, or another shape as desired.

In examples, the first side surface 2866 may include a single bend as shown in FIG. 208, or may include multiple bends as desired.

The first jaw 2852 may include a second side surface 2880 that may be configured to face proximally and opposite the first side surface 2866. The second side surface 2880 may face away from a wall of the heart, including the outer wall 20 of the left atrium 10 as shown in FIG. 208 for example. The second side surface 2880 may extend vertically and join the compression surface 2860 to the outer surface 2862 in examples. The first jaw 2852 may further have end surfaces 2882, 2884 positioned at the respective first end portion 2856 and second end portion 2858 of the clip 2850. The end surfaces 2882, 2884 may join the compression surface 2860 to the outer surface 2862 at the end portions 2856, 2858.

The second side surface 2880 may follow the contour of the first side surface 2866 in examples. For example, as shown in FIG. 208, the second side surface 2880 may be angled to match the angle of the first side surface 2866 and accordingly a thickness 2886 of the first jaw 2852 may be uniform along the length of the first jaw 2852. In examples, the second side surface 2880 may have a varied shape that does not follow the contour of the first side surface 2866.

Referring to FIG. 207, a height 2888 of the first jaw 2852 may be uniform along the length of the first jaw 2852.

The second jaw 2854 may be configured similarly as the first jaw 2852 in examples. For example, the second jaw 2854 may extend from a first end portion 2890 to a second end portion 2892 along a length. The second jaw 2854 may include a compression surface 2894 (marked in FIG. 210) and an outer surface 2893 (marked in FIG. 207) facing opposite the compression surface 2894. The second jaw 2854 may include a first side surface extending along the length of the second jaw 2854 and configured to face distally, similar to the first side surface 2866 of the first jaw 2852. The second jaw 2854 may include a second side surface 2895 (marked in FIG. 207) configured to face proximally and opposite the first side surface of the second jaw 2854. The second jaw 2854 may include end surfaces 2896, 2897 (marked in FIG. 207) at respective first and second end portions 2890, 2892 of the second jaw 2854.

The first side surface of the second jaw 2854 may be angled along the length of the second jaw 2854. In examples, the first side surface of the second jaw 2854 may be angled in a similar manner as the first side surface 2866 of the first jaw 2852. For example, the first side surface of the second jaw 2854 may follow the contour of the first side surface 2866 of the first jaw 2852. In examples, the first side surface of the second jaw 2854 may have a different profile as desired.

The second side surface 2895 of the second jaw 2854 may be configured similarly as the second side surface 2880 of the first jaw 2852. The second side surface 2895 may have a same profile as the second side surface 2880 of the first jaw 2852 or may have a different profile as desired.

The compression surface 2894 of the second jaw 2854 may be curved and may have a convex profile along the length of the second jaw 2854. The curvature of the compression surface of the second jaw 2854 may match the curvature of the compression surface of the first jaw 2852. The second jaw 2854 may be bowed upward in the plane of closure 2864 marked in FIG. 208.

In examples, the compression surface 2894 of the second jaw 2854 may match the curvature of the heart. Further, the compression surface 2860 of the first jaw 2852 may match the curvature of the heart. In examples, the first jaw 2852 may have a greater curvature than the second jaw 2854 (e.g., more concave).

The length of the second jaw 2854 may be the same as the length of the first jaw 2852 or may differ in examples.

At least one spring 2899 may be configured to force the first jaw 2852 and the second jaw 2854 together to compress the portion of the heart between the compression surface 2860 of the first jaw 2852 and the compression surface 2894 of the second jaw 2854.

The at least one spring 2899 may include a first end portion 2901 coupled to the first jaw 2852 and a second end portion 2898 coupled to the second jaw 2854 and a loop portion 2900 positioned between the first end portion 2901 and the second end portion 2898.

Referring to FIG. 208, the at least one spring 2899 may be laterally offset proximally from the second side surface 2880 of the first jaw 2852. The spring 2899, for example, may extend adjacent with and parallel with the first elongate portion 2872 of the first jaw 2852. The spring 2899 may be laterally offset such that the spring 2899 does not cover the outer surface 2862 of the first jaw 2852. The spring 2899 may be laterally offset from the second side surface 2895 of the second jaw 2854 in examples.

A lateral offset of the spring 2899 may improve visibility of a closure line of the clip 2850 during deployment.

The first end portion 2901 of the spring 2899 may couple to the central portion 2870 (marked in FIG. 208) of the first jaw 2852. The second end portion 2898 of the spring 2899 may couple to the first end portion 2890 of the second jaw 2854. The second end portion 2898 of the spring 2899 may extend upward from the first end portion 2890 of the second jaw 2854 to reduce the overall length of the clip 2850 in examples. The spring 2899 may be raised above the outer surface 2893 of the second jaw 2854 and may not cover the outer surface 2893 of the second jaw 2854. The spring 2899, and particularly the loop portion 2900, may be bowed upward in a plane of closure 2864 of the clip 2850 above the outer surface 2862 of the first jaw 2852.

The first end portion 2901 of the spring 2899 may couple to the first jaw 2852 with a pivot coupler 2902. The pivot coupler 2902 may comprise a hinge (e.g., a pin and aperture) that may allow the first jaw 2852 to pivot within the plane of closure 2864 of the clip 2850. FIGS. 210-212, for example illustrate a pivot of the first jaw 2852 in the plane of closure 2864. The first jaw 2852 may be able to pivot relative to the second jaw 2854. Such a feature may facilitate tissue capture (e.g., LAA capture) and may provide for a tip-first closure of the clip 2850. In examples, the second end portion 2898 of the spring 2899 may couple to the first end portion 2890 of the second jaw 2854 with a pivot coupler or with a static coupler. In examples, the first end portion 2901 of the spring 2899 may couple to the first jaw 2852 with a static coupler. In examples, the coupling location of the spring 2899 to the first jaw 2852 or the second jaw 2854 may be varied as desired.

In examples, the spring 2899 may comprise a flat spring to reduce the possibility of twisting of the spring 2899. Other forms of springs may be utilized in examples.

In examples, the clip 2850 may include coupling features to couple the clip 2850 to a delivery apparatus 2904. The coupling features, for example, may comprise one or more recesses 2906, 2908 in the respective first jaw 2852 or second jaw 2854 for receiving a protrusion of a delivery apparatus 2904. The coupling features may have other forms in examples.

Referring to FIG. 209, for example, a delivery apparatus 2904 may include respective protrusions 2910, 2912 that may insert into the recesses 2906, 2908 and may be moved to move the clip 2850 between an opened configuration and a closed configuration.

FIGS. 210-212 illustrate an exemplary deployment sequence of the clip 2850 to close a portion of a heart. The deployment sequence may be varied (e.g., steps added, substituted, removed, modified) as desired. Referring to FIG. 210, the clip 2850 may be retained by a delivery apparatus 2904 (not shown in FIG. 210) that may move the first jaw 2852 away from the second jaw 2854. The clip 2850 may be held in an opened configuration. The delivery apparatus 2904 may move the first jaw 2852 relative to the second jaw 2854 such that a channel 2911 between the first jaw 2852 and the second jaw 2854 is wider at the second end portions 2858, 2892 than the first end portions 2856, 2890 of the jaws 2852, 2854. The jaws 2852, 2854 may be angled relative to each other such that tips 2914, 2916 of the respective jaws 2852, 2854 at the end portions 2858, 2892 are angled away from each other, with the spring 289 closing the channel 2911 at an opposite end of the channel 2911.

The clip 2850 may be slid over the LAA 18 such that the LAA 18 is received in the channel 2911.

Referring to FIG. 211, the clip 2850 may be moved to a closed configuration by the tips 2914, 2916 being moved towards each other, such that the channel 2911 between the first jaw 2852 and the second jaw 2854 is narrower at the second end portions 2858, 2892 than the first end portions 2856, 2890 of the jaws 2852, 2854. A tip-first closure of the clip 2850 may result. The first jaw 2852 may pivot such that the first end portion 2856 is raised above the spring 2899. The first jaw 2852 may be advanced forward in a direction along the length of the first jaw 2852 as well. The first jaw 2852 may have two degrees of freedom (e.g., pivotal motion and a vertical translation).

Referring to FIG. 212, the clip 2850 may continue to be closed in a direction towards the first end portions 2856, 2890 of the jaws 2852, 2854. The clip 2850 may be in a closed configuration. The first jaw 2852 may pivot about the pivot coupler 2902 such that the first end portions 2856, 2890 of the jaws 2852, 2854 are moved towards each other. The jaws 2852, 2854 may extend parallel with each other in examples.

In examples, the first jaw 2852 may be moveable while the second jaw 2854 remains static. As such, reduced complexity of deployment may result. In examples, the clip 2850 may be recaptured for removal or repositioning as desired. For example, the delivery apparatus 2904 may recapture the clip 2850 to move the jaws 2852, 2854 from the closed configuration. The clip 2850 may be removed or repositioned as desired.

Variations in the clip 2850 may be provided in examples. The features of the clip 2850 disclosed herein may be utilized solely or in combination with any other example disclosed herein.

The clips as disclosed herein may be utilized to close the LAA or may be utilized to close another portion of a heart. In examples, the clips may be utilized to close other portions of a body, including other tubular vessels or other portions of a body. Deployment may be via a delivery apparatus or via another method as desired. Deployment may be via surgical methods and may be transcatheter or via non-invasive surgery in methods.

Variations in the clips and methods disclosed herein may be provided. Features across examples may be combined. Features may be excluded or added to in various examples disclosed herein. Combinations of features of examples may be provided. The features may be utilized solely or in methods disclosed herein, or in combination with other features disclosed herein.

FIG. 213 illustrates an example of a clip 3000 that may be utilized for a portion of a heart. The clip 3000 may be configured to close a LAA 18, to reduce the possibility of clots or other undesired materials stemming from the LAA 18 from traveling into the bloodstream.

The clip 3000 may include a first jaw 3002 and a second jaw 3004. The first jaw 3002 may extend from a proximal end portion 3006 to a distal end portion 3008 and may have an elongate shape. The distal end portion 3008 of the first jaw 3002 may comprise a distal tip 3010 of the first jaw 3002 in examples. The first jaw 3002 may be configured as an elongate beam or may have another configuration as desired.

FIG. 216 illustrates a front view of the clip 3000 showing a configuration of the first jaw 3002. The first jaw 3002 may comprise a U-shaped beam having a U-shaped cross section, including side walls 3011 and a horizontal wall 3012 extending between the side walls 3011. A channel 3014 is positioned between the side walls 3011 and is bounded on a lower side by the horizontal wall 3012 and is opened on an upper end of the channel 3014.

The side walls 3011 may include exterior side surfaces 3013 that may face outward from the channel 3014. The side walls 3011 may include interior side surfaces 3015 that may face towards the channel 3014.

The horizontal wall 3012 of the first jaw 3002 may include a compression surface 3016 that faces the second jaw 3004 and is configured to apply a compressive force to the portion of the body to be occluded. The horizontal wall 3012 may include an outer surface 3018 that faces towards the channel 3014.

Referring to FIG. 216, the compression surface 3016 may have a flat shape in examples. In examples, the compression surface may be angled or curved. For example, referring to FIG. 222, a cross sectional version of the first jaw 3002 is shown, including a compression surface 3020 having a V-shape. The V-shape may have an apex 3022 at a center of the compression surface 3020.

Referring to FIG. 223, a cross sectional version of the first jaw 3002 is shown, including a compression surface 3024 having a gradual curve shape. The gradual curve shape may have an apex 3026 at a center of the compression surface 3024. A gradual curve or flat shape may distribute compression force more evenly than the V-shape shown in FIG. 222. The V-shape may provide a more narrow line of force than the gradual curve or flat shape.

The first jaw 3002 may be stamped or otherwise bent into a U-shape. The side walls 3011 may provide rigidity and stiffness for the first jaw 3002. The first jaw 3002 may be cut from a single piece of material and stamped or otherwise bent into the U-shape.

Referring to FIG. 217, a rear perspective view of the first jaw 3002 is shown. The proximal end portion 3006 of the first jaw 3002 may include a cut out portion 3028 in the horizontal wall 3012 of the first jaw 3002. The cut out portion 3028 may allow for a portion of the spring 3030 shown in FIG. 213 to pass into, such that the side walls 3011 of the first jaw 3002 protrude proximally from the spring 3030.

The second jaw 3004 may be configured similarly as the first jaw 3002. Referring to FIG. 213, the second jaw 3004 may extend from a proximal end portion 3032 to a distal end portion 3034 and may have an elongate shape. The distal end portion 3034 of the second jaw 3004 may comprise a distal tip 3036 of the second jaw 3004 in examples. The second jaw 3004 may be configured as an elongate beam or may have another configuration as desired.

FIG. 216 illustrates a front view of the clip 3000 showing a configuration of the second jaw 3004 relative to the first jaw 3002. The second jaw 3004 may be configured similarly as the first jaw 3002, and may comprise a U-shaped beam having a U-shaped cross section, with side walls 3038 and a horizontal wall 3040. A channel 3042 is positioned between the side walls 3038 and is bounded on a lower side by the horizontal wall 3040 and is opened on an upper end of the channel 3042.

The side walls 3038 may include exterior side surfaces 3039 that may face outward from the channel 3042. The side walls 3038 may include interior side surfaces 3041 that may face towards the channel 3042.

The horizontal wall 3040 of the second jaw 3004 may include a compression surface 3044 that faces the first jaw 3002 and is configured to apply a compressive force to the portion of the body to be occluded. The horizontal wall 3040 may include an outer surface 3046 that faces towards the channel 3042.

The compression surface 3044 may have shapes including the shapes described herein for the compression surface 3016 of the first jaw 3002. The second jaw 3004 may include a cut out portion at a proximal end portion 3032 of the second jaw 3004, similar to the cut out portion 3028 of the first jaw 3002.

The second jaw 3004 may be stamped or otherwise bent into a U-shape. The side walls 3038 may provide rigidity and stiffness for the second jaw 3004. The second jaw 3004 may be cut from a single piece of material and stamped or otherwise bent into the U-shape.

The distal end portions 3008, 3034 of the respective first jaw 3002 and second jaw 3004 may form an axial opening for a space between the jaws 3002, 3004 for receiving an object to be compressed. The distal end portions 3008, 3034 may comprise an open end of the space and the proximal end portions 3006, 3032 may comprise a closed end of the space. The object to be compressed (e.g., a portion of a heart such as a left atrial appendage (LAA)) may be inserted into the space through the opening or the object may be slid into the space transverse to the respective axes that the jaws 3002, 3004 of the clip 3000 extend along.

The jaws 3002, 3004 may extend parallel with each other, with a space for receiving the object to be compressed positioned between the jaws 3002, 3004.

The spring 3030 may comprise a c-shaped spring 3030 that may have a first end 3048 and a second end 3050. A loop portion 3052 may extend proximally from the first end 3048 and the second end 3050 and may loop around the proximal end portions 3006, 3032 of the respective first jaw 3002 and the second jaw 3004. The loop portion 3052 may extend along the channels 3014, 3042 of the respective first jaw 3002 and the second jaw 3004. The channels 3014, 3042 may be configured to allow for movement of the loop portion 3052 within the channels 3014, 3042.

FIG. 218 illustrates the spring 3030 in isolation from the remainder of the clip 3000. The first end 3048 of the spring 3030 may include two bends 3054, 3056 forming an S-shaped curve. Similarly, the second end 3050 of the spring 3030 may include two bends 3058, 3060 forming an S-shaped curve. The bends 3054, 3056 may increase a stiffness of the spring 3030 at the first end 3048. The bends 3058, 3060 may increase a stiffness of the spring 3030 at the second end 3050.

The loop portion 3052 may include straightened portions 3062, 3064 and a curved portion 3066 that may couple the straightened portions 3062, 3064 to each other.

Referring to FIG. 213, the first end 3048 of the spring 3030 may be configured to apply a compressive force to the distal end portion 3008 of the first jaw 3002, and the second end 3050 of the spring 3030 may be configured to apply a compressive force to the distal end portion 3034 of the second jaw 3004. The force from the spring 3030 may be applied at the respective tips 3010, 3036 of the first jaw 3002 and the second jaw 3004. The spring 3030 may be configured to force the first jaw 3002 and the second jaw 3004 together.

The first end 3048 of the spring 3030 may have a fixed connection to the distal end portion 3008 of the first jaw 3002 in examples. The fixation may be via staking, riveting, or welding, among other methods of fixation, as desired. A rivet hole 3067 is shown in FIG. 213 for example, yet may be excluded when other methods of fixation (e.g., welding) are utilized. The second end 3050 of the spring 3030 may have a fixed connection to the distal end portion 3034 of the second jaw 3004 in examples. The fixation may include similar methods as with the first jaw 3002.

The first end 3048 of the spring 3030 may be coupled to the outer surface 3018 of the first jaw 3002. The second end 3050 of the spring 3030 may be coupled to the outer surface 3046 of the second jaw 3004.

The proximal end portions 3006, 3032 of the first jaw 3002 and the second jaw 3004 may be unconnected directly to the spring 3030 or to each other, and as such may be able to pivot with respect to the spring 3030 and with respect to each other. The first jaw 3002 accordingly may comprise a cantilever beam fixed to the spring 3030 at the distal end portion 3008 of the first jaw 3002. The second jaw 3004 accordingly may comprise a cantilever beam fixed to the spring 3030 at the distal end portion 3034 of the second jaw 3004.

The loop portion 3052 of the spring 3030 may extend into the cut out portions of the proximal end portions 3006, 3032 of the jaws 3002, 3004. Such a feature may reduce the possibility of a portion of the body to be occluded from extending into a gap between the spring 3030 and the proximal end portions 3006, 3032 of the jaws 3002, 3004. The loop portion 3052 of the spring 3030 may form a boundary or closed end portion of a space between the jaws 3002, 3004.

FIG. 214 illustrates a side view of the clip 3000. The side wall 3011 of the first jaw 3002 may include one or more apertures 3068 for receiving sutures or another form of tether for causing the clip 3000 to open. The apertures 3068 may be configured to receive other devices, such as prongs of a delivery apparatus for control of the clip 3000 in examples. The side wall 3038 of the second jaw 3004 may include one or more apertures 3070 that may be configured similarly as the apertures 3068 of the first jaw 3002.

FIG. 215 illustrates a top view of the clip 3000. FIG. 219 illustrates a side view of the clip 3000 with the first jaw 3002 and the second jaw 3004 shown in transparency.

The spring 3030 may be made of a variety of materials including a metal such as cobalt chromium, among other forms of metal. A material such as cobalt-chromium (or an alloy such as Elgiloy—Co—Cr—Ni Alloy, or a MP35n alloy) may provide high stiffness and strength for the spring. The spring 3030 may be spring tempered or spring tempered and aged to provide a high stiffness and strength. The spring 3030 may be stamped or otherwise formed from a strip of material or a sheet of material. In examples, the jaws 3002, 3004 may be made from the same material as desired. In examples, the spring 3030 and/or jaws 3002, 3004 may be made from another material such as titanium, stainless steel, or a shape memory material as desired.

In examples, the spring 3030 may provide the force that moves the clip 3000 from an opened configuration to closed configuration.

In operation, the clip 3000 may be placed in position in an opened configuration. In the opened configuration, the jaws 3002, 3004 may open and extend parallel with each other as represented in solid lines in FIG. 220. The cantilever of the jaws 3002, 3004 may allow the jaws 3002, 3004 to extend parallel with each other as the spring 3030 opens. Upon an opening force being applied only to the tips 3010, 3036 of the jaws 3002, 3004, the jaws 3002, 3004 may angle with respect to each other as shown in solid lines in FIG. 221.

The clip 3000 may close with force applied at the distal end portions 3008, 3034 of the jaws 3002, 3004 and transmitted along the jaws 3002, 3004 proximally. The force load may be distributed evenly along the clip 3000 in examples.

Variations in the clip 3000 may be provided. FIG. 224, for example, illustrates a perspective view of a first jaw 3070 of a clip that may be configured similarly as the first jaw 3002 unless stated otherwise. The first jaw 3070 may include a protrusion 3072 that may protrude from an exterior side surface 3074 of a side wall 3076. An opposite side wall 3078 of the first jaw 3070 may similarly include a protrusion 3080 that may protrude from an exterior side surface 3082 of the first jaw 3070. A horizontal wall 3079 may span the side walls 3076, 3078 and may include a compression surface 3081 of the first jaw 3070.

The respective protrusions 3072, 3080 may be elongate in examples, and may extend in a direction from a proximal end portion 3084 of the first jaw 3070 to a distal end portion 3086 of the first jaw 3070. The respective protrusions 3072, 3080 may be positioned at an upper end of the first jaw 3070 at the opening of the channel 3088 or may have another position as desired.

The respective protrusions 3072, 3080 may comprise rails in examples and may be configured for the first jaw 3070 to slide along a sliding jaw or other feature of a delivery apparatus in examples. The respective protrusions 3072, 3080 accordingly may be utilized for deployment or recapture of the first jaw 3070. A delivery apparatus may engage the protrusions 3072, 3080 or the first jaw 3070 may slide off of or onto the protrusions 3072, 3080 during a deployment or recapture procedure.

The respective protrusions 3072, 3080 may comprise a folded piece of material forming the upper end of the first jaw 3070 as shown in FIG. 225 for example.

The first jaw 3070 may include one or more apertures 3090, 3092 on side walls 3076, 3078 that may be configured similarly as the apertures 3068 shown in FIG. 214 for example.

In examples, a second jaw of the clip may be configured similarly as the first jaw 3070. A delivery apparatus may be configured to apply a force to the protrusions 3072, 3080 of the first jaw 3070 and protrusions of the second jaw to open the clip or may release the force to allow the clip to close.

FIG. 225 illustrates a cross sectional view of the first jaw 3070 shown in FIG. 224.

The clip 3000 may be utilized to close the LAA 18, yet in examples other portions of a heart may be clipped or closed via use of the clip 3000. In examples, other portions of a body, such as a tubular vessel or other portions of a body may be closed with the clip 3000. Deployment may be via a delivery apparatus or via another method as desired. Deployment may be via surgical methods and may be transcatheter or via non-invasive surgery in methods.

Variations in the clips and methods disclosed herein may be provided. Features across examples may be combined. Features may be excluded or added to in various examples disclosed herein. Combinations of features of examples may be provided. The features may be utilized solely or in methods disclosed herein, or in combination with other features disclosed herein.

In addition, the methods herein are not limited to the methods specifically described, and may include methods of utilizing the systems and apparatuses disclosed herein. The steps of the methods may be modified, excluded, or added to, with systems, apparatuses, and methods disclosed herein.

For purposes of this description, certain aspects, advantages, and novel features of the examples of this disclosure are described herein. The disclosed methods, apparatuses, and systems should not be construed as limiting in anyway. Instead, the present disclosure is directed toward all novel and nonobvious features and aspects of the various disclosed examples, alone and in various combinations and sub-combinations with one another. The methods, apparatuses, and systems are not limited to any specific aspect or feature or combination thereof, nor do the disclosed examples require that any one or more specific advantages be present or problems be solved. Features, elements, or components of one example can be combined into other examples herein.

Example 1: A clip for a portion of a heart, the clip comprising: a first jaw including a first arm and a second arm separated from the first arm with a gap, the first arm and the second arm each configured to apply a compressive force to the portion of the heart; and a second jaw positioned offset from the first arm and the second arm and aligned with the gap and configured to apply a compressive force to the portion of the heart in a direction towards the gap.

Example 2: The clip of any example herein, in particular Example 1, wherein the first jaw extends from a first end portion to a second end portion, and the second jaw extends from a first end portion to a second end portion, and the first end portion of the first jaw is coupled to the first end portion of the second jaw.

Example 3: The clip of any example herein, in particular Example 2, wherein the second end portion of the first jaw is coupled to the second end portion of the second jaw.

Example 4: The clip of any example herein, in particular Example 2 or Example 3, further comprising a first spring positioned at the first end portion of the first jaw and the first end portion of the second jaw and configured to force the first jaw and the second jaw together to compress the portion of the heart between the first jaw and the second jaw.

Example 5: The clip of any example herein, in particular Example 4, further comprising a second spring positioned at the second end portion of the first jaw and the second end portion of the second jaw and configured to force the first jaw and the second jaw together to compress the portion of the heart between the first jaw and the second jaw.

Example 6: The clip of any example herein, in particular Example 4 or Example 5, wherein the first spring comprises a loop of material coupling the first jaw to the second jaw.

Example 7: The clip of any example herein, in particular Examples 2-6, wherein the second end portion of the first jaw and the second end portion of the second jaw form an axial opening for a space between the first jaw and the second jaw.

Example 8: The clip of any example herein, in particular Examples 1-7, wherein a space between the first jaw and the second jaw is configured to receive the portion of the heart in a direction transverse to an axis of the first jaw and an axis of the second jaw.

Example 9: The clip of any example herein, in particular Examples 1-8, wherein the first jaw is integral with the second jaw.

Example 10: The clip of any example herein, in particular Examples 1-9, wherein the second jaw is configured to pass into the gap.

Example 11: A method comprising: deploying a clip to close a portion of a heart, the clip including: a first jaw including a first arm and a second arm separated from the first arm with a gap, the first arm and the second arm each configured to apply a compressive force to the portion of the heart, and a second jaw positioned offset from the first arm and the second arm and aligned with the gap and configured to apply a compressive force to the portion of the heart in a direction towards the gap.

Example 12: The method of any example herein, in particular Example 11, wherein the portion of the heart is a left atrial appendage.

Example 13: The method of any example herein, in particular Example 11 or Example 12, wherein the first jaw extends from a first end portion to a second end portion, and the second jaw extends from a first end portion to a second end portion, and the first end portion of the first jaw is coupled to the first end portion of the second jaw.

Example 14: The method of any example herein, in particular Example 13, wherein the second end portion of the first jaw is coupled to the second end portion of the second jaw.

Example 15: The method of any example herein, in particular Example 13 or Example 14, further comprising a first spring positioned at the first end portion of the first jaw and the first end portion of the second jaw and configured to force the first jaw and the second jaw together to compress the portion of the heart between the first jaw and the second jaw.

Example 16: The method of any example herein, in particular Example 15, further comprising a second spring positioned at the second end portion of the first jaw and the second end portion of the second jaw and configured to force the first jaw and the second jaw together to compress the portion of the heart between the first jaw and the second jaw.

Example 17: The method of any example herein, in particular Example 15 or Example 16, wherein the first spring comprises a loop of material coupling the first jaw to the second jaw.

Example 18: The method of any example herein, in particular Examples 13-17, wherein the second end portion of the first jaw and the second end portion of the second jaw form an axial opening for a space between the first jaw and the second jaw.

Example 19: The method of any example herein, in particular Examples 11-18, wherein a space between the first jaw and the second jaw is configured to receive the portion of the heart in a direction transverse to an axis of the first jaw and an axis of the second jaw.

Example 20: The method of any example herein, in particular Examples 11-19, wherein the first jaw is integral with the second jaw.

Example 21: A clip for a portion of a heart, the clip comprising: a first jaw extending from a first end portion to a second end portion and including a central portion between the first end portion and the second end portion; a second jaw extending from a first end portion to a second end portion and including a central portion between the first end portion and the second end portion; and a spring coupled to the central portion of the first jaw and the central portion of the second jaw and configured to force the first jaw and the second jaw together to compress the portion of the heart between the first jaw and the second jaw.

Example 22: The clip of any example herein, in particular Example 21, wherein the first jaw includes a compression surface facing towards the second jaw and an outer surface facing opposite the compression surface, and includes a coupler protruding outward from the outer surface and configured to couple to the spring.

Example 23: The clip of any example herein, in particular Example 22, wherein the first jaw includes a first side surface extending between the compression surface and the outer surface, and the coupler protrudes from the first side surface.

Example 24: The clip of any example herein, in particular Examples 21-23, wherein the first jaw includes a compression surface facing towards the second jaw and an outer surface facing opposite the compression surface, and a first side surface extending between the compression surface and the outer surface and a second side surface facing opposite the first side surface, and includes a coupler positioned between the first side surface and the second side surface and configured to couple to the spring.

Example 25: The clip of any example herein, in particular Example 24, wherein the coupler is positioned between the compression surface and the outer surface.

Example 26: The clip of any example herein, in particular Examples 21-25, wherein the spring includes a first loop extending towards the first end portion of the first jaw and the first end portion of the second jaw, and a second loop extending towards the second end portion of the first jaw and the second end portion of the second jaw.

Example 27: The clip of any example herein, in particular Example 26, wherein the first jaw includes a channel and the second jaw includes a channel, and the first loop extends along the channel of the first jaw and the channel of the second jaw, and the second loop extends along the channel of the first jaw and the channel of the second jaw.

Example 28: The clip of any example herein, in particular Example 26 or Example 27, wherein the first loop forms a boundary of a space between the first jaw and the second jaw, and the second loop forms a boundary of the space between the first jaw and the second jaw.

Example 29: The clip of any example herein, in particular Example 28, wherein the space is configured to receive the portion of the heart in a direction transverse to an axis of the first jaw and an axis of the second jaw.

Example 30: The clip of any example herein, in particular Examples 21-29, wherein the spring includes a first spring body extending towards the first end portion of the first jaw and the first end portion of the second jaw, and a second spring body extending towards the second end portion of the first jaw and the second end portion of the second jaw, with a gap between the first spring body and the second spring body.

Example 31: The clip of any example herein, in particular Examples 21-30, wherein the first jaw includes an aperture for receiving a portion of a delivery apparatus for the clip.

Example 32: The clip of any example herein, in particular Example 31, wherein the second jaw includes an aperture for receiving a portion of the delivery apparatus for the clip.

Example 33: The clip of any example herein, in particular Examples 21-32, wherein the spring includes a first flat plate portion positioned at the central portion of the first jaw, and a second flat plate portion positioned at the central portion of the second jaw.

Example 34: The clip of any example herein, in particular Examples 21-33, wherein: the spring includes a first loop extending towards the first end portion of the first jaw and the first end portion of the second jaw, and a second loop extending towards the second end portion of the first jaw and the second end portion of the second jaw, the first end portion of the first jaw and the first end portion of the second jaw are each positioned at or protrude from the first loop, and the second end portion of the first jaw and the second end portion of the second jaw are each positioned at or protrude from the second loop.

Example 35: The clip of any example herein, in particular Examples 21-34, wherein the first jaw includes a compression surface facing towards the second jaw and curved away from the second jaw, and the second jaw includes a compression surface facing towards the first jaw and curved away from the first jaw.

Example 36: A method comprising: deploying a clip to close a portion of a heart, the clip including: a first jaw extending from a first end portion to a second end portion and including a central portion between the first end portion and the second end portion, a second jaw extending from a first end portion to a second end portion and including a central portion between the first end portion and the second end portion, and a spring coupled to the central portion of the first jaw and the central portion of the second jaw and configured to force the first jaw and the second jaw together to compress the portion of the heart between the first jaw and the second jaw.

Example 37: The method of any example herein, in particular Example 36, wherein the portion of the heart is a left atrial appendage.

Example 38: The method of any example herein, in particular Example 36 or Example 37, wherein the first jaw includes a compression surface facing towards the second jaw and an outer surface facing opposite the compression surface, and includes a coupler protruding outward from the outer surface and configured to couple to the spring.

Example 39: The method of any example herein, in particular Example 38, wherein the first jaw includes a first side surface extending between the compression surface and the outer surface, and the coupler protrudes from the first side surface.

Example 40: The method of any example herein, in particular Examples 36-39, wherein the first jaw includes a compression surface facing towards the second jaw and an outer surface facing opposite the compression surface, and a first side surface extending between the compression surface and the outer surface and a second side surface facing opposite the first side surface, and includes a coupler positioned between the first side surface and the second side surface and configured to couple to the spring.

Example 41: The method of any example herein, in particular Example 40, wherein the coupler is positioned between the compression surface and the outer surface.

Example 42: The method of any example herein, in particular Examples 36-41, wherein the spring includes a first loop extending towards the first end portion of the first jaw and the first end portion of the second jaw, and a second loop extending towards the second end portion of the first jaw and the second end portion of the second jaw.

Example 43: The method of any example herein, in particular Example 42, wherein the first jaw includes a channel and the second jaw includes a channel, and the first loop extends along the channel of the first jaw and the channel of the second jaw, and the second loop extends along the channel of the first jaw and the channel of the second jaw.

Example 44: The method of any example herein, in particular Example 42 or Example 43, wherein the first loop forms a boundary of a space between the first jaw and the second jaw, and the second loop forms a boundary of the space between the first jaw and the second jaw.

Example 45: The method of any example herein, in particular Example 44, wherein the space is configured to receive the portion of the heart in a direction transverse to an axis of the first jaw and an axis of the second jaw.

Example 46: The method of any example herein, in particular Examples 36-45, wherein the first jaw includes an aperture for receiving a portion of a delivery apparatus for the clip.

Example 47: The method of any example herein, in particular Example 46, wherein the second jaw includes an aperture for receiving a portion of the delivery apparatus for the clip.

Example 48: The method of any example herein, in particular Examples 36-47, wherein the spring includes a first flat plate portion positioned at the central portion of the first jaw, and a second flat plate portion positioned at the central portion of the second jaw.

Example 49: The method of any example herein, in particular Examples 36-48, wherein: the spring includes a first loop extending towards the first end portion of the first jaw and the first end portion of the second jaw, and a second loop extending towards the second end portion of the first jaw and the second end portion of the second jaw, the first end portion of the first jaw and the first end portion of the second jaw are each positioned at or protrude from the first loop, and the second end portion of the first jaw and the second end portion of the second jaw are each positioned at or protrude from the second loop.

Example 50: The method of any example herein, in particular Examples 36-49, wherein the first jaw includes a compression surface facing towards the second jaw and curved away from the second jaw, and the second jaw includes a compression surface facing towards the first jaw and curved away from the first jaw.

Example 51: A clip for a portion of a heart, the clip comprising: a first jaw extending from a first end portion to a second end portion and including a first cantilever beam extending from the second end portion towards the first end portion; and a second jaw extending from a first end portion to a second end portion and including a second cantilever beam extending from the second end portion towards the first end portion, the first end portion of the second jaw being pivotally coupled to the first end portion of the first jaw such that a distance between the first cantilever beam and the second cantilever beam may be reduced to compress the portion of the heart between the first cantilever beam and the second cantilever beam.

Example 52: The clip of any example herein, in particular Example 51, further comprising a spring configured to force the first cantilever beam towards the second cantilever beam.

Example 53: The clip of any example herein, in particular Example 52, wherein the spring is positioned at the first end portion of the first jaw and at the first end portion of the second jaw.

Example 54: The clip of any example herein, in particular Example 52 or Example 53, wherein the spring comprises an arcuate body integral with one or more of the first jaw or the second jaw.

Example 55: The clip of any example herein, in particular Examples 51-54, wherein: the first jaw includes a first arm positioned outward of the first cantilever beam and extending from the first end portion of the first jaw to the second end portion of the first jaw; and the second jaw includes a second arm positioned outward of the second cantilever beam and extending from the first end portion of the second jaw to the second end portion of the second jaw.

Example 56: The clip of any example herein, in particular Example 55, wherein: the first arm includes a first end at the second end portion of the first jaw and the first cantilever beam includes a first end at the second end portion of the first jaw that is coupled to the first end of the first arm; and the second arm includes a first end at the second end portion of the second jaw and the second cantilever beam includes a first end at the second end portion of the second jaw that is coupled to the first end of the second arm.

Example 57: The clip of any example herein, in particular Example 51-56, wherein a thickness of the first cantilever beam tapers downward in a direction towards the first end portion of the first jaw, and a thickness of the second cantilever beam tapers downward in a direction towards the first end portion of the second jaw.

Example 58: The clip of any example herein, in particular Example 51-57, wherein the second end portion of the first jaw is a tip of the first jaw, and the second end portion of the second jaw is a tip of the second jaw, and the first end portion of the second jaw is pivotally coupled to the first end portion of the first jaw such that a distance between the tip of the first jaw and the tip of the second jaw varies.

Example 59: The clip of any example herein, in particular Examples 51-58, wherein the first end portion of the second jaw is pivotally coupled to the first end portion of the first jaw such that the second jaw pivots in an are relative to the first jaw.

Example 60: The clip of any example herein, in particular Examples 51-59, wherein the first cantilever beam and the second cantilever beam are configured to deflect outward upon compression of the portion of the heart between the first cantilever beam and the second cantilever beam.

Example 61: A method comprising: deploying a clip to close a portion of a heart, the clip including: a first jaw extending from a first end portion to a second end portion and including a first cantilever beam extending from the second end portion towards the first end portion, and a second jaw extending from a first end portion to a second end portion and including a second cantilever beam extending from the second end portion towards the first end portion, the first end portion of the second jaw being pivotally coupled to the first end portion of the first jaw such that a distance between the first cantilever beam and the second cantilever beam may be reduced to compress the portion of the heart between the first cantilever beam and the second cantilever beam.

Example 62: The method of any example herein, in particular Example 61, wherein the portion of the heart is a left atrial appendage.

Example 63: The method of any example herein, in particular Example 61 or Example 62, wherein a spring forces the first cantilever beam towards the second cantilever beam.

Example 64: The method of any example herein, in particular Example 63, wherein the spring is positioned at the first end portion of the first jaw and at the first end portion of the second jaw.

Example 65: The method of any example herein, in particular Example 63 or Example 64, wherein the spring comprises an arcuate body integral with one or more of the first jaw or the second jaw.

Example 66: The method of any example herein, in particular Examples 61-65, wherein: the first jaw includes a first arm positioned outward of the first cantilever beam and extending from the first end portion of the first jaw to the second end portion of the first jaw; and the second jaw includes a second arm positioned outward of the second cantilever beam and extending from the first end portion of the second jaw to the second end portion of the second jaw.

Example 67: The method of any example herein, in particular Example 66, wherein: the first arm includes a first end at the second end portion of the first jaw and the first cantilever beam includes a first end at the second end portion of the first jaw that is coupled to the first end of the first arm; and the second arm includes a first end at the second end portion of the second jaw and the second cantilever beam includes a first end at the second end portion of the second jaw that is coupled to the first end of the second arm.

Example 68: The method of any example herein, in particular Examples 61-67, wherein a thickness of the first cantilever beam tapers downward in a direction towards the first end portion of the first jaw, and a thickness of the second cantilever beam tapers downward in a direction towards the first end portion of the second jaw.

Example 69: The method of any example herein, in particular Examples 61-68, wherein the second end portion of the first jaw is a tip of the first jaw, and the second end portion of the second jaw is a tip of the second jaw, and the first end portion of the second jaw is pivotally coupled to the first end portion of the first jaw such that a distance between the tip of the first jaw and the tip of the second jaw varies.

Example 70: The method of any example herein, in particular Examples 61-69, wherein the first end portion of the second jaw is pivotally coupled to the first end portion of the first jaw such that the second jaw pivots in an are relative to the first jaw.

Example 71: A clip for a portion of a heart, the clip comprising: a first jaw; a second jaw; and a four-link mechanism configured to move the first jaw relative to the second jaw to compress the portion of the heart between the first jaw and the second jaw.

Example 72: The clip of any example herein, in particular Example 71, wherein the first jaw forms a shortest link of the four-link mechanism and the second jaw forms a third shortest link of the four-link mechanism.

Example 73: The clip of any example herein, in particular Example 71 or Example 72, wherein a first bar extends from the first jaw to the second jaw to form a second shortest link of the four-link mechanism, and a second bar extends from the second jaw to the first jaw to form a longest link of the four-link mechanism.

Example 74: The clip of any example herein, in particular Example 73, wherein the second bar is curved.

Example 75: The clip of any example herein, in particular Examples 71-74, wherein the first jaw extends from a first pivot between a longest link and a shortest link of the four-link mechanism, and the second jaw extends from a second pivot between a third shortest link and the longest link of the four-link mechanism.

Example 76: The clip of any example herein, in particular Examples 71-75, further comprising a spring configured to apply a compressive force to the four-link mechanism and force the first jaw towards the second jaw.

Example 77: The clip of any example herein, in particular Examples 71-76, wherein the first jaw extends from an end portion to a tip of the first jaw, and the second jaw extends from an end portion to a tip of the second jaw, and the four-link mechanism is configured to cause the tip of the first jaw and the tip of the second jaw to close prior to the end portion of the first jaw and the end portion of the second jaw.

Example 78: The clip of any example herein, in particular Example 77, wherein a bar of the four-link mechanism is positioned at the end portion of the first jaw and the end portion of the second jaw.

Example 79: The clip of any example herein, in particular Examples 71-78, wherein the four-link mechanism is configured to allow the first jaw to move in a plane, and the first jaw is configured to bend in a direction transverse to the plane.

Example 80: The clip of any example herein, in particular Examples 71-79, wherein the four-link mechanism is a non-Grashof four-link mechanism or a Grashof four-link mechanism.

Example 81: A method comprising: deploying a clip to close a portion of a heart, the clip including: a first jaw, a second jaw, and a four-link mechanism configured to move the first jaw relative to the second jaw to compress the portion of the heart between the first jaw and the second jaw.

Example 82: The method of any example herein, in particular Example 81, wherein the portion of the heart is a left atrial appendage.

Example 83: The method of any example herein, in particular Example 81 or Example 82, wherein the first jaw forms a shortest link of the four-link mechanism and the second jaw forms a third shortest link of the four-link mechanism.

Example 84: The method of any example herein, in particular Examples 81-83, wherein a first bar extends from the first jaw to the second jaw to form a second shortest link of the four-link mechanism, and a second bar extends from the second jaw to the first jaw to form a longest link of the four-link mechanism.

Example 85: The method of any example herein, in particular Example 84, wherein the second bar is curved.

Example 86: The method of any example herein, in particular Examples 81-85, wherein the first jaw extends from a first pivot between a longest link and a shortest link of the four-link mechanism, and the second jaw extends from a second pivot between a third shortest link and the longest link of the four-link mechanism.

Example 87: The method of any example herein, in particular Examples 81-86, further comprising a spring configured to apply a compressive force to the four-link mechanism and force the first jaw towards the second jaw.

Example 88: The method of any example herein, in particular Examples 81-87, wherein the first jaw extends from an end portion to a tip of the first jaw, and the second jaw extends from an end portion to a tip of the second jaw, and the four-link mechanism is configured to cause the tip of the first jaw and the tip of the second jaw to close prior to the end portion of the first jaw and the end portion of the second jaw.

Example 89: The method of any example herein, in particular Example 88, wherein a bar of the four-link mechanism is positioned at the end portion of the first jaw and the end portion of the second jaw.

Example 90: The method of any example herein, in particular Examples 81-89, wherein the four-link mechanism is a non-Grashof four-link mechanism or a Grashof four-link mechanism.

Example 91: A clip for a portion of a heart, the clip comprising: a first jaw extending from a first end portion to a second end portion and including a central portion between the first end portion and the second end portion; a second jaw extending from a first end portion to a second end portion and including a central portion between the first end portion and the second end portion; and a spring coupled to the central portion of the first jaw and the central portion of the second jaw and configured to force the first jaw and the second jaw together to compress the portion of the heart between the first jaw and the second jaw.

Example 92: The clip of any example herein, in particular Example 91, wherein the first jaw includes a compression surface facing towards the second jaw and an outer surface facing opposite the compression surface, and includes a coupler protruding outward from the outer surface and configured to couple to the spring.

Example 93: The clip of any example herein, in particular Example 92, wherein the first jaw includes a first side surface extending between the compression surface and the outer surface, and the coupler protrudes from the first side surface.

Example 94: The clip of any example herein, in particular Example 93, wherein the coupler comprises a pivot coupler that the spring is configured to pivot about.

Example 95: The clip of any example herein, in particular Example 91-94, wherein the spring includes a first end coupled to the central portion of the first jaw and a second end coupled to the central portion of the second jaw, and a loop extending towards the first end portion of the first jaw and the first end portion of the second jaw.

Example 96: The clip of any example herein, in particular Example 95, wherein the loop forms a boundary of a space between the first jaw and the second jaw.

Example 97: The clip of any example herein, in particular Example 96, wherein the second end portion of the first jaw and the second end portion of the second jaw form an axial opening for the space.

Example 98: The clip of any example herein, in particular Example 96 or Example 97, wherein the loop protrudes from the first end portion of the first jaw and the first end portion of the second jaw.

Example 99: The clip of any example herein, in particular Examples 91-98, wherein the first jaw has a hollow center and the second jaw has a hollow center.

Example 100: The clip of any example herein, in particular Examples 91-99, wherein the first jaw has an oval cross section and the second jaw has an oval cross section.

Example 101: A method comprising: deploying a clip to close a portion of a heart, the clip including: a first jaw extending from a first end portion to a second end portion and including a central portion between the first end portion and the second end portion, a second jaw extending from a first end portion to a second end portion and including a central portion between the first end portion and the second end portion, and a spring coupled to the central portion of the first jaw and the central portion of the second jaw and configured to force the first jaw and the second jaw together to compress the portion of the heart between the first jaw and the second jaw.

Example 102: The method of any example herein, in particular Example 101, wherein the portion of the heart is a left atrial appendage.

Example 103: The method of any example herein, in particular Example 101 or Example 102, wherein the first jaw includes a compression surface facing towards the second jaw and an outer surface facing opposite the compression surface, and includes a coupler protruding outward from the outer surface and configured to couple to the spring.

Example 104: The method of any example herein, in particular Example 103, wherein the first jaw includes a first side surface extending between the compression surface and the outer surface, and the coupler protrudes from the first side surface.

Example 105: The method of any example herein, in particular Example 104, wherein the coupler comprises a pivot coupler that the spring is configured to pivot about.

Example 106: The method of any example herein, in particular Examples 101-105, wherein the spring includes a first end coupled to the central portion of the first jaw and a second end coupled to the central portion of the second jaw, and a loop extending towards the first end portion of the first jaw and the first end portion of the second jaw.

Example 107: The method of any example herein, in particular Example 106, wherein the loop forms a boundary of a space between the first jaw and the second jaw.

Example 108: The method of any example herein, in particular Example 107, wherein the second end portion of the first jaw and the second end portion of the second jaw form an axial opening for the space.

Example 109: The method of any example herein, in particular Example 107 or Example 108, wherein the loop protrudes from the first end portion of the first jaw and the first end portion of the second jaw.

Example 110: The method of any example herein, in particular Examples 101-109, wherein the first jaw has a hollow center and the second jaw has a hollow center.

Example 11: A clip for a portion of a heart, the clip comprising: a first jaw extending from a first end portion to a second end portion; a second jaw extending from a first end portion to a second end portion and extending along the first jaw to form a channel for receiving the portion of the heart; a first spring coupling the first jaw to the second jaw and covering a first end of the channel at the first end portion of the first jaw and the first end portion of the second jaw, the first spring configured to force the first jaw towards the second jaw to compress the portion of the heart within the channel; and a second spring coupling the first jaw to the second jaw and covering a second end of the channel at the second end portion of the first jaw and the second end portion of the second jaw, the second spring configured to force the first jaw towards the second jaw to compress the portion of the heart within the channel.

Example 112: The clip of any example herein, in particular Example 111, wherein the first spring includes a loop protruding from the first end portion of the first jaw and the first end portion of the second jaw, and the second spring includes a loop protruding from the second end portion of the first jaw and the second end portion of the second jaw.

Example 113: The clip of any example herein, in particular Example in or Example 112, wherein the first spring includes a first end coupled to the first jaw and a second end coupled to the second jaw, and the second spring includes a first end coupled to the first jaw and a second end coupled to the second jaw.

Example 114: The clip of any example herein, in particular Examples 111-113, further comprising an aperture on the first jaw and configured to receive a device for applying an expansion force to the first spring and the second spring.

Example 115: The clip of any example herein, in particular Examples 111-114, wherein the first spring is spaced from the first end of the channel with a first gap, and the second spring is spaced from the second end of the channel with a second gap.

Example 116: The clip of any example herein, in particular Examples 111-115, further comprising a first deflectable arm coupling the first jaw to the first spring, and a second deflectable arm coupling the first jaw to the second spring.

Example 117: The clip of any example herein, in particular Example 116, wherein the first deflectable arm is coupled to a first end of the first jaw, and the second deflectable arm is coupled to a second end of the first jaw.

Example 118: The clip of any example herein, in particular Example 116, wherein the first deflectable arm is coupled to a central portion of the first jaw, and the second deflectable arm is coupled to the central portion of the first jaw.

Example 119: The clip of any example herein, in particular Examples 111-118, further comprising a first support arm positioned outward of the first jaw and coupling the first spring to the second spring, and a second support arm positioned outward of the second jaw and coupling the first spring to the second spring, and wherein the first spring, the second spring, the first support arm, and the second support arm form a continuous perimeter around the first jaw and the second jaw.

Example 120: The clip of any example herein, in particular Examples 111-119, further comprising a third spring positioned inward of the first spring and coupling the first jaw to the second jaw, and a fourth spring positioned inward of the second spring and coupling the first jaw to the second jaw.

Example 121: The clip of any example herein, in particular Examples 111-120, further comprising: an aperture on the first jaw and configured to receive a device for applying an expansion force to the first spring and the second spring; and a first support arm positioned outward of the first jaw and coupling the first spring to the second spring and coupled to the aperture.

Example 122: The clip of any example herein, in particular Examples 111-121, further comprising: an aperture on the first jaw and configured to receive a device for applying an expansion force to the first spring and the second spring; and a first support arm positioned outward of the first jaw and coupling the first spring to the second spring and extending over the aperture.

Example 123: The clip of any example herein, in particular Examples 111-122, further comprising a first support arm positioned outward of the first jaw and coupling the first spring to the second spring and bowed towards the first jaw.

Example 124: The clip of any example herein, in particular Examples 111-123, further comprising an aperture on the first jaw and configured to receive a device for applying an expansion force to the first spring and the second spring, and the aperture comprises a raised portion of the first jaw.

Example 125: The clip of any example herein, in particular Example 124, wherein the aperture comprises a curved portion of the first jaw.

Example 126: A method comprising: deploying a clip to close a portion of a heart, the clip including: a first jaw extending from a first end portion to a second end portion, a second jaw extending from a first end portion to a second end portion and extending along the first jaw to form a channel for receiving the portion of the heart, a first spring coupling the first jaw to the second jaw and covering a first end of the channel at the first end portion of the first jaw and the first end portion of the second jaw, the first spring configured to force the first jaw towards the second jaw to compress the portion of the heart within the channel, and a second spring coupling the first jaw to the second jaw and covering a second end of the channel at the second end portion of the first jaw and the second end portion of the second jaw, the second spring configured to force the first jaw towards the second jaw to compress the portion of the heart within the channel.

Example 127: The method of any example herein, in particular Example 126, wherein the portion of the heart is a left atrial appendage.

Example 128: The method of any example herein, in particular Example 126 or Example 127, wherein the first spring includes a loop protruding from the first end portion of the first jaw and the first end portion of the second jaw, and the second spring includes a loop protruding from the second end portion of the first jaw and the second end portion of the second jaw.

Example 129: The method of any example herein, in particular Examples 126-128, wherein the first spring includes a first end coupled to the first jaw and a second end coupled to the second jaw, and the second spring includes a first end coupled to the first jaw and a second end coupled to the second jaw.

Example 130: The method of any example herein, in particular Examples 126-129, further comprising an aperture on the first jaw and configured to receive a device for applying an expansion force to the first spring and the second spring.

Example 131: The method of any example herein, in particular Examples 126-130, wherein the first spring is spaced from the first end of the channel with a first gap, and the second spring is spaced from the second end of the channel with a second gap.

Example 132: The method of any example herein, in particular Examples 126-131, further comprising a first deflectable arm coupling the first jaw to the first spring, and a second deflectable arm coupling the first jaw to the second spring.

Example 133: The method of any example herein, in particular Example 132, wherein the first deflectable arm is coupled to a first end of the first jaw, and the second deflectable arm is coupled to a second end of the first jaw.

Example 134: The method of any example herein, in particular Example 132, wherein the first deflectable arm is coupled to a central portion of the first jaw, and the second deflectable arm is coupled to the central portion of the first jaw.

Example 135: The method of any example herein, in particular Examples 126-134, further comprising a first support arm positioned outward of the first jaw and coupling the first spring to the second spring, and a second support arm positioned outward of the second jaw and coupling the first spring to the second spring, and wherein the first spring, the second spring, the first support arm, and the second support arm form a continuous perimeter around the first jaw and the second jaw.

Example 136: The method of any example herein, in particular Examples 126-135, further comprising: an aperture on the first jaw and configured to receive a device for applying an expansion force to the first spring and the second spring; and a first support arm positioned outward of the first jaw and coupling the first spring to the second spring and coupled to the aperture.

Example 137: The method of any example herein, in particular Examples 126-136, further comprising: an aperture on the first jaw and configured to receive a device for applying an expansion force to the first spring and the second spring; and a first support arm positioned outward of the first jaw and coupling the first spring to the second spring and extending over the aperture.

Example 138: The method of any example herein, in particular Examples 126-137, further comprising a first support arm positioned outward of the first jaw and coupling the first spring to the second spring and bowed towards the first jaw.

Example 139: The method of any example herein, in particular Examples 126-138, further comprising an aperture on the first jaw and configured to receive a device for applying an expansion force to the first spring and the second spring, and the aperture comprises a raised portion of the first jaw.

Example 140: The method of any example herein, in particular Examples 126-139, further comprising recapturing the clip.

Example 141: A clip for a portion of a heart, the clip comprising: a first jaw extending from a first end portion to a second end portion; a second jaw extending from a first end portion to a second end portion and extending along the first jaw to form a channel for receiving the portion of the heart, and the second end portion of the second jaw being separable from the second end portion of the first jaw to form an axial opening for the channel; and a spring positioned at the first end portion of the first jaw and the first end portion of the second jaw and configured to force the first jaw and the second jaw together to compress the portion of the heart.

Example 142: The clip of any example herein, in particular Example 141, wherein the spring comprises an arcuate body protruding outward from the first end portion of the first jaw and the first end portion of the second jaw.

Example 143: The clip of any example herein, in particular Example 141 or Example 142, wherein: the first jaw includes a first cantilever beam extending from the second end portion towards the first end portion of the first jaw; and the second jaw includes a second cantilever beam extending from the second end portion towards the first end portion of the second jaw.

Example 144: The clip of any example herein, in particular Examples 141-143, wherein the first jaw includes a central portion between the first end portion and the second end portion of the first jaw, and the spring couples to the central portion of the first jaw; and the second jaw includes a central portion between the first end portion and the second end portion of the second jaw, and the spring couples to the central portion of the second jaw.

Example 145: The clip of any example herein, in particular Examples 141-144, wherein the first jaw includes a first arm extending from the second end portion towards the first end portion of the first jaw; the second jaw includes a second arm extending from the second end portion towards the first end portion of the second jaw; and the spring comprises a first spring, and the clip further comprises a second spring coupled to an end of the first arm and an end of the second arm.

Example 146: The clip of any example herein, in particular Example 145, wherein the first spring extends around the second spring.

Example 147: The clip of any example herein, in particular Example 145 or Example 146, wherein the second spring is an arcuate body.

Example 148: The clip of any example herein, in particular Examples 141-147, wherein the first jaw includes at least one cut-out portion, and the second jaw includes at least one cut-out portion.

Example 149: The clip of any example herein, in particular Example 148, wherein the first jaw includes a plurality of cut-out portions and the second jaw includes a plurality of cut-out portions.

Example 150: The clip of any example herein, in particular Examples 141-149, wherein the first jaw tapers downward in a direction towards the second end portion of the first jaw; and the second jaw tapers downward in a direction towards the second end portion of the second jaw.

Example 151: A method comprising: deploying a clip to close a portion of a heart, the clip including: a first jaw extending from a first end portion to a second end portion, a second jaw extending from a first end portion to a second end portion and extending along the first jaw to form a channel for receiving the portion of the heart, and the second end portion of the second jaw being separable from the second end portion of the first jaw to form an axial opening for the channel, and a spring positioned at the first end portion of the first jaw and the first end portion of the second jaw and configured to force the first jaw and the second jaw together to compress the portion of the heart.

Example 152: The method of any example herein, in particular Example 151, wherein the portion of the heart is a left atrial appendage.

Example 153: The method of any example herein, in particular Example 151 or Example 152, wherein the spring comprises an arcuate body protruding outward from the first end portion of the first jaw and the first end portion of the second jaw.

Example 154: The method of any example herein, in particular Examples 151-153, wherein: the first jaw includes a first cantilever beam extending from the second end portion towards the first end portion of the first jaw; and the second jaw includes a second cantilever beam extending from the second end portion towards the first end portion of the second jaw.

Example 155: The method of any example herein, in particular Examples 151-154, wherein the first jaw includes a central portion between the first end portion and the second end portion of the first jaw, and the spring couples to the central portion of the first jaw; and the second jaw includes a central portion between the first end portion and the second end portion of the second jaw, and the spring couples to the central portion of the second jaw.

Example 156: The method of any example herein, in particular Examples 151-155, wherein the first jaw includes a first arm extending from the second end portion towards the first end portion of the first jaw; the second jaw includes a second arm extending from the second end portion towards the first end portion of the second jaw; and the spring comprises a first spring, and the clip further comprises a second spring coupled to an end of the first arm and an end of the second arm.

Example 157: The method of any example herein, in particular Example 156, wherein the first spring extends around the second spring.

Example 158: The method of any example herein, in particular Example 156 or Example 157, wherein the second spring is an arcuate body.

Example 159: The method of any example herein, in particular Examples 151-158, wherein the first jaw includes at least one cut-out portion, and the second jaw includes at least one cut-out portion.

Example 160: The method of any example herein, in particular Examples 151-159, wherein the clip is deployed to a left atrial appendage and the clip has a curvature that matches a curvature of the left atrial appendage.

Example 161: A clip for a portion of a heart, the clip comprising: a first jaw extending from a first end portion to a second end portion; a second jaw extending from a first end portion to a second end portion; and a spring coupling the first jaw to the second jaw and configured to force the first jaw towards the second jaw to compress the portion of the heart between the first jaw and the second jaw, the spring being configured to slide relative to the second jaw to allow movement of the first jaw relative to the second jaw.

Example 162: The clip of any example herein, in particular Example 161, wherein the spring includes an end coupled to the second jaw and configured to slide relative to the second jaw.

Example 163: The clip of any example herein, in particular Example 161 or Example 162, wherein the second jaw includes a track for the spring to slide along.

Example 164: The clip of any example herein, in particular Example 163, wherein the second jaw includes a compression surface and an outer surface facing opposite the compression surface and a side surface extending between the compression surface and the outer surface, and the track is on the side surface.

Example 165: The clip of any example herein, in particular Example 164, wherein the spring protrudes from the side surface.

Example 166: The clip of any example herein, in particular Example 163, wherein the second jaw includes a compression surface and an outer surface facing opposite the compression surface, and the track is on the outer surface.

Example 167: The clip of any example herein, in particular Examples 161-166, wherein the spring includes a first spring body extending towards the first end portion of the first jaw and the first end portion of the second jaw, and a second spring body extending towards the second end portion of the first jaw and the second end portion of the second jaw.

Example 168: The clip of any example herein, in particular Examples 161-166, wherein the spring comprises a continuous body forming a first loop extending towards the first end portion of the first jaw and the first end portion of the second jaw and a second loop extending towards the second end portion of the first jaw and the second end portion of the second jaw.

Example 169: The clip of any example herein, in particular Examples 161-168, further comprising a slide bar positioned outward of the second jaw and coupled to the spring and configured to slide relative to the second jaw.

Example 170: The clip of any example herein, in particular Example 169, wherein a first end of the spring is coupled to the second jaw and a second end of the spring is coupled to the slide bar.

Example 171: A method comprising: deploying a clip to close a portion of a heart, the clip including: a first jaw extending from a first end portion to a second end portion, a second jaw extending from a first end portion to a second end portion, and a spring coupling the first jaw to the second jaw and configured to force the first jaw towards the second jaw to compress the portion of the heart between the first jaw and the second jaw, the spring being configured to slide relative to the second jaw to allow movement of the first jaw relative to the second jaw.

Example 172: The method of any example herein, in particular Example 171, wherein the portion of the heart is a left atrial appendage.

Example 173: The method of any example herein, in particular Example 171 or Example 172, wherein the spring includes an end coupled to the second jaw and configured to slide relative to the second jaw.

Example 174: The method of any example herein, in particular Examples 171-173, wherein the second jaw includes a track for the spring to slide along.

Example 175: The method of any example herein, in particular Example 174, wherein the second jaw includes a compression surface and an outer surface facing opposite the compression surface and a side surface extending between the compression surface and the outer surface, and the track is on the side surface.

Example 176: The method of any example herein, in particular Example 175, wherein the spring protrudes from the side surface.

Example 177: The method of any example herein, in particular Example 174, wherein the second jaw includes a compression surface and an outer surface facing opposite the compression surface, and the track is on the outer surface.

Example 178: The method of any example herein, in particular Examples 171-177, wherein the spring includes a first spring body extending towards the first end portion of the first jaw and the first end portion of the second jaw, and a second spring body extending towards the second end portion of the first jaw and the second end portion of the second jaw.

Example 179: The method of any example herein, in particular Examples 171-177, wherein the spring comprises a continuous body forming a first loop extending towards the first end portion of the first jaw and the first end portion of the second jaw and a second loop extending towards the second end portion of the first jaw and the second end portion of the second jaw.

Example 180: The method of any example herein, in particular Examples 171-179, further comprising a slide bar positioned outward of the second jaw and coupled to the spring and configured to slide relative to the second jaw.

Example 181: A clip for a portion of a heart, the clip comprising: a first jaw; a second jaw; and a slotted pin joint coupled to the first jaw and the second jaw and configured to allow the first jaw to move relative to the second jaw to compress the portion of the heart between the first jaw and the second jaw.

Example 182: The clip of any example herein, in particular Example 181, wherein the first jaw has a distal end portion and a proximal end portion, and the second jaw has a distal end portion and a proximal end portion, and the slotted pin joint is positioned at the proximal end portions of the first jaw and the second jaw.

Example 183: The clip of any example herein, in particular Example 182, wherein the distal end portion of the first jaw and the distal end portion of the second jaw define an opening for a channel for receiving the portion of the heart.

Example 184: The clip of any example herein, in particular Example 183, wherein the slotted pin joint is configured to allow the first jaw to angle relative to the second jaw such that the channel has a width at the proximal end portions of the first jaw and the second jaw that is greater than a width at the distal end portions of the first jaw and the second jaw.

Example 185: The clip of any example herein, in particular Examples 182-184, wherein the slotted pin joint is configured to allow the distal end portions of the first jaw and the second jaw to close prior to the proximal end portions of the first jaw and the second jaw.

Example 186: The clip of any example herein, in particular Examples 181-185, wherein the slotted pin joint is configured to allow the first jaw to extend parallel with the second jaw.

Example 187: The clip of any example herein, in particular Examples 181-186, wherein the first jaw extends along an axis, and the slotted pin joint includes a slot on the first jaw extending transverse to the axis and a pin on the second jaw configured to slide along the slot.

Example 188: The clip of any example herein, in particular Example 187, wherein the slot extends perpendicular to the axis.

Example 189: The clip of any example herein, in particular Example 187 or Example 188, wherein the slot includes an upper end and a lower end, and the pin is configured to slide along the slot towards the upper end to increase a distance between the first jaw and the second jaw and is configured to slide along the slot towards the lower end to reduce a distance between the first jaw and the second jaw.

Example 190: The clip of any example herein, in particular Examples 181-189, further comprising a spring configured to force the first jaw towards the second jaw.

Example 191: A method comprising: deploying a clip to close a portion of a heart, the clip including: a first jaw, a second jaw, and a slotted pin joint coupled to the first jaw and the second jaw and configured to allow the first jaw to move relative to the second jaw to compress the portion of the heart between the first jaw and the second jaw.

Example 192: The method of any example herein, in particular Example 191, wherein the portion of the heart is a left atrial appendage.

Example 193: The method of any example herein, in particular Example 191 or Example 192, wherein the first jaw has a distal end portion and a proximal end portion, and the second jaw has a distal end portion and a proximal end portion, and the slotted pin joint is positioned at the proximal end portions of the first jaw and the second jaw.

Example 194: The method of any example herein, in particular Example 193, wherein the distal end portion of the first jaw and the distal end portion of the second jaw define an opening for a channel for receiving the portion of the heart.

Example 195: The method of any example herein, in particular Example 194, wherein the slotted pin joint is configured to allow the first jaw to angle relative to the second jaw such that the channel has a width at the proximal end portions of the first jaw and the second jaw that is greater than a width at the distal end portions of the first jaw and the second jaw.

Example 196: The method of any example herein, in particular Examples 193-195, wherein the slotted pin joint is configured to allow the distal end portions of the first jaw and the second jaw to close prior to the proximal end portions of the first jaw and the second jaw.

Example 197: The method of any example herein, in particular Examples 191-196, wherein the slotted pin joint is configured to allow the first jaw to extend parallel with the second jaw.

Example 198: The method of any example herein, in particular Examples 191-197, wherein the first jaw extends along an axis, and the slotted pin joint includes a slot on the first jaw extending transverse to the axis and a pin on the second jaw configured to slide along the slot.

Example 199: The method of any example herein, in particular Example 198, wherein the slot includes an upper end and a lower end, and the pin is configured to slide along the slot towards the upper end to increase a distance between the first jaw and the second jaw and is configured to slide along the slot towards the lower end to reduce a distance between the first jaw and the second jaw.

Example 200: The method of any example herein, in particular Examples 191-199, wherein a spring is configured to force the first jaw towards the second jaw.

Example 201: A clip system for a left atrial appendage of a heart, the clip system comprising: a first clip configured to be deployed proximate an ostium of the left atrial appendage to occlude a portion of the left atrial appendage; a second clip configured to be deployed between the first clip and a wall of a left atrium of the heart to occlude a portion of the heart between the first clip and the wall of the left atrium; and a delivery apparatus configured to deploy one or more of the first clip or the second clip to the respective portions.

Example 202: The clip system of any example herein, in particular Example 201, wherein the second clip is configured to draw a portion of the wall of the left atrium into the portion of the heart occluded by the second clip.

Example 203: The clip system of any example herein, in particular Example 201 or Example 202, wherein the first clip comprises a V-clip.

Example 204: The clip system of any example herein, in particular Examples 201-203, wherein the second clip comprises a V-clip.

Example 205: The clip system of any example herein, in particular Examples 201-204, wherein the first clip comprises a loop body including a first elongate jaw, a second elongate jaw, a closed end, and an opened end, with a channel between the first elongate jaw and the second elongate jaw for the portion of the left atrial appendage to be positioned within.

Example 206: The clip system of any example herein, in particular Examples 201-205, wherein the second clip comprises a loop body including a first elongate jaw, a second elongate jaw, a closed end, and an opened end, with a channel between the first elongate jaw and the second elongate jaw for the portion of the heart to be positioned within.

Example 207: The clip system of any example herein, in particular Examples 201-206, wherein the second clip is configured to draw pits or divots of the left atrial appendage into the portion of the heart occluded by the second clip.

Example 208: The clip system of any example herein, in particular Examples 201-207, wherein the delivery apparatus is configured to deploy the first clip and the second clip to the respective portion.

Example 209: The clip system of any example herein, in particular Examples 201-208, wherein the delivery apparatus includes a deployment head configured to retain the first clip parallel with the second clip.

Example 210: The clip system of any example herein, in particular Examples 201-209, wherein the delivery apparatus is configured to deploy the first clip and the second clip sequentially, with deployment of the second clip following deployment of the first clip.

Example 211: A method comprising: deploying a first clip proximate an ostium of a left atrial appendage to occlude a portion of the left atrial appendage; and deploying a second clip between the first clip and a wall of a left atrium of a heart to occlude a portion of the heart between the first clip and the wall of the left atrium.

Example 212: The method of any example herein, in particular Example 211, further comprising utilizing a delivery apparatus to deploy one or more of the first clip or the second clip to the respective portion.

Example 213: The method of any example herein, in particular Example 212, further comprising deploying the first clip and the second clip to the respective portion utilizing the delivery apparatus.

Example 214: The method of any example herein, in particular Example 212 or Example 213, wherein the delivery apparatus includes a deployment head configured to retain the first clip parallel with the second clip.

Example 215: The method of any example herein, in particular Examples 211-214, further comprising deploying the first clip and the second clip sequentially, with deployment of the second clip following deployment of the first clip.

Example 216: The method of any example herein, in particular Examples 211-215, further comprising drawing a portion of the wall of the left atrium into the portion of the heart occluded by the second clip.

Example 217: The method of any example herein, in particular Examples 211-216, wherein the first clip comprises a V-clip.

Example 218: The method of any example herein, in particular Examples 211-217, wherein the second clip comprises a V-clip.

Example 219: The method of any example herein, in particular Examples 211-218, wherein the first clip comprises a loop body including a first elongate jaw, a second elongate jaw, a closed end, and an opened end, with a channel between the first elongate jaw and the second elongate jaw for the portion of the left atrial appendage to be positioned within.

Example 220: The method of any example herein, in particular Examples 211-219, wherein the second clip comprises a loop body including a first elongate jaw, a second elongate jaw, a closed end, and an opened end, with a channel between the first elongate jaw and the second elongate jaw for the portion of the heart to be positioned within.

Example 221: A clip for a portion of a heart, the clip comprising: an elongate upper jaw extending along a first axis and having a first outer surface, the elongate upper jaw configured for the first outer surface to rotate about the first axis in a first rotational direction; and an elongate lower jaw extending along a second axis and having a second outer surface, the elongate lower jaw configured for the second outer surface to rotate about the second axis in a second rotational direction that is opposite the first rotational direction to draw a portion of the heart between the first outer surface and the second outer surface.

Example 222: The clip of any example herein, in particular Example 221, wherein the elongate upper jaw includes an elongate axle coincident with the first axis, the first outer surface configured to rotate about the elongate axle.

Example 223: The clip of any example herein, in particular Example 221 or Example 222, wherein the elongate upper jaw includes an elongate axle that is eccentric with respect to the first outer surface.

Example 224: The clip of any example herein, in particular Examples 221-223, further comprising a clip body configured to be inserted into the elongate upper jaw and the elongate lower jaw to cause the first outer surface and the second outer surface to rotate.

Example 225: The clip of any example herein, in particular Example 224, wherein the clip body include a first elongate arm, a second elongate arm, and a connecting portion extending between the first elongate arm and the second elongate arm, with the first elongate arm configured to extend into the elongate upper jaw and the second elongate arm configured to extend into the elongate lower jaw.

Example 226: The clip of any example herein, in particular Example 224 or Example 225, wherein the clip body has a “U” shape.

Example 227: The clip of any example herein, in particular Examples 224-226, wherein the elongate upper jaw includes a first channel for receiving the clip body, and the elongate lower jaw includes a second channel for receiving the clip body.

Example 228: The clip of any example herein, in particular Example 227, wherein the elongate upper jaw includes a first roller surrounding a first axle and a second roller surrounding a second axle, the first roller including the first channel and the second roller including the second channel.

Example 229: The clip of any example herein, in particular Examples 221-228, wherein the second outer surface is configured to rotate about the second axis in a second rotational direction to draw pits or divots of a left atrial appendage into the portion of the heart occluded by the clip.

Example 230: The clip of any example herein, in particular Examples 221-229, wherein the clip is configured to be deployed proximate an ostium of a left atrial appendage.

Example 231: A method comprising: deploying a clip to close a portion of a heart, the clip including: an elongate upper jaw extending along a first axis and having a first outer surface, the elongate upper jaw configured for the first outer surface to rotate about the first axis in a first rotational direction, and an elongate lower jaw extending along a second axis and having a second outer surface, the elongate lower jaw configured for the second outer surface to rotate about the second axis in a second rotational direction that is opposite the first rotational direction to draw a portion of the heart between the first outer surface and the second outer surface.

Example 232: The method of any example herein, in particular Example 231, wherein the elongate upper jaw includes an elongate axle coincident with the first axis, the first outer surface configured to rotate about the elongate axle.

Example 233: The method of any example herein, in particular Example 231 or Example 232, wherein the elongate upper jaw includes an elongate axle that is eccentric with respect to the first outer surface.

Example 234: The method of any example herein, in particular Examples 231-233, further comprising inserting a clip body into the elongate upper jaw and the elongate lower jaw to cause the first outer surface and the second outer surface to rotate.

Example 235: The method of any example herein, in particular Example 234, wherein the clip body include a first elongate arm, a second elongate arm, and a connecting portion extending between the first elongate arm and the second elongate arm, with the first elongate arm configured to extend into the elongate upper jaw and the second elongate arm configured to extend into the elongate lower jaw.

Example 236: The method of any example herein, in particular Example 234 or Example 235, wherein the clip body has a “U” shape.

Example 237: The method of any example herein, in particular Examples 234-236, wherein the elongate upper jaw includes a first channel for receiving the clip body, and the elongate lower jaw includes a second channel for receiving the clip body.

Example 238: The method of any example herein, in particular Example 237, wherein the elongate upper jaw includes a first roller surrounding a first axle and a second roller surrounding a second axle, the first roller including the first channel and the second roller including the second channel.

Example 239: The method of any example herein, in particular Examples 231-238, wherein the second outer surface is configured to rotate about the second axis in a second rotational direction to draw pits or divots of a left atrial appendage into the portion of the heart occluded by the clip.

Example 240: The method of any example herein, in particular Examples 231-239, wherein the clip is configured to be deployed proximate an ostium of a left atrial appendage.

Example 241: A clip for a portion of a heart, the clip comprising: a retainer body including: an upper receiver configured to be positioned on a first side of the portion of the heart and including a first pair of opposed tracks, a lower receiver configured to be positioned on a second side of the portion of the heart that is opposite the first side and including a second pair of opposed tracks, and a loop body coupling the upper receiver to the lower receiver; an upper jaw configured to apply a compressive force to the first side of the portion of the heart, at least a portion of the upper jaw configured to be slidably engaged with the first pair of opposed tracks; and a lower jaw configured to apply a compressive force to the second side of the portion of the heart, at least a portion of the lower jaw configured to be slidably engaged with the second pair of opposed tracks.

Example 242: The clip of any example herein, in particular Example 241, wherein the upper receiver includes a distal portion and a proximal portion, and the at least the portion of the upper jaw is configured to slide along the first pair of opposed tracks in a direction from the proximal portion towards the distal portion.

Example 243: The clip of any example herein, in particular Example 242, wherein the loop body is positioned at the proximal portion of the upper receiver.

Example 244: The clip of any example herein, in particular Example 242 or Example 243, wherein the first pair of opposed tracks each comprise a channel including an opening for entry of the at least the portion of the upper jaw into the respective channel at the proximal portion of the upper receiver.

Example 245: The clip of any example herein, in particular Examples 241-244, wherein the upper jaw includes: a compressive surface configured to apply the compressive force to the first side of the portion of the heart, an upper surface facing opposite the compressive surface, and one or more side surfaces positioned between the compressive surface and the upper surface; and the upper receiver forms a loop around an outer perimeter of the one or more side surfaces.

Example 246: The clip of any example herein, in particular Examples 241-245, wherein the upper jaw includes: a compressive surface configured to apply the compressive force to the first side of the portion of the heart, an upper surface facing opposite the compressive surface, and one or more side surfaces positioned between the compressive surface and the upper surface and including a flange configured to slide along the first pair of opposed tracks.

Example 247: The clip of any example herein, in particular Example 245 or Example 246, wherein the upper receiver has a “U” shape extending around the outer perimeter of the one or more side surfaces.

Example 248: The clip of any example herein, in particular Examples 241-247, wherein the upper jaw has a wedge shape.

Example 249: The clip of any example herein, in particular Examples 241-248, wherein the upper jaw includes a first compressive surface configured to apply the compressive force to the first side of the portion of the heart, and the lower jaw includes a second compressive surface configured to apply the compressive force to the second side of the portion of the heart, the first compressive surface and the second compressive surface each comprising a flat surface extending parallel with each other.

Example 250: The clip of any example herein, in particular Examples 241-249, further comprising one or more locks configured to lock the at least the portion of the upper jaw within the first pair of opposed tracks and lock the at least the portion of the lower jaw within the second pair of opposed tracks.

Example 251: A method comprising: deploying a clip to close a portion of a heart, the clip including: a retainer body including: an upper receiver configured to be positioned on a first side of the portion of the heart and including a first pair of opposed tracks, a lower receiver configured to be positioned on a second side of the portion of the heart that is opposite the first side and including a second pair of opposed tracks, and a loop body coupling the upper receiver to the lower receiver, an upper jaw configured to apply a compressive force to the first side of the portion of the heart, at least a portion of the upper jaw configured to be slidably engaged with the first pair of opposed tracks, and a lower jaw configured to apply a compressive force to the second side of the portion of the heart, at least a portion of the lower jaw configured to be slidably engaged with the second pair of opposed tracks.

Example 252: The method of any example herein, in particular Example 251, wherein the portion of the heart is a left atrial appendage.

Example 253: The method of any example herein, in particular Example 251 or Example 252, wherein the upper receiver includes a distal portion and a proximal portion, and the at least the portion of the upper jaw is configured to be slid along the first pair of opposed tracks in a direction from the proximal portion towards the distal portion.

Example 254: The method of any example herein, in particular Example 253, wherein the loop body is positioned at the proximal portion of the upper receiver.

Example 255: The method of any example herein, in particular Examples 251-254, wherein the upper jaw includes: a compressive surface configured to apply the compressive force to the first side of the portion of the heart, an upper surface facing opposite the compressive surface, and one or more side surfaces positioned between the compressive surface and the upper surface; and the upper receiver forms a loop around an outer perimeter of the one or more side surfaces.

Example 256: The method of any example herein, in particular Examples 251-255, wherein the upper jaw includes: a compressive surface configured to apply the compressive force to the first side of the portion of the heart, an upper surface facing opposite the compressive surface, and one or more side surfaces positioned between the compressive surface and the upper surface and including a flange configured to slide along the first pair of opposed tracks.

Example 257: The method of any example herein, in particular Example 255 or Example 256, wherein the upper receiver has a “U” shape extending around the outer perimeter of the one or more side surfaces.

Example 258: The method of any example herein, in particular Examples 251-257, wherein the upper jaw has a wedge shape.

Example 259: The method of any example herein, in particular Examples 251-258, wherein the upper jaw includes a first compressive surface configured to apply the compressive force to the first side of the portion of the heart, and the lower jaw includes a second compressive surface configured to apply the compressive force to the second side of the portion of the heart, the first compressive surface and the second compressive surface each comprising a flat surface extending parallel with each other.

Example 260: The method of any example herein, in particular Examples 251-259, further comprising one or more locks configured to lock the at least the portion of the upper jaw within the first pair of opposed tracks and lock the at least the portion of the lower jaw within the second pair of opposed tracks.

Example 261: A clip for a portion of a heart, the clip comprising: an upper jaw forming a plate and including a first aperture and a first compressive surface configured to apply a compressive force to a first side of the portion of the heart; a lower jaw forming a plate and including a second aperture and a second compressive surface configured to apply a compressive force to a second side of the portion of the heart; and a loop body configured to insert into the first aperture and the second aperture and configured to compress the upper jaw and the lower jaw towards each other.

Example 262: The clip of any example herein, in particular Example 261, wherein the first compressive surface and the second compressive surface each comprise a flat surface extending parallel with each other.

Example 263: The clip of any example herein, in particular Example 261 or Example 262, wherein the loop body has a “U” shape.

Example 264: The clip of any example herein, in particular Examples 261-263, wherein the loop body includes: a first portion configured to insert into the first aperture, a second portion configured to insert into the second aperture, and a connecting portion connecting the first portion and the second portion.

Example 265: The clip of any example herein, in particular Example 264, wherein the first portion comprises a first elongate arm and the second portion comprises a second elongate arm.

Example 266: The clip of any example herein, in particular Example 265, wherein the first aperture forms an entrance to a first elongate channel configured to receive the first elongate arm, and the second aperture forms an entrance to a second elongate channel configured to receive the second elongate arm.

Example 267: The clip of any example herein, in particular Examples 264-266, wherein the first portion of the loop body is configured to be slid along the first aperture, and the second portion of the loop body is configured to be slid along the second aperture.

Example 268: The clip of any example herein, in particular Examples 261-267, wherein the upper jaw includes an upper surface facing opposite the first compressive surface and one or more side surfaces positioned between the upper surface and the first compressive surface, and the first aperture is positioned on the one or more side surfaces.

Example 269: The clip of any example herein, in particular Examples 261-268, wherein the loop body is spring biased to compress the upper jaw and the lower jaw towards each other.

Example 270: The clip of any example herein, in particular Examples 261-269, wherein the upper jaw has a rectangular shape and the lower jaw has a rectangular shape.

Example 271: A method comprising: deploying a clip to close a portion of a heart, the clip including: an upper jaw forming a plate and including a first aperture and a first compressive surface configured to apply a compressive force to a first side of the portion of the heart, a lower jaw forming a plate and including a second aperture and a second compressive surface configured to apply a compressive force to a second side of the portion of the heart, and a loop body configured to insert into the first aperture and the second aperture and configured to compress the upper jaw and the lower jaw towards each other.

Example 272: The method of any example herein, in particular Example 271, wherein the portion of the heart is a left atrial appendage.

Example 273: The method of any example herein, in particular Example 271 or Example 272, wherein the first compressive surface and the second compressive surface each comprise a flat surface extending parallel with each other.

Example 274: The method of any example herein, in particular Examples 271-273, wherein the loop body includes: a first portion configured to insert into the first aperture, a second portion configured to insert into the second aperture, and a connecting portion connecting the first portion and the second portion.

Example 275: The method of any example herein, in particular Example 274, wherein the first portion comprises a first elongate arm and the second portion comprises a second elongate arm.

Example 276: The method of any example herein, in particular Example 275, wherein the first aperture forms an entrance to a first elongate channel configured to receive the first elongate arm, and the second aperture forms an entrance to a second elongate channel configured to receive the second elongate arm.

Example 277: The method of any example herein, in particular Examples 274-276, wherein the first portion of the loop body is configured to be slid along the first aperture, and the second portion of the loop body is configured to be slid along the second aperture.

Example 278: The method of any example herein, in particular Examples 271-277, wherein the upper jaw includes an upper surface facing opposite the first compressive surface and one or more side surfaces positioned between the upper surface and the first compressive surface, and the first aperture is positioned on the one or more side surfaces.

Example 279: The method of any example herein, in particular Examples 271-278, wherein the loop body is spring biased to compress the upper jaw and the lower jaw towards each other.

Example 280: The method of any example herein, in particular Examples 271-279, wherein the upper jaw has a rectangular shape and the lower jaw has a rectangular shape.

Example 281: A clip for a portion of a heart, the clip comprising: a first loop body including a first elongate arm, a second elongate arm, a first closed end, a first channel between the first elongate arm and the second elongate arm and a first opened end forming an opening for the first channel; and a second loop body including a third elongate arm, a fourth elongate arm, a second closed end, a second channel between the third elongate arm and the fourth elongate arm and a second opened end forming an opening for the second channel, the second loop body configured to couple to the first loop body with the second opened end positioned proximate the first closed end and the second closed end positioned proximate the first opened end, the first elongate arm and the third elongate arm forming a first jaw for compressing a first side of the portion of the heart, and the second elongate arm and the fourth elongate arm forming a second jaw for compressing a second side of the portion of the heart.

Example 282: The clip of any example herein, in particular Example 281, wherein the first elongate arm includes a first elongate cavity for receiving the third elongate arm, and the second elongate arm includes a second elongate cavity for receiving the fourth elongate arm.

Example 283: The clip of any example herein, in particular Example 282, wherein the first elongate cavity is continuous with the second elongate cavity.

Example 284: The clip of any example herein, in particular Examples 281-283, wherein the first elongate arm is configured to be positioned closer to the first side of the portion of the heart than the third elongate arm.

Example 285: The clip of any example herein, in particular Examples 281-284, wherein the second loop body is configured to be slid along the first loop body in a direction from the first opened end towards the first closed end to couple to the first loop body.

Example 286: The clip of any example herein, in particular Examples 281-285, wherein the first elongate arm and the second elongate arm are both curved.

Example 287: The clip of any example herein, in particular Examples 281-286, further comprising one or more locks for locking the first loop body to the second loop body.

Example 288: The clip of any example herein, in particular Examples 281-287, wherein the first elongate arm is configured to extend parallel with the third elongate arm, and the second elongate arm is configured to extend parallel with the fourth elongate arm.

Example 289: The clip of any example herein, in particular Examples 281-288, wherein the first closed end and the second closed end are configured to bound the portion of the heart positioned within the first channel.

Example 290: The clip of any example herein, in particular Examples 281-289, wherein the first loop body and the second loop body are configured to form a ring extending around the portion of the heart.

Example 291: A method comprising: deploying a clip to close a portion of a heart, the clip including: a first loop body including a first elongate arm, a second elongate arm, a first closed end, a first channel between the first elongate arm and the second elongate arm and a first opened end forming an opening for the first channel; and a second loop body including a third elongate arm, a fourth elongate arm, a second closed end, a second channel between the third elongate arm and the fourth elongate arm and a second opened end forming an opening for the second channel, the second loop body configured to couple to the first loop body with the second opened end positioned proximate the first closed end and the second closed end positioned proximate the first opened end, the first elongate arm and the third elongate arm forming a first jaw for compressing a first side of the portion of the heart, and the second elongate arm and the fourth elongate arm forming a second jaw for compressing a second side of the portion of the heart.

Example 292: The method of any example herein, in particular Example 291, wherein the portion of the heart is a left atrial appendage.

Example 293: The method of any example herein, in particular Example 291 or Example 292, wherein the first elongate arm includes a first elongate cavity for receiving the third elongate arm, and the second elongate arm includes a second elongate cavity for receiving the fourth elongate arm.

Example 294: The method of any example herein, in particular Example 293, wherein the first elongate cavity is continuous with the second elongate cavity.

Example 295: The method of any example herein, in particular Examples 291-294, wherein the first elongate arm is configured to be positioned closer to the first side of the portion of the heart than the third elongate arm.

Example 296: The method of any example herein, in particular Examples 291-295, wherein the second loop body is configured to be slid along the first loop body in a direction from the first opened end towards the first closed end to couple to the first loop body.

Example 297: The method of any example herein, in particular Examples 291-296, further comprising one or more locks for locking the first loop body to the second loop body.

Example 298: The method of any example herein, in particular Examples 291-297, wherein the first elongate arm is configured to extend parallel with the third elongate arm, and the second elongate arm is configured to extend parallel with the fourth elongate arm.

Example 299: The method of any example herein, in particular Examples 291-298, wherein the first closed end and the second closed end are configured to bound the portion of the heart positioned within the first channel.

Example 300: The method of any example herein, in particular Examples 291-299, wherein the first loop body and the second loop body are configured to form a ring extending around the portion of the heart.

Example 301: A method comprising: forming a fold of a first portion of a left atrial appendage of a heart with a second portion of the heart; and bonding the first portion to the second portion to maintain the fold.

Example 302: The method of any example herein, in particular Example 301, further comprising bonding the first portion to the second portion with an adhesive to maintain the fold.

Example 303: The method of any example herein, in particular Example 301 or Example 302, further comprising bonding the first portion to the second portion with sutures to maintain the fold.

Example 304: The method of any example herein, in particular Examples 301-303, further comprising forming the fold to occlude the left atrial appendage.

Example 305: The method of any example herein, in particular Examples 301-304, further comprising forming the fold to reduce a volume of an inner chamber of the left atrial appendage.

Example 306: The method of any example herein, in particular Example 305, further comprising forming the fold to eliminate the inner chamber of the left atrial appendage.

Example 307: The method of any example herein, in particular Examples 301-306, wherein forming the fold includes facing an outer surface of the first portion of the left atrial appendage with an outer surface of the second portion of the heart.

Example 308: The method of any example herein, in particular Examples 301-307, wherein the second portion of the heart comprises a wall of a left atrium of the heart.

Example 309: The method of any example herein, in particular Examples 301-308, wherein the first portion of the left atrial appendage comprises a portion of a wall of the left atrial appendage and the second portion of the heart comprises a portion of a wall of the left atrial appendage.

Example 310: The method of any example herein, in particular Examples 301-309, further comprising pressing the left atrial appendage inward towards an inner chamber of the left atrial appendage to form the fold.

Example 311: The method of any example herein, in particular Examples 301-310, further comprising pressing the left atrial appendage inward into an ostium of the left atrial appendage.

Example 312: The method of any example herein, in particular Examples 301-311, further comprising bonding an adhesive body to the first portion and the second portion to maintain the fold.

Example 313: The method of any example herein, in particular Example 312, wherein the adhesive body is coupled to an applicator device, and the method further comprises releasing the adhesive body from the applicator device.

Example 314: The method of any example herein, in particular Example 313, wherein the applicator device comprises an elongate applicator shaft and the adhesive body comprises a tip of the elongate applicator shaft.

Example 315: The method of any example herein, in particular Examples 301-314, further comprising forming multiple folds of the left atrial appendage to reduce a volume of an inner chamber of the left atrial appendage.

Example 316: A system for bonding a first portion of a left atrial appendage of a heart to a second portion of the heart, the system comprising: an elongate applicator shaft; and an implant comprising a tip of the elongate applicator shaft and configured to release from the elongate applicator shaft, the implant including an outer surface having an adhesive configured to bond the first portion of the left atrial appendage of the heart to the second portion of the heart.

Example 317: The system of any example herein, in particular Example 316, wherein the outer surface is configured to be pressed against the left atrial appendage to bond the first portion of the left atrial appendage of the heart to the second portion of the heart.

Example 318: The system of any example herein, in particular Example 316 or Example 317, wherein the outer surface is configured to be pressed against the left atrial appendage to fold the first portion of the left atrial appendage of the heart with the second portion of the heart.

Example 319: The system of any example herein, in particular Examples 316-318, wherein the implant is configured to release from the elongate applicator shaft with the elongate applicator shaft being retracted from the left atrial appendage.

Example 320: The system of any example herein, in particular Examples 316-319, wherein the implant comprises an adhesive pad.

Example 321: The system of any example herein, in particular Examples 316-320, wherein the outer surface of the implant includes a layer of the adhesive.

Example 322: The system of any example herein, in particular Examples 316-321, wherein the implant comprises a biocompatible material.

Example 323: The system of any example herein, in particular Examples 316-322, wherein the implant surrounds a central shaft.

Example 324: The system of any example herein, in particular Examples 316-323, wherein the implant has an elongate shape.

Example 325: The system of any example herein, in particular Examples 316-324, wherein the implant has a cylindrical shape.

Example 326: A clip for a portion of a heart, the clip comprising: a first jaw extending from a first end portion to a second end portion along a length, the first jaw including a compression surface, an outer surface facing opposite the compression surface, a first side surface extending along the length and configured to face distally and being angled along the length, and a second side surface configured to face proximally and opposite the first side surface; a second jaw extending from a first end portion to a second end portion along a length, the second jaw including a compression surface, an outer surface facing opposite the compression surface, a first side surface extending along the length of the second jaw and configured to face distally, and a second side surface configured to face proximally and opposite the first side surface of the second jaw; and at least one spring having a first end portion coupled to the first jaw and a second end portion coupled to the second jaw and a loop portion protruding proximally from the first end portion and the second end portion of the at least one spring, the at least one spring configured to force the first jaw and the second jaw together to compress the portion of the heart between the compression surface of the first jaw and the compression surface of the second jaw.

Example 327: The clip of any example herein, in particular Example 326, wherein the length of the first jaw is less than the length of the second jaw.

Example 328: The clip of any example herein, in particular Example 326 or Example 327, wherein the first jaw includes a central portion positioned between the first end portion and the second end portion of the first jaw, and the first side surface of the first jaw protrudes at the central portion relative to the first side surface at the first end portion or at the second end portion of the first jaw.

Example 329: The clip of any example herein, in particular Example 328, wherein the first side surface of the first jaw protrudes at the central portion relative to the first side surface at the first end portion and the second end portion of the first jaw.

Example 330: The clip of any example herein, in particular Examples 326-329, wherein the first jaw includes a central portion positioned between the first end portion and the second end portion of the first jaw, and the first side surface of the first jaw has a first curvature at the central portion and a second curvature at the first end portion that is different than the first curvature.

Example 331: The clip of any example herein, in particular Example 330, wherein the first curvature is less than the second curvature.

Example 332: The clip of any example herein, in particular Examples 326-331, wherein a thickness of the first jaw varies along the length of the first jaw.

Example 333: The clip of any example herein, in particular Examples 326-332, wherein the first side surface of the second jaw is angled along the length of the second jaw.

Example 334: The clip of any example herein, in particular Examples 326-333, wherein the at least one spring includes a first spring and a second spring together forming an hourglass shape.

Example 335: The clip of any example herein, in particular Examples 326-334, wherein the at least one spring includes a first spring and a second spring, the first spring being coupled to the first end portion of the first jaw and the first end portion of the second jaw, and the second spring being coupled to the second end portion of the first jaw and the second end portion of the second jaw.

Example 336: The clip of any example herein, in particular Example 335, wherein the first spring includes an angled portion that is angled towards the second spring, and the second spring includes an angled portion that is angled towards the first spring.

Example 337: The clip of any example herein, in particular Example 336, wherein the angled portion of the first spring and the angled portion of the second spring are each biased towards each other to apply a compressive force to a delivery apparatus.

Example 338: The clip of any example herein, in particular Examples 326-337, wherein the at least one spring couples to the first jaw with a pivot coupler.

Example 339: The clip of any example herein, in particular Example 338, wherein the pivot coupler comprises a ball joint.

Example 340: The clip of any example herein, in particular Examples 326-339, wherein the compression surface of the first jaw and the compression surface of the second jaw are each configured to compress a left atrial appendage (LAA) and the first side surface of the first jaw and the first side surface of the second jaw are each configured to face a wall of the heart.

Example 341: A method comprising: deploying a clip to close a portion of a heart, the clip including: a first jaw extending from a first end portion to a second end portion along a length, the first jaw including a compression surface, an outer surface facing opposite the compression surface, a first side surface extending along the length and configured to face distally and being angled along the length, and a second side surface configured to face proximally and opposite the first side surface, a second jaw extending from a first end portion to a second end portion along a length, the second jaw including a compression surface, an outer surface facing opposite the compression surface, a first side surface extending along the length of the second jaw and configured to face distally, and a second side surface configured to face proximally and opposite the first side surface of the second jaw, and at least one spring having a first end portion coupled to the first jaw and a second end portion coupled to the second jaw and a loop portion protruding proximally from the first end portion and the second end portion of the at least one spring, the at least one spring configured to force the first jaw and the second jaw together to compress the portion of the heart between the compression surface of the first jaw and the compression surface of the second jaw.

Example 342: The method of any example herein, in particular Example 341, wherein the length of the first jaw is less than the length of the second jaw.

Example 343: The method of any example herein, in particular Example 341 or Example 342, wherein the first jaw includes a central portion positioned between the first end portion and the second end portion of the first jaw, and the first side surface of the first jaw protrudes at the central portion relative to the first side surface at the first end portion or at the second end portion of the first jaw.

Example 344: The method of any example herein, in particular Examples 341-343, wherein a thickness of the first jaw varies along the length of the first jaw.

Example 345: The method of any example herein, in particular Examples 341-344, wherein the at least one spring includes a first spring and a second spring together forming an hourglass shape.

Example 346: The method of any example herein, in particular Examples 341-345, wherein the at least one spring includes a first spring and a second spring, the first spring being coupled to the first end portion of the first jaw and the first end portion of the second jaw, and the second spring being coupled to the second end portion of the first jaw and the second end portion of the second jaw.

Example 347: The method of any example herein, in particular Example 346, wherein the first spring includes an angled portion that is angled towards the second spring, and the second spring includes an angled portion that is angled towards the first spring.

Example 348: The method of any example herein, in particular Example 347, wherein the angled portion of the first spring and the angled portion of the second spring each apply a compressive force to a delivery apparatus.

Example 349: The method of any example herein, in particular Examples 341-348, wherein the at least one spring couples to the first jaw with a pivot coupler.

Example 350: The method of any example herein, in particular Examples 341-349, wherein the portion of the heart is a left atrial appendage.

Example 351: A clip for a portion of a heart, the clip comprising: a first jaw extending from a first end portion to a second end portion along a length, the first jaw including a compression surface, an outer surface facing opposite the compression surface, a first side surface extending along the length and configured to face distally and being angled along the length, and a second side surface configured to face proximally and opposite the first side surface; a second jaw extending from a first end portion to a second end portion along a length, the second jaw including a compression surface and an outer surface facing opposite the compression surface; and at least one spring laterally offset proximally from the second side surface of the first jaw, the at least one spring configured to force the first jaw and the second jaw together to compress the portion of the heart between the compression surface of the first jaw and the compression surface of the second jaw.

Example 352: The clip of any example herein, in particular Example 351, wherein the first jaw includes a first elongate portion, a second elongate portion, and a bend positioned between the first elongate portion and the second elongate portion and angling the first elongate portion relative to the second elongate portion, and the at least one spring is adjacent to the first elongate portion.

Example 353: The clip of any example herein, in particular Example 352, wherein the at least one spring extends parallel with the first elongate portion.

Example 354: The clip of any example herein, in particular Example 352 or Example 353, wherein the first elongate portion has a first length and the second elongate portion has a second length that is less than the first length.

Example 355: The clip of any example herein, in particular Examples 351-354, wherein the first jaw has a uniform thickness along the length of the first jaw.

Example 356: The clip of any example herein, in particular Examples 351-355, wherein the first jaw has a uniform height along the length of the first jaw.

Example 357: The clip of any example herein, in particular Examples 351-356, wherein the first side surface includes a single bend.

Example 358: The clip of any example herein, in particular Examples 351-357, wherein the second jaw includes a first side surface extending along the length of the second jaw and configured to face distally and being angled along the length of the second jaw, and a second side surface configured to face proximally and opposite the first side surface of the second jaw.

Example 359: The clip of any example herein, in particular Examples 351-358, wherein the compression surface of the first jaw has a concave profile along the length of the first jaw, and the compression surface of the second jaw has a convex profile along the length of the second jaw.

Example 360: The clip of any example herein, in particular Examples 351-359, wherein the first jaw is bowed upward in a plane of closure of the clip, and the second jaw is bowed upward in the plane of closure.

Example 361: The clip of any example herein, in particular Examples 351-360, wherein the at least one spring includes a spring having a first end portion coupled to the first jaw and a second end portion coupled to the second jaw.

Example 362: The clip of any example herein, in particular Example 361, wherein the first jaw includes a central portion positioned between the first end portion and the second end portion of the first jaw, and the first end portion of the spring is coupled to the central portion and the second end portion of the spring is coupled to the first end portion of the second jaw.

Example 363: The clip of any example herein, in particular Example 361 or Example 362, wherein the first end portion of the spring couples to the first jaw with a pivot coupler.

Example 364: The clip of any example herein, in particular Examples 361-363, wherein the spring is bowed upward in a plane of closure of the clip above the outer surface of the first jaw.

Example 365: The clip of any example herein, in particular Examples 351-364, wherein the compression surface of the first jaw and the compression surface of the second jaw are each configured to compress a left atrial appendage (LAA) and the first side surface of the first jaw is configured to face a wall of the heart.

Example 366: A method comprising: deploying a clip to close a portion of a heart, the clip including: a first jaw extending from a first end portion to a second end portion along a length, the first jaw including a compression surface, an outer surface facing opposite the compression surface, a first side surface extending along the length and configured to face distally and being angled along the length, and a second side surface configured to face proximally and opposite the first side surface; a second jaw extending from a first end portion to a second end portion along a length, the second jaw including a compression surface and an outer surface facing opposite the compression surface; and at least one spring laterally offset proximally from the second side surface of the first jaw, the at least one spring configured to force the first jaw and the second jaw together to compress the portion of the heart between the compression surface of the first jaw and the compression surface of the second jaw.

Example 367: The method of any example herein, in particular Example 366, wherein the first jaw includes a first elongate portion, a second elongate portion, and a bend positioned between the first elongate portion and the second elongate portion and angling the first elongate portion relative to the second elongate portion, and the at least one spring is adjacent to the first elongate portion.

Example 368: The method of any example herein, in particular Example 367, wherein the first elongate portion has a first length and the second elongate portion has a second length that is less than the first length.

Example 369: The method of any example herein, in particular Examples 366-368, wherein the first side surface includes a single bend.

Example 370: The method of any example herein, in particular Examples 366-369, wherein the second jaw includes a first side surface extending along the length of the second jaw and configured to face distally and being angled along the length of the second jaw, and a second side surface configured to face proximally and opposite the first side surface of the second jaw.

Example 371: The method of any example herein, in particular Examples 366-370, wherein the at least one spring includes a spring having a first end portion coupled to the first jaw and a second end portion coupled to the second jaw.

Example 372: The method of any example herein, in particular Example 371, wherein the first jaw includes a central portion positioned between the first end portion and the second end portion of the first jaw, and the first end portion of the spring is coupled to the central portion and the second end portion of the spring is coupled to the first end portion of the second jaw.

Example 373: The method of any example herein, in particular Example 371 or Example 372, wherein the first end portion of the spring couples to the first jaw with a pivot coupler.

Example 374: The method of any example herein, in particular Examples 366-373, wherein the at least one spring is bowed upward in a plane of closure of the clip above the outer surface of the first jaw.

Example 375: The method of any example herein, in particular Examples 366-374, wherein the portion of the heart is a left atrial appendage.

Example 376: A clip for a portion of a heart, the clip comprising: a first jaw extending from a proximal end portion to a distal end portion; a second jaw extending from a proximal end portion to a distal end portion; and a c-shaped spring having a first end and a second end and a loop portion that loops around the proximal end portion of the first jaw and the proximal end portion of the second jaw, the first end of the c-shaped spring configured to apply a compressive force to the distal end portion of the first jaw and the second end of the c-shaped spring configured to apply a compressive force to the distal end portion of the second jaw, the c-shaped spring configured to force the first jaw and the second jaw together.

Example 377: The clip of any example herein, in particular Example 376, wherein the first jaw comprises a cantilever beam.

Example 378: The clip of any example herein, in particular Example 376 or Example 377, wherein the second jaw comprises a cantilever beam.

Example 379: The clip of any example herein, in particular Examples 376-378, wherein the first jaw comprises a U-shaped beam.

Example 380: The clip of any example herein, in particular Examples 376-379, wherein the first end of the c-shaped spring has a fixed connection to the distal end portion of the first jaw.

Example 381: The clip of any example herein, in particular Examples 376-380, wherein the proximal end portion of the first jaw has a cut-out portion that that loop portion extends into.

Example 382: The clip of any example herein, in particular Examples 376-381, wherein the c-shaped spring is made of cobalt chromium.

Example 383: The clip of any example herein, in particular Examples 376-382, wherein the first jaw includes a compression surface facing towards the second jaw and an outer surface facing opposite the compression surface, and the first end of the c-shaped spring is coupled to the outer surface of the first jaw.

Example 384: The clip of any example herein, in particular Examples 376-383, wherein the distal end portion of the first jaw and the distal end portion of the second jaw form an axial opening for a space between the first jaw and the second jaw.

Example 385: The clip of any example herein, in particular Example 384, wherein the loop portion forms a boundary of the space between the first jaw and the second jaw.

Example 386: A method comprising: deploying a clip to close a portion of a heart, the clip including: a first jaw extending from a proximal end portion to a distal end portion; a second jaw extending from a proximal end portion to a distal end portion; and a c-shaped spring having a first end and a second end and a loop portion that loops around the proximal end portion of the first jaw and the proximal end portion of the second jaw, the first end of the c-shaped spring configured to apply a compressive force to the distal end portion of the first jaw and the second end of the c-shaped spring configured to apply a compressive force to the distal end portion of the second jaw, the c-shaped spring configured to force the first jaw and the second jaw together.

Example 387: The method of any example herein, in particular Example 386, wherein the first jaw comprises a cantilever beam.

Example 388: The method of any example herein, in particular Example 386 or Example 387, wherein the second jaw comprises a cantilever beam.

Example 389: The method of any example herein, in particular Examples 386-388, wherein the first jaw comprises a U-shaped beam.

Example 390: The method of any example herein, in particular Examples 386-389, wherein the first end of the c-shaped spring has a fixed connection to the distal end portion of the first jaw.

Example 391: The method of any example herein, in particular Examples 386-390, wherein the proximal end portion of the first jaw has a cut-out portion that that loop portion extends into.

Example 392: The method of any example herein, in particular Examples 386-391, wherein the c-shaped spring is made of cobalt chromium.

Example 393: The method of any example herein, in particular Examples 386-392, wherein the first jaw includes a compression surface facing towards the second jaw and an outer surface facing opposite the compression surface, and the first end of the c-shaped spring is coupled to the outer surface of the first jaw.

Example 394: The method of any example herein, in particular Examples 386-393, wherein the distal end portion of the first jaw and the distal end portion of the second jaw form an axial opening for a space between the first jaw and the second jaw.

Example 395: The method of any example herein, in particular Example 394, wherein the loop portion forms a boundary of the space between the first jaw and the second jaw.

Any of the features of any of the examples, including but not limited to any of the first through 395 examples referred to above, is applicable to all other aspects and examples identified herein, including but not limited to any examples of any of the first through 395 examples referred to above. Moreover, any of the features of an example of the various examples, including but not limited to any examples of any of the first through 395 examples referred to above, is independently combinable, partly or wholly with other examples described herein in any way, e.g., one, two, or three or more examples may be combinable in whole or in part. Further, any of the features of the various examples, including but not limited to any examples of any of the first through 395 examples referred to above, may be made optional to other examples. Any example of a method can be performed by a system or apparatus of another example, and any aspect or example of a system or apparatus can be configured to perform a method of another aspect or example, including but not limited to any examples of any of the first through 395 examples referred to above.

In closing, it is to be understood that although aspects of the present specification are highlighted by referring to specific examples, one skilled in the art will readily appreciate that these disclosed examples are only illustrative of the principles of the subject matter disclosed herein. Therefore, it should be understood that the disclosed subject matter is in no way limited to a particular methodology, protocol, and/or reagent, etc., described herein. As such, various modifications or changes to or alternative configurations of the disclosed subject matter can be made in accordance with the teachings herein without departing from the spirit of the present specification. Lastly, the terminology used herein is for the purpose of describing particular examples only, and is not intended to limit the scope of systems, apparatuses, and methods as disclosed herein, which is defined solely by the claims. Accordingly, the systems, apparatuses, and methods are not limited to that precisely as shown and described.

Certain examples of systems, apparatuses, and methods are described herein, including the best mode known to the inventors for carrying out the same. Of course, variations on these described examples will become apparent to those of ordinary skill in the art upon reading the foregoing description. The inventor expects skilled artisans to employ such variations as appropriate, and the inventors intend for the systems, apparatuses, and methods to be practiced otherwise than specifically described herein. Accordingly, the systems, apparatuses, and methods include all modifications and equivalents of the subject matter recited in the claims appended hereto as permitted by applicable law. Moreover, any combination of the above-described examples in all possible variations thereof is encompassed by the systems, apparatuses, and methods unless otherwise indicated herein or otherwise clearly contradicted by context.

Groupings of alternative examples, elements, or steps of the systems, apparatuses, and methods are not to be construed as limitations. Each group member may be referred to and claimed individually or in any combination with other group members disclosed herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Unless otherwise indicated, all numbers expressing a characteristic, item, quantity, parameter, property, term, and so forth used in the present specification and claims are to be understood as being modified in all instances by the term “about.” As used herein, the term “about” means that the characteristic, item, quantity, parameter, property, or term so qualified encompasses an approximation that may vary, yet is capable of performing the desired operation or process discussed herein.

The terms “a,” “an,” “the” and similar referents used in the context of describing the systems, apparatuses, and methods (especially in the context of the following claims) are to be construed to cover both the singular and the plural, unless otherwise indicated herein or clearly contradicted by context. All methods described herein can be performed in any suitable order unless otherwise indicated herein or otherwise clearly contradicted by context. The use of any and all examples, or exemplary language (e.g., “such as”) provided herein is intended merely to better illuminate the systems, apparatuses, and methods and does not pose a limitation on the scope of the systems, apparatuses, and methods otherwise claimed. No language in the present specification should be construed as indicating any non-claimed element essential to the practice of the systems, apparatuses, and methods.

All patents, patent publications, and other publications referenced and identified in the present specification are individually and expressly incorporated herein by reference in their entirety for the purpose of describing and disclosing, for example, the compositions and methodologies described in such publications that might be used in connection with the systems, apparatuses, and methods. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

What is claimed is:

1. A clip for a portion of a heart, the clip comprising:
a first jaw having a distal end portion and a proximal end portion;
a second jaw having a distal end portion and a proximal end portion, and a groove at the proximal end portion of the second jaw for receiving the proximal end portion of the first jaw; and
a slotted pin joint coupled to the first jaw and the second jaw and configured to allow the first jaw to move relative to the second jaw to compress the portion of the heart between the first jaw and the second jaw.

2. The clip of claim 1, wherein the slotted pin joint is positioned at the proximal end portions of the first jaw and the second jaw.

3. The clip of claim 2, wherein the distal end portion of the first jaw and the distal end portion of the second jaw define an opening for a channel for receiving the portion of the heart.

4. The clip of claim 3, wherein the slotted pin joint is configured to allow the first jaw to angle relative to the second jaw such that the channel has a width at the proximal end portions of the first jaw and the second jaw that is greater than a width at the distal end portions of the first jaw and the second jaw.

5. The clip of claim 2, wherein the slotted pin joint is configured to allow the distal end portions of the first jaw and the second jaw to close prior to the proximal end portions of the first jaw and the second jaw.

6. The clip of claim 1, wherein the slotted pin joint is configured to allow the first jaw to extend parallel with the second jaw.

7. The clip of claim 1, wherein the first jaw extends along an axis, and the slotted pin joint includes a slot on the first jaw extending transverse to the axis and a pin on the second jaw configured to slide along the slot.

8. The clip of claim 7, wherein the slot extends perpendicular to the axis.

9. The clip of claim 7, wherein the slot includes an upper end and a lower end, and the pin is configured to slide along the slot towards the upper end to increase a distance between the first jaw and the second jaw and is configured to slide along the slot towards the lower end to reduce a distance between the first jaw and the second jaw.

10. The clip of claim 1, further comprising a spring configured to force the first jaw towards the second jaw.

11. A method comprising:
deploying a clip to close a portion of a heart, the clip including:
a first jaw having a distal end portion and a proximal end portion,
a second jaw having a distal end portion and a proximal end portion, and a groove at the proximal end portion of the second jaw for receiving the proximal end portion of the first jaw, and
a slotted pin joint coupled to the first jaw and the second jaw and configured to allow the first jaw to move relative to the second jaw to compress the portion of the heart between the first jaw and the second jaw.

12. The method of claim 11, wherein the portion of the heart is a left atrial appendage.

13. The method of claim 11, wherein the slotted pin joint is positioned at the proximal end portions of the first jaw and the second jaw.

14. The method of claim 13, wherein the distal end portion of the first jaw and the distal end portion of the second jaw define an opening for a channel for receiving the portion of the heart.

15. The method of claim 14, wherein the slotted pin joint is configured to allow the first jaw to angle relative to the second jaw such that the channel has a width at the proximal end portions of the first jaw and the second jaw that is greater than a width at the distal end portions of the first jaw and the second jaw.

16. The method of claim 13, wherein the slotted pin joint is configured to allow the distal end portions of the first jaw and the second jaw to close prior to the proximal end portions of the first jaw and the second jaw.

17. The method of claim 11, wherein the slotted pin joint is configured to allow the first jaw to extend parallel with the second jaw.

18. The method of claim 11, wherein the first jaw extends along an axis, and the slotted pin joint includes a slot on the first jaw extending transverse to the axis and a pin on the second jaw configured to slide along the slot.

19. The method of claim 18, wherein the slot includes an upper end and a lower end, and the pin is configured to slide along the slot towards the upper end to increase a distance between the first jaw and the second jaw and is configured to slide along the slot towards the lower end to reduce a distance between the first jaw and the second jaw.

20. The method of claim 11, wherein a spring is configured to force the first jaw towards the second jaw.
